SEQUENCE LISTING (29 sequences)

SEQ ID NO: 1 - protein, 362 aa, unidentified
  source 1..362: note = murine
KISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 2 - protein, 362 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 3 - protein, 349 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 4 - protein, 349 aa, synthetic construct
  VARIANT 32: note = X is a non-canonical amino acid
KISQYAAQRRTTLNNYNKKFTDALDILAENAXLRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 5 - protein, 349 aa, synthetic construct
  VARIANT 152: note = X is a non-canonical amino acid
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLXSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 6 - protein, 349 aa, synthetic construct
  VARIANT 154: note = X is a non-canonical amino acid
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSXVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 7 - protein, 349 aa, synthetic construct
KISQYAAQRRTTLNNYNKKFTDALDILAENACLRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 8 - protein, 361 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFCLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERN

SEQ ID NO: 9 - protein, 349 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLCSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 10 - protein, 349 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSCVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 11 - protein, 362 aa, synthetic construct
KISQYAAQRRTTLNNYNKKFTDALDILAENACLRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 12 - protein, 362 aa, synthetic construct
KISQYAAQRRTTLNNYNKKFTDALDILACNAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 13 - protein, 362 aa, synthetic construct
KISQYAAQRRTTLNNYNKKFTDALDILAENAELRGNCGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 14 - skipped

SEQ ID NO: 15 - protein, 362 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFCLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 16 - protein, 349 aa, synthetic construct
KISQYAAQRRTTLNNYNKKFTDALDILACNAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 17 - protein, 349 aa, synthetic construct
KISQYAAQRRTTLNNYNKKFTDALDILAENAELRGNCGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 18 - protein, 349 aa, synthetic construct
KISQYAAQRRTTLNNYNKKFTDALDILAENAELRGNEGSALAFCRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 19 - protein, 349 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFCLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 20 - protein, 617 aa, synthetic construct
MGSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKGSSHHHHHHHHHHGSGKISQYAAQRRTTLNNYNKKFTDALDILAENAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 21 - protein, 617 aa, synthetic construct
MGSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKGSSHHHHHHHHHHGSGKISQYAAQRRTTLNNYNKKFTDALDILAENACLRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 22 - protein, 617 aa, synthetic construct
MGSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKGSSHHHHHHHHHHGSGKISQYAAQRRTTLNNYNKKFTDALDILACNAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 23 - protein, 617 aa, synthetic construct
MGSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKGSSHHHHHHHHHHGSGKISQYAAQRRTTLNNYNKKFTDALDILAENAELRGNCGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 24 - protein, 617 aa, synthetic construct
MGSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKGSSHHHHHHHHHHGSGKISQYAAQRRTTLNNYNKKFTDALDILAENAELRGNEGSALAFRRAASVLKSLPFPITSMKDTEGIPNLGDKVKRIIQEILEDGESSEAKAVLNDERYQAFCLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 25 - protein, 362 aa, synthetic construct
KISQYAAQRRTTLNNYNKKFTCALDILAENAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 26 - protein, 362 aa, synthetic construct
KISQYAAQRRTTLNNYNKKFTDALDILAENAELRGNEGSALAFCRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 27 - protein, 362 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVCNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 28 - protein, 362 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSCVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 29 - protein, 362 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERCA

(19) United States
(12) Patent Application Publication
(10) Pub. No.: US 2026/0347898 A1
(43) Pub. Date: Nov. 5, 2026

(54) COVALENTLY MODIFIED TEMPLATE-INDEPENDENT DNA POLYMERASE AND METHODS OF USE THEREOF

(71) Applicant: The Charles Stark Draper Laboratory, Inc., Cambridge, MA (US)

(72) Inventors: Melissa M. Sprachman, Cambridge, MA (US); Cassie M. Bryan, Cambridge, MA (US); Kettner John Frederick Griswold, Jr., Boston, MA (US); Robert E. Doe, Medfield, MA (US); Isaac Moran, Maynard, MA (US); William C. Records, Arlington, MA (US); Andrew P. Magyar, Arlington, MA (US); Juan Carlos Rodriguez, Medford, MA (US); Rachel Coleman; Robert Doyle, Worcester, MA (US)

(21) Appl. No.: 19/112,161

(22) PCT Filed: Sep. 14, 2023
(86) PCT No.: PCT/US2023/074179

§ 371 Date: Mar. 14, 2025

Related U.S. Application Data
(60) Provisional application No. 63/375,944, filed on Sep. 16, 2022.

(51) Int. Cl. C12P 19/34; C12N 9/12; C25B 3/05; C25B 3/09; C25B 3/20

(52) U.S. Cl. CPC C12P 19/34; C12N 9/1264; C12Y 207/07031; C25B 3/05; C25B 3/09; C25B 3/20; C07K 2319/21

(57) ABSTRACT

Provided herein are engineered terminal deoxynucleotidyl transferase (TdT) proteins with certain modifications, including mutations to confer thermal stability and to install an exposed amino acid residue to which a small molecule can be covalently tethered via bioconjugate chemistries such as click chemistry. Also provided herein are methods of nucleic acid molecule synthesis using engineered TdTs and nucleotide molecules attached to redox-cleavable linkers, wherein the engineered TdT incorporates the nucleotide molecule into a nucleic acid strand and is separated from the nucleotide molecule when the redoxcleavable linker is cleaved upon exposure to suitable electrochemical conditions. Also provided herein are engineered TdTs covalently attached to a nucleotide molecule via a tether and also nucleotide molecules comprising a redox-cleavable linker. Further provided herein are systems for enzymatic DNA synthesis comprising an engineered TdT, a redox-cleavable linker a redox shuttle solution, and two or more electrodes.

### CROSS-REFERENCE TO RELATED APPLICATIONS

This application claims the benefit of priority of U.S. Provisional Application No. 63/375,944, filed Sep. 16, 2022, which is incorporated by reference herein in its entirety for any purpose.

### STATEMENT OF GOVERNMENT RIGHTS

This invention was made with Government support. The Government has certain rights in the invention.

### SEQUENCE LISTING

The instant application contains a Sequence Listing which has been submitted electronically in XML format and is hereby incorporated by reference in its entirety. Said XML copy, created on Aug. 21, 2023, is named “2023-08-21-01343-0001-00PCT-ST26” and is 37,935 bytes in size.

### DESCRIPTION

### Field

Provided herein are engineered terminal deoxynucleotidyl transferase (TdT) proteins with certain modifications, including mutations to confer thermal stability and to install an exposed amino acid residue to which a small molecule can be covalently tethered via bioconjugate chemistries such as click chemistry. Also provided herein are methods of nucleic acid molecule synthesis using engineered TdTs and nucleotide molecules attached to redox-cleavable linkers, wherein the engineered TdT incorporates the nucleotide molecule into a nucleic acid strand and is separated from the nucleotide molecule when the redox-cleavable linker is cleaved upon exposure to suitable electrochemical conditions. Also provided herein are engineered TdTs covalently attached to a nucleotide molecule via a tether and also nucleotide molecules comprising a redox-cleavable linker. Further provided herein are systems for enzymatic DNA synthesis comprising an engineered TdT, a redox-cleavable linker a redox shuttle solution, and two or more electrodes.

### BACKGROUND

DNA polymerases are enzymes responsible for the replication of genetic material in vivo and in vitro. Specifically, these enzymes are responsible for catalyzing the addition of nucleotide triphosphates (e.g., dNTPs and analogs thereof) to the three-prime end of a primer or seed strand of DNA. The majority of DNA polymerases replicate DNA in a largely template-dependent manner. That is: synthesizing the reverse complement strand of a DNA strand. However, a few polymerases have template-independent activity, wherein they can synthesize random sequences of DNA without the influence or need of a template strand.

Terminal deoxynucleotidyl transferase (hereinto referred to as TdT) is a DNA polymerase capable of catalyzing the random addition of nucleotides. In vivo, specifically in premature immune cells undergoing antibody and T cell receptor recombination, TdT acts in conjunction with DNA repair pathways to generate highly diverse sequences at VDJ junction sites. In vitro, TdT also displays template-independent activity, enabling its widespread use for applications such as poly A tailing of DNA. Because TdT does not require a DNA primer strand for DNA synthesis it is an ideal enzyme for in vitro DNA synthesis (see, e.g., Gouge, Jérôme, et al. “Structures of intermediates along the catalytic cycle of terminal deoxynucleotidyl transferase: dynamical aspects of the two-metal ion mechanism.” Journal of Molecular Biology 425.22 (2013): 4334-4352).

However, TdT adds nucleotides in an uncontrolled manner. While this template independence provides a means to synthesize entirely novel sequences of DNA from scratch, to do so in a highly controlled and sequence specific manner requires the control of the enzyme.

The availability of a TdT enzyme capable of controllably adding or inserting a single nucleotide (mononucleotide) at a time would enable new DNA synthesis strategies not previously possible, with benefits over existing strategies, and in particular would enable control of the enzyme for the synthesis of sequence-specified DNA, RNA, or other nucleic acid molecules.

### SUMMARY

The present disclosure addresses, among other things, the problem of synthesizing any polymer wherein an enzyme (e.g., terminal deoxynucleotidyl transferase, referred to herein as TdT or Tdt) can mediate addition of monomers to a growing oligomer chain. Provided herein, for example, are methods for controlling the sequence in which the monomer units are added. The present disclosure addresses, among other things, the problem of de novo DNA synthesis with sequence control, using enzymatic methods. Current methods for DNA/RNA synthesis rely on classical phosphoramidite chemistry, but this chemistry is typically useful for sequences less than 200 base pairs in length and is prone to errors. Moreover, phosphoramidite chemistry requires use of toxic solvents and reagents.

Alternatively, use of enzymes to generate long sequences of DNA or RNA in a sequence-controlled manner could enable synthesis of long DNA/RNA sequences with limited error rates. De novo enzymatic DNA synthesis is enabling for many applications, including gene synthesis and biodefense (rapid bio-threat identification and countermeasure development). Enzymatic synthesis methods are performed in aqueous solutions, also providing an environmentally conscious solution for nucleic acid synthesis.

Provided herein, among other things, are compositions and methods wherein a small molecule is linked to an enzyme via a linker that can be electrochemically cleaved, e.g., by a redox electrochemical reaction. In some embodiments, the small molecule is a nucleotide, nucleotide triphosphate or a nucleotide analog tethered to TdT via a redox-cleavable linker such as a quinone oxime ether linker; in some embodiments the conjugation to the enzyme is affected by click chemistry (after introducing a click chemistry moiety via reacting a site-specific cysteine residue with an appropriately functionalized maleimide). In some embodiments, the TdT enzyme is incubated with a single-stranded DNA molecule; upon incubation, the enzyme-tethered nucleotide is covalently attached to the ssDNA. Upon cleavage of the quinone oxime ether residue via reduction chemistry, the ssDNA with a newly incorporated base is cleaved from the enzyme.

Embodiment 1 is a method of nucleic acid molecule synthesis comprising the steps of:
(a) providing an engineered terminal deoxynucleotidyl transferase (TdT), wherein the engineered TdT comprises a nucleotide molecule covalently attached to the TdT via a redox-cleavable linker;
(b) providing a single-stranded nucleic acid molecule comprising a 5′ and a 3′ end;
(c) contacting the engineered TdT and the single-stranded nucleic acid molecule under conditions suitable for the TdT to bind to the 3′ end of the nucleic acid molecule and form a TdT-nucleic acid strand complex, thereby incorporating the nucleotide molecule covalently linked to the TdT into the nucleic acid strand, resulting in the nucleic acid molecule tethered to the TdT and thereby blocking subsequent additions of a nucleotide molecule to the nucleic acid strand; and
(d) exposing the TdT-nucleic acid strand complex to suitable electrochemical conditions, wherein the redox-cleavable linker is cleaved thereby separating the TdT-redox-cleavable linker molecule from the nucleic acid strand with the newly incorporated nucleotide molecule, resulting in a nucleic acid strand with the newly incorporated nucleotide molecule; and
(e) repeating steps (a) through (d) thereby synthesizing a nucleic acid molecule.

Embodiment 2 is the method of embodiment 1, wherein the redox-cleavable linker is a quinone oxime ether linker.

Embodiment 3 is the method of embodiment 1 or embodiment 2, wherein the electrochemical conditions of step (d) comprise one or more suitable soluble redox shuttles in the presence of two or more electrodes, wherein the two or more electrodes control the oxidation state of the soluble redox shuttles.

Embodiment 4 is the method of any one of embodiments 1-3, wherein the soluble redox shuttles comprise a thiazine, a viologen, dinucleotides, a flavin, a quinone, peroxide, bromide, vanadium and/or a metal complex.

Embodiment 5 is the method of embodiment 4, wherein the soluble redox shuttles comprise a thiazine.

Embodiment 6 is the method of embodiment 4, wherein the soluble redox shuttles comprise a viologen.

Embodiment 7 is the method of embodiment any one of embodiments 1-3, wherein the soluble redox shuttles comprise dinucleotides.

Embodiment 8 is the method of embodiment 7, wherein the dinucleotides comprise NAD/NADH, nicotinamide adenine dinucleotide, and/or reduced form.

Embodiment 9 is the method of embodiment 4, wherein the soluble redox shuttles comprise a flavin.

Embodiment 10 is the method of embodiment 4, wherein the soluble redox shuttles comprise a quinone.

Embodiment 11 is the method of embodiment 4, wherein the soluble redox shuttles comprise peroxide.

Embodiment 12 is the method of embodiment 4 wherein the soluble redox shuttles comprise bromide.

Embodiment 13 is the method of embodiment 4, wherein the soluble redox shuttles comprise vanadium.

Embodiment 14 is the method of embodiment 4, wherein the soluble redox shuttles comprise a metal complex.

Embodiment 15 is the method of embodiment 5, wherein the thiazine is methylene blue.

Embodiment 16 is the method of embodiment 4, wherein the quinone is 2,5-dihydroxy-1,4-benzoquinone (DHBQ), 9,10-anthraquinone-2,7-disulfonic acid (AQDS), 4,5-dihydroxybenzene-1,3-disulfonic acid (BQDS), or 2,3,5,6-tetrakis((dimethylamino)methyl)hydroquinone (FQH₂).

Embodiment 17 is the method of embodiment 9, wherein the flavin is riboflavin (RF), flavin mononucleotide (FMN), or flavin adenine dinucleotide (FAD).

Embodiment 18 is the method of embodiment 6, wherein the viologen is methyl viologen (MV) or bis-(trimethylammonio) propyl viologen (BTMAP-Vi).

Embodiment 19 is the method of embodiment 14, wherein the metal complex is TEMPO (2,2,6,6-tetramethylpiperidin-1-yl)oxyl, (2,2,6,6-tetramethylpiperidin-1-yl) oxidanyl).

Embodiment 20 is the method of embodiment 14, wherein the metal complex is an organometallic coordination complex.

Embodiment 21 is the method of embodiment 20, wherein the organometallic coordination complex is a ferrocene, bis-(trimethylammonio) propyl ferrocene (BTMAP-Fc), ammonium ferricyanide ((NH₄)₄Fe(CN)₆), potassium ferricyanide (K₄Fe(CN)₆), hexaamineruthenium (III) chloride (RuHex), permanganate, or dichromate.

Embodiment 22 is the method of any one of embodiments 1-21, wherein the electrochemical conditions of step (d) further comprise a buffer, a supporting electrolyte, and/or a hydrotropic agent.

Embodiment 23 is the method of embodiment 22, wherein the buffer comprises sodium citrate, potassium hydrogen phosphate, and/or potassium dihydrogen phosphate.

Embodiment 24 is the method of embodiment 22, wherein the supporting electrolyte comprises sodium chloride, sodium salts, lithium salts, potassium salts, and/or magnesium salts.

Embodiment 25 is the method of embodiment 22, wherein the hydrotropic agent comprises caffeine, urea, and/or nicotinamide (NA).

Embodiment 26 is the method of any one of embodiments 1-3, wherein the electrochemical conditions of step (d) comprise a redox shuttle solution comprising methylene blue, citrate-phosphate buffer, and sodium chloride.

Embodiment 27 is the method of any one of embodiments 1-3, wherein the electrochemical conditions of step (d) comprise a redox shuttle solution comprising methylene blue, citrate-phosphate buffer, sodium chloride, and nicotinamide.

Embodiment 28 is the method of any one of embodiments 1-3, wherein the electrochemical conditions of step (d) comprise a redox shuttle solution comprising flavin mononucleotide, citrate-phosphate buffer, sodium chloride, and nicotinamide.

Embodiment 29 is the method of any one of embodiments 1-3, wherein the electrochemical conditions of step (d) comprise a redox shuttle solution comprising 10 mM methylene blue, 25 mM citrate-phosphate buffer at pH 3.4, and 100 mM sodium chloride.

Embodiment 30 is the method of any one of embodiments 1-3, wherein the electrochemical conditions of step (d) comprise a redox shuttle solution comprising 10 mM methylene blue, 25 mM citrate-phosphate buffer at pH 3.4, 100 mM sodium chloride, and 1 M nicotinamide.

Embodiment 31 is the method of any one of embodiments 1-3, wherein the electrochemical conditions of step (d) comprise a redox shuttle solution comprising 50 mM flavin mononucleotide, 25 mM citrate-phosphate buffer at pH 3.4, 1 M sodium chloride, and 1 M nicotinamide.

Embodiment 32 is the method of any one of embodiments 1-31, wherein the potential for cleavage is about +1.05 vs. SHE to −0.18V vs. SHE at 25 °C and pH 3; +0.84 V vs. SHE to −0.38 V vs. SHE at 25 °C and pH 6.5; or +0.64V vs. SHE to −0.59V vs. SHE at 25 °C and pH 10.

Embodiment 33 is the method of any one of embodiments 1-32, wherein the engineered TdT comprises one or more mutations to a wild-type TdT of SEQ ID NO: 1, wherein:
a. the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E, wherein position numbers are relative to SEQ ID NO: 1; or
b. the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353E, wherein position numbers are relative to SEQ ID NO: 1; or
c. the engineered TdT has at least 85, 90, 91, 92, 93, 94, 95, 96, 97, 98, 99, or 100% identity to SEQ ID NO: 2 or 3.

Embodiment 34 is an engineered terminal deoxynucleotidyl transferase (TdT) comprising one or more mutations to a wild-type TdT of SEQ ID NO: 1, wherein:
a. the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E, wherein position numbers are relative to SEQ ID NO: 1; or
b. the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353E, wherein position numbers are relative to SEQ ID NO: 1; or
c. the engineered TdT has at least 85, 90, 91, 92, 93, 94, 95, 96, 97, 98, 99, or 100% identity to SEQ ID NO: 2 or 3.

Embodiment 35 is the engineered TdT of embodiment 34, wherein the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E.

Embodiment 36 is the engineered TdT of embodiment 34 or embodiment 35, wherein the one or more mutations comprise 10, 15, 20, 25, 30, 35, 40, 45, 50, or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E.

Embodiment 37 is the engineered TdT of any one of embodiments 34-36, wherein the one or more mutations comprise 50 or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E.

Embodiment 38 is the engineered TdT of any one of embodiments 34-37, wherein the one or more mutations comprise all of the following mutations: C7A, Q18K, L19K, D31A, E35G, C40A, M44R, S47A, C68N, S75R, E78Q, G79B, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E.

Embodiment 39 is the engineered TdT of any one of embodiments 34-38, wherein the one or more mutations consist of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q. Q242L, C256A, D263H, G265Q, H268D, E270G, C290A, D293E, T317R, M323L, R335N, V336T, E339R, D353E.

Embodiment 40 is the engineered TdT of any one of embodiments 34-39, wherein the engineered TdT comprises an amino acid sequence with 85, 90, 91, 92, 93, 94, 95, 96, 97, 98, 99, or 100% identity to SEQ ID NO: 2.

Embodiment 41 is the engineered TdT of any one of embodiments 34-40, wherein the engineered TdT comprises the amino acid sequence of SEQ ID NO: 2.

Embodiment 42 is the engineered TdT of any one of embodiments 34-41, wherein the amino acid sequence of the engineered TdT consists of the amino acid sequence of SEQ ID NO: 2.

Embodiment 43 is the engineered TdT of any one of embodiments 34-41, wherein the amino acid sequence of the engineered TdT consists of the amino acid sequence of SEQ ID NO: 2 and a fluorescent protein label, optionally a green fluorescent protein (GFP) label.

Embodiment 44 is the engineered TdT of any one of embodiments 34-41, wherein the amino acid sequence of the engineered TdT consists of the amino acid sequence of SEQ ID NO: 2 and an affinity tag, optionally a His-tag.

Embodiment 45 is the engineered TdT of any one of embodiments 34-41, wherein the engineered TdT further comprises a fluorescent protein label, optionally a GFP label.

Embodiment 46 is the engineered TdT of any one of embodiments 34-41, wherein the engineered TdT further comprises an affinity tag, optionally a His-tag.

Embodiment 47 is the engineered TdT of any one of embodiments 34-46, wherein the TT further comprises the conjugation mutation E32C.

Embodiment 48 is the engineered TdT of any one of embodiments 34-46, wherein the TT further comprises the conjugation mutation E29C.

Embodiment 49 is the engineered TdT of any one of embodiments 34-46, wherein the TdT further comprises the conjugation mutation E37C.

Embodiment 50 is the engineered TdT of any one of embodiments 34-46, wherein the TdT further comprises the conjugation mutation V152C.

Embodiment 51 is the engineered TdT of any one of embodiments 34-46, wherein the TdT further comprises the conjugation mutation K102C.

Embodiment 52 is the engineered TdT of any one of embodiments 34-46, wherein the TT further comprises the conjugation mutation D22C.

Embodiment 53 is the engineered TdT of any one of embodiments 34-46, wherein the TdT further comprises the conjugation mutation R44C.

Embodiment 54 is the engineered TdT of any one of embodiments 34-46, wherein the TdT further comprises the conjugation mutation L93C.

Embodiment 55 is the engineered TdT of any one of embodiments 34-46, wherein the TdT further comprises the conjugation mutation P154C.

Embodiment 56 is the engineered TdT of any one of embodiments 34-46, wherein the TdT further comprises the conjugation mutation N361C.

Embodiment 57 is the engineered TdT of any one of embodiments 34-38, wherein the TdT comprises the amino acid sequence of any one of SEQ ID NOs: 11, 12, 13, and/or 15.

Embodiment 58 is the engineered TdT of any one of embodiments 34-38, wherein the TdT comprises the amino acid sequence of SEQ ID NO: 15.

Embodiment 59 is the engineered TdT of any one of embodiments 34-38, wherein the TdT comprises the amino acid sequence of any one of SEQ ID NOs: 25, 26, 27, 28, and 29.

Embodiment 60 is an engineered TdT comprising the amino acid sequence of any one of SEQ ID NOs: 20, 21, 22, 23, and 24.

Embodiment 61 is an engineered terminal deoxynucleotidyl transferase (TdT) consisting of the amino acid sequence of SEQ ID NO: 15.

Embodiment 62 is the engineered TdT of embodiment 34, wherein the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353B, wherein position numbers are relative to SEQ ID NO: 1.

Embodiment 63 is the engineered TdT of embodiment 34 or 62, wherein the one or more mutations comprise 10, 15, 20, 25, 30, 35, 40, 45, 50, or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353E, wherein position numbers are relative to SEQ ID NO: 1.

Embodiment 64 is the engineered TdT of embodiments 34, 62 or 63, wherein the one or more mutations comprise 50 or more of the following mutations: C7A, Q18K, L19K, D31A, E35G, C40A, M44R, S47A, C68N, S75R, E78Q, G79E, I81L, K99Q, $100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353E, wherein position numbers are relative to SEQ ID NO: 1.

Embodiment 65 is the engineered TdT of any one of embodiments 34 or 62-64, wherein the one or more mutations comprise the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353E, wherein position numbers are relative to SEQ ID NO: 1.

Embodiment 66 is the engineered TdT of any one of embodiments 34 or 62-65, wherein the one or more mutations consist of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R. D353E, wherein position numbers are relative to SEQ ID NO: 1.

Embodiment 67 is the engineered TdT of any one of embodiments 34 or 62-66, wherein the engineered TdT comprises an amino acid sequence with 85, 90, 91, 92, 93, 94, 95, 96, 97, 98, 99, or 100% identity to SEQ ID NO: 3.

Embodiment 68 is the engineered TdT of any one of embodiments 34 or 62-67, wherein the engineered TdT comprises the amino acid sequence of SEQ ID NO: 3.

Embodiment 69 is the engineered TdT of any one of embodiments 34 or 62-68, wherein the amino acid sequence of the engineered TdT consists of the amino acid sequence of SEQ ID NO: 3.

Embodiment 70 is the engineered TdT of any one of embodiments 34 or 62-68, wherein the amino acid sequence of the engineered TdT consists of the amino acid sequence of SEQ ID NO: 3 and a fluorescent protein label, optionally a GFP label.

Embodiment 71 is the engineered TdT of any one of embodiments 34 or 62-68, wherein the amino acid sequence of the engineered TdT consists of the amino acid sequence of SEQ ID NO: 3 and an affinity tag, optionally a His-tag.

Embodiment 72 is the engineered TdT of any one of embodiments 34 or 62-68, wherein the engineered TdT further comprises a fluorescent protein label, optionally a green fluorescent protein (GFP) label.

Embodiment 73 is the engineered TdT of any one of embodiments 34 or 62-68, wherein the engineered TdT further comprises an affinity tag, optionally a His-tag.

Embodiment 74 is the engineered TdT of any one of embodiments 34 or 62-73, wherein the TT further comprises the conjugation mutation E32nc, wherein ne is a non-canonical amino acid.

Embodiment 75 is the engineered TdT of any one of embodiments 34 or 62-73, wherein the TdT further comprises the conjugation mutation V152nc, wherein ne is a non-canonical amino acid.

Embodiment 76 is the engineered TdT of any one of embodiments 34 or 62-73, wherein the TdT further comprises the conjugation mutation P154nc, wherein ne is a non-canonical amino acid.

Embodiment 77 is the engineered TdT of any one of embodiments 34 or 62-73, wherein the TdT further comprises the conjugation mutation E32C.

Embodiment 78 is the engineered TdT of any one of embodiments 34 or 62-73, wherein the TdT further comprises the conjugation mutation V152C.

Embodiment 79 is the engineered TdT of any one of embodiments 34 or 62-73, wherein the TdT further comprises the conjugation mutation P154C.

Embodiment 80 is the engineered TdT of any one of embodiments 34 or 62-73, wherein the TdT further comprises the conjugation mutation E29C.

Embodiment 81 is the engineered TdT of any one of embodiments 34 or 62-73, wherein the TdT further comprises the conjugation mutation E37C.

Embodiment 82 is the engineered TdT of any one of embodiments 34 or 62-73, wherein the TdT further comprises the conjugation mutation R44C.

Embodiment 83 is the engineered TdT of any one of embodiments 34 or 62-73, wherein the TdT further comprises the conjugation mutation K102C.

Embodiment 84 is the engineered TdT of any one of embodiments 34 or 62-64, wherein the TdT comprises the amino acid sequence of SEQ ID NO: 4.

Embodiment 85 is the engineered TdT of any one of embodiments 34 or 62-64, wherein the TdT comprises the amino acid sequence of SEQ ID NO: 5.

Embodiment 86 is the engineered TdT of any one of embodiments 34 or 62-64, wherein the TdT comprises the amino acid sequence of SEQ ID NO: 6.

Embodiment 87 is the engineered TdT of any one of embodiments 34 or 62-64, wherein the TdT comprises the amino acid sequence of SEQ ID NO: 7.

Embodiment 88 is the engineered TdT of any one of embodiments 34 or 62-64, wherein the TdT comprises the amino acid sequence of SEQ ID NO: 9.

Embodiment 89 is the engineered TdT of any one of embodiments 34 or 62-64, wherein the TdT comprises the amino acid sequence of SEQ ID NO: 10.

Embodiment 90 is the engineered TdT of any one of embodiments 34 or 62-64, wherein the TdT comprises the amino acid sequence of SEQ ID NO: 16.

Embodiment 91 is the engineered TdT of any one of embodiments 34 or 62-64, wherein the TdT comprises the amino acid sequence of SEQ ID NO: 17.

Embodiment 92 is the engineered TdT of any one of embodiments 34 or 62-64, wherein the TdT comprises the amino acid sequence of SEQ ID NO: 18

Embodiment 93 is the engineered TdT of any one of embodiments 34 or 62-64, wherein the TdT comprises the amino acid sequence of SEQ ID NO: 19.

Embodiment 94 is the engineered TdT of any of embodiments 34-93, wherein the TdT is capable of covalently binding to a nucleotide molecule at a conjugation residue of the engineered TdT via a tether, wherein:
(i) the conjugation residue comprises a cysteine residue, a non-canonical amino acid residue, or a lysine residue capable of reacting with a bifunctional crosslinker or a redox-cleavable linker; and
(ii) wherein the nucleotide molecule comprises a nucleotide, nucleotide triphosphate, or a nucleotide analog.

Embodiment 95 is the engineered TdT of embodiment 94, wherein the TdT, tether, and nucleotide molecule comprise any one of the following structures:

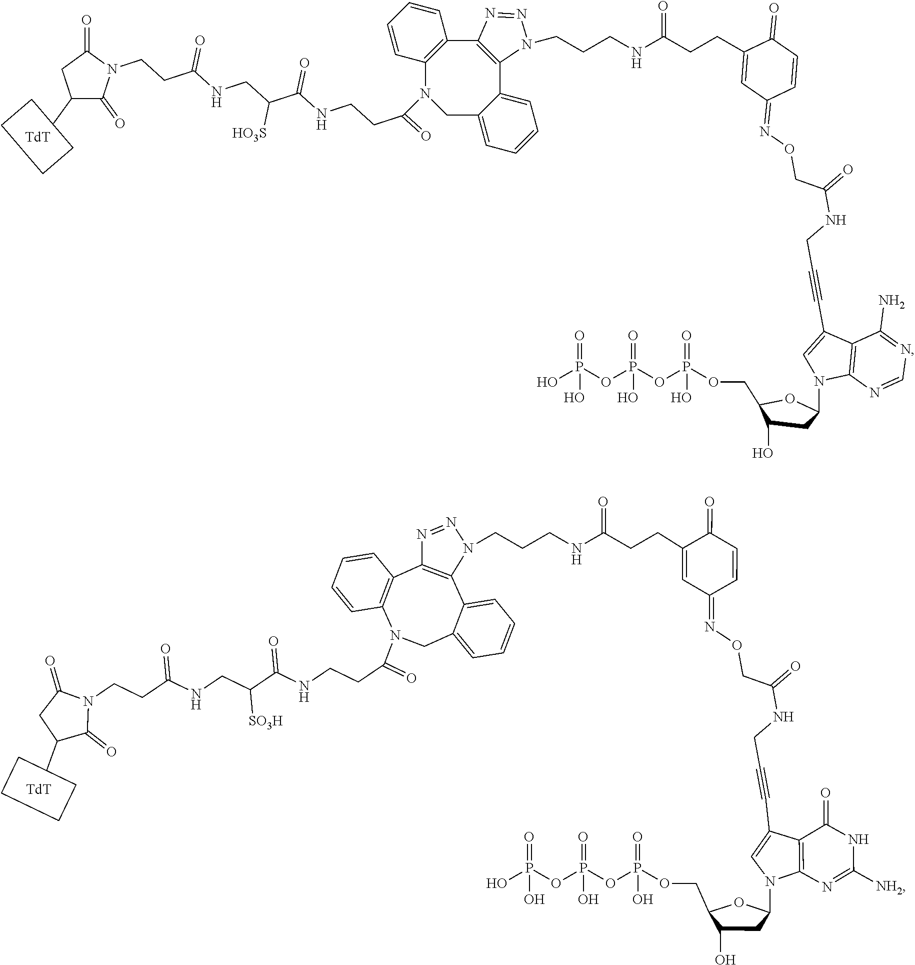

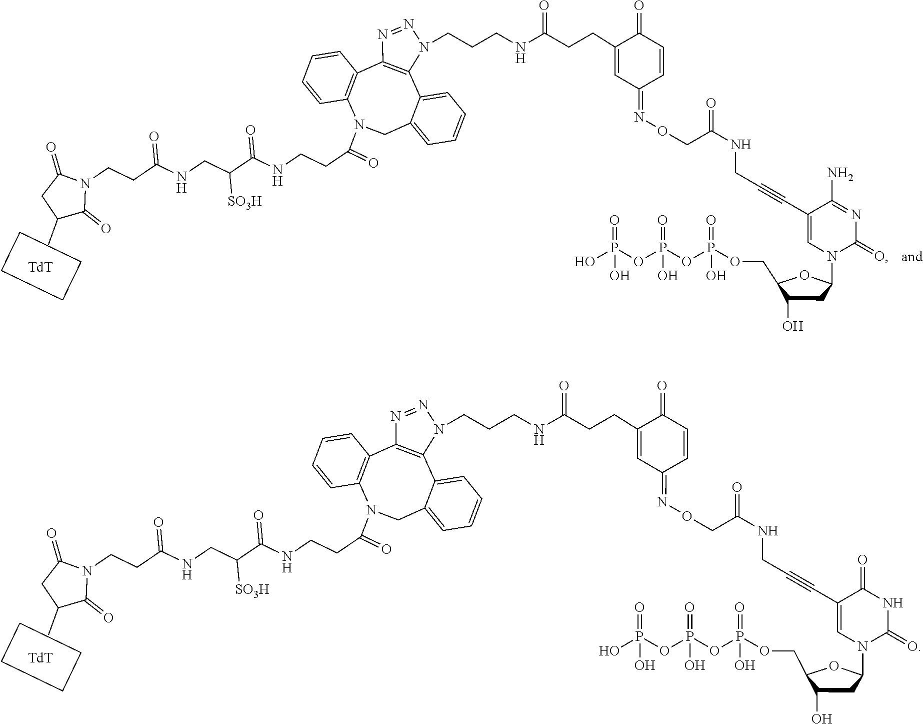

Embodiment 96 is the engineered TdT of embodiment 94, wherein the TdT, tether, and nucleotide molecule comprise any one of the following structures:

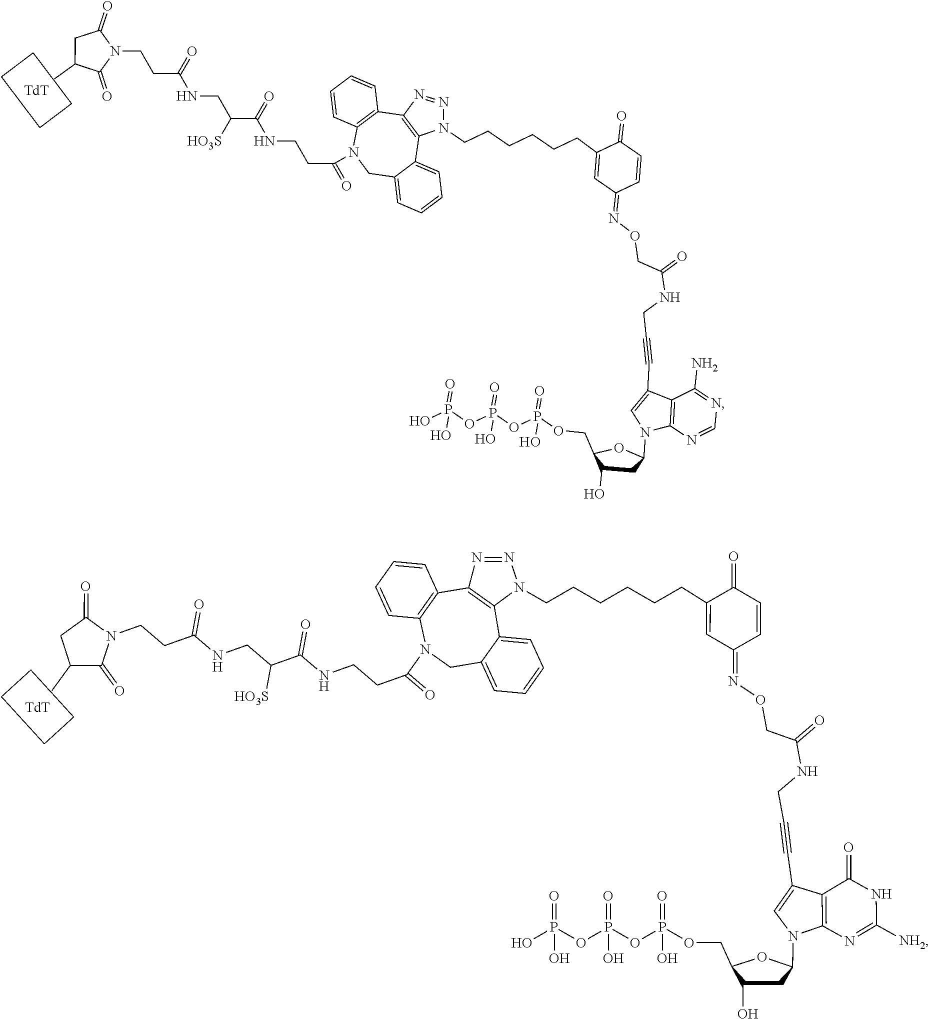

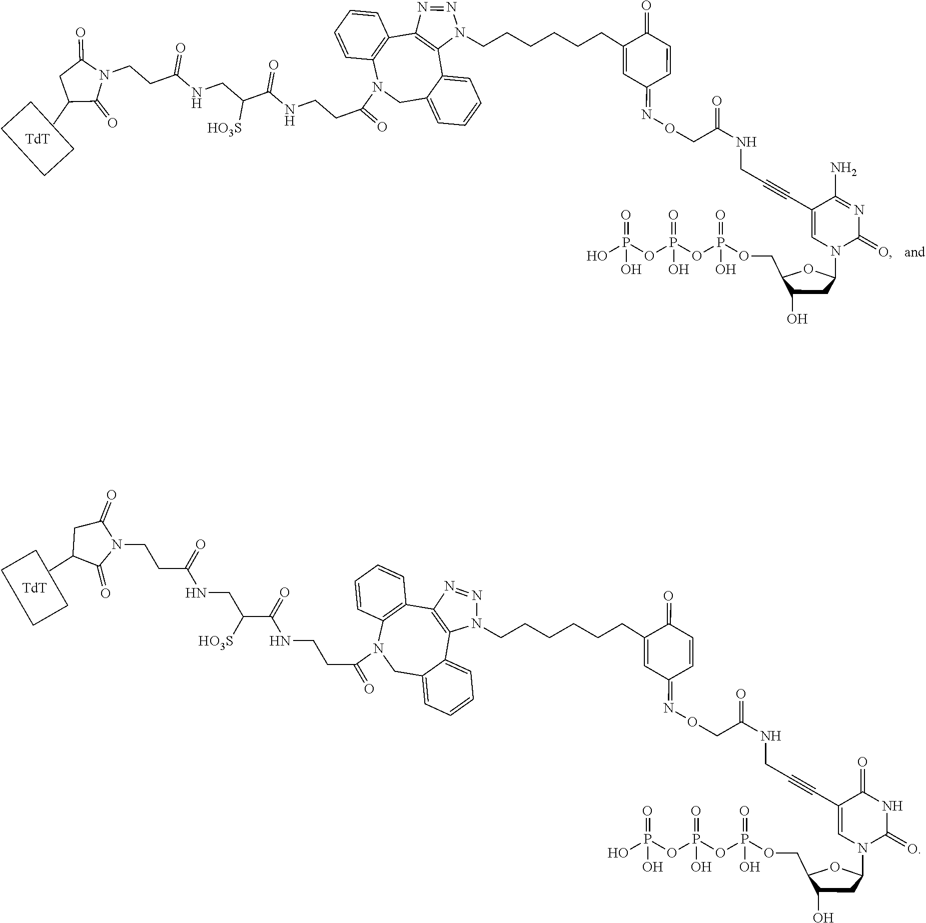

Embodiment 97 is the engineered TdT of embodiment 94, wherein the TdT, tether, and nucleotide molecule comprise any one of the following structures:

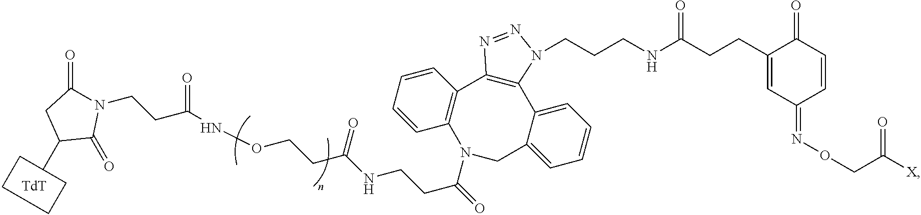

wherein X is any one of:

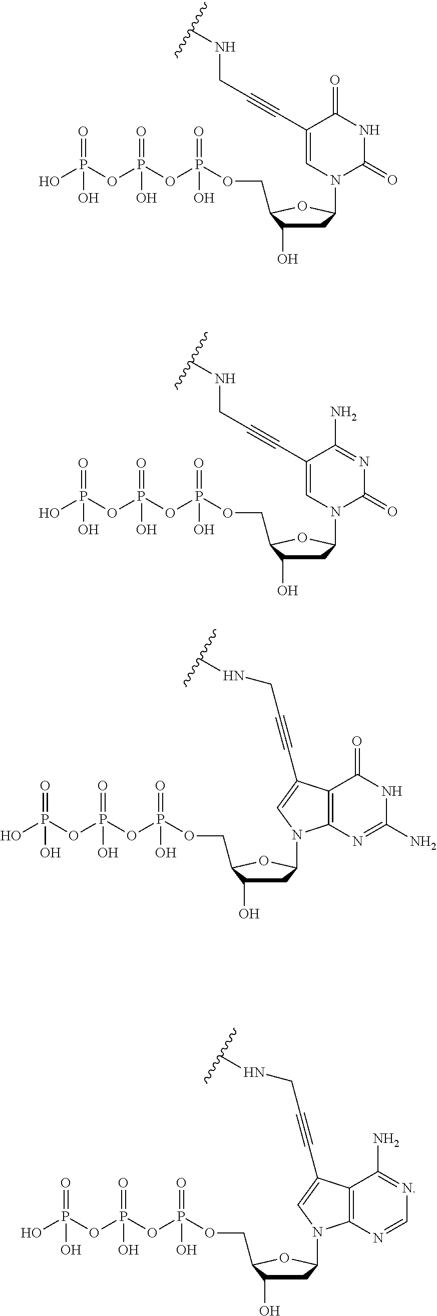

Embodiment 98 is the engineered TdT of embodiment 94, wherein the TdT, tether, and nucleotide molecule comprise any one of the following structures:

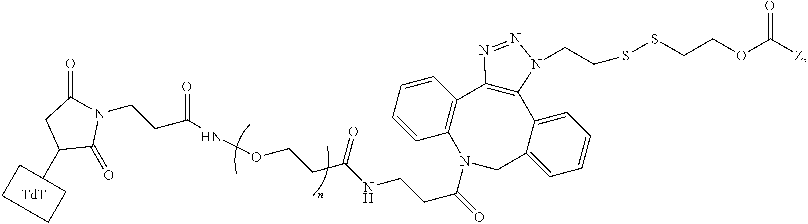

wherein Z is any one of:

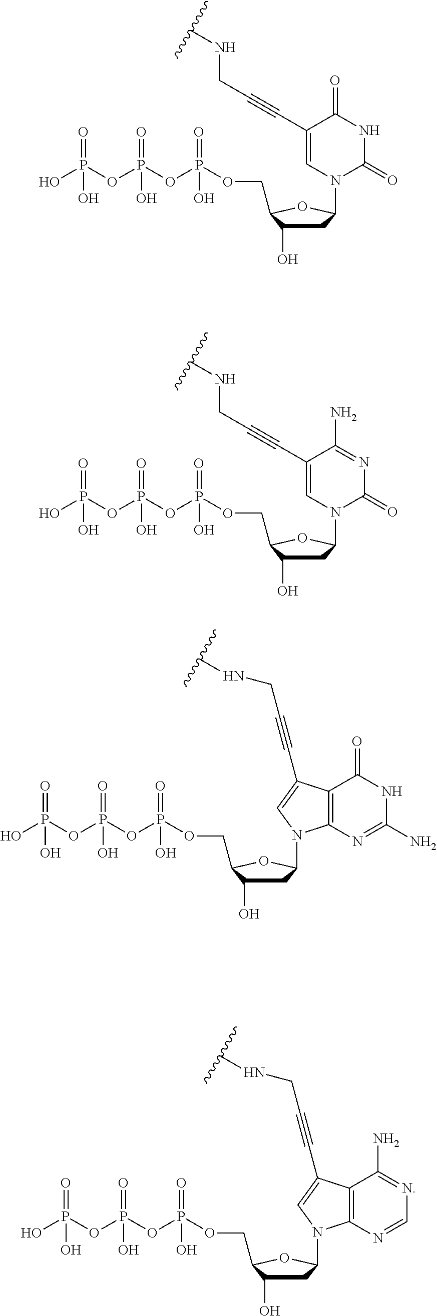

Embodiment 99 is the engineered TdT of embodiment 94, wherein the TdT, tether, and nucleotide molecule comprise any one of the following structures:

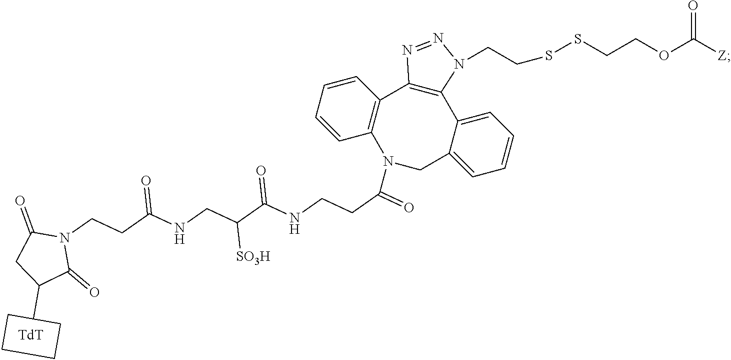

wherein Z is any one of:

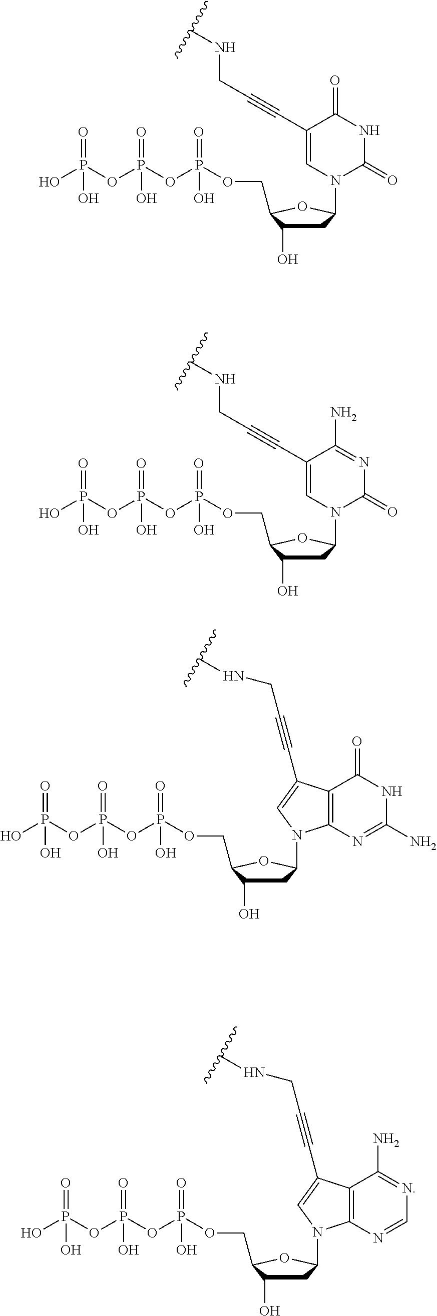

Embodiment 100 is the engineered TdT of embodiment 94, wherein the TdT, tether, and nucleotide molecule comprise any one of the following structures:

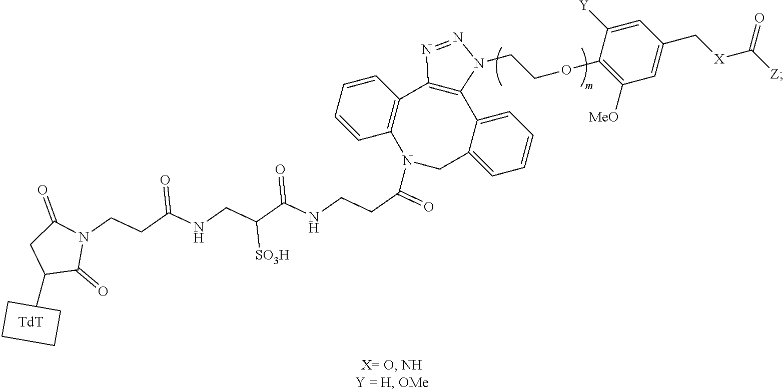

wherein X is O or NH;
wherein Y is H or OMe; and
wherein Z is any one of:

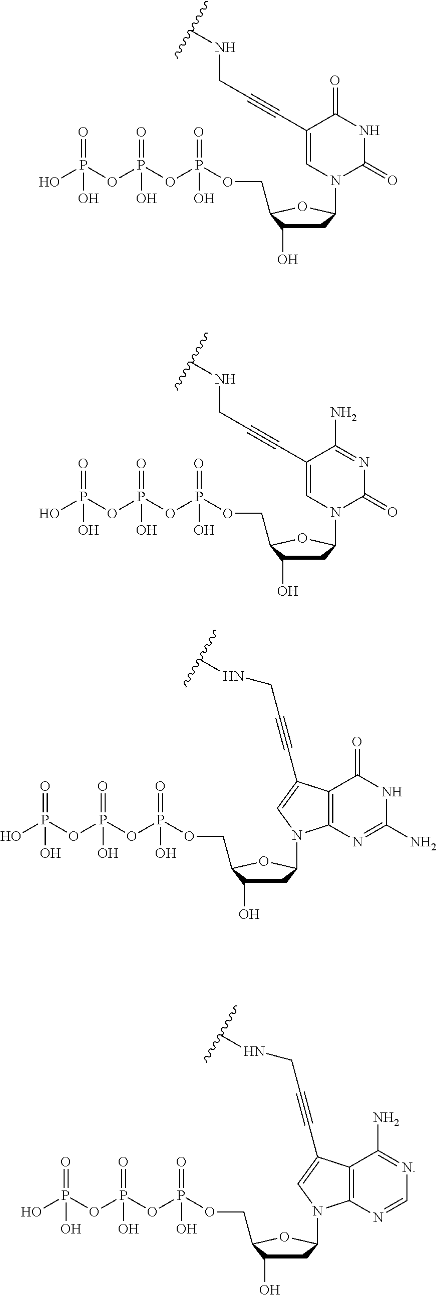

Embodiment 101 is the engineered TdT of embodiment 94, wherein the TdT, tether, and nucleotide molecule comprise any one of the following structures:

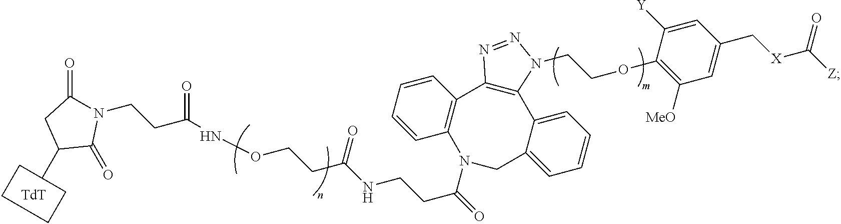

wherein X is O or NH;
wherein Y is H or OMe; and
wherein Z is any one of:

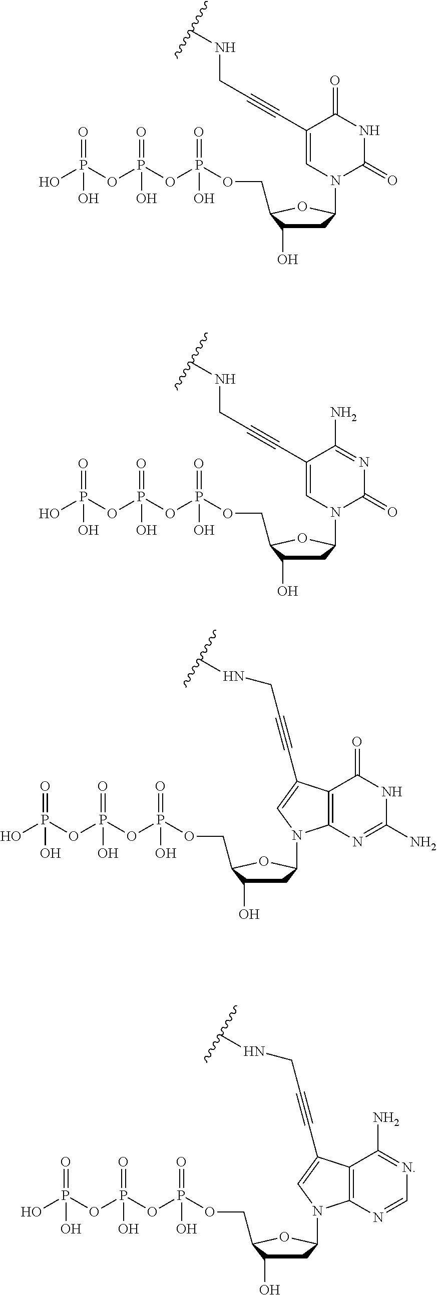

Embodiment 102 is the engineered TdT of embodiment 94, wherein the TdT, tether, and nucleotide molecule comprise any one of the following structures:

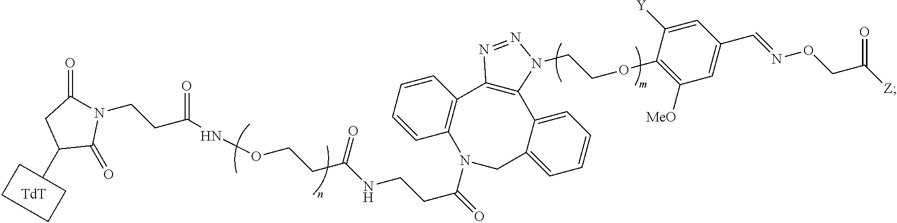

wherein Y is H or OMe; and
wherein Z is any one of:

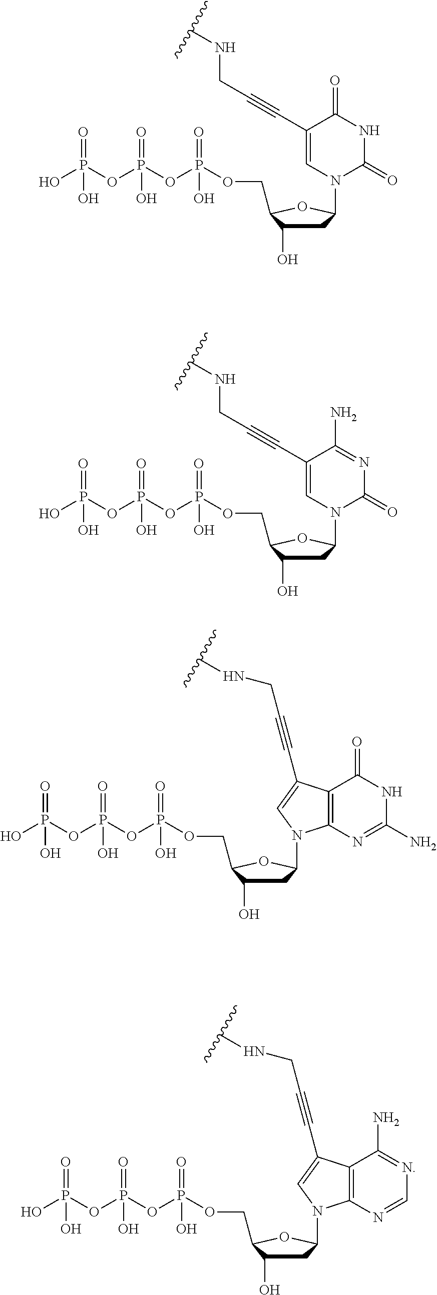

Embodiment 103 is the engineered TdT of embodiment 94, wherein the TdT, tether, and nucleotide molecule comprise any one of the following structures:

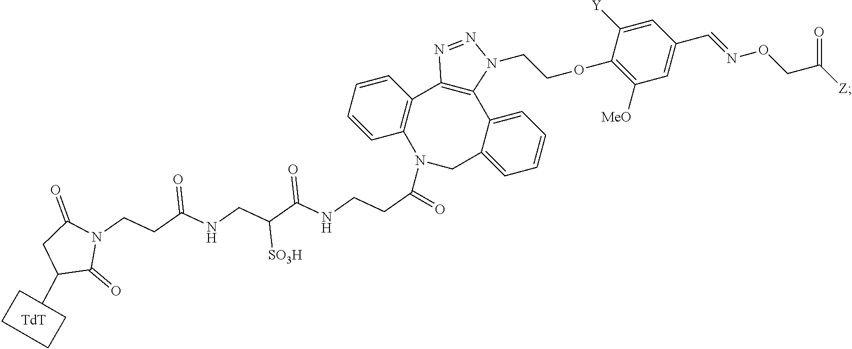

wherein Y is H or OMe; and
wherein Z is any one of:

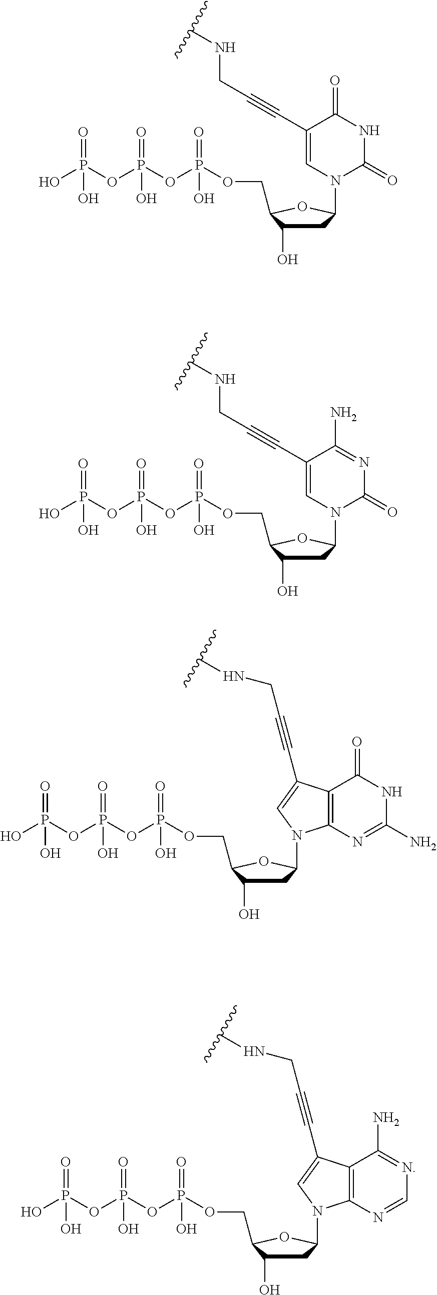

Embodiment 104 is a kit comprising the engineered TdT of any one of embodiments 34-93, wherein the TdT is capable of covalently binding to a nucleotide molecule at a conjugation residue of the engineered TdT via a tether, wherein the kit further comprises:
(i) a bifunctional crosslinker and/or a redox-cleavable linker comprising a functional group for covalent conjugation to a conjugation residue of the TdT; and
(ii) a nucleotide molecule.

Embodiment 105 is the kit of embodiment 104, wherein the kit further comprises a spacer.

Embodiment 106 is the kit of any one of embodiments 104-106, wherein the functional group for covalent conjugation comprises a maleimide, N-hydroxysuccinimidyl group, or a dibenzylcyclooctyne (DBCO).

Embodiment 107 is the kit of any one of embodiments 104-106, wherein the functional group for covalent conjugation comprises a maleimide.

Embodiment 108 is the kit of embodiment 107, wherein the maleimide is capable of attaching to a conjugation residue of the engineered TdT, wherein the conjugation residue is a cysteine residue in the TdT.

Embodiment 109 is the kit of any one of embodiments 104-106, wherein the functional group for covalent conjugation comprises a DBCO.

Embodiment 110 is the kit of embodiment 109, wherein the DBCO is capable of attaching to a conjugation residue of the engineered TdT, and wherein the conjugation residue is a non-canonical amino acid in the TdT.

Embodiment 111 is the kit of embodiment 110, wherein the non-canonical amino acid is 4-azidophenylalanine.

Embodiment 112 is the kit of any one of embodiments 104-111, wherein the redox-cleavable linker comprises a quinone oxime ether linker.

Embodiment 113 is the kit of any one of embodiments 104-111, wherein the redox-cleavable linker comprises a disulfide linker.

Embodiment 114 is the kit of any one of embodiments 104-111, wherein the redox-cleavable linker comprises a syringic acid-based linker or vanillin-based linker.

Embodiment 115 is the kit of any one of embodiments 105-114, wherein the spacer comprises one or more polyethylene glycol molecules (PEG) n, optionally wherein “n” is 1-12.

Embodiment 116 is the kit of any one of embodiments 105-114, wherein the spacer comprises a C1-C10 alkyl.

Embodiment 117 is the kit of any one of embodiments 105-114, wherein the spacer comprises a hydrocarbon chain wherein one or more of the carbons comprises a substituted sulfonate group.

Embodiment 118 is the kit of any one of embodiments 105-114, wherein the spacer comprises a hydrocarbon chain comprising 1-6 carbons wherein one or more of the carbons comprises a substituted sulfonate group.

Embodiment 119 is the kit of any one of embodiments 105-114, wherein the spacer comprises a C1-C6 alkyl.

Embodiment 120 is the kit of embodiment 104, wherein the kit comprises a bifunctional crosslinker capable of being covalently linked to the engineered TdT and comprising a DBCO, wherein the DBCO of the bifunctional crosslinker is capable of reacting with any one of the following molecules:

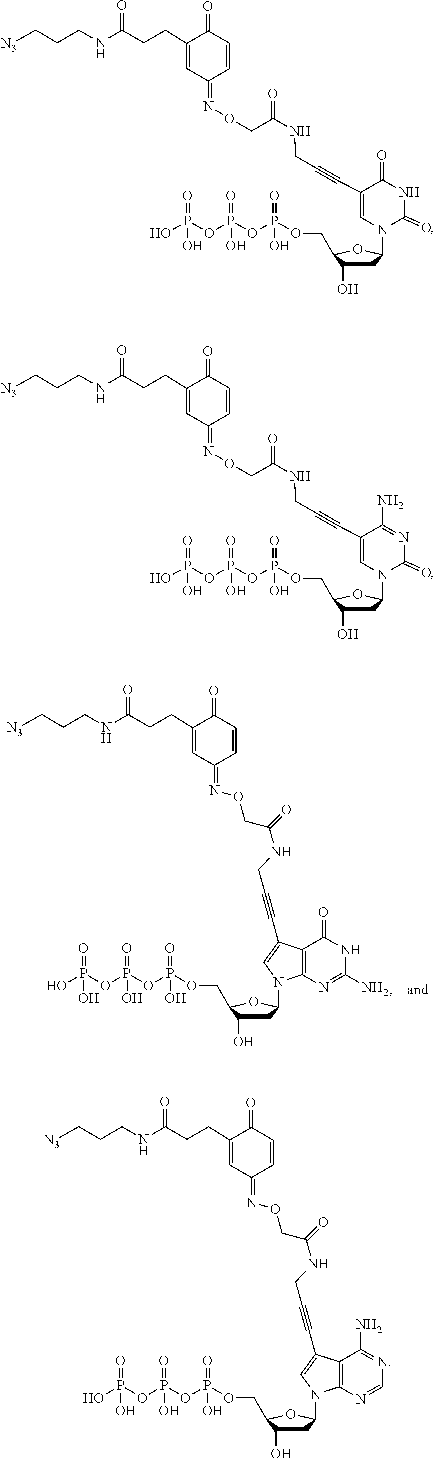

Embodiment 121 is the kit of embodiment 104, wherein the kit comprises a bifunctional crosslinker capable of being covalently linked to the engineered TdT and comprising a DBCO, wherein the DBCO of the bifunctional crosslinker is capable of reacting with any one of the following molecules:

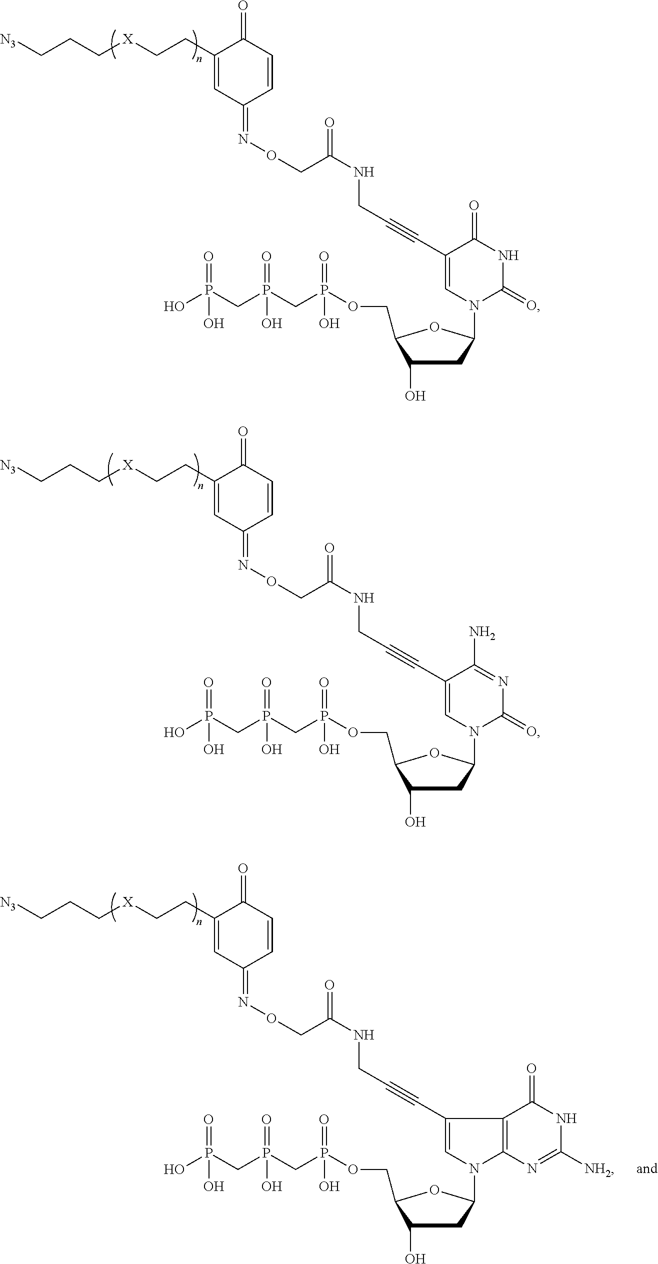

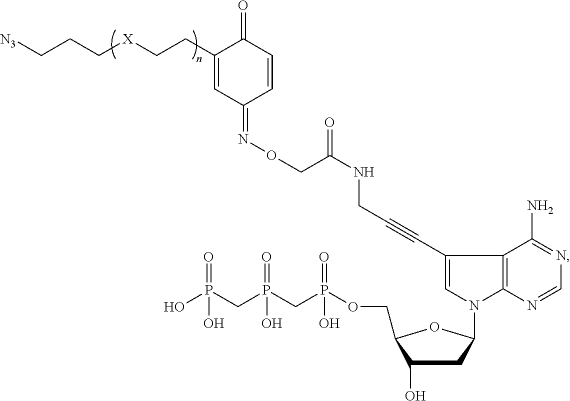

wherein X is C(O), C(O)NH, CH2, or O; and wherein n=1 for X═C(O), n=1 for C(O)NH, n=1 for CH2, and n=1-4 for X═O, wherein n is an integer.

Embodiment 122 is the kit of embodiment 104, wherein the kit comprises a bifunctional crosslinker capable of being covalently linked to the engineered TdT and comprising a DBCO, wherein the DBCO of the bifunctional crosslinker is capable of reacting with any one of the following molecules:

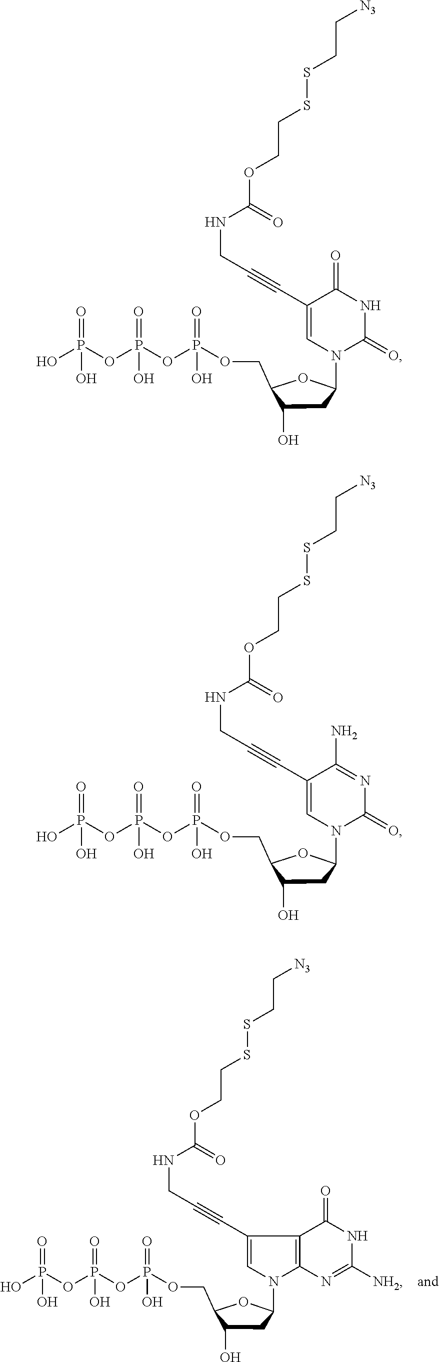

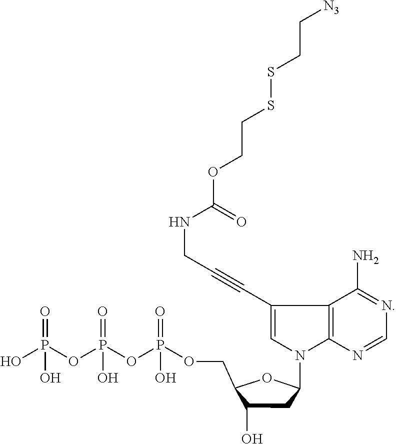

Embodiment 123 is the kit of embodiment 104, wherein the kit comprises a bifunctional crosslinker capable of being covalently linked to the engineered TdT and comprising a DBCO, wherein the DBCO of the bifunctional crosslinker is capable of reacting with any one of the following molecules:

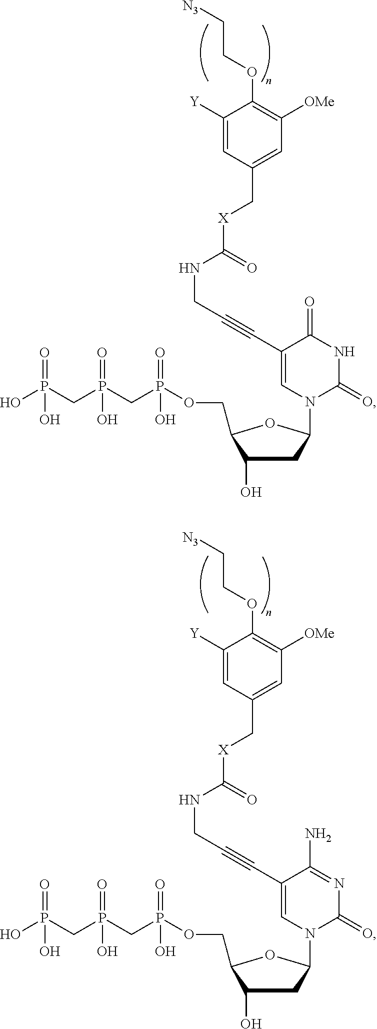

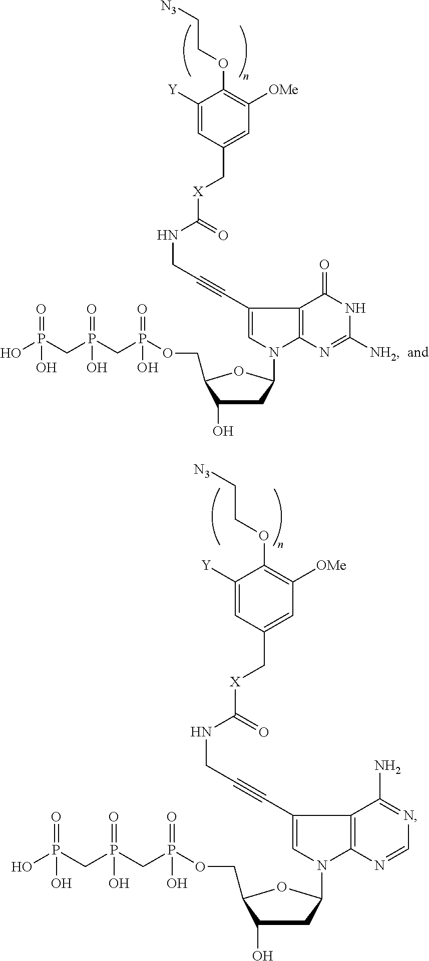

wherein X is O or NH, and wherein Y is H or OMe.

Embodiment 124 is the kit of embodiment 104, wherein the kit comprises one or more of the following molecules capable of being reacted with the engineered TdT, wherein the conjugation residue is a cysteine residue in the TdT:

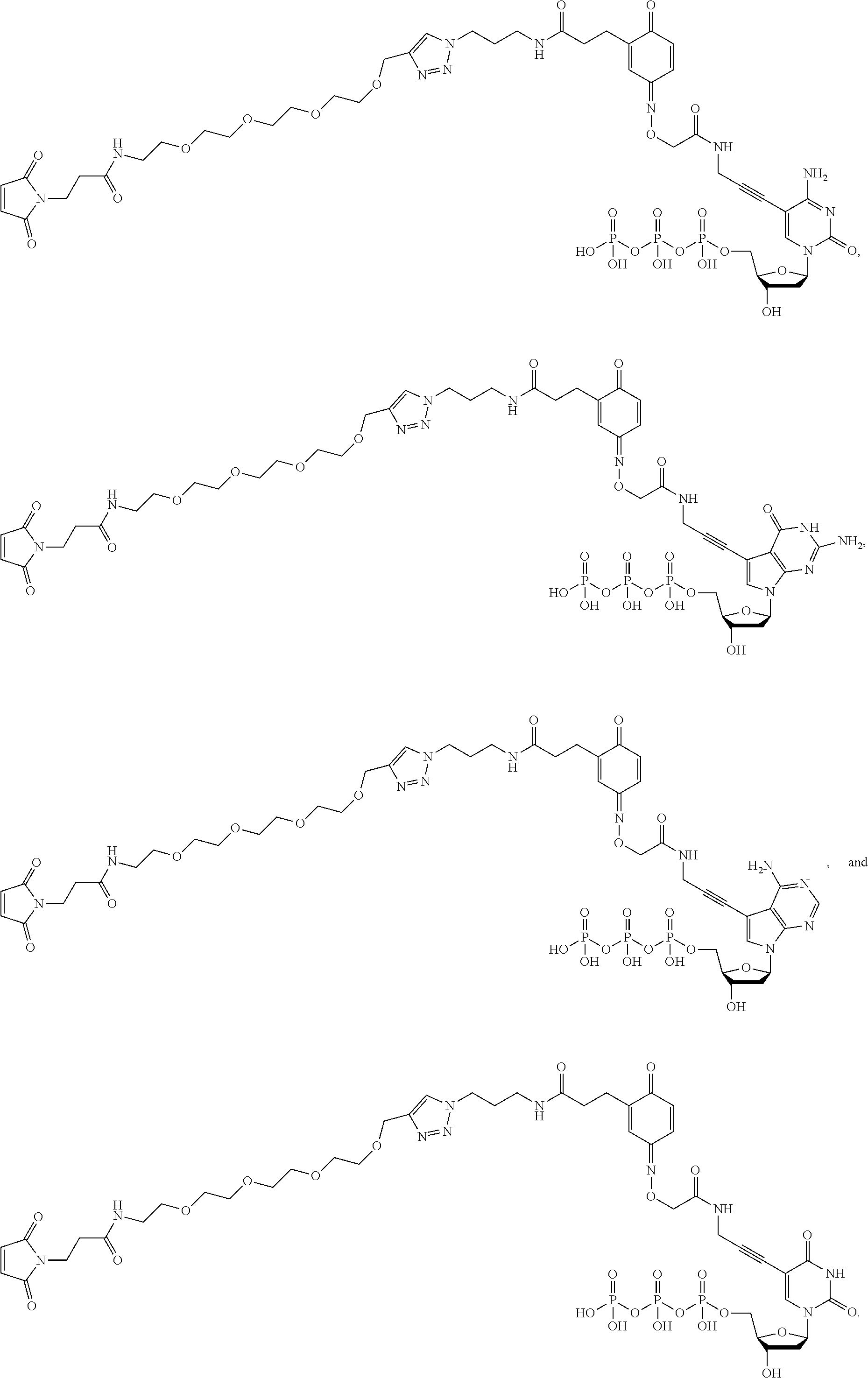

Embodiment 125 is the kit of embodiment 104, wherein the kit comprises one or more of the following molecules capable of being reacted with the engineered TdT, wherein the conjugation residue is a cysteine residue in the TdT:

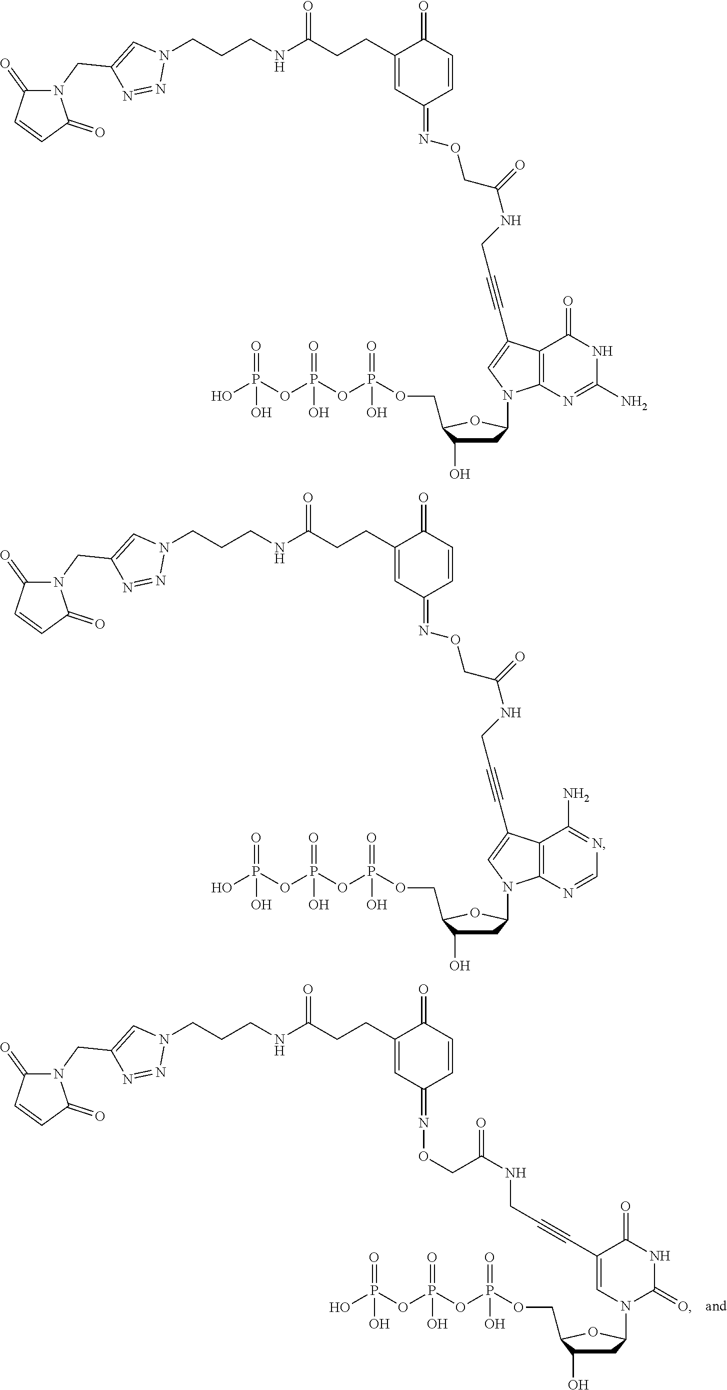

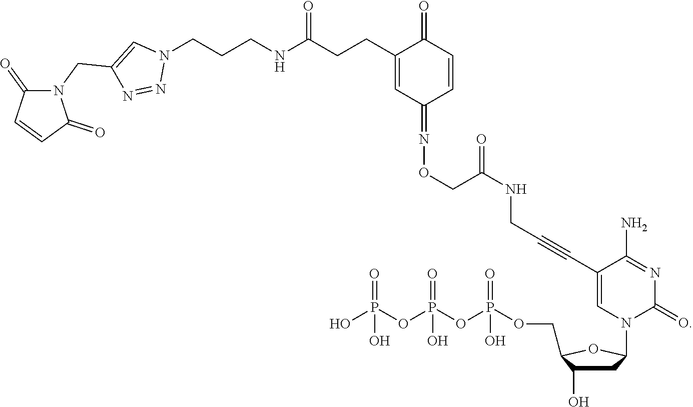

Embodiment 126 is the kit of embodiment 104, wherein the kit comprises one or more of the following molecules capable of being reacted with the engineered TdT, wherein the conjugation residue is a non-canonical amino acid residue in the TdT:

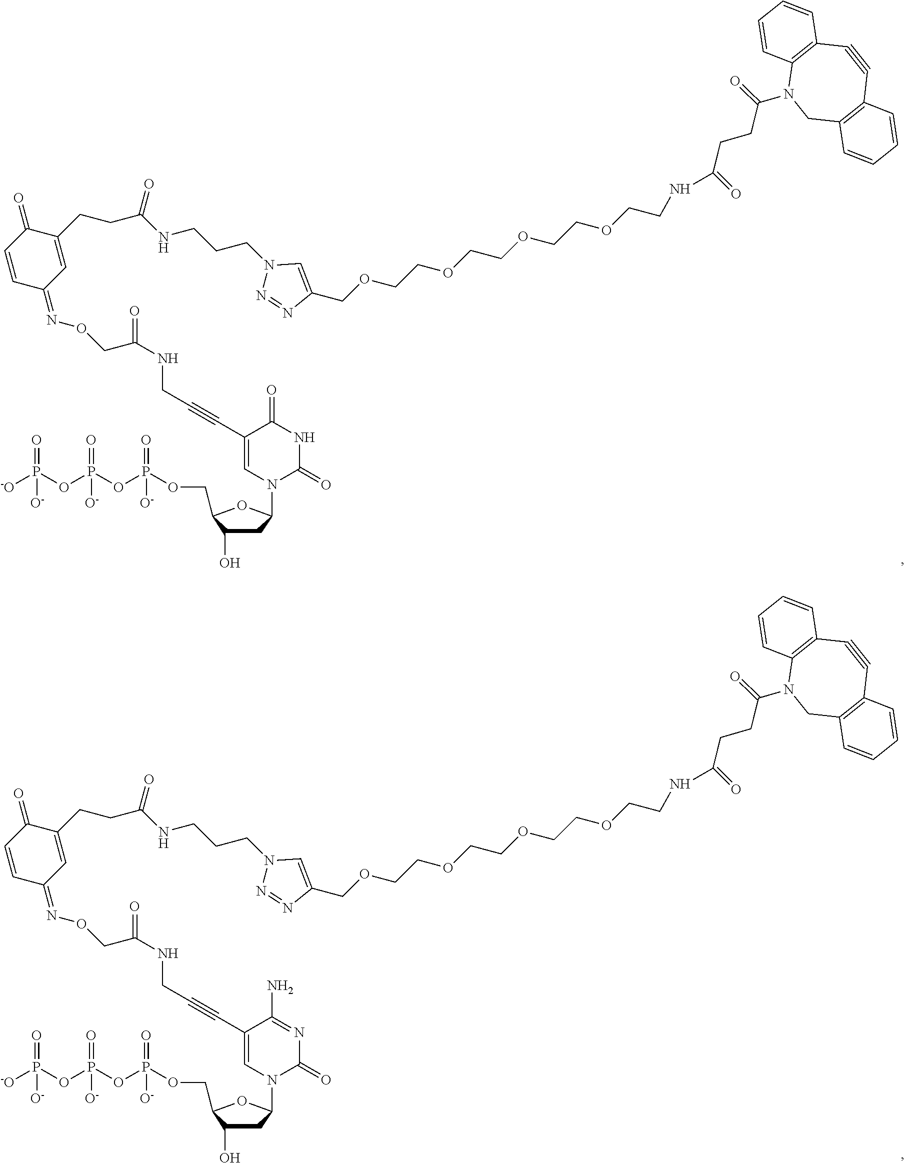

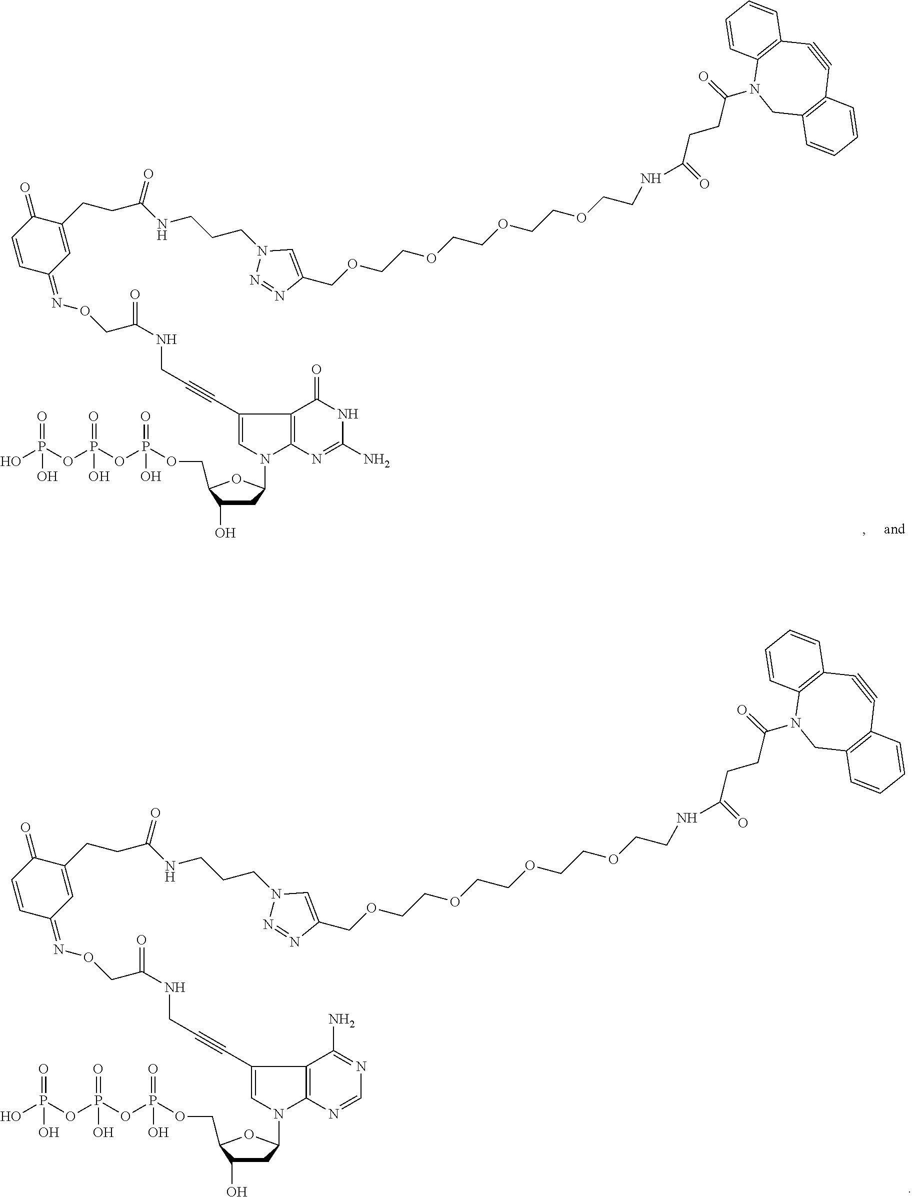 [SMILES: Nc1nc2c(c(C#CCNC(=O)CO/N=C3/C=CC(=O)C(CCC(=O)NCCCn4cc(COCCOCCOCCOCCNC(=O)CCC(=O)N5Cc6ccccc6C#Cc6ccccc65)nn4)=C3)cn2[C@H]2C[C@@H](O)[C@@H](COP(=O)(O)OP(=O)(O)OP(=O)(O)O)O2)c(=O)[nH]1 ; Nc1ncnc2c1c(C#CCNC(=O)CO/N=C1/C=CC(=O)C(CCC(=O)NCCCn3cc(COCCOCCOCCOCCNC(=O)CCC(=O)N4Cc5ccccc5C#Cc5ccccc54)nn3)=C1)cn2[C@H]1C[C@@H](O)[C@@H](COP(=O)(O)OP(=O)(O)OP(=O)(O)O)O1]

Embodiment 127 is the kit of embodiment 104, wherein the kit comprises one or more of the following molecules capable of being reacted with the engineered TdT, wherein the conjugation residue is a lysine residue in the TdT:

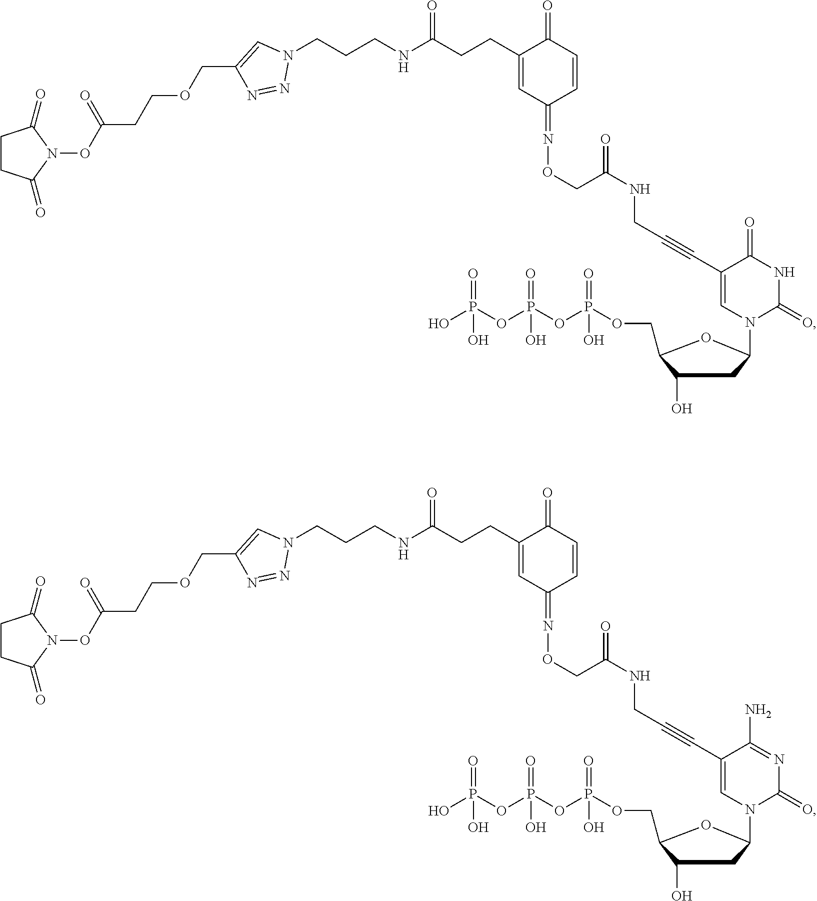 [SMILES: O=C(CCC1=CC(=NOCC(=O)NCC#Cc2cn([C@H]3C[C@@H](O)[C@@H](COP(=O)(O)OP(=O)(O)OP(=O)(O)O)O3)c(=O)[nH]c2=O)C=CC1=O)NCCCn1cc(COCCC(=O)ON2C(=O)CCC2=O)nn1 ; Nc1nc(=O)n([C@H]2C[C@@H](O)[C@@H](COP(=O)(O)OP(=O)(O)OP(=O)(O)O)O2)cc1C#CCNC(=O)CON=C1C=CC(=O)C(CCC(=O)NCCCn2cc(COCCC(=O)ON3C(=O)CCC3=O)nn2)=C1]

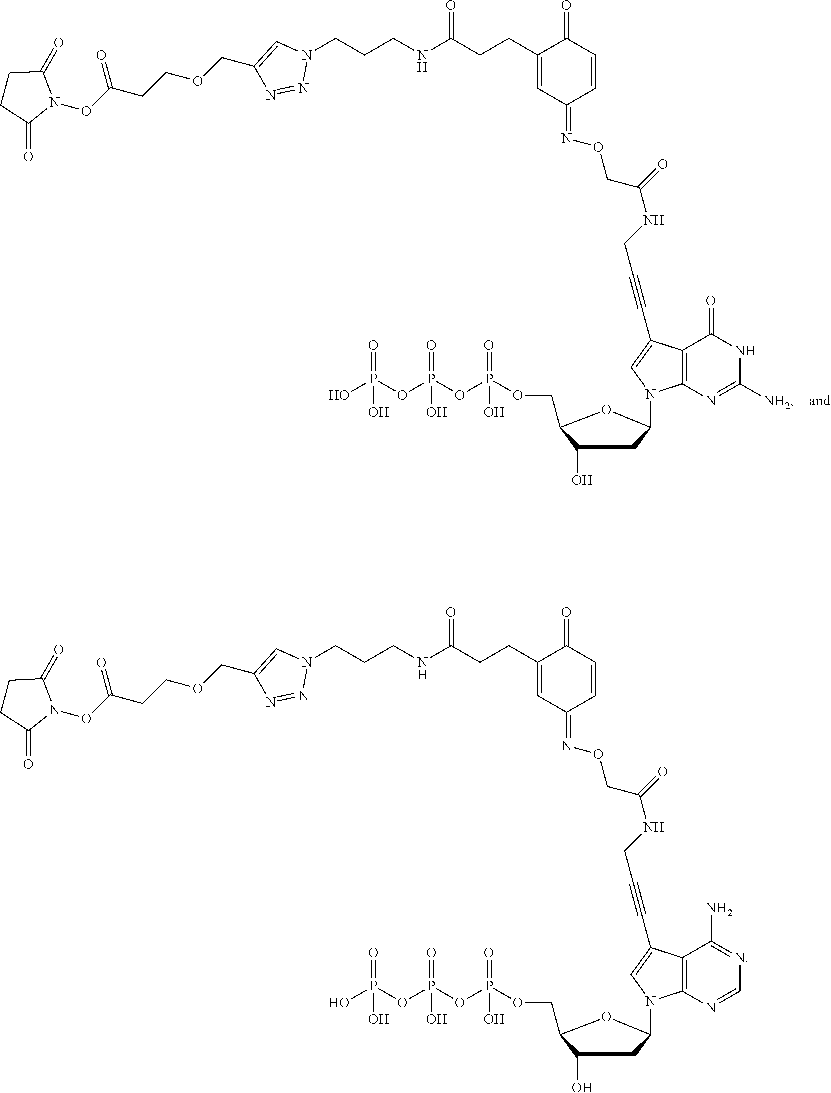 [SMILES: Nc1nc2c(c(C#CCNC(=O)CO/N=C3\C=CC(=O)C(CCC(=O)NCCCn4cc(COCCC(=O)ON5C(=O)CCC5=O)nn4)=C3)cn2[C@H]2C[C@@H](O)[C@@H](COP(=O)(O)OP(=O)(O)OP(=O)(O)O)O2)c(=O)[nH]1 ; Nc1ncnc2c1c(C#CCNC(=O)CO/N=C1\C=CC(=O)C(CCC(=O)NCCCn3cc(COCCC(=O)ON4C(=O)CCC4=O)nn3)=C1)cn2[C@H]1C[C@@H](O)[C@@H](COP(=O)(O)OP(=O)(O)OP(=O)(O)O)O1]

Embodiment 128 is a system for enzymatic DNA synthesis comprising:
a. the engineered TdT of any one of embodiments 34-93;
b. a redox-cleavable linker;
c. a redox shuttle solution; and
d. two or more electrodes on a surface.

Embodiment 129 is the system of embodiment 128, wherein the electrodes enable an applied potential or galvanic field to be supplied locally to the device or system thus enabling generation of active redox shuttle via reduction or oxidation at one or more electrodes.

Embodiment 130 is the system of embodiment 128 or embodiment 129, wherein the electrode where the soluble redox shuttle is generated is comprised of a smaller surface area than the other electrode to which it is electrically coupled.

Embodiment 131 is the system of any one of embodiments 128-130, wherein if the soluble redox shuttle is activated via cathodic process, then the cathode may be comprised of less active surface area than the anode by (a) having less electroactive area, or (b) being coupled to multiple anodes which in combination provide substantially more area than the cathode.

Embodiment 132 is the system of any one of embodiments 128-131, wherein the surface, or electroactive, or area aspect ratios for the counter electrode relative to the working electrode, or C/E aspect ratio is at least: 3:1, 6:1, 10:1, 100:1, 1000:1, or higher.

Embodiment 133 is the system of any one of embodiments 128-132, wherein all the electrodes lie in a single plane.

Embodiment 134 is the system of any one of embodiments 128-132, wherein a combination of electrodes may lie in multiple planes.

Embodiment 135 is the system of any one of embodiments 128-132, wherein electrodes are oriented parallel to perpendicular with respect to one another during the current path.

Embodiment 136 is the system of any one of embodiments 128-135, wherein the system has an inter-electrode gap (i.e., distance between electrodes included in the current path) of ≤100 μm, ≤10 μm, ≤1 μm, ≤100 μm, ≤10 nm, ≤1 nm, ≤100 μm, ≤10 μm, or ≤1 μm.

Embodiment 137 is the system of any one of embodiments 128-136, wherein the redox shuttle solution comprises at least one soluble redox shuttle in combination with a buffer, a supporting electrolyte, and/or a hydrotropic agent.

Embodiment 138 is the system of embodiment 137, wherein the buffer is sodium citrate, potassium hydrogen phosphate, or potassium dihydrogen phosphate.

Embodiment 139 is the system of embodiment 137 or 138, wherein the supporting electrolyte is sodium chloride, sodium salts, lithium salts, potassium salts, or magnesium salts.

Embodiment 140 is the system of any one of embodiments 137-139, wherein the hydrotropic agent is caffeine, urea, and/or nicotinamide (NA).

Embodiment 141 is the system of any one of embodiments 128-136, wherein the redox shuttle solution comprises methylene blue, citrate-phosphate buffer, and sodium chloride.

Embodiment 142 is the system of any one of embodiments 128-136, wherein the redox shuttle solution comprises methylene blue, citrate-phosphate buffer, sodium chloride, and nicotinamide.

Embodiment 143 is the system of any one of embodiments 128-136, wherein the redox shuttle solution comprises flavin mononucleotide, citrate-phosphate buffer, sodium chloride, and nicotinamide.

Embodiment 144 is the system of any one of embodiments 128-136, wherein the redox shuttle solution comprises 10 mM methylene blue, 25 mM citrate-phosphate buffer at pH 3.4, and 100 mM sodium chloride.

Embodiment 145 is the system of any one of embodiments 128-136, wherein the redox shuttle solution comprises 10 mM methylene blue, 25 mM citrate-phosphate buffer at pH 3.4, 100 mM sodium chloride, and 1 M nicotinamide.

Embodiment 146 is the system of any one of embodiments 128-136, wherein the redox shuttle solution comprises 50 mM flavin mononucleotide, 25 mM citrate-phosphate buffer at pH 3.4, 1 M sodium chloride, and 1 M nicotinamide.

Embodiment 147 is a redox shuttle solution comprising at least one soluble redox shuttle in combination with one or more of the following: supporting electrolyte, buffer, and hydrotrope.

Embodiment 148 is the redox shuttle solution of embodiment 147, comprising: 10 mM methylene blue, 25 mM citrate-phosphate buffer at pH 3.4, and 100 mM sodium chloride.

Embodiment 149 is the redox shuttle solution of embodiment 147, comprising: 10 mM methylene blue, 25 mM citrate-phosphate buffer at pH 3.4, 100 mM sodium chloride, and 1 M nicotinamide.

Embodiment 150 is the redox shuttle solution of embodiment 147, comprising: 50 mM flavin mononucleotide, 25 mM citrate-phosphate buffer at pH 3.4, 1 M sodium chloride, and 1 M nicotinamide.

Embodiment 151 is a molecule comprising any one of the following

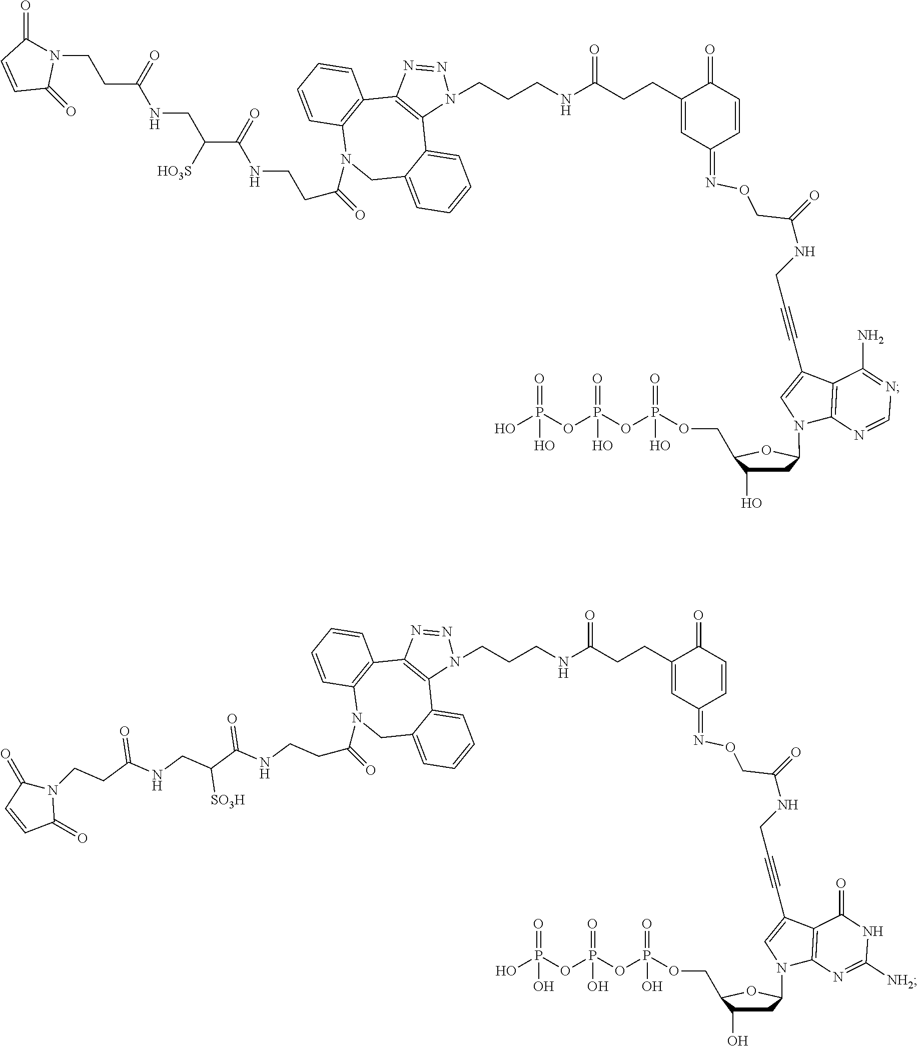

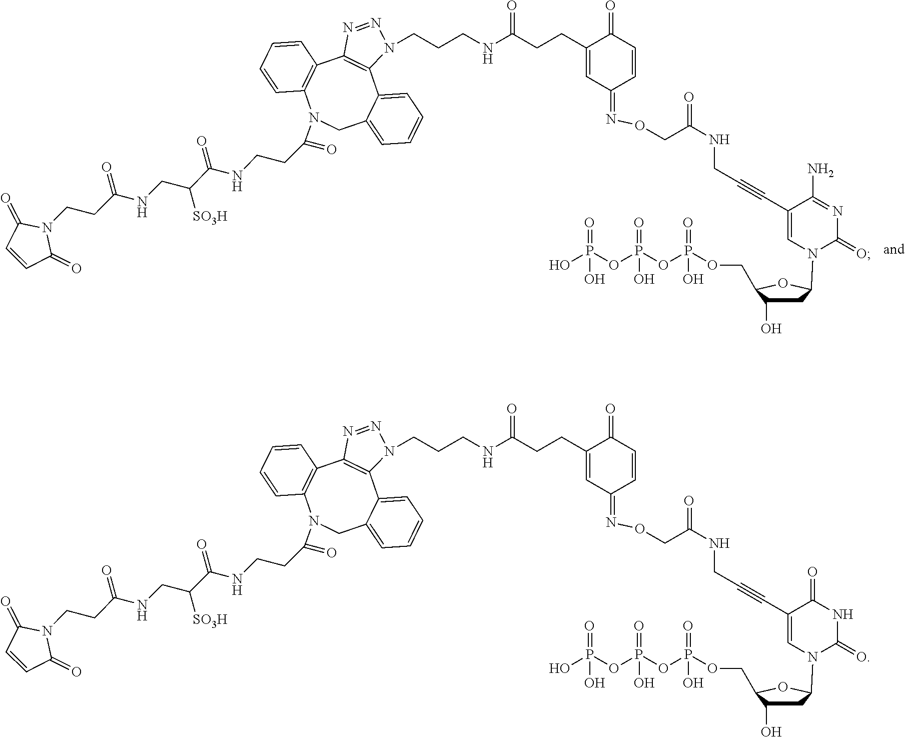

Embodiment 152 is a molecule comprising any one of the following

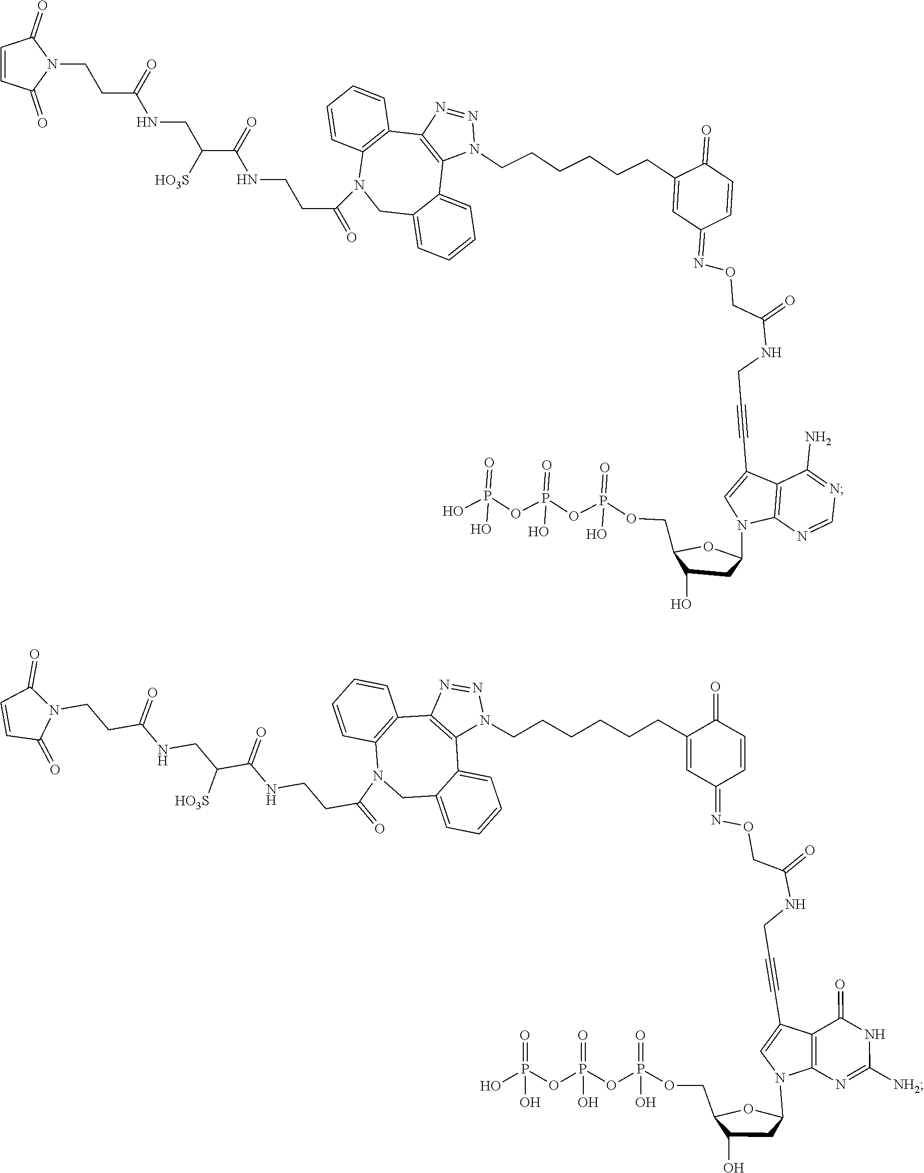 [SMILES: Nc1ncnc2c1c(C#CCNC(=O)CO/N=C1\C=CC(=O)C(CCCCCCn3nnc4c3-c3ccccc3CN(C(=O)CCNC(=O)C(CNC(=O)CCN3C(=O)C=CC3=O)S(=O)(=O)O)c3ccccc3-4)=C1)cn2[C@H]1C[C@@H](O)[C@@H](COP(=O)(O)OP(=O)(O)OP(=O)(O)O)O1 ; Nc1nc2c(c(C#CCNC(=O)CO/N=C3\C=CC(=O)C(CCCCCCn4nnc5c4-c4ccccc4CN(C(=O)CCNC(=O)C(CNC(=O)CCN4C(=O)C=CC4=O)S(=O)(=O)O)c4ccccc4-5)=C3)cn2[C@H]2C[C@@H](O)[C@@H](COP(=O)(O)OP(=O)(O)OP(=O)(O)O)O2)c(=O)[nH]1]

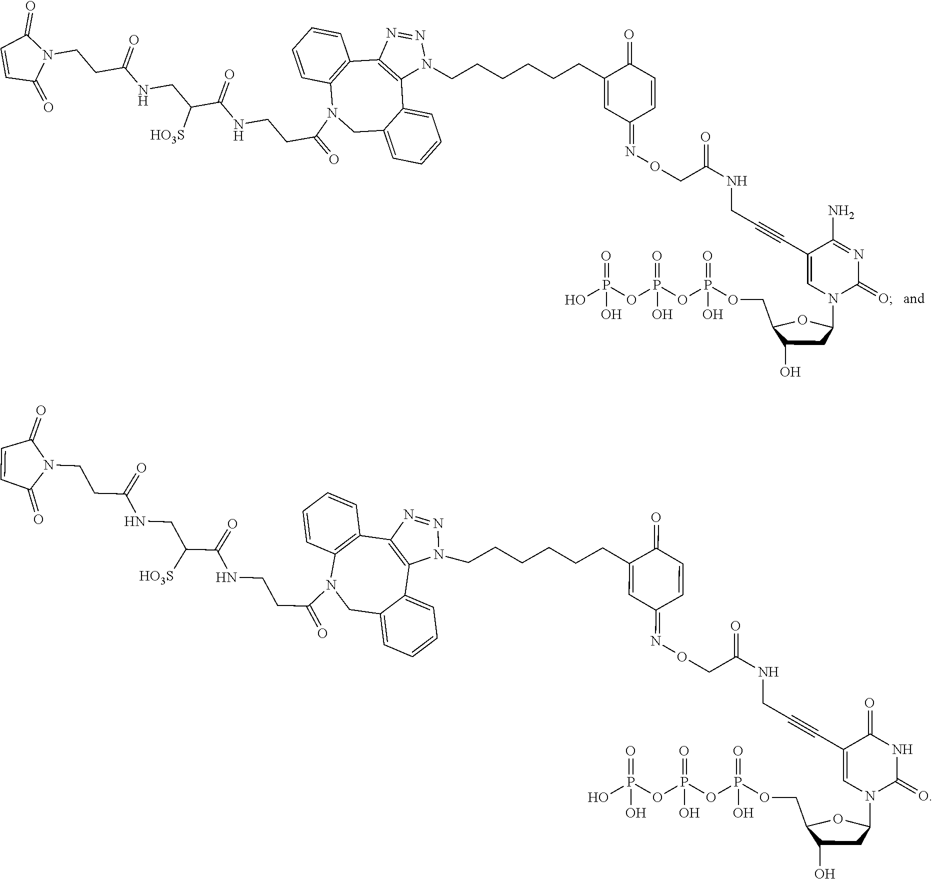 [SMILES: Nc1nc(=O)n([C@H]2C[C@@H](O)[C@@H](COP(=O)(O)OP(=O)(O)OP(=O)(O)O)O2)cc1C#CCNC(=O)CO/N=C1\C=CC(=O)C(CCCCCCn2nnc3c2-c2ccccc2CN(C(=O)CCNC(=O)C(CNC(=O)CCN2C(=O)C=CC2=O)S(=O)(=O)O)c2ccccc2-3)=C1 ; "and" ; O=C(CCN1C(=O)C=CC1=O)NCC(C(=O)NCCC(=O)N1Cc2ccccc2-c2c(nnn2CCCCCCC2=C/C(=N/OCC(=O)NCC#Cc3cn([C@H]4C[C@@H](O)[C@@H](COP(=O)(O)OP(=O)(O)OP(=O)(O)O)O4)c(=O)[nH]c3=O)C=CC2=O)-c2ccccc21)S(=O)(=O)O]

Embodiment 153 is a molecule comprising the following structure:

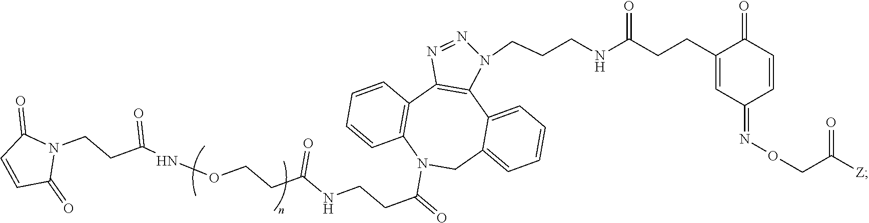

wherein Z is any one of the following:

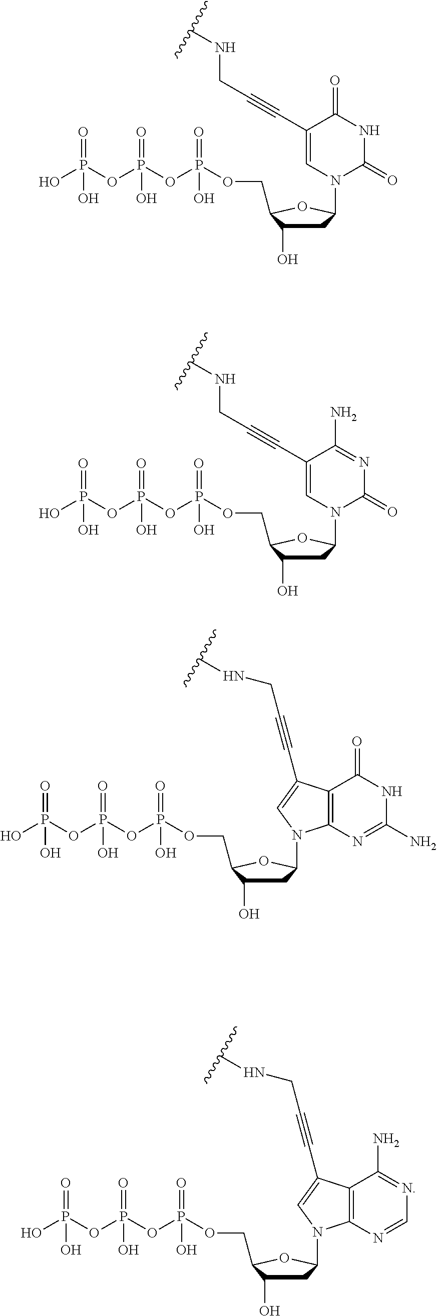

Embodiment 154 is a molecule comprising the following structure:

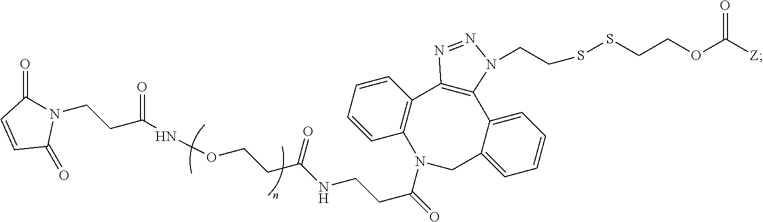

wherein Z is any one of the following:

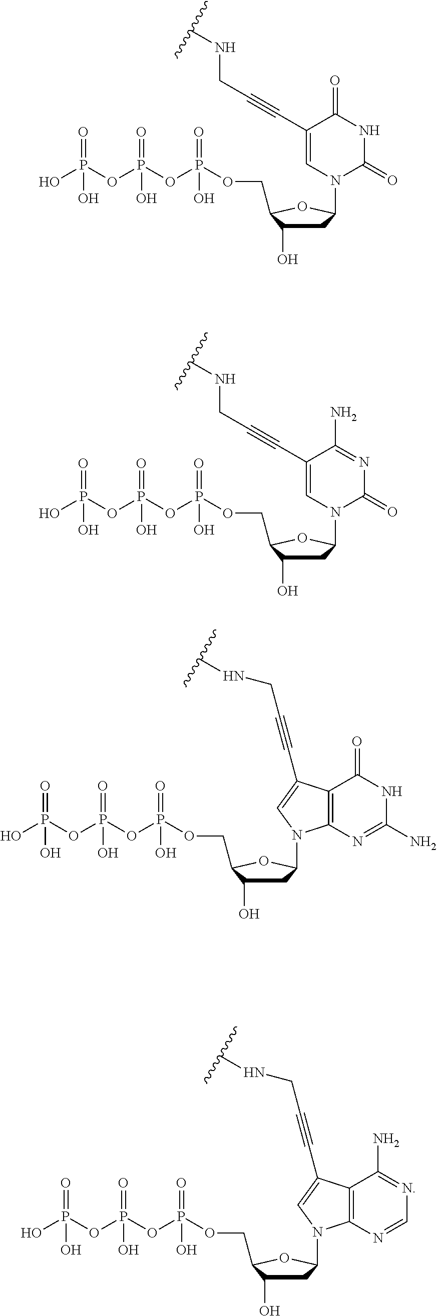

Embodiment 155 is a molecule comprising the following structure:

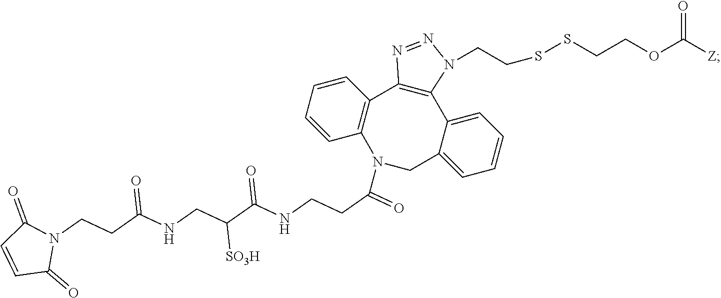

wherein Z is any one of the following:

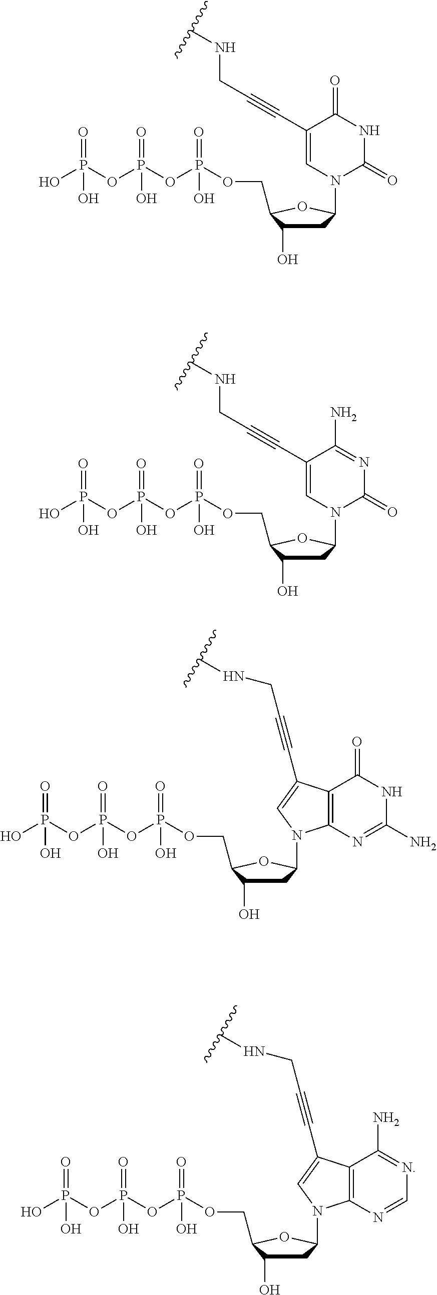

Embodiment 156 is a molecule comprising the following structure:

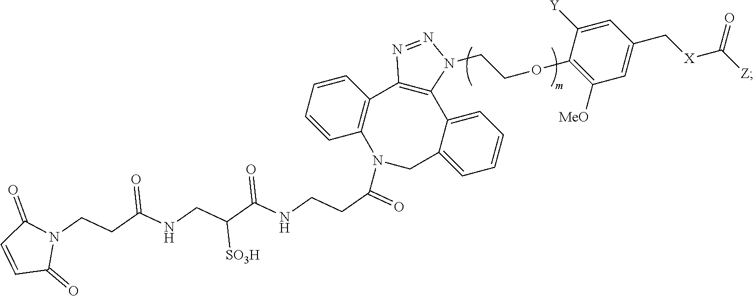

wherein X is O or NH;
wherein Y is H or OMe; and
wherein Z is any one of:

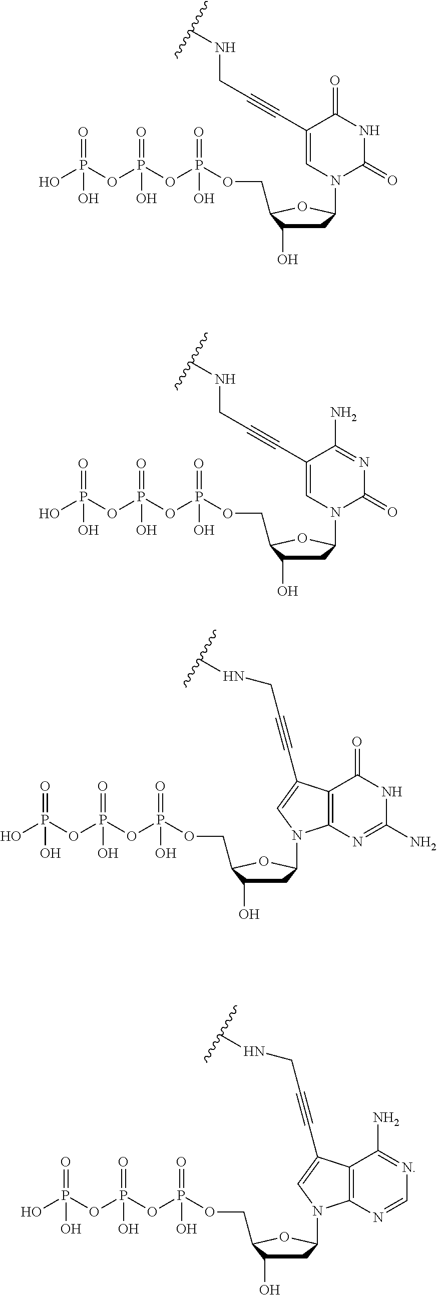

Embodiment 157 is a molecule comprising the following structure:

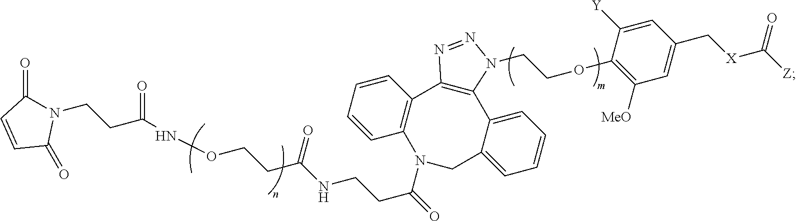

wherein X is O or NH;
wherein Y is H or OMe; and
wherein Z is any one of:

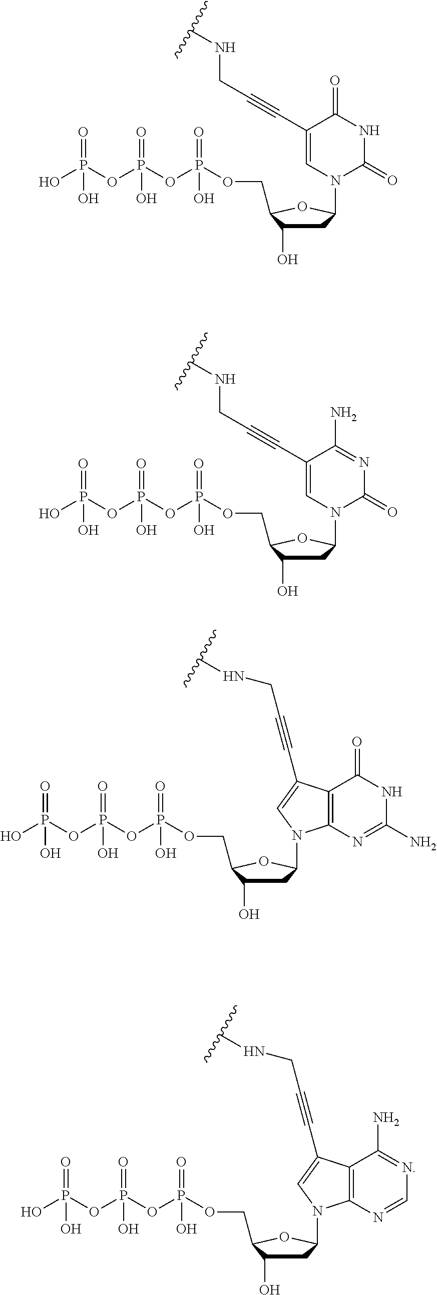

Embodiment 158 is a molecule comprising the following structure:

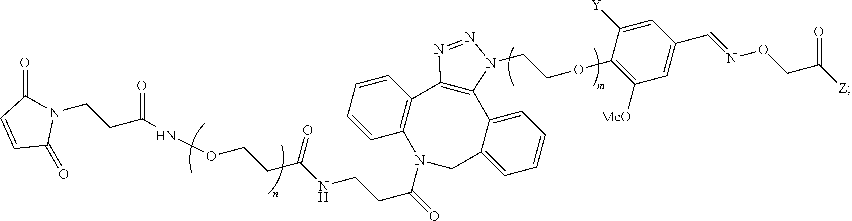

wherein Y is H or OMe; and
wherein Z is any one of:

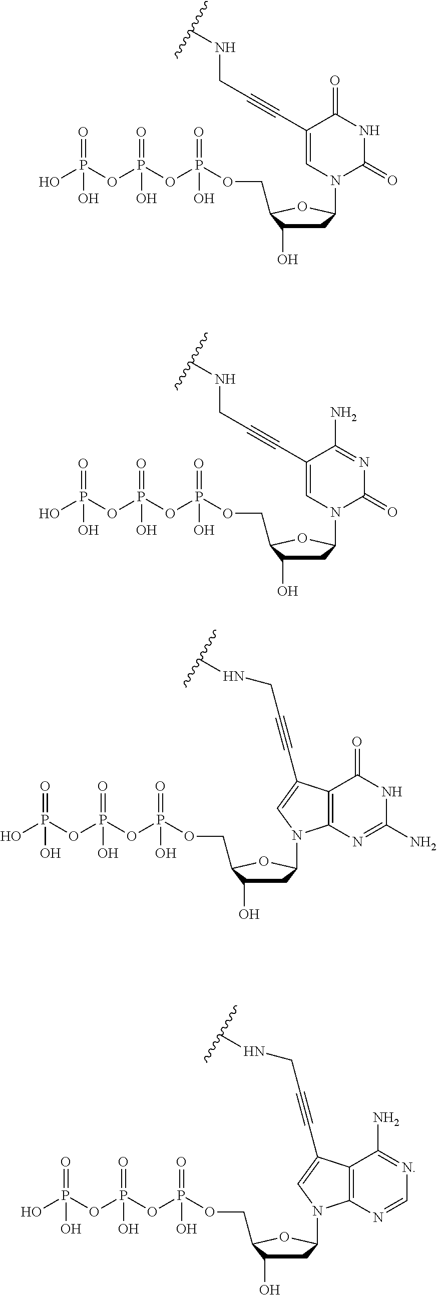

Embodiment 159 is a molecule covalently linked to a tether, comprising the following structure:

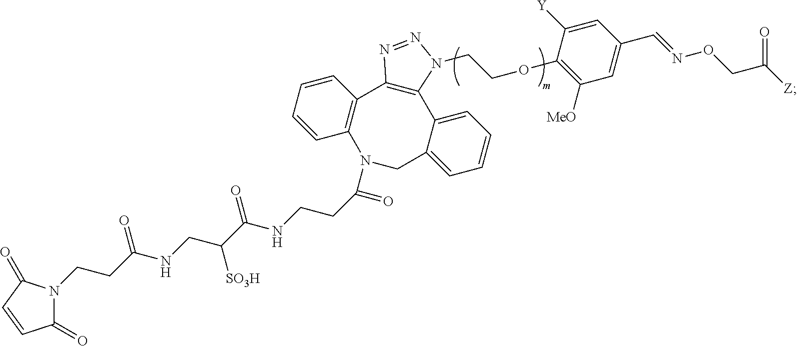

wherein Y is H or OMe; and
wherein Z is any one of:

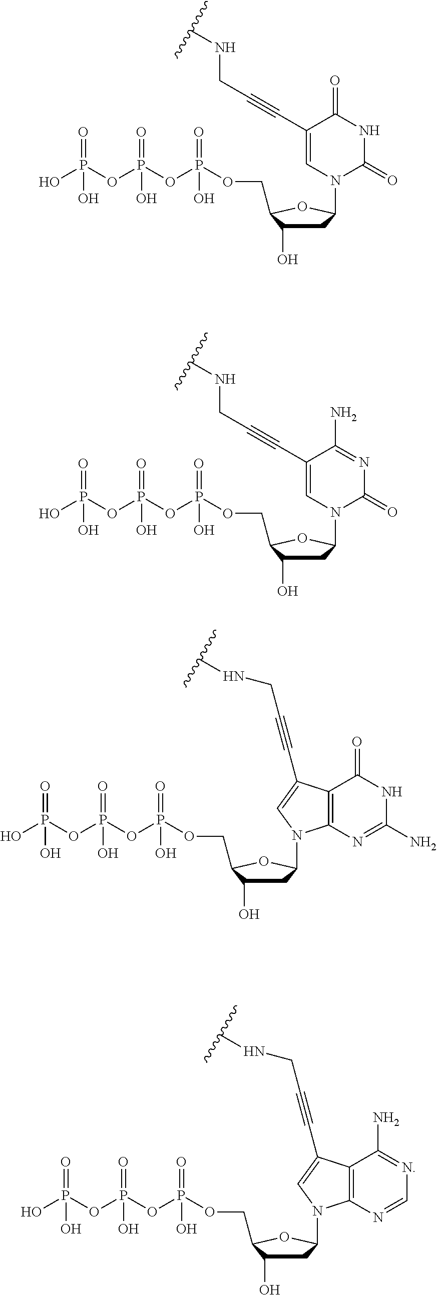

Embodiment 160 is a molecule comprising any one of the following

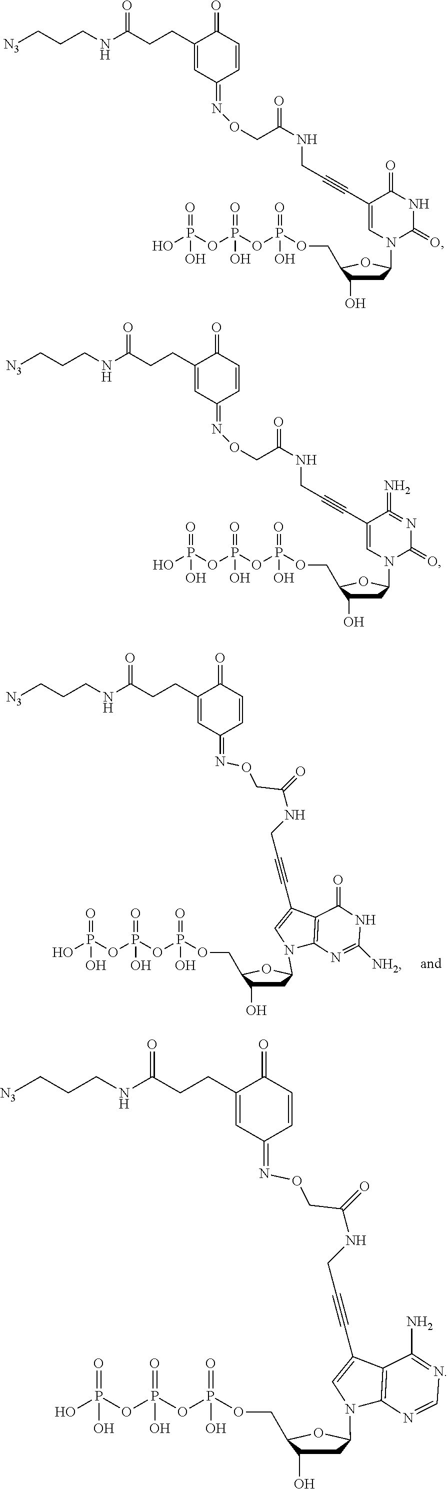

Embodiment 161 is a molecule comprising any one of the following

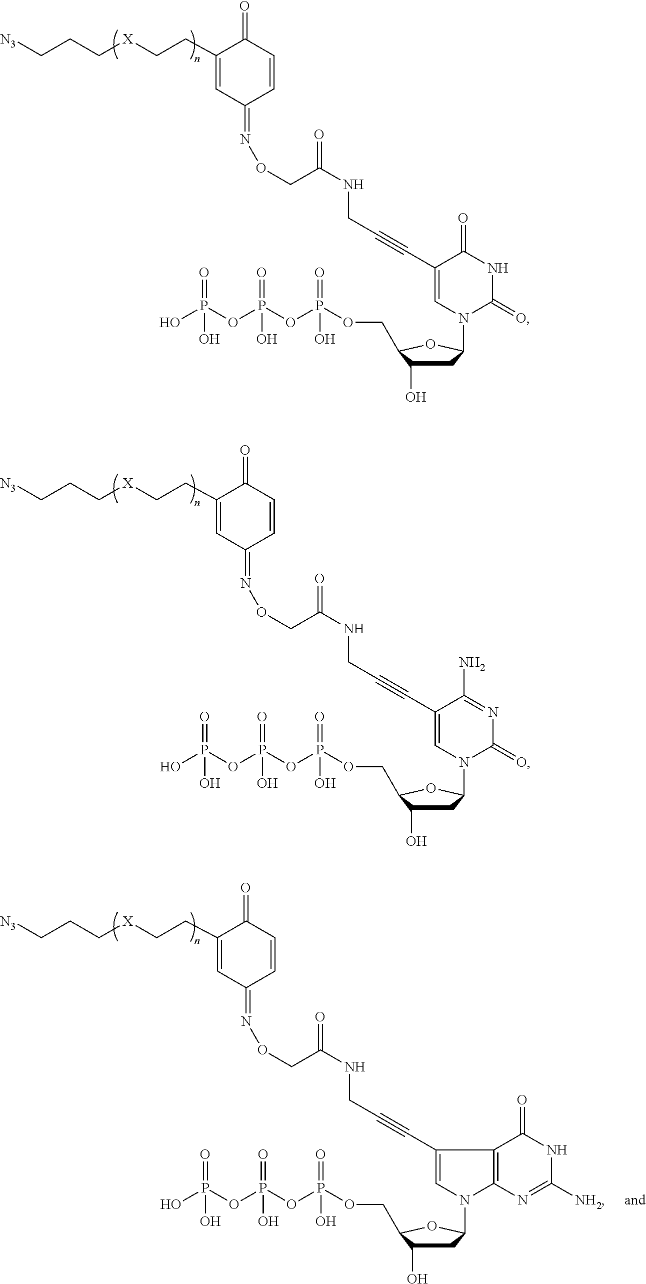

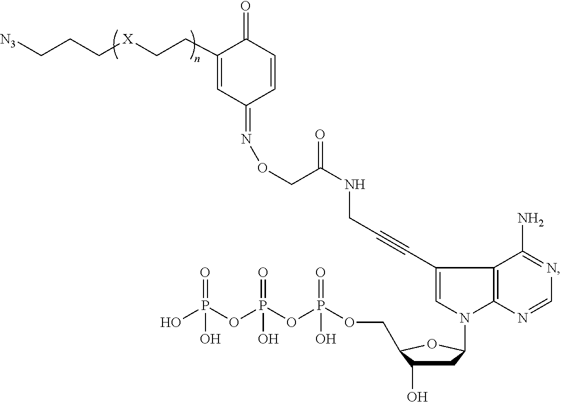

wherein X is C(O), C(O)NH, CH2, or O; and wherein n=1 for X═C(O), n=1 for C(O)NH, n=1 for CH2, and n=1-4 for X═O, wherein n is an integer.

Embodiment 162 is a molecule comprising any one of the following

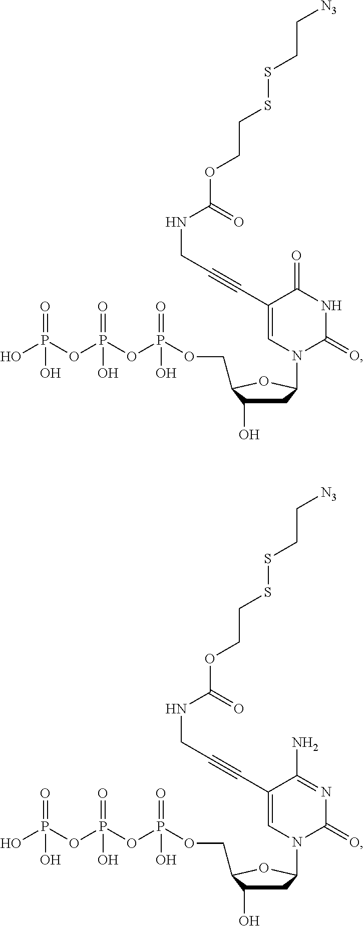

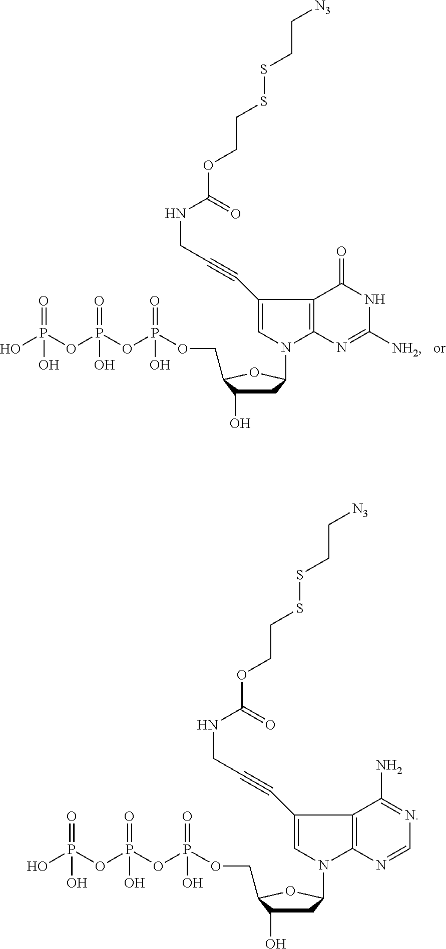

Embodiment 163 is a molecule comprising any one of the following

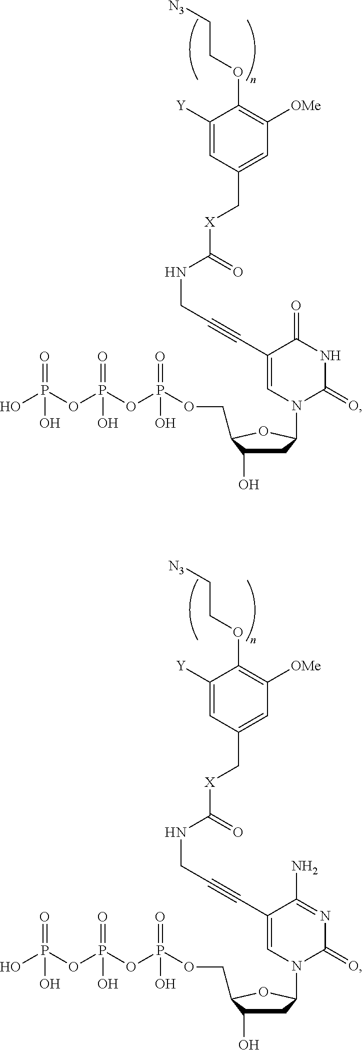

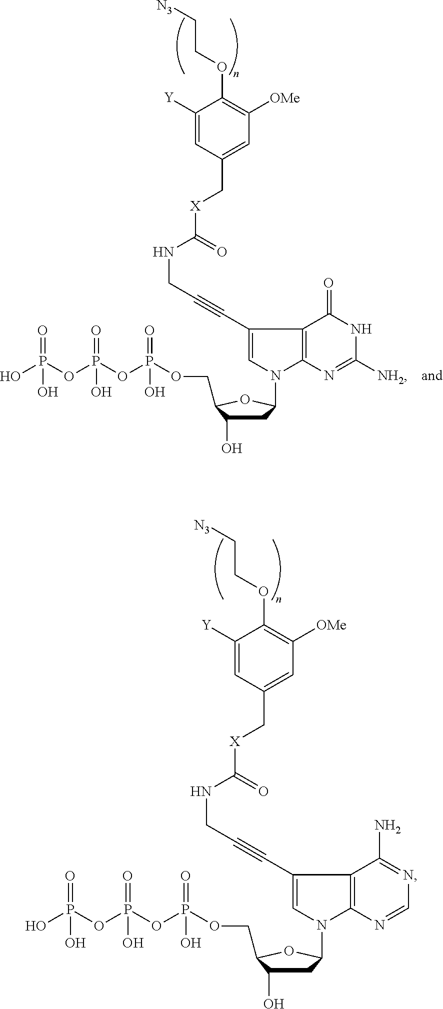

wherein X is O or NH, and wherein Y is H or OMe.

Embodiment 164 is a molecule comprising any one of the following

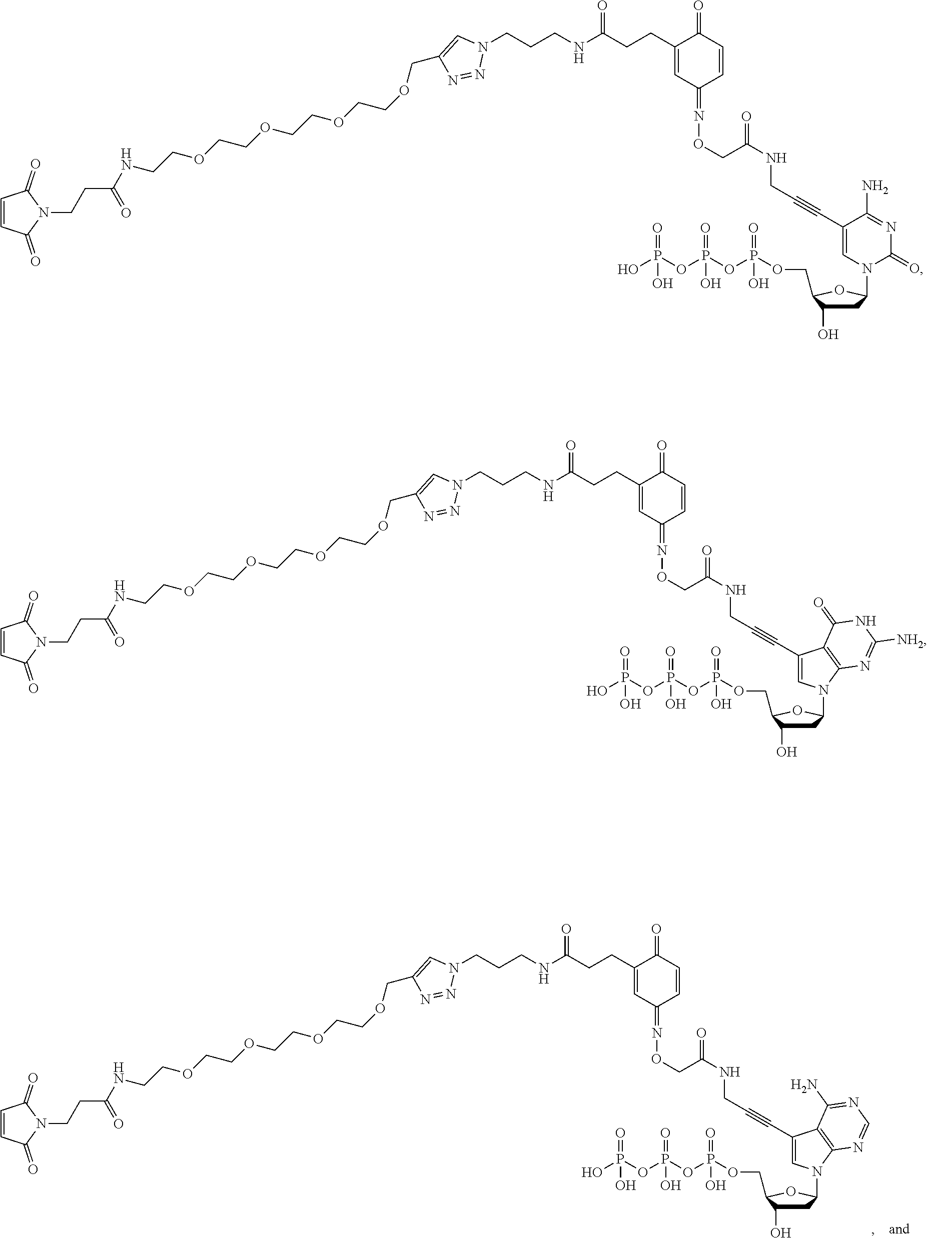

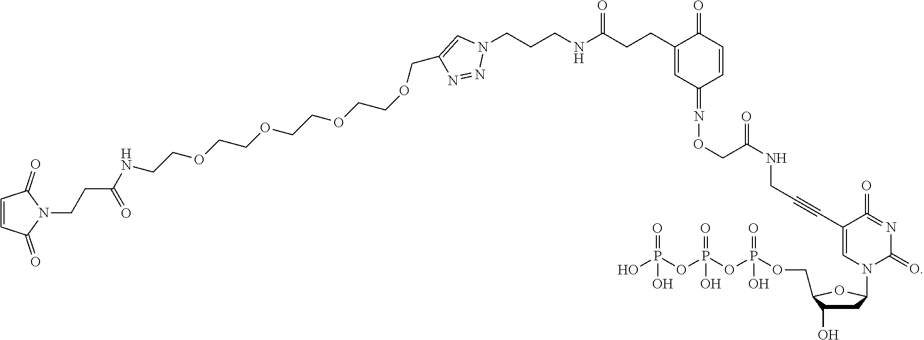

Embodiment 165 is a molecule comprising any one of the following

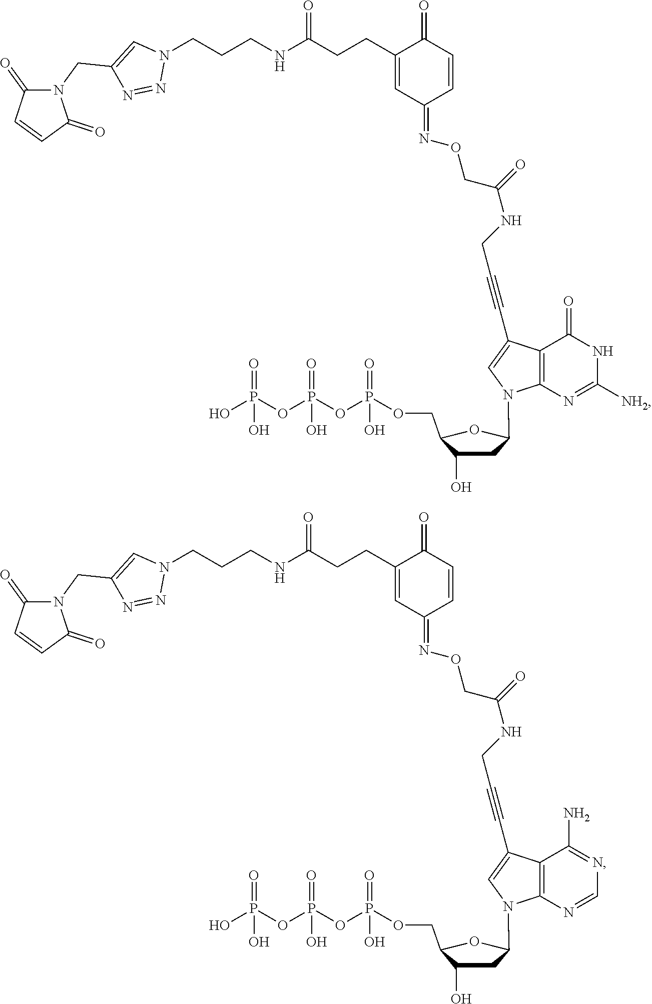

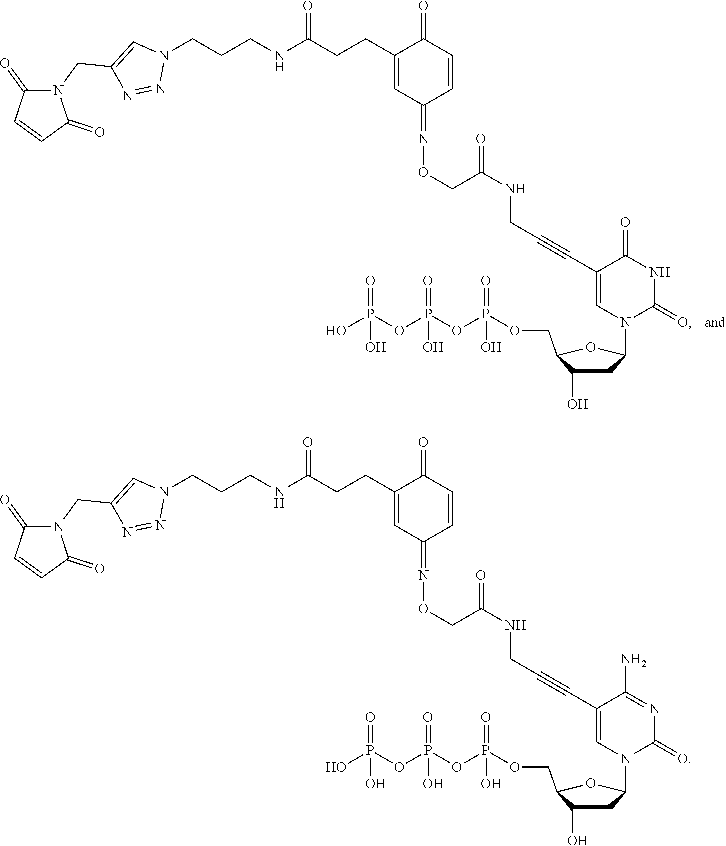 [SMILES: O=C(CCC1=CC(=NOCC(=O)NCC#Cc2cn([C@H]3C[C@@H](O)[C@@H](COP(=O)(O)OP(=O)(O)OP(=O)(O)O)O3)c(=O)[nH]c2=O)C=CC1=O)NCCCn1cc(CN2C(=O)C=CC2=O)nn1 ; "and" ; Nc1nc(=O)n([C@H]2C[C@@H](O)[C@@H](COP(=O)(O)OP(=O)(O)OP(=O)(O)O)O2)cc1C#CCNC(=O)CON=C1C=CC(=O)C(CCC(=O)NCCCn2cc(CN3C(=O)C=CC3=O)nn2)=C1]

Embodiment 166 is a molecule comprising any one of the following

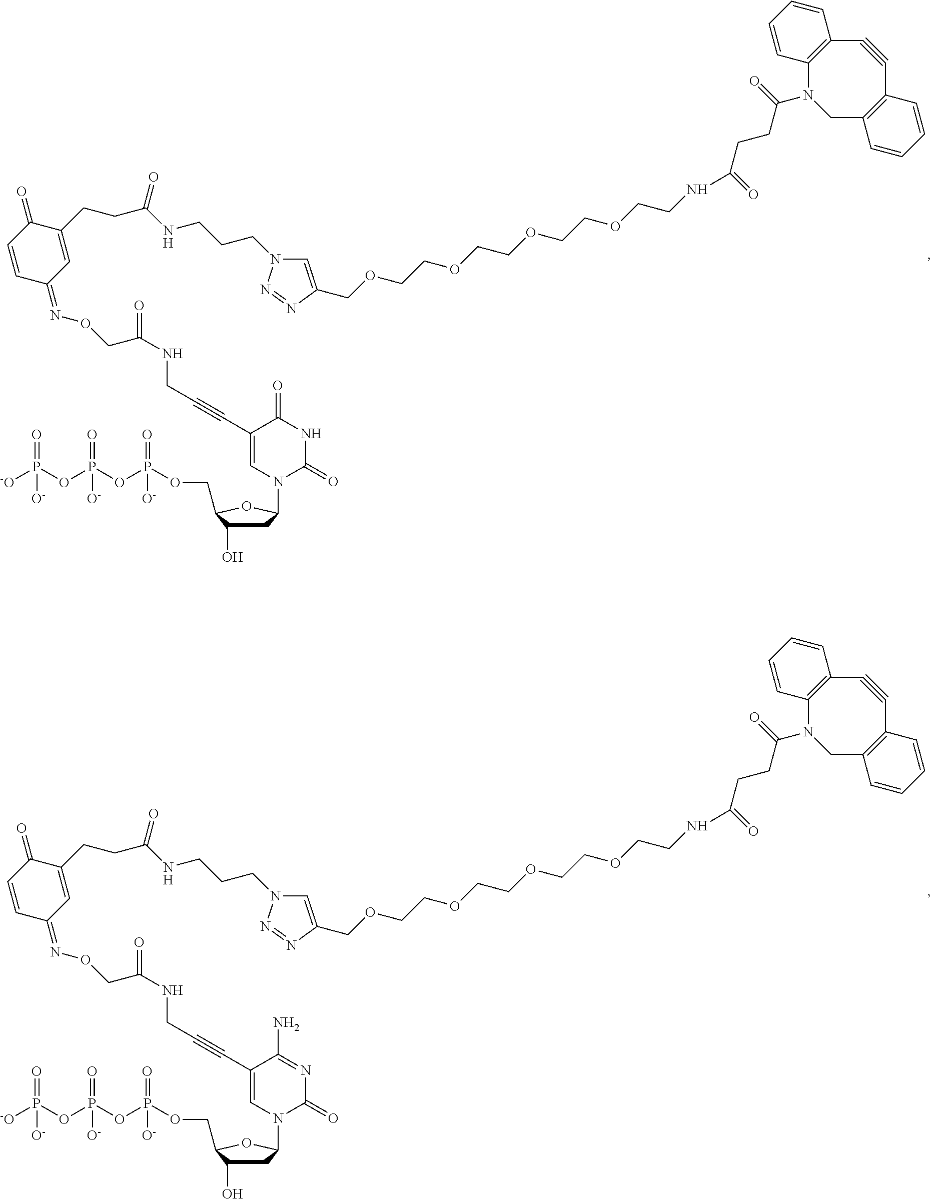

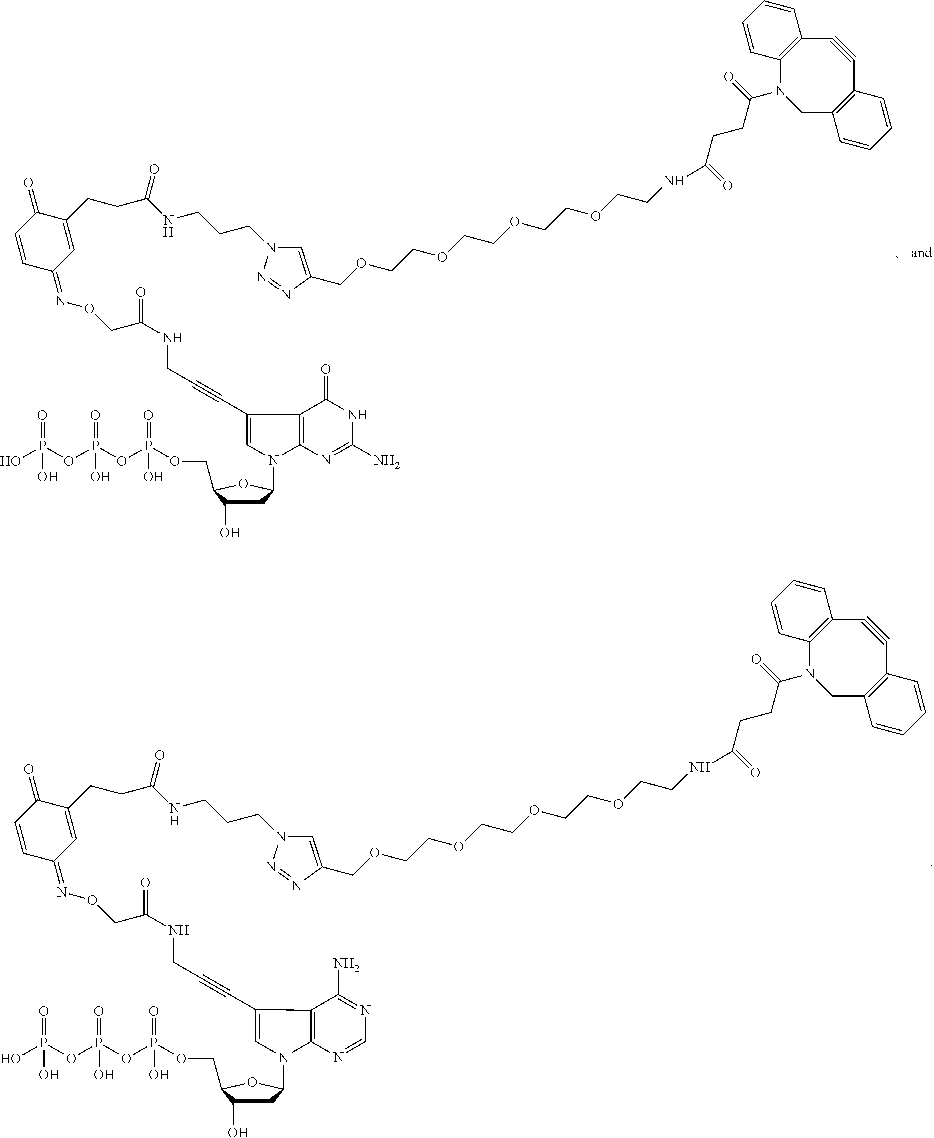

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims,

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIGS. 1A-C show a schematic of covalently attaching nucleotides to TdT via a redox cleavable linker (a quinone). FIG. 1A, FIG. 1B, and FIG. 1C refer to different time points. Upon incubation with ssDNA, the enzyme-tethered nucleotide is added to the ssDNA strand, creating a covalent enzyme-ssDNA complex. Reductive cleavage of the quinone cleaves the ssDNA from the enzyme.

FIG. 2 shows conjugation sites in a PROSS-stabilized enzyme. Structures of the wild type mTdT shown in white (PDB 4127) aligned with the PROSS-stabilized enzyme (Pross1 or P1) shown in black. Conjugation sites D22, E29, E32, E37, R44, L93, K102, V152, P154, and N361 are labeled on the structure as well as the position on the bound nucleotide that will be tethered to the enzyme conjugation sites via a linker and attachment moiety on the nucleotide molecule.

FIGS. 3A-B show expression and purification of construct C1. FIG. 3A shows an SDS-PAGE gel of expression and purification in-process samples. Expected molecular weight of C1 is 41.7 kDa. Lanes: 1=Protein Ladder, 2=Total cell lysate from 15 °C expression, 3=Soluble cell lysate from 15 °C expression, 4=Total cell lysate from 30 °C expression, 5=Soluble cell lysate from 30 °C expression, 6=final purified protein. FIG. 3B shows an SEC chromatogram from C1 on Superdex200 10/300 GL column. The peak at 6.83 ml is at the void volume of the column and contains aggregate; the peak eluting at 15.78 ml is the expected elution volume for a 42 kDa monomer.

FIGS. 4A-B show stabilized TdT variants have increased activity at elevated temperatures. FIG. 4A: shows a schematic of a free extension assay to assess TdT activity. Enzyme is incubated with fluorophore-conjugated seed oligo and dNTPs at 37 °C for 20 minutes. Reactions are then analyzed via SDS-PAGE to assess the extension rate of the enzyme as seen by the migration of the extended oligo on the gel, larger fragments running slower at the top of the gel. FIG. 4B: shows results from a free extension assay run with wild type murine TdT (gray), the P1 stabilized variant (blue), and the P2 stabilized variant (red) at the indicated temperatures. The left-most lane shows that the starting fluorescent seed oligo runs at the bottom of the gel. Loss of activity for the WT enzyme is observed at 50 °C and above as seen by the shift in signal towards the bottom of the gel in those lanes. Contrarily, significant loss of activity is only observed for P1 above 55 °C and for P2 above 50 °C.

FIG. 5 shows an example of using a bifunctional linking molecule to install a reactive functional group on an enzyme. In this case a maleimide-alkyne crosslinker is reacted with a cysteine residue. The enzyme is now modified with an alkyne.

FIG. 6 shows an example of using an enzyme that has been modified with an alkyne through a bifunctional crosslinker. A molecule with a nucleotide and cleavable linker can be attached via click chemistry. In the case of adding a DBCO moiety (dibenzylcyclooctyne), the conjugation occurs without additional reagents.

FIGS. 7A-F show deconvoluted mass spectra for the P1 C7 variant before and after conjugation with sulfo-DBCO-maleimide and the azide-quinone-nucleotides. FIG. 7A shows a P1 C7 variant, unconjugated; FIG. 7B shows a C7 variant reacted with sulfo-DBCO-maleimide; FIG. 7C shows a C7 variant reacted with sulfo-DBCO-maleimide followed by azide-quinone-dATP; FIG. 7D shows a C7 variant reacted with sulfo-DBCO-maleimide followed by azide-quinone-dGTP; FIG. 7E shows a C7 variant reacted with sulfo-DBCO-maleimide followed by azide-quinone-dTTP; and FIG. 7F shows a sulfo-DBCO-maleimide followed by azide-quinone-dCTP.

FIG. 8 shows a graphic showing the potential and pH window over which an activated mediator could be expected to cleave the redox-cleavable linker while avoiding the oxidation and reduction of water.

FIG. 9 shows cyclic voltammograms for a selection of soluble redox shuttles (1 mM methylene blue or riboflavin) at 37 °C in a buffered solution of 1× TdT Storage Buffer (200 mM potassium phosphate pH 6.5, 100 mM sodium chloride). The working and counter electrodes are noble metals (platinum or gold) while the reference electrode is a silver/silver chloride (Ag/AgCl) couple. A 100 mV/sec scan rate is employed in the above example.

FIG. 10 shows cleavage of small molecule azide-quinone-nucleotide with leucomethylene blue. The reaction was monitored by LC-MS on an Agilent 1260 Infinity instrument with an Agilent 6120 Quadropole MS. Separations were performed using an Agilent Infinity Lab Poroshell EC-C18 column (4.6×100 mm, 2.7 μm) using the following solvent system at a flow rate of 0.5 mL/min: solvent A=0.05 M triethylammonium acetate (TEAA); solvent B=20% MeCN/0.05M TEAA; gradient method: 90% A/10% B for 3 min; linear gradient form 90% A/10% B to 20% A/80% B from 3-5 min; linear gradient from 20% A/80% B from 5 min to 8 min; maintained at 100% B from 8 min to 20 min. Compounds were detected by UV absorption at 210 nm, 254 nm, 280 nm, or 320 nm. Molecular weight range 400-2000; capillary voltage 3750 (pos) and 3500 (neg).

FIGS. 11A-B show ESI-MS (ESI-neg) data for the starting azide-quinone-nucleotide (azide-quinone-dCTP) (FIG. 11A) and the resulting product (“cleaved pdt”) after treatment with leucomethylene blue (FIG. 11B).

FIGS. 12A-B show ESI-MS (ESI-neg) data for the starting azide-quinone-nucleotide (azide-quinone-dCTP) (FIG. 12A) and the resulting product (“cleaved pdt”) after treatment with leucomethylene blue (FIG. 12B).

FIG. 13 shows cyclic voltammograms for three example formulations of redox shuttle (Formulation A: 10 mM methylene blue, 100 mM sodium chloride, 25 mM citrate-phosphate buffer, pH 3.4; Formulation B: 10 mM methylene blue, 100 mM sodium chloride, 1 M nicotinamide, 25 mM citrate-phosphate buffer, pH 3.4; Formulation C: 50 mM flavin mononucleotide, 1 M sodium chloride, 1 M nicotinamide, 25 mM citrate-phosphate buffer, pH 3.4) at room temperature. The working and counter electrodes are platinum while the reference electrode is a silver/silver chloride (Ag/AgCl) couple. A 100 mV/sec scan rate is employed in the above example.

FIG. 14 shows UV-Vis absorbance spectra for two variations of Formulation B (Dilution 1:0.25 mM methylene blue, 100 mM sodium chloride, 2.5 mM nicotinamide, 25 mM citrate-phosphate buffer, pH 3.4; Dilution 2:0.25 mM methylene blue, 100 mM sodium chloride, 100 mM nicotinamide, 25 mM citrate-phosphate buffer, pH 3.4).

FIG. 15 shows 1H-NMR spectra for 1 M nicotinamide in D₂O with peak assignments.

FIG. 16 shows 1H-NMR spectra for 1 M nicotinamide with 100 mM methylene blue in D₂O with peak assignments. Peaks B and E show broadening in the presence of methylene blue. Note, the doublet at 7.17 ppm is from methylene blue and is shown in greater detail in FIG. 18.

FIG. 17 shows 1H-NMR spectra for 100 mM methylene blue in D₂O with peak assignments.

FIG. 18 shows 1H-NMR spectra for 100 mM methylene blue with 1 M nicotinamide in D₂O with peak assignments. Peaks B′, C′, and D′ broadening in the presence of nicotinamide. Note, the peaks at 7.32 ppm are from nicotinamide and are shown in greater detail in FIG. 16.

FIGS. 19A-C shows deconvoluted mass spectra for batch C7 protein conjugated to sulfo-DBCO-maleimide and azide-quinone-dTTP. The protein was split into batches and treated with either leucomethylene blue or methylene blue. FIG. 19A shows deconvoluted mass spectra of the conjugated C7 variant. FIG. 19B shows a C7-protein conjugated to sulfo-DBCO-maleimide/azide-quinone-dTTP and treated with leucomethylene blue. FIG. 19C shows C7-protein conjugated to sulfo-DBCO-maleimide/azide-quinone-dTTP and treated with methylene blue.

FIG. 20 shows use of soluble redox shuttle to cleave the linker from the complexed DNA-enzyme complex. The redox mediator (soluble redox shuttle) is introduced into a system in one particular oxidation state, in this case, at a higher oxidation state. Applying a potential at the electrodes reduces the mediator. The reduced mediator interacts with the redox-cleavable linker (a quinone-oxime-ether is shown), cleaving the enzyme from the ssDNA. The newly attached nucleotide is left at the end of the ssDNA.

FIG. 21 shows a tris-glycine gel indicating binding of ssDNA to protein conjugates. The fluorescent oligonucleotide shifts upward on gel upon covalent protein binding due to increase in molecular weight.

FIG. 22 shows a representation of experiment to demonstrate addition of nucleotides to an oligonucleotide using the TdT-nucleotide conjugates. A surface is functionalized with a capture oligo (Step 1), followed by hybridization of a seed oligo with an accessible 3′ end (Step 2), Conjugated TdT-dCTP is introduced and covalently binds the seed oligo via addition of its tethered nucleotide (Step 3). Unbound enzyme is then washed away (Step 4) and a soluble reducing agent (leucomethylene blue) is introduced to cleave the enzyme from the extended DNA (Step 5). The cleaved enzyme is then washed away, leaving the seed on the surface with a single +C extension on its 3′ end (Step 6). TdT conjugated to the next nucleotide to be added (here, TdT-dATP) is then introduced, resulting in a total addition of “CA” to the seed oligonucleotide.

FIGS. 23A-B show a distribution of synthesized sequences from a 3-cycle, electrochemically controlled synthesis run. FIG. 23A shows a schematic of the 3-cycle synthesis workflow adding “GCT.” FIG. 23B shows average fraction target sequences, sequences with insertions, and sequences with deletions is shown with error bars representing the standard deviation across three replicates. 37% target sequence of “GCT” was achieved.

FIG. 24 shows a 1H NMR spectrum of

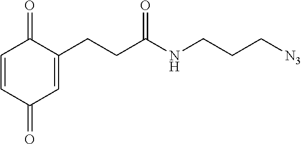 [SMILES: [N-]=[N+]=NCCCNC(=O)CCC1=CC(=O)C=CC1=O]

### (400 MHz, DMSO-d6).

FIGS. 25A-C. FIG. 25A shows an HPLC chromatogram of the following compound I:

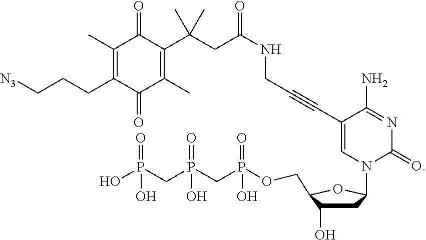

FIG. 25B shows an HPLC chromatogram of compound II (a propargyl-amine substituted nucleotide analog) (expected cleavage product). FIG. 25C shows an HPLC chromatogram of a crude reaction mixture when compound I was treated with leucomethylene blue (reducing agent).

FIG. 26 shows ESI-MS (negative mode) data for Boc-NH2O-PA-dGTP analog 5:

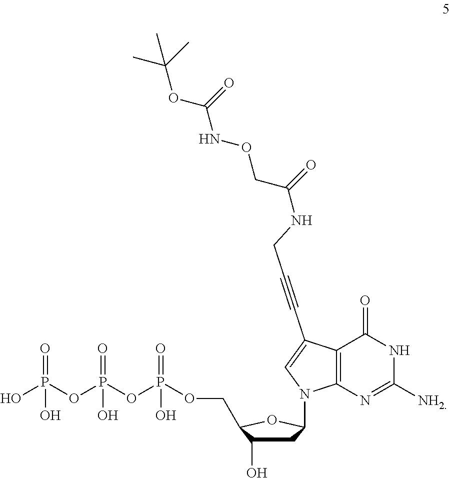

FIG. 27 shows ¹ESI-MS (negative mode) for NH2O-PA-dGTP analog 6:

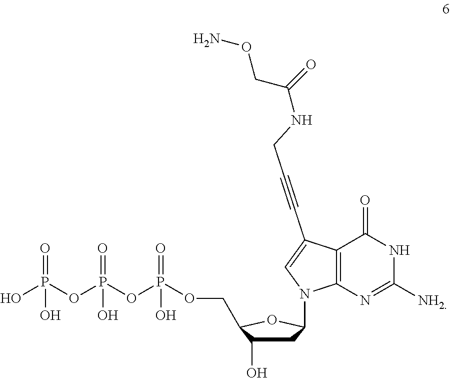

FIG. 28 shows ESI-MS (negative mode) data for the azide-quinone-dATP analog 21:

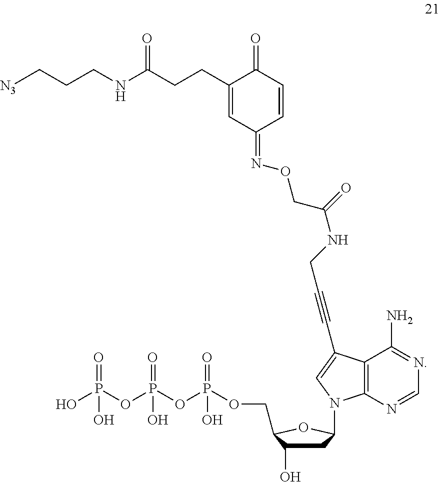

FIG. 29 shows ESI-MS (negative mode) data for the azide-quinone-dGTP analog 8:

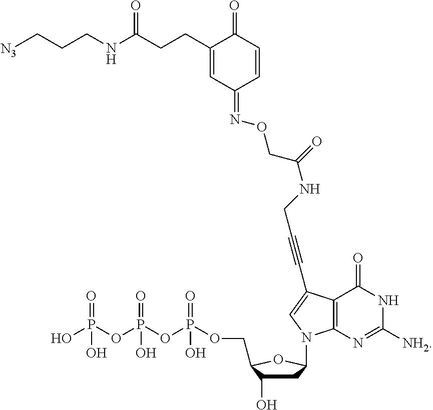

FIG. 30 shows ESI-MS (negative mode) data for the azide-quinone-dCTP compound 20:

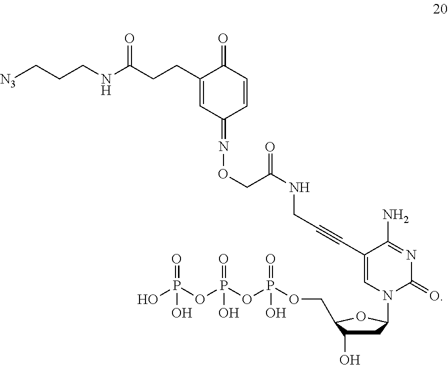

FIG. 31 shows ESI-MS (negative mode) data for the azide-quinone-dTTP analog 22:

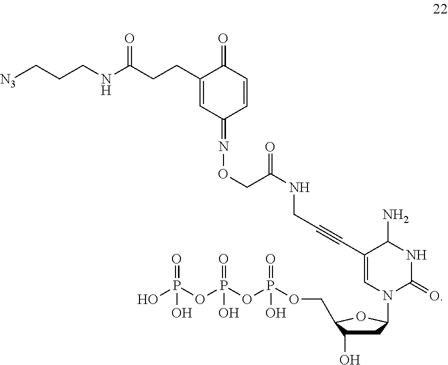

### DESCRIPTION OF THE SEQUENCES

TABLE 1
Table 1 provides a listing of certain sequences referenced herein.
Description of the Sequences

| | Parent | | | |
|---|---|---|---|---|
| | of the | | | |
| | En- | | | |
| | gineered | | | SEQ |
| Construct | Variant | Mutation | Sequence | ID NO |
| wt | mTdT | wt | KISQYACQRRTTLNNYNQLFTDALDILAENDELREN | 1 |
| | | | EGSCLAFMRASSVLKSLPFPITSMKDTEGIPCLGDKV | |
| | | | KSIIEGIIEDGESSEAKAVLNDERYKSFKLFTSVFGVG | |
| | | | LKTAEKWFRMGFRTLSKIQSDKSLRFTQMQKAGFL | |
| | | | YYEDLVSCVNRPEAEAVSMLVKEAVVTFLPDALVT | |
| | | | MTGGFRRGKMTGHDVDFLITSPEATEDEEQQLLHK | |
| | | | VTDFWKQQGLLLYCDILESTFEKFKQPSRKVDALDH | |
| | | | FQKCFLILKLDHGRVHSEKSGQQEGKGWKAIRVDL | |
| | | | VMCPYDRRAFALLGWTGSRQFERDLRRYATHERK | |
| | | | MMLDNHALYDRTKRVFLEAESEEEIFAHLGLDYIEP | |
| | | | WERNA | |
| P1 | Pross1 | Kettner | KISQYAAQRRTTLNNYNKKFTDALDILAENAELRGN | 2 |
| | | stabilized | EGSALAFRRAASVLKSLPFPITSMKDTEGIPNLGDKV | |
| | | | KRIIQEILEDGESSEAKAVLNDERYQAFKLFTSVFGV | |
| | | | GPKTAEKWYRMGFRTLEDIRSDKTLRFTRMQKAGF | |
| | | | LYYEDLVSPVTRPEAEAIEQLVKEAVWQFLPGATVT | |
| | | | MTGGFRRGKKTGHDVDFLITSPEATEEEEKQLLHKV | |
| | | | TDWWKKQGLLLYEDIQESTFEKFKLPSRKVDALDH | |
| | | | FQKAFLILKLHHQRVDSGKSGQQEGKGWKAIRVDL | |
| | | | VMAPYERRAFALLGWTGSRQFERDLRRYARHERK | |
| | | | MLLDNHALYDRTKNTFLRAESEEEIFAHLGLEYIEP | |
| | | | WERNA | |
| P2 | Pross2 | loop | KISQYAAQRRTTLNNYNKKFTDALDILAENAELRGN | 3 |
| | | redesign | EGSALAFRRAASVLKSLPFPITSMKDTEGIPNLGDKV | |
| | | | KRIIQEILEDGESSEAKAVLNDERYQAFKLFTSVFGV | |
| | | | GPKTAEKWYRMGFRTLEDIRSDKTLRFTRMQKAGF | |
| | | | LYYEDLVSPVTRPEAEAIEQLVKEAVWQFLPGATVT | |
| | | | MTGGFRRGKKTGHDVDFLITSPEATEEBEKQLLHKV | |
| | | | TDWWKKQGLLLYEDIQESTFEKFKLPSRKVDALDH | |
| | | | FQKAFLILKLGPSPGKAIRVDLVMAPYERRAFALLG | |
| | | | WTGSRQFERDLRRYARHERKMLLDNHALYDRTKN | |
| | | | TFLRAESEEEIFAHLGLEYIEPWERNA | |
| N1 | Pross2 | E32nc | KISQYAAQRRTTLNNYNKKFTDALDILAENAXLRG | 4 |
| | | (wherein X | NEGSALAFRRAASVLKSLPFPITSMKDTEGIPNLGDK | |
| | | stands for | VKRIIQEILEDGESSEAKAVLNDERYQAFKLFTSVFG | |
| | | a non- | VGPKTAEKWYRMGFRTLEDIRSDKTLRFTRMQKAG | |
| | | canonical | FLYYEDLVSPVTRPEAEAIEQLVKEAVWQFLPGATV | |
| | | AA) | TMTGGFRRGKKTGHDVDFLITSPEATEEEEKQLLHK | |
| | | | VTDWWKKQGLLLYEDIQESTFEKFKLPSRKVDALD | |
| | | | HFQKAFLILKLGPSPGKAIRVDLVMAPYERRAFALL | |
| | | | GWTGSRQFERDLRRYARHERKMLLDNHALYDRTK | |
| | | | NTFLRAESEEEIFAHLGLEYIEPWERNA | |
| N2 | Pross2 | V152nc | KISQYAAQRRTTLNNYNKKFTDALDILAENAELRGN | 5 |
| | | (wherein X | EGSALAFRRAASVLKSLPFPITSMKDTEGIPNLGDKV | |
| | | stands for | KRIIQEILEDGESSEAKAVLNDERYQAFKLFTSVFGV | |
| | | a non- | GPKTAEKWYRMGFRTLEDIRSDKTLRFTRMQKAGF | |
| | | canonical | LYYEDLXSPVTRPEAEAIEQLVKEAVWQFLPGATVT | |
| | | AA) | MTGGFRRGKKTGHDVDFLITSPEATEEEEKQLLHKV | |
| | | | TDWWKKQGLLLYEDIQESTFEKFKLPSRKVDALDH | |
| | | | FQKAFLILKLGPSPGKAIRVDLVMAPYERRAFALLG | |
| | | | WTGSRQFERDLRRYARHERKMLLDNHALYDRTKN | |
| | | | TFLRAESEEEIFAHLGLEYIEPWERNA | |
| N3 | Pross2 | P154nc | KISQYAAQRRTTLNNYNKKFTDALDILAENAELRGN | 6 |
| | | (wherein X | EGSALAFRRAASVLKSLPFPITSMKDTEGIPNLGDKV | |
| | | stands for | KRIIQEILEDGESSEAKAVLNDERYQAFKLFTSVFGV | |
| | | a non- | GPKTAEKWYRMGFRTLEDIRSDKTLRFTRMQKAGF | |
| | | canonical | LYYEDLVSXVTRPEAEAIEQLVKEAVWQFLPGATV | |
| | | AA) | TMTGGFRRGKKTGHDVDFLITSPEATEEEEKQLLHK | |
| | | | VTDWWKKQGLLLYEDIQESTFEKFKLPSRKVDALD | |
| | | | HFQKAFLILKLGPSPGKAIRVDLVMAPYERRAFALL | |
| | | | GWTGSRQFERDLRRYARHERKMLLDNHALYDRTK | |
| | | | NTFLRAESEEEIFAHLGLEYIEPWERNA | |
| C1 | Pross2 | E32C | KISQYAAQRRTTLNNYNKKFTDALDILAENACLRG | 7 |
| | | | NEGSALAFRRAASVLKSLPFPITSMKDTEGIPNLGDK | |
| | | | VKRIIQEILEDGESSEAKAVLNDERYQAFKLFTSVFG | |
| | | | VGPKTAEKWYRMGFRTLEDIRSDKTLRFTRMQKAG | |
| | | | FLYYEDLVSPVTRPEAEAIEQLVKEAVWQFLPGATV | |
| | | | TMTGGFRRGKKTGHDVDFLITSPEATEEEEKQLLHK | |
| | | | VTDWWKKQGLLLYEDIQESTFEKFKLPSRKVDALD | |
| | | | HFQKAFLILKLGPSPGKAIRVDLVMAPYERRAFALL | |
| | | | GWTGSRQFERDLRRYARHERKMLLDNHALYDRTK | |
| | | | NTFLRAESEEEIFAHLGLEYIEPWERNA | |
| C7 | Pross1 | K102C | KISQYAAQRRTTLNNYNKKFTDALDILAENAELRGN | 8 |
| | | | EGSALAFRRAASVLKSLPFPITSMKDTEGIPNLGDKV | |
| | | | KRIIQEILEDGESSEAKAVLNDERYQAF{*}C{*}LFTS | |
| | | | VFGVGPKTAEKWYRMGFRTLEDIRSDKTLRFTRMQ | |
| | | | KAGFLYYEDLVSPVTRPEAEAIEQLVKEAVWQFLPG | |
| | | | ATVTMTGGFRRGKKTGHDVDFLITSPEATEEEEKQL | |
| | | | LHKVTDWWKKQGLLLYEDIQESTFEKFKLPSRKVD | |
| | | | ALDHFQKAFLILKLHHQRVDSGKSGQQEGKGWKAI | |
| | | | RVDLVMAPYERRAFALLGWTGSRQFERDLRRYAR | |
| | | | HERKMLLDNHALYDRTKNTFLRAESEEEIFAHLGLE | |
| | | | YIEPWERN | |
| C2 | Pross2 | V152C | KISQYAAQRRTTLNNYNKKFTDALDILAENAELRGN | 9 |
| | | | EGSALAFRRAASVLKSLPFPITSMKDTEGIPNLGDKV | |
| | | | KRIIQEILEDGESSEAKAVLNDERYQAFKLFTSVFGV | |
| | | | GPKTAEKWYRMGFRTLEDIRSDKTLRFTRMQKAGF | |
| | | | LYYEDLCSPVTRPEAEAIEQLVKEAVWQFLPGATVT | |
| | | | MTGGFRRGKKTGHDVDFLITSPEATEEEEKQLLHKV | |
| | | | TDWWKKQGLLLYEDIQESTFEKFKLPSRKVDALDH | |
| | | | FQKAFLILKLGPSPGKAIRVDLVMAPYERRAFALLG | |
| | | | WTGSRQFERDLRRYARHERKMLLDNHALYDRTKN | |
| | | | TFLRAESEEEIFAHLGLEYIEPWERNA | |
| C3 | Pross2 | P154C | KISQYAAQRRTTLNNYNKKFTDALDILAENAELRGN | 10 |
| | | | EGSALAFRRAASVLKSLPFPITSMKDTEGIPNLGDKV | |
| | | | KRIIQEILEDGESSEAKAVLNDERYQAFKLFTSVFGV | |
| | | | GPKTAEKWYRMGFRTLEDIRSDKTLRFTRMQKAGF | |
| | | | LYYEDLVSCVTRPEABAIEQLVKEAVWQFLPGATVT | |
| | | | MTGGFRRGKKTGHDVDFLITSPEATEEEEKQLLHKV | |
| | | | TDWWKKQGLLLYEDIQESTFEKFKLPSRKVDALDH | |
| | | | FQKAFLILKLGPSPGKAIRVDLVMAPYERRAFALLG | |
| | | | WTGSRQFERDLRRYARHERKMLLDNHALYDRTKN | |
| | | | TFLRAESEEEIFAHLGLEYIEPWERNA | |
| C9 | Pross1 | E32C | KISQYAAQRRTTLNNYNKKFTDALDILAENACLRG | 11 |
| | | | NEGSALAFRRAASVLKSLPFPITSMKDTEGIPNLGDK | |
| | | | VKRIIQEILEDGESSEAKAVLNDERYQAFKLFTSVFG | |
| | | | VGPKTAEKWYRMGFRTLEDIRSDKTLRFTRMQKAG | |
| | | | FLYYEDLVSPVTRPEAEAIEQLVKEAVWQFLPGATV | |
| | | | TMTGGFRRGKKTGHDVDFLITSPEATEEEEKQLLHK | |
| | | | VTDWWKKQGLLLYEDIQESTFEKFKLPSRKVDALD | |
| | | | HFQKAFLILKLHHQRVDSGKSGQQEGKGWKAIRVD | |
| | | | LVMAPYERRAFALLGWTGSRQFERDLRRYARHERK | |
| | | | MLLDNHALYDRTKNTFLRAESEEEIFAHLGLEYIEP | |
| | | | WERNA | |
| C4 | Pross1 | E29C | KISQYAAQRRTTLNNYNKKFTDALDILACNAELRG | 12 |
| | | | NEGSALAFRRAASVLKSLPFPITSMKDTEGIPNLGDK | |
| | | | VKRIIQEILEDGESSEAKAVLNDERYQAFKLFTSVFG | |
| | | | VGPKTAEKWYRMGFRTLEDIRSDKTLRFTRMQKAG | |
| | | | FLYYEDLVSPVTRPEAEAIEQLVKEAVWQFLPGATV | |
| | | | TMTGGFRRGKKTGHDVDFLITSPEATEEEEKQLLHK | |
| | | | VTDWWKKQGLLLYEDIQESTFEKFKLPSRKVDALD | |
| | | | HFQKAFLILKLHHQRVDSGKSGQQEGKGWKAIRVD | |
| | | | LVMAPYERRAFALLGWTGSRQFERDLRRYARHERK | |
| | | | MLLDNHALYDRTKNTFLRAESEEEIFAHLGLEYIEP | |
| | | | WERNA | |
| C5 | Pross1 | E37C | KISQYAAQRRTTLNNYNKKFTDALDILAENAELRGN | 13 |
| | | | CGSALAFRRAASVLKSLPFPITSMKDTEGIPNLGDKV | |
| | | | KRIIQEILEDGESSEAKAVLNDERYQAFKLFTSVFGV | |
| | | | GPKTAEKWYRMGFRTLEDIRSDKTLRFTRMQKAGF | |
| | | | LYYEDLVSPVTRPEAEAIEQLVKEAVWQFLPGATVT | |
| | | | MTGGFRRGKKTGHDVDFLITSPEATEEEEKQLLHKV | |
| | | | TDWWKKQGLLLYEDIQESTFEKFKLPSRKVDALDH | |
| | | | FQKAFLILKLHHQRVDSGKSGQQEGKGWKAIRVDL | |
| | | | VMAPYERRAFALLGWTGSRQFERDLRRYARHERK | |
| | | | MLLDNHALYDRTKNTFLRAESEEEIFAHLGLEYIEP | |
| | | | WERNA | |
| C7 | Pross1 | K102C | KISQYAAQRRTTLNNYNKKFTDALDILAENAELRGN | 15 |
| | | | EGSALAFRRAASVLKSLPFPITSMKDTEGIPNLGDKV | |
| | | | KRIIQEILEDGESSEAKAVLNDERYQAFCLFTSVFGV | |
| | | | GPKTAEKWYRMGFRTLEDIRSDKTLRFTRMQKAGF | |
| | | | LYYEDLVSPVTRPEAEAIEQLVKEAVWQFLPGATVT | |
| | | | MTGGFRRGKKTGHDVDFLITSPEATEEEEKQLLHKV | |
| | | | TDWWKKQGLLLYEDIQESTFEKFKLPSRKVDALDH | |
| | | | FQKAFLILKLHHQRVDSGKSGQQEGKGWKAIRVDL | |
| | | | VMAPYERRAFALLGWTGSRQFERDLRRYARHERK | |
| | | | MLLDNHALYDRTKNTFLRAESEEEIFAHLGLEYIEP | |
| | | | WERNA | |
| C8 | Pross2 | E29C | KISQYAAQRRTTLNNYNKKFTDALDILACNAELRG | 16 |
| | | | NEGSALAFRRAASVLKSLPFPITSMKDTEGIPNLGDK | |
| | | | VKRIIQEILEDGESSEAKAVLNDERYQAFKLFTSVFG | |
| | | | VGPKTAEKWYRMGFRTLEDIRSDKTLRFTRMQKAG | |
| | | | FLYYEDLVSPVTRPEAEAIEQLVKEAVWQFLPGATV | |
| | | | TMTGGFRRGKKTGHDVDFLITSPEATEEEEKQLLHK | |
| | | | VTDWWKKQGLLLYEDIQESTFEKFKLPSRKVDALD | |
| | | | HFQKAFLILKLGPSPGKAIRVDLVMAPYERRAFALL | |
| | | | GWTGSRQFERDLRRYARHERKMLLDNHALYDRTK | |
| | | | NTFLRAESEEEIFAHLGLEYIEPWERNA | |
| C10 | Pross2 | E37C | KISQYAAQRRTTLNNYNKKFTDALDILAENAELRGN | 17 |
| | | | CGSALAFRRAASVLKSLPFPITSMKDTEGIPNLGDKV | |
| | | | KRIIQEILEDGESSEAKAVLNDERYQAFKLFTSVFGV | |
| | | | GPKTAEKWYRMGFRTLEDIRSDKTLRFTRMQKAGF | |
| | | | LYYEDLVSPVTRPEAEAIEQLVKEAVWQFLPGATVT | |
| | | | MTGGFRRGKKTGHDVDFLITSPEATEEEEKQLLHKV | |
| | | | TDWWKKQGLLLYEDIQESTFEKFKLPSRKVDALDH | |
| | | | FQKAFLILKLGPSPGKAIRVDLVMAPYERRAFALLG | |
| | | | WTGSRQFERDLRRYARHERKMLLDNHALYDRTKN | |
| | | | TFLRAESEEEIFAHLGLEYIEPWERNA | |
| C11 | Pross2 | R44C | KISQYAAQRRTTLNNYNKKFTDALDILAENAELRGN | 18 |
| | | | EGSALAFCRAASVLKSLPFPITSMKDTEGIPNLGDKV | |
| | | | KRIIQEILEDGESSEAKAVLNDERYQAFKLFTSVFGV | |
| | | | GPKTAEKWYRMGFRTLEDIRSDKTLRFTRMQKAGF | |
| | | | LYYEDLVSPVTRPEAEAIEQLVKEAVWQFLPGATVT | |
| | | | MTGGFRRGKKTGHDVDFLITSPEATEEEEKQLLHKV | |
| | | | TDWWKKQGLLLYEDIQESTFEKFKLPSRKVDALDH | |
| | | | FQKAFLILKLGPSPGKAIRVDLVMAPYERRAFALLG | |
| | | | WTGSRQFERDLRRYARHERKMLLDNHALYDRTKN | |
| | | | TFLRAESEEEIFAHLGLEYIEPWERNA | |
| C12 | Pross2 | K102C | KISQYAAQRRTTLNNYNKKFTDALDILAENAELRGN | 19 |
| | | | EGSALAFRRAASVLKSLPFPITSMKDTEGIPNLGDKV | |
| | | | KRIIQEILEDGESSEAKAVLNDERYQAFCLFTSVFGV | |
| | | | GPKTAEKWYRMGFRTLEDIRSDKTLRFTRMQKAGF | |
| | | | LYYEDLVSPVTRPEAEAIEQLVKEAVWQFLPGATVT | |
| | | | MTGGFRRGKKTGHDVDFLITSPEATEEEEKQLLHKV | |
| | | | TDWWKKQGLLLYEDIQESTFEKFKLPSRKVDALDH | |
| | | | FQKAFLILKLGPSPGKAIRVDLVMAPYERRAFALLG | |
| | | | WTGSRQFERDLRRYARHERKMLLDNHALYDRTKN | |
| | | | TFLRAESEEEIFAHLGLEYIEPWERNA | |
| P1-GFP, | Pross1 | Kettner | MGSKGEELFTGVVPILVELDGDVNGHKFSVSGEG | 20 |
| His Tag | | stabilized | EGDATYGKLTLKFICTTGKLPVPWPTLVTTFSYG | |
| GFP in | | | VQCFSRYPDHMKRHDFFKSAMPEGYVQERTIFF | |
| bold and | | | KDDGNYKTRAEVKFEGDTLVNRIELKGIDFKEDG | |
| His-Tag | | | NILGHKLEYNYNSHNVYIMADKQKNGIKVNFKIR | |
| in italic | | | HNIEDGSVQLADHYQQNTPIGDGPVLLPDNHYLS | |
| | | | TQSALSKDPNEKRDHMVLLEFVTAAGITHGMDE | |
| | | | LYKGSSHHHHHHHHHHGSGKISQYAAQRRTTLNNY | |
| | | | NKKFTDALDILAENAELRGNEGSALAFRRAASVLKS | |
| | | | LPFPITSMKDTEGIPNLGDKVKRIIQEILEDGESSEAK | |
| | | | AVLNDERYQAFKLFTSVFGVGPKTAEKWYRMGFRT | |
| | | | LEDIRSDKTLRFTRMQKAGFLYYEDLVSPVTRPEAE | |
| | | | AIEQLVKEAVWQFLPGATVTMTGGFRRGKKTGHD | |
| | | | VDFLITSPEATEEEEKQLLHKVTDWWKKQGLLLYE | |
| | | | DIQESTFEKFKLPSRKVDALDHFQKAFLILKLHHQR | |
| | | | VDSGKSGQQEGKGWKAIRVDLVMAPYERRAFALL | |
| | | | GWTGSRQFERDLRRYARHERKMLLDNHALYDRTK | |
| | | | NTFLRAESEEEIFAHLGLEYIEPWERNA | |
| C9-GFP, | Pross1 | E32C | MGSKGEELFTGVVPILVELDGDVNGHKFSVSGEG | 21 |
| His-Tag | | | EGDATYGKLTLKFICTTGKLPVPWPTLVTTFSYG | |
| GFP in | | | VQCFSRYPDHMKRHDFFKSAMPEGYVQERTIFF | |
| bold and | | | KDDGNYKTRAEVKFEGDTLVNRIELKGIDFKEDG | |
| His-Tag | | | NILGHKLEYNYNSHNVYIMADKQKNGIKVNFKIR | |
| in italic | | | HNIEDGSVQLADHYQQNTPIGDGPVLLPDNHYLS | |
| | | | TQSALSKDPNEKRDHMVLLEFVTAAGITHGMDE | |
| | | | LYKGSSHHHHHHHHHHGSGKISQYAAQRRTTLNNY | |
| | | | NKKFTDALDILAENACLRGNEGSALAFRRAASVLKS | |
| | | | LPFPITSMKDTEGIPNLGDKVKRIIQEILEDGESSEAK | |
| | | | AVLNDERYQAFKLFTSVFGVGPKTAEKWYRMGFRT | |
| | | | LEDIRSDKTLRFTRMQKAGFLYYEDLVSPVTRPEAE | |
| | | | AIEQLVKEAVWQFLPGATVTMTGGFRRGKKTGHD | |
| | | | VDFLITSPEATEEEEKQLLHKVTDWWKKQGLLLYE | |
| | | | DIQESTFEKFKLPSRKVDALDHFQKAFLILKLHHQR | |
| | | | VDSGKSGQQEGKGWKAIRVDLVMAPYERRAFALL | |
| | | | GWTGSRQFERDLRRYARHERKMLLDNHALYDRTK | |
| | | | NTFLRAESEEEIFAHLGLEYIEPWERNA | |
| C4-GFP, | Pross1 | E29C | MGSKGEELFTGVVPILVELDGDVNGHKFSVSGEG | 22 |
| His-Tag | | | EGDATYGKLTLKFICTTGKLPVPWPTLVTTFSYG | |
| GFP in | | | VQCFSRYPDHMKRHDFFKSAMPEGYVQERTIFF | |
| bold and | | | KDDGNYKTRAEVKFEGDTLVNRIELKGIDFKEDG | |
| His-Tag | | | NILGHKLEYNYNSHNVYIMADKQKNGIKVNFKIR | |
| in italic | | | HNIEDGSVQLADHYQQNTPIGDGPVLLPDNHYLS | |
| | | | TQSALSKDPNEKRDHMVLLEFVTAAGITHGMDE | |
| | | | LYKGSSHHHHHHHHHHGSGKISQYAAQRRTTLNNY | |
| | | | NKKFTDALDILACNAELRGNEGSALAFRRAASVLKS | |
| | | | LPFPITSMKDTEGIPNLGDKVKRIIQEILEDGESSEAK | |
| | | | AVLNDERYQAFKLFTSVFGVGPKTAEKWYRMGFRT | |
| | | | LEDIRSDKTLRFTRMQKAGFLYYEDLVSPVTRPEAE | |
| | | | AIEQLVKEAVWQFLPGATVTMTGGFRRGKKTGHD | |
| | | | VDFLITSPEATEEEEKQLLHKVTDWWKKQGLLLYE | |
| | | | DIQESTFEKFKLPSRKVDALDHFQKAFLILKLHHQR | |
| | | | VDSGKSGQQEGKGWKAIRVDLVMAPYERRAFALL | |
| | | | GWTGSRQFERDLRRYARHERKMLLDNHALYDRTK | |
| | | | NTFLRAESEEEIFAHLGLEYIEPWERNA | |
| C5-GFP, | Pross1 | E37C | MGSKGEELFTGVVPILVELDGDVNGHKFSVSGEG | 23 |
| His-Tag | | | EGDATYGKLTLKFICTTGKLPVPWPTLVTTFSYG | |
| GFP in | | | VQCFSRYPDHMKRHDFFKSAMPEGYVQERTIFF | |
| bold and | | | KDDGNYKTRAEVKFEGDTLVNRIELKGIDFKEDG | |
| His-Tag | | | NILGHKLEYNYNSHNVYIMADKQKNGIKVNFKIR | |
| in italic | | | HNIEDGSVQLADHYQQNTPIGDGPVLLPDNHYLS | |
| | | | TQSALSKDPNEKRDHMVLLEFVTAAGITHGMDE | |
| | | | LYKGSSHHHHHHHHHHGSGKISQYAAQRRTTLNNY | |
| | | | NKKFTDALDILAENAELRGNCGSALAFRRAASVLKS | |
| | | | LPFPITSMKDTEGIPNLGDKVKRIIQEILEDGESSEAK | |
| | | | AVLNDERYQAFKLFTSVFGVGPKTAEKWYRMGFRT | |
| | | | LEDIRSDKTLRFTRMQKAGFLYYEDLVSPVTRPEAE | |
| | | | AIEQLVKEAVWQFLPGATVTMTGGFRRGKKTGHD | |
| | | | VDFLITSPEATEEEEKQLLHKVTDWWKKQGLLLYE | |
| | | | DIQESTFEKFKLPSRKVDALDHFQKAFLILKLHHQR | |
| | | | VDSGKSGQQEGKGWKAIRVDLVMAPYERRAFALL | |
| | | | GWTGSRQFERDLRRYARHERKMLLDNHALYDRTK | |
| | | | NTFLRAESEEEIFAHLGLEYIEPWERNA | |
| C7-GFP, | Pross1 | K102C | MGSKGEELFTGVVPILVELDGDVNGHKFSVSGEG | 24 |
| His-Tag | | | EGDATYGKLTLKFICTTGKLPVPWPTLVTTFSYG | |
| GFP in | | | VQCFSRYPDHMKRHDFFKSAMPEGYVQERTIFF | |
| bold and | | | KDDGNYKTRAEVKFEGDTLVNRIELKGIDFKEDG | |
| His-Tag | | | NILGHKLEYNYNSHNVYIMADKQKNGIKVNFKIR | |
| in italic | | | HNIEDGSVQLADHYQQNTPIGDGPVLLPDNHYLS | |
| | | | TQSALSKDPNEKRDHMVLLEFVTAAGITHGMDE | |
| | | | LYKGSSHHHHHHHHHHGSGKISQYAAQRRTTLNNY | |
| | | | NKKFTDALDILAENAELRGNEGSALAFRRAASVLKS | |
| | | | LPFPITSMKDTEGIPNLGDKVKRIIQEILEDGESSEAK | |
| | | | AVLNDERYQAFCLFTSVFGVGPKTAEKWYRMGFRT | |
| | | | LEDIRSDKTLRFTRMQKAGFLYYEDLVSPVTRPEAE | |
| | | | AIEQLVKEAVWQFLPGATVTMTGGFRRGKKTGHD | |
| | | | VDFLITSPEATEEEEKQLLHKVTDWWKKQGLLLYE | |
| | | | DIQESTFEKFKLPSRKVDALDHFQKAFLILKLHHQR | |
| | | | VDSGKSGQQEGKGWKAIRVDLVMAPYERRAFALL | |
| | | | GWTGSRQFERDLRRYARHERKMLLDNHALYDRTK | |
| | | | NTFLRAESEEEIFAHLGLEYIEPWERNA | |
| C13 | Prossl | D22C | KISQYAAQRRTTLNNYNKKFTCALDILAENAELRGN | 25 |
| | | | EGSALAFRRAASVLKSLPFPITSMKDTEGIPNLGDKV | |
| | | | KRIIQEILEDGESSEAKAVLNDERYQAFKLFTSVFGV | |
| | | | GPKTAEKWYRMGFRTLEDIRSDKTLRFTRMQKAGF | |
| | | | LYYEDLVSPVTRPEAEAIEQLVKEAVWQFLPGATVT | |
| | | | MTGGFRRGKKTGHDVDFLITSPEATEEEEKQLLHKV | |
| | | | TDWWKKQGLLLYEDIQESTFEKFKLPSRKVDALDH | |
| | | | FQKAFLILKLHHQRVDSGKSGQQEGKGWKAIRVDL | |
| | | | VMAPYERRAFALLGWTGSRQFERDLRRYARHERK | |
| | | | MLLDNHALYDRTKNTFLRAESEEEIFAHLGLEYIEP | |
| | | | WERNA | |
| C14 | Pross1 | R44C | KISQYAAQRRTTLNNYNKKFTDALDILAENAELRGN | 26 |
| | | | EGSALAFCRAASVLKSLPFPITSMKDTEGIPNLGDKV | |
| | | | KRIIQEILEDGESSEAKAVLNDERYQAFKLFTSVFGV | |
| | | | GPKTAEKWYRMGFRTLEDIRSDKTLRFTRMQKAGF | |
| | | | LYYEDLVSPVTRPEAEAIEQLVKEAVWQFLPGATVT | |
| | | | MTGGFRRGKKTGHDVDFLITSPEATEEEEKQLLHKV | |
| | | | TDWWKKQGLLLYEDIQESTFEKFKLPSRKVDALDH | |
| | | | FQKAFLILKLHHQRVDSGKSGQQEGKGWKAIRVDL | |
| | | | VMAPYERRAFALLGWTGSRQFERDLRRYARHERK | |
| | | | MLLDNHALYDRTKNTFLRAESEEEIFAHLGLEYIEP | |
| | | | WERNA | |
| C15 | Pross1 | L93C | KISQYAAQRRTTLNNYNKKFTDALDILAENAELRGN | 27 |
| | | | EGSALAFRRAASVLKSLPFPITSMKDTEGIPNLGDKV | |
| | | | KRIIQEILEDGESSEAKAVCNDERYQAFKLFTSVFGV | |
| | | | GPKTAEKWYRMGFRTLEDIRSDKTLRFTRMQKAGF | |
| | | | LYYEDLVSPVTRPEAEAIEQLVKEAVWQFLPGATVT | |
| | | | MTGGFRRGKKTGHDVDFLITSPEATEEEEKQLLHKV | |
| | | | TDWWKKQGLLLYEDIQESTFEKFKLPSRKVDALDH | |
| | | | FQKAFLILKLHHQRVDSGKSGQQEGKGWKAIRVDL | |
| | | | VMAPYERRAFALLGWTGSRQFERDLRRYARHERK | |
| | | | MLLDNHALYDRTKNTFLRAESEEEIFAHLGLEYIEP | |
| | | | WERNA | |
| C17 | Prossl | P154C | KISQYAAQRRTTLNNYNKKFTDALDILAENAELRGN | 28 |
| | | | EGSALAFRRAASVLKSLPFPITSMKDTEGIPNLGDKV | |
| | | | KRIIQEILEDGESSEAKAVLNDERYQAFKLFTSVFGV | |
| | | | GPKTAEKWYRMGFRTLEDIRSDKTLRFTRMQKAGF | |
| | | | LYYEDLVSCVTRPEAEAIEQLVKEAVWQFLPGATVT | |
| | | | MTGGFRRGKKTGHDVDFLITSPEATEEEEKQLLHKV | |
| | | | TDWWKKQGLLLYEDIQESTFEKFKLPSRKVDALDH | |
| | | | FQKAFLILKLHHQRVDSGKSGQQEGKGWKAIRVDL | |
| | | | VMAPYERRAFALLGWTGSRQFERDLRRYARHERK | |
| | | | MLLDNHALYDRTKNTFLRAESEEEIFAHLGLEYIEP | |
| | | | WERNA | |
| C18 | Pross 1 | N361C | KISQYAAQRRTTLNNYNKKFTDALDILAENAELRGN | 29 |
| | | | EGSALAFRRAASVLKSLPFPITSMKDTEGIPNLGDKV | |
| | | | KRIIQEILEDGESSEAKAVLNDERYQAFKLFTSVFGV | |
| | | | GPKTAEKWYRMGFRTLEDIRSDKTLRFTRMQKAGF | |
| | | | LYYEDLVSPVTRPEAEAIEQLVKEAVWQFLPGATVT | |
| | | | MTGGFRRGKKTGHDVDFLITSPEATEEEEKQLLHKV | |
| | | | TDWWKKQGLLLYEDIQESTFEKFKLPSRKVDALDH | |
| | | | FQKAFLILKLHHQRVDSGKSGQQEGKGWKAIRVDL | |
| | | | VMAPYERRAFALLGWTGSRQFERDLRRYARHERK | |
| | | | MLLDNHALYDRTKNTFLRAESEEEIFAHLGLEYIEP | |
| | | | WERCA | |

### DESCRIPTION OF THE EMBODIMENTS

### I. Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, such as for example, within 15%, 10%, or 5%.

The section headings used herein are for organizational purposes only and are not to be construed as limiting the subject matter described.

As used herein, “nucleotide molecule” refers to components of nucleic acids comprising a base, sugar, and phosphate moieties, both natural and unnatural, including nucleotides, ribonucleotides, and nucleotide analogs. As used herein, a nucleotide refers to a molecule comprising a nucleoside and one or more phosphate groups. As used herein, a nucleoside refers to a molecule comprising a nucleobase (e.g., adenine, thymine, cytosine, guanine, or uracil) and a five-carbon sugar (e.g., ribose or 2′-deoxyribose). Exemplary natural nucleotides include, without limitation, ATP, UTP, CTP, GTP, ADP, UDP, CDP, GDP, AMP, UMP, CMP, GMP, dATP, dTTP, dCTP, dGTP, dADP, dTDP, dCDP, dGDP, dAMP, dTMP, dCMP, and dGMP. Exemplary natural deoxyribonucleotides include dATP, dTTP, dCTP, dGTP, dADP, dTDP, dCDP, dGDP, dAMP, dTMP, dCMP, and dGMP. Exemplary natural ribonucleotides include ATP, UTP, CTP, GTP, ADP, UDP, CDP, GDP, AMP, UMP, CMP, and GMP. For natural RNA, the uracil base is uridine. A nucleotide analog, or unnatural nucleotide, comprises a nucleotide which contains some type of modification to either the base, sugar, or phosphate moieties, such as, for example, a chemical modification. All chemical structures showing protonated triphosphates and/or sulfonates should be interpreted to include the protonated and the ionized salt forms in different buffers.

As used herein, “affinity tag” refers to a protein tag that can aid in purifying or detecting the protein it is attached to. Affinity tags are typically inserted into a target gene at the point of coding for expression at either the N or C terminus of the protein to be expressed.

As used herein, “His-tag” or “polyhistidine tag” refers to a string of generally four, five, six, seven, eight, nine, ten, eleven, or twelve histidine residues, and in some cases, more. As used herein, “His-tag” may refer to either the DNA sequence encoding the string of histidine residues or the string of histidine residues itself. His-tags may be useful for facilitating easy purification and detection of recombinant proteins. His-tags are typically inserted into a target gene at the point of coding for expression at either the N or C terminus of the protein to be expressed.

As used herein, “GFP label” or “GFP tag” or green fluorescent protein label” or “green fluorescent protein tag” refers to a fluorescent protein from Aequorea victoria (water jellyfish). GFP labels can be N-terminally and/or C-terminally fused to a wide variety of proteins and are frequently used as a fluorescent marker. A non-limiting example of a GFP amino acid sequence can be found, for example, in UniProt Entry P42212⋅GFP_AEQVI. It emits a green fluorescent signal if exposed to light.

As used herein, “redox-cleavable linker” refers to a molecule that is cleaved in response to either oxidation or reduction conditions. Redox-cleavable linkers may also include additional groups that increase the solubility of the redox-cleavable linker, such as polar functional groups (e.g., alcohol, amine, amide, carboxylic acid, sulfonic acid, and phosphate groups).

As used herein, “conjugation residue” refers to an amino acid residue with a functional group capable of attaching or covalently linking to another molecule, such as a crosslinker or tether. Examples of conjugation residues include cysteine residues, lysine residues, arginine residues, or non-canonical amino acids residues.

As used herein, “spacer” refers to a molecule that may be used to link two other molecules, although spacers may be present by themselves or attached to only one other molecule. A spacer may be an organic spacer (e.g., an aliphatic spacer, an alkyl spacer, an aromatic spacer, an alkylene glycol, a polyethylene glycol, a carbohydrate such as a sugar, and the like). In some embodiments, the spacer is a PEG spacer, a well-known inert spacer used in many biotechnological applications.

As used herein, a “bifunctional crosslinker” or “crosslinker” or “bifunctional linker” refers to a molecule that has at least two functional groups and is capable of reacting with one or more other molecules to covalently link them. In some embodiments, the crosslinker has a functional group at each end. Examples of functional groups include, for example, maleimide groups and alkyne groups.

As used herein, a “tether” or “tether molecule” refers to the molecule covalently linking/connecting/attaching a TdT to a nucleotide molecule, comprising a redox-cleavable linker, optionally comprising a spacer, and optionally comprising a bifunctional crosslinker.

As used herein, a “soluble redox shuttle” refers to a molecule that is soluble, and when in a particular oxidation state, is capable of reacting with a redox-cleavable linker, facilitating cleavage of the redox-cleavable linker.

As used herein, a “redox shuttle solution” is a solution that comprises a soluble redox shuttle and other components/formulants/additives, such as a supporting electrolyte, a buffer, and/or a hydrotrope/hydrotropic agent.

The disclosure provides nucleic acid sequences and amino acid sequences having a certain degree of identity to a given nucleic acid sequence or amino acid sequence, respectively (a reference sequence). “Sequence identity” between first and second nucleic acid sequences indicates the percentage of nucleotides that are identical between the sequences; for example, if a first nucleic acid sequence is 95% identical to a second nucleic acid sequence, then the first nucleic acid sequence contains matches to 95% of the nucleotides in the second nucleic acid sequence. “Sequence identity” between two amino acid sequences indicates the percentage of amino acids that are identical between the sequences; for example, if a first amino acid sequence is 95% identical to a second amino acid sequence, then the first amino acid sequence contains matches to 95% of the nucleotides in the second amino acid sequence. The terms “% identical”, “% identity” or similar terms are intended to refer, in particular, to the percentage of nucleotides or amino acids which are determined to be identical using an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences after optimal alignment. The optimal alignment for a comparison may be carried out manually or with the aid of an appropriate algorithm such as the alignment algorithm by Needleman and Wunsch, 1970, J. Mol. Biol. 48, 443, or with the aid of computer programs using said algorithms (e.g., GAP, BESTFIT, and FASTA in Wisconsin Genetics Software Package, Genetics Computer Group, 575 Science Drive, Madison, Wis.).

As used herein, the terms “non-canonical amino acid” or “ncAA” or “non-canonical amino acid residue” or “nc” refer to an amino acid other than one of the 20 naturally occurring amino acids. Exemplary non-canonical amino acids are described in Young et al., “Beyond the canonical 20 amino acids: expanding the genetic lexicon,” J. of Biological Chemistry 285(15): 11039-11044 (2010), the disclosure of which is herein incorporated by reference.

As used herein, the terms “n” and “m” as they appear in chemical structures throughout the application refer to an integer from 1-12, unless defined otherwise.

### II. TdT Enzymes

Described herein are various biologically active, genetically engineered terminal deoxynucleotidyl transferases (TdT) comprising one or more mutations, wherein the one or more mutations are introduced into the TdT and wherein, in some embodiments, some of the mutation(s) confer increased thermal stability to the TdT relative to the stability of the wild-type TdT (SEQ ID NO: 1). In some embodiments, a different naturally occurring amino acid residue, or a non-canonical amino acid (ncAA) residue, is substituted for an amino acid residue in the wild-type TdT. As described herein, the genetically engineered TdT retains its enzymatic biological activity as a DNA polymerase to catalyze the addition of a nucleotide to a strand of nucleotides, thus extending a nucleotide strand in a controlled manner. Examples of TdTs with increased thermal stability are provided herein and are shown, for example, as sequences P1 and P2 in Table 1. The genetically engineered TdT enzymes are also referred to herein as TdT variants with increased thermal stability. In some embodiments, the engineered TdT is capable of adding a single nucleotide molecule to a single stranded DNA molecule in an electrochemically controlled manner/reaction.

In some embodiments, mutations to the wild-type TdT (SEQ ID NO: 1) are notated as, e.g., C7A, wherein the number represents the position/residue in the wild-type TdT amino acid sequence, the first letter (in this example, “C”) represents the amino acid residue at that position in the wild-type TdT amino acid sequence, and the second letter (in this example, “A”) represents the amino acid residue at that position in the mutant/engineered TdT.

In some embodiments, the engineered TdT comprises one or more mutations to a wild-type TdT of SEQ ID NO: 1, wherein: a) the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127B, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E, wherein position numbers are relative to SEQ ID NO: 1; or b) the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353E, wherein position numbers are relative to SEQ ID NO: 1; or c) the engineered TdT has at least 85, 90, 91, 92, 93, 94, 95, 96, 97, 98, 99, or 100% identity to SEQ ID NO: 2 or 3.

In some embodiments, the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E.

In some embodiments, the one or more mutations comprise 10, 15, 20, 25, 30, 35, 40, 45, 50, or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E.

In some embodiments, the one or more mutations comprise 50 or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E.

In some embodiments, the one or more mutations comprise all of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E.

In some embodiments, the one or more mutations consist of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E.

In some embodiments, the engineered TdT comprises an amino acid sequence with 85, 90, 91, 92, 93, 94, 95, 96, 97, 98, 99, or 100% identity to SEQ ID NO: 2. In some embodiments, the engineered TdT comprises the amino acid sequence of SEQ ID NO: 2. In some embodiments, the amino acid sequence of the engineered TdT consists of the amino acid sequence of SEQ ID NO: 2.

In some embodiments, the amino acid sequence of the engineered TdT consists of the amino acid sequence of SEQ ID NO: 2 and a fluorescent protein label, optionally a green fluorescent protein (GFP) label. In some embodiments, the amino acid sequence of the engineered TdT consists of the amino acid sequence of SEQ ID NO: 2 and an affinity tag, optionally a His-tag. In some embodiments, the engineered TdT further comprises a fluorescent protein label, optionally a GFP label. In some embodiments, the engineered TdT further comprises an affinity tag, optionally a His-tag.

In some embodiments, the engineered TdT comprises a conjugation mutation. In some embodiments, the conjugation mutation allows a cysteine residue, a non-canonical amino acid residue, a lysine residue, or an arginine residue, all comprising a reactive functional group, to be installed at the recited position to allow one skilled in the art to conjugate a bifunctional crosslinker or a redox-cleavable linker to the amino acid at that position. In some embodiments, the conjugation mutation replaces the amino acid present at the recited engineered TdT sequence. For example, in “E32C,” the amino acid at position 32 in the P1 sequence (SEQ ID NO: 2) is E, and the TdT further comprises the conjugation mutation comprises a “C” at position 32. In some embodiments, the recited engineered TdT sequence is the amino acid sequence of P1 (SEQ ID NO: 2). In some embodiments, the TdT further comprises the conjugation mutation E32C. In some embodiments, the TdT further comprises the conjugation mutation E29C. In some embodiments, the TdT further comprises the conjugation mutation E37C. In some embodiments, the TdT further comprises the conjugation mutation V152C. In some embodiments, the TdT further comprises the conjugation mutation K102C. In some embodiments, the TdT further comprises the conjugation mutation D22C. In some embodiments, the TdT further comprises the conjugation mutation R44C. In some embodiments, the TdT further comprises the conjugation mutation L93C. In some embodiments, the TdT further comprises the conjugation mutation P154C. In some embodiments, the TdT further comprises the conjugation mutation N361C.

In some embodiments, the engineered TdT comprises the amino acid sequence of any one of SEQ ID NOs: 11, 12, 13, and/or 15. In some embodiments, the engineered TdT consists of the amino acid sequence of any one of SEQ ID NOs: 11, 12, 13, and/or 15. In some embodiments, the engineered TdT comprises the amino acid sequence of SEQ ID NO: 15. In some embodiments, the engineered TdT consists of the amino acid sequence of SEQ ID NO: 15. In some embodiments, the TdT comprises the amino acid sequence of any one of SEQ ID NOs: 25, 26, 27, 28, and 29. In some embodiments, the TdT consists of the amino acid sequence of any one of SEQ ID NOs: 25, 26, 27, 28, and 29.

In some embodiments, an engineered TdT is provided comprising the amino acid sequence of any one of SEQ ID NOs: 20, 21, 22, 23, and 24.

In some embodiments, the engineered TdT comprises one or more more mutations to a wild-type TdT of SEQ ID NO: 1, wherein the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353E, wherein position numbers are relative to SEQ ID NO: 1.

In some embodiments, the one or more mutations comprise 10, 15, 20, 25, 30, 35, 40, 45, 50, or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353E, wherein position numbers are relative to SEQ ID NO: 1.

In some embodiments, the one or more mutations comprise 50 or more of the following mutations: C7A, Q18K, L19K, D31A, E35G, C40A, M44R, S47A, C68N, S75R, E78Q, G79E, I81L, K99Q, $100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R. M323L, R335N, V336T, E339R, D353E, wherein position numbers are relative to SEQ ID NO: 1.

In some embodiments, the one or more mutations comprise the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353E, wherein position numbers are relative to SEQ ID NO: 1.

In some embodiments, the one or more mutations consist of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353E, wherein position numbers are relative to SEQ ID NO: 1.

In some embodiments, the engineered TdT comprises an amino acid sequence with 85, 90, 91, 92, 93, 94, 95, 96, 97, 98, 99, or 100% identity to SEQ ID NO: 3. In some embodiments, the engineered TdT comprises the amino acid sequence of SEQ ID NO: 3. In some embodiments, the amino acid sequence of the engineered TdT consists of the amino acid sequence of SEQ ID NO: 3.

In some embodiments, the amino acid sequence of the engineered TdT consists of the amino acid sequence of SEQ ID NO: 3 and a fluorescent protein label, optionally a GFP label. In some embodiments, the amino acid sequence of the engineered TdT consists of the amino acid sequence of SEQ ID NO: 3 and an affinity tag, optionally a His-tag. In some embodiments, the engineered TdT further comprises a fluorescent protein label, optionally a green fluorescent protein (GFP) label. In some embodiments, the engineered TdT further comprises an affinity tag, optionally a His-tag.

In some embodiments, the engineered TdT comprises a conjugation mutation. In some embodiments, the conjugation mutation allows a cysteine residue, a non-canonical amino acid residue, a lysine residue, or an arginine residue, all comprising a reactive functional group, to be installed at the recited position to allow one skilled in the art to conjugate a bifunctional crosslinker or a redox-cleavable linker to the amino acid at that position. In some embodiments, the conjugation mutation replaces the amino acid present at the recited engineered TdT sequence. For example, in “V152C,” the amino acid at position 152 in the P2 sequence (SEQ ID NO: 3) is V, and the TdT further comprises the conjugation mutation comprises a “C” at position 32. In some embodiments, the recited engineered TdT sequence is the amino acid sequence of P2 (SEQ ID NO: 3). In some embodiments, the TdT further comprises the conjugation mutation E32nc, wherein “nc” is a non-canonical amino acid. In some embodiments, the TT further comprises the conjugation mutation V152nc, wherein nc is a non-canonical amino acid. In some embodiments, the TdT further comprises the conjugation mutation P154nc, wherein nc is a non-canonical amino acid. In some embodiments, the TdT further comprises the conjugation mutation E32C. In some embodiments, the TdT further comprises the conjugation mutation V152C. In some embodiments, the TdT further comprises the conjugation mutation P154C. In some embodiments, the TdT further comprises the conjugation mutation E29C. In some embodiments, the TdT further comprises the conjugation mutation E37C. In some embodiments, the TdT further comprises the conjugation mutation R44C. In some embodiments, the TdT further comprises the conjugation mutation K102C.

In some embodiments, the TdT comprises the amino acid sequence of SEQ ID NO: 4. In some embodiments, the TT comprises the amino acid sequence of SEQ ID NO: 5. In some embodiments, the TdT comprises the amino acid sequence of SEQ ID NO: 6. In some embodiments, the TdT comprises the amino acid sequence of SEQ ID NO: 7. In some embodiments, the TdT comprises the amino acid sequence of SEQ ID NO: 9. In some embodiments, the TdT comprises the amino acid sequence of SEQ ID NO: 10. In some embodiments, the TdT comprises the amino acid sequence of SEQ ID NO: 16. In some embodiments, the TdT comprises the amino acid sequence of SEQ ID NO: 17. In some embodiments, the TT comprises the amino acid sequence of SEQ ID NO: 18. In some embodiments, the TdT comprises the amino acid sequence of SEQ ID NO: 19.

### III. Tether Molecules

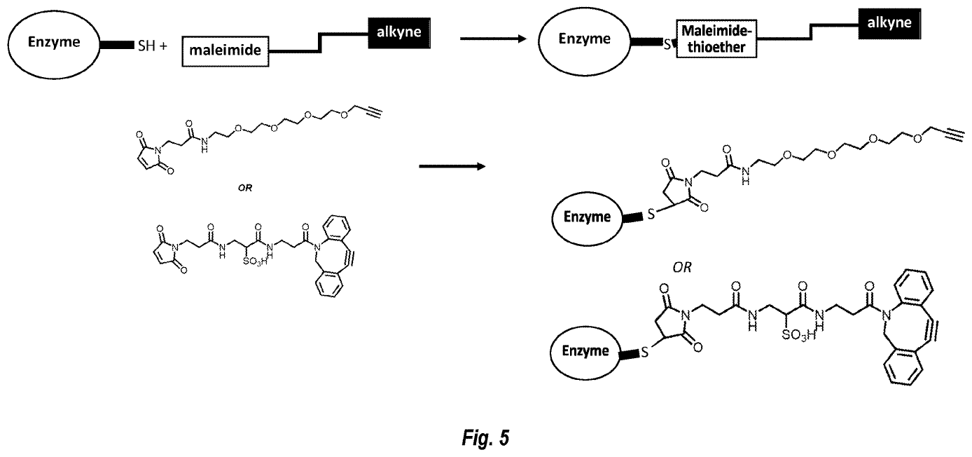

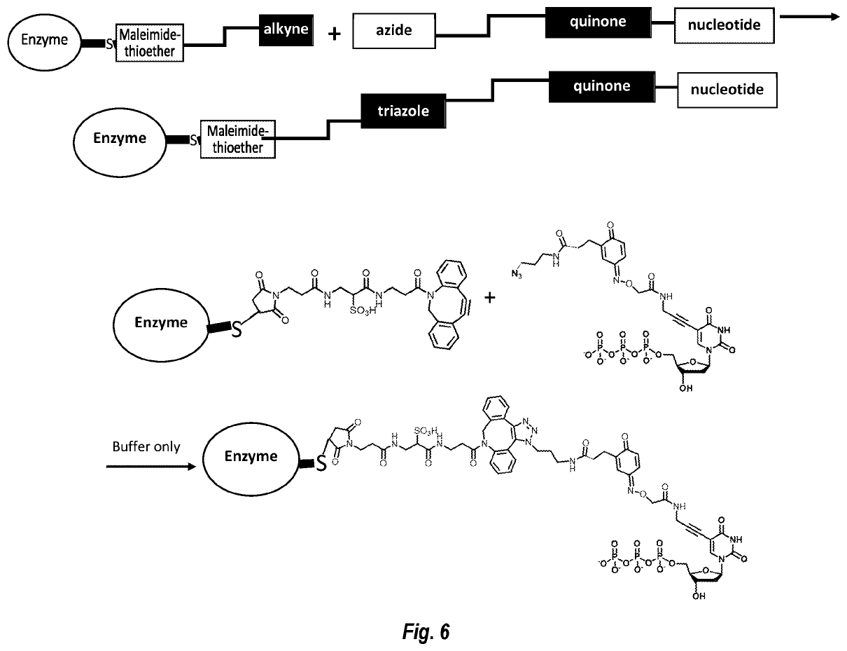

In some embodiments, an engineered TdT is capable of covalently binding to a nucleotide molecule at a conjugation residue of the engineered TdT via a tether. In some embodiments, the engineered TdT is covalently bound to a nucleotide molecule via a tether using a two-step conjugation. In some embodiments, in the first step, a bifunctional crosslinker (e.g., a maleimide-DBCO bifunctional crosslinker) is reacted with a cysteine residue on the engineered TdT; the maleimide reacts with the cysteine residue, leaving a free DBCO. In some embodiments, in the second step, an azide-redox-cleavable linker covalently bound to a nucleotide molecule is reacted with the free DBCO on the TdT-bifunctional crosslinker molecule, forming a triazole moiety and completing the tether binding the engineered TdT to the nucleotide molecule. An example of a two-step reaction described in these embodiments is shown in FIGS. 5 and 6.

In some embodiments, molecules that can be directly conjugated to a cysteine residue on the TT enzyme via a tether using a one-step conjugation without a bifunctional crosslinker are provided.

In some embodiments, an engineered TdT is provided, wherein the TdT is capable of covalently binding to a nucleotide molecule at a conjugation residue of the engineered TdT via a tether, wherein: (i) the conjugation residue comprises a cysteine residue, a non-canonical amino acid residue, a lysine residue, or an arginine residue capable of reacting with a bifunctional crosslinker or a redox-cleavable linker; and; (ii) wherein the nucleotide molecule comprises a nucleotide, nucleotide triphosphate, or a nucleotide analog.

All chemical structures showing protonated triphosphates and/or sulfonates should be interpreted to include the protonated and the ionized salt forms in different buffers.

In some embodiments, the TdT, tether, tether, and nucleotide molecule comprise any one of the following structures:

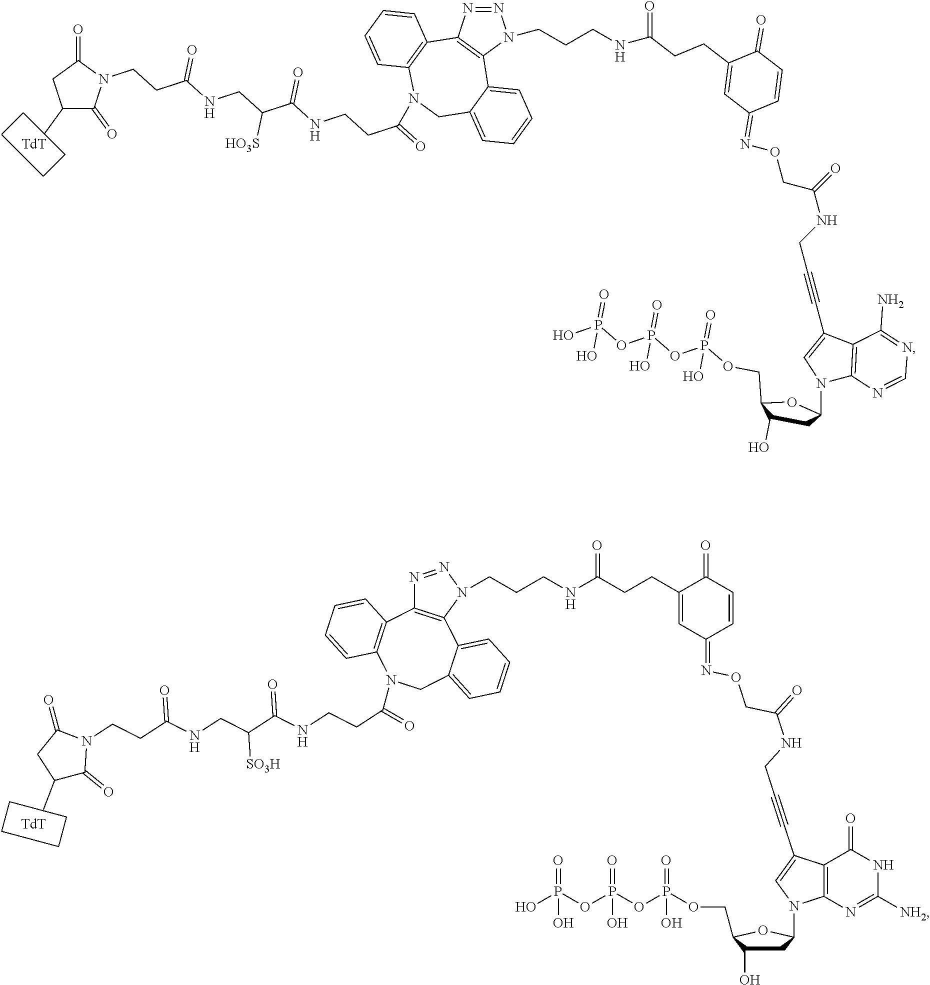

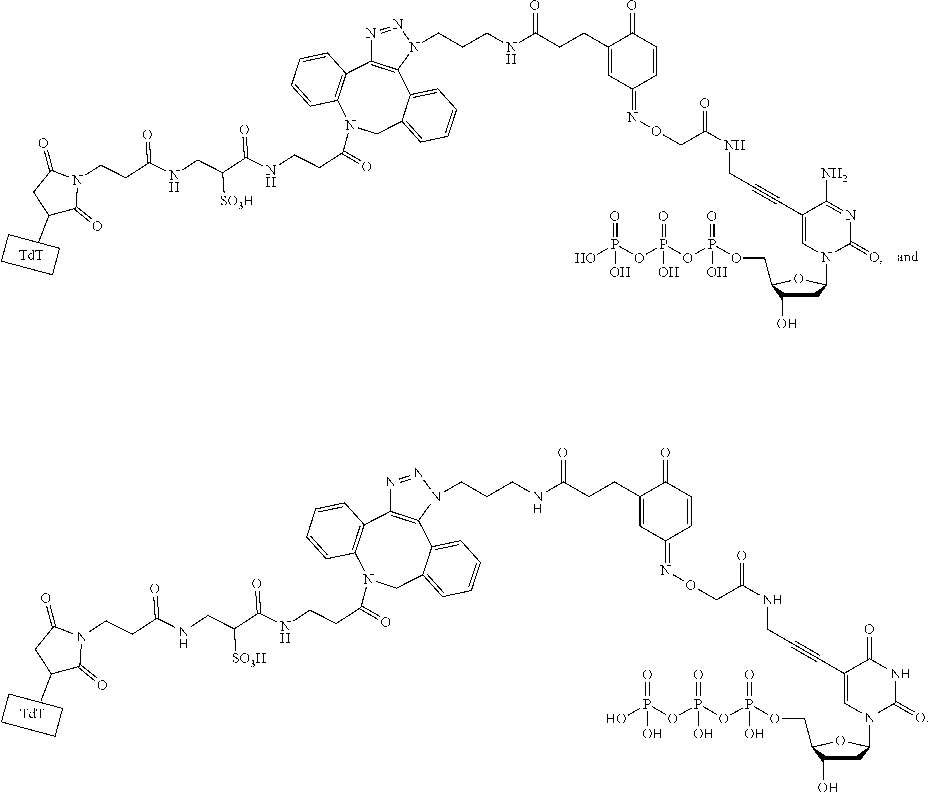

In some embodiments, the TdT, tether, and nucleotide molecule comprise any one of the following structures:

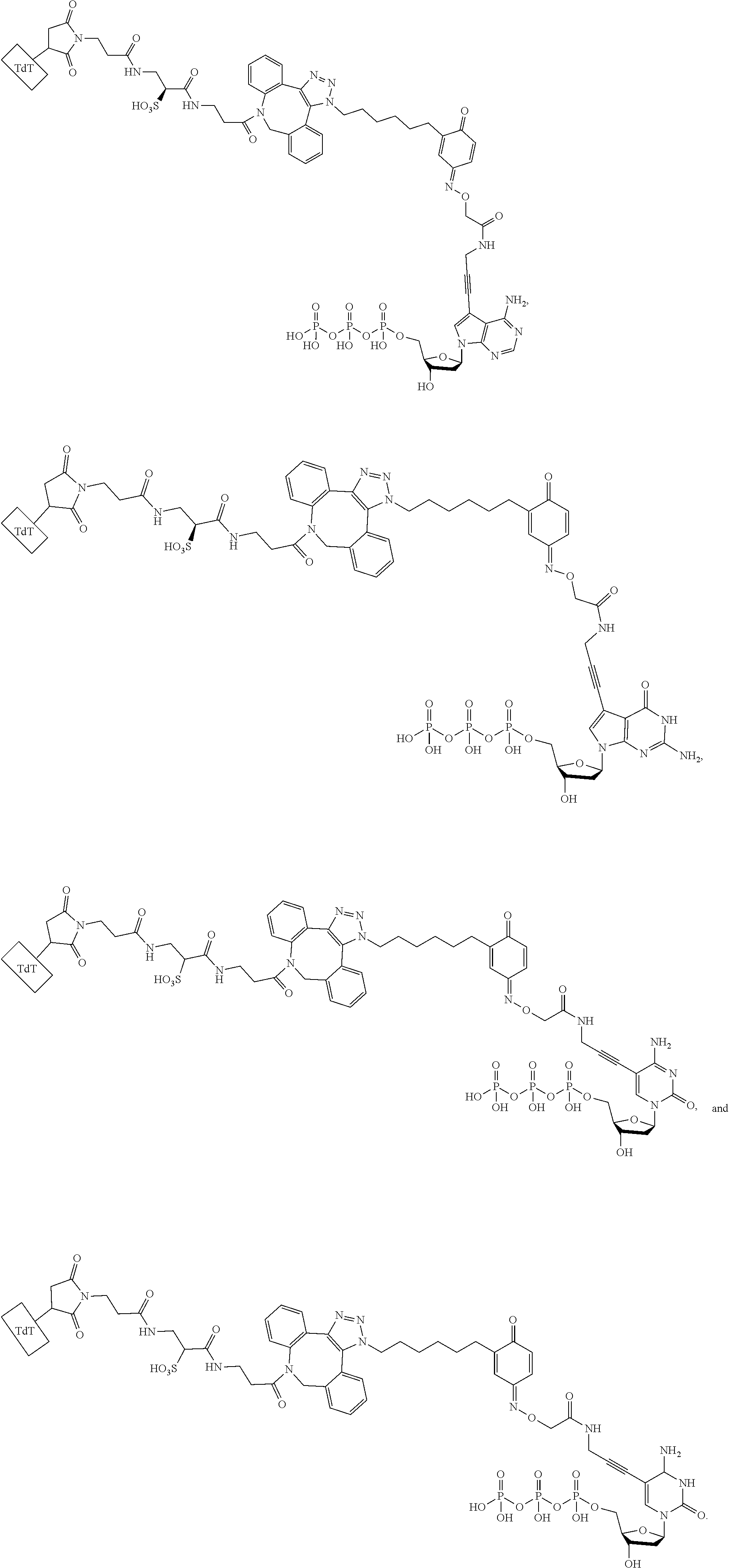

In some embodiments, the TdT, tether, and nucleotide molecule comprise any one of the following structures:

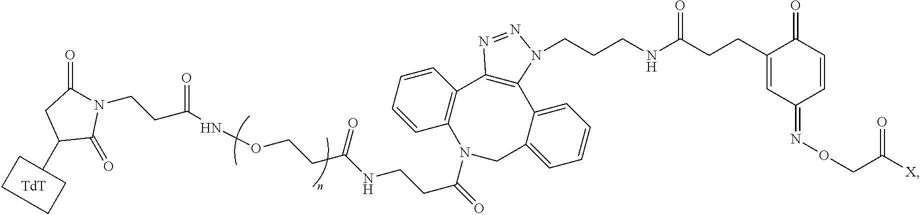

wherein X is any one of:

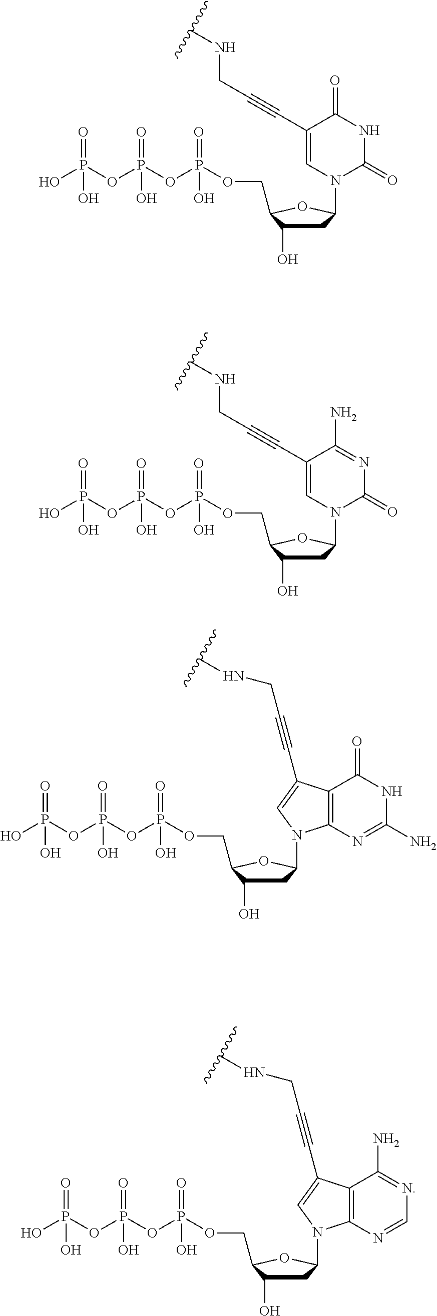

In some embodiments, the TT, tether, and nucleotide molecule comprise any one of the following structures:

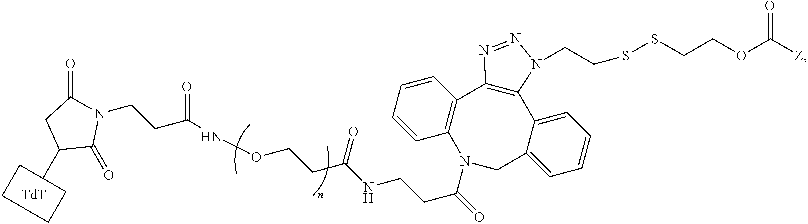

wherein Z is any one of:

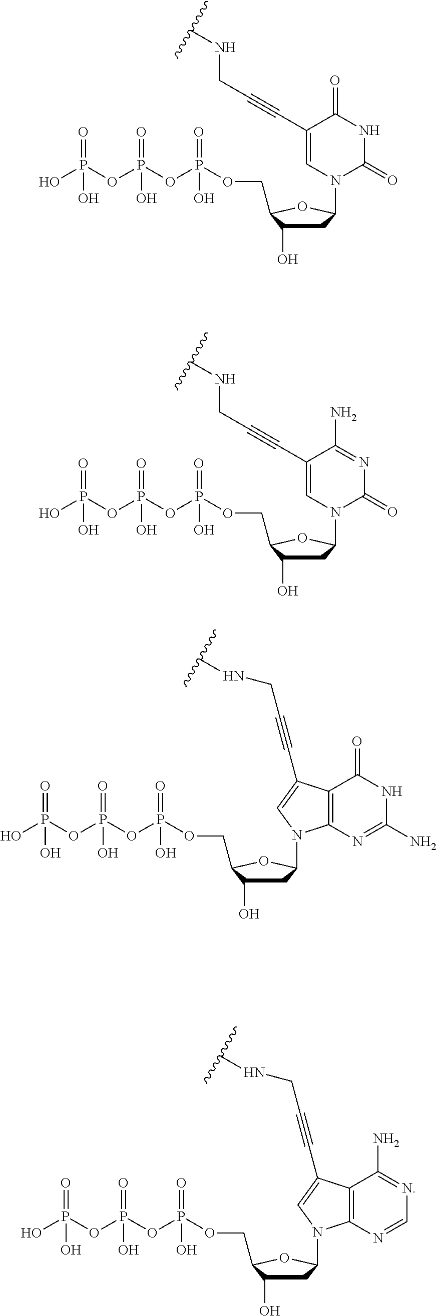

In some embodiments, the TdT, tether, and nucleotide molecule comprise any one of the following structures:

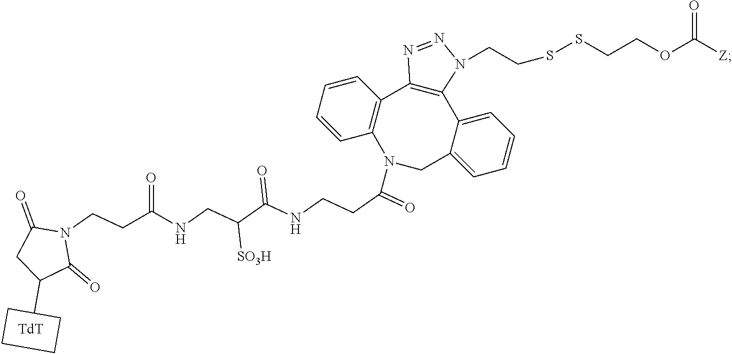

wherein Z is any one of:

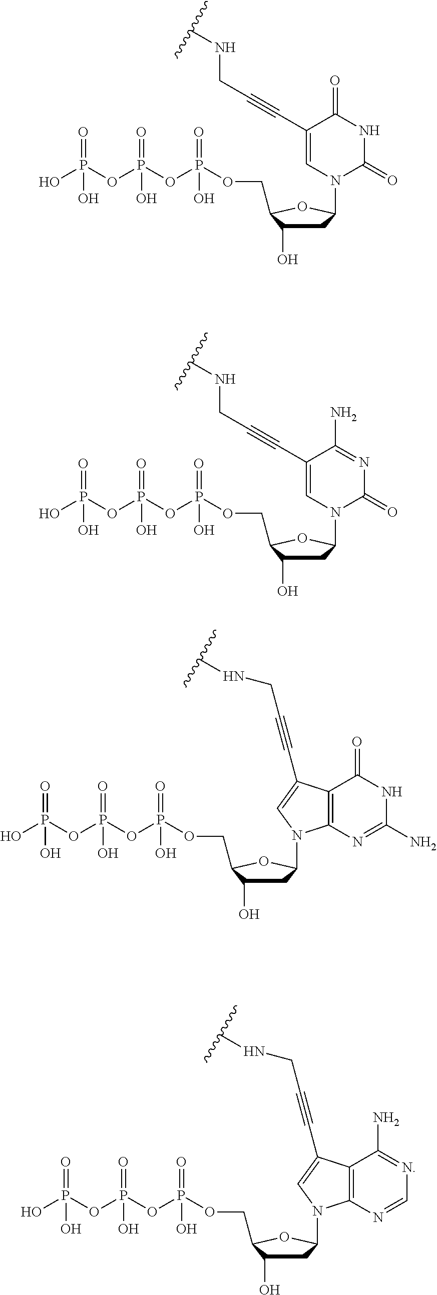

In some embodiments, the TdT, tether, and nucleotide molecule comprise any one of the following structures:

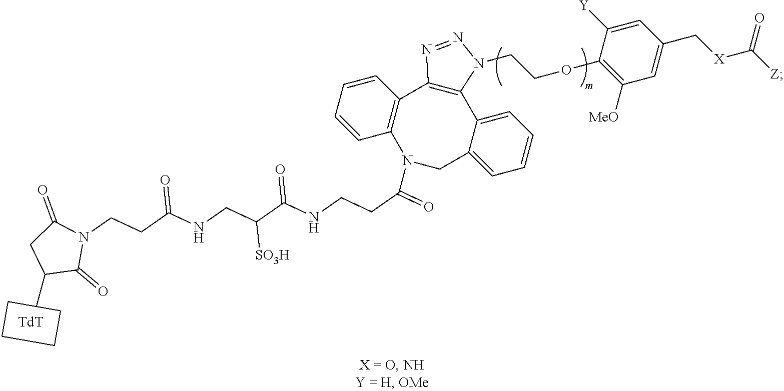

wherein X is O or NH;
wherein Y is H or OMe; and
wherein Z is any one of:

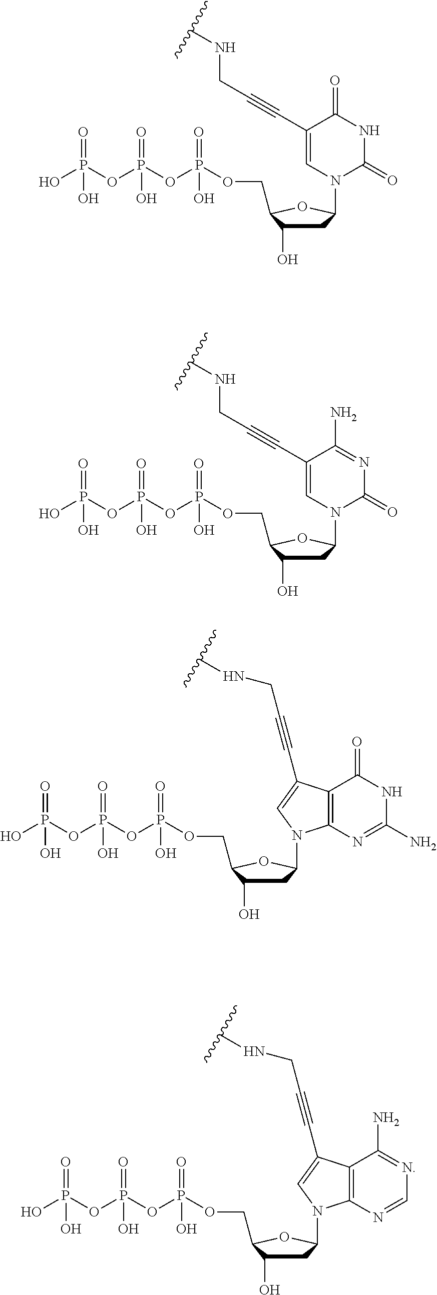

In some embodiments, the TdT, tether, and nucleotide molecule comprise any one of the following structures:

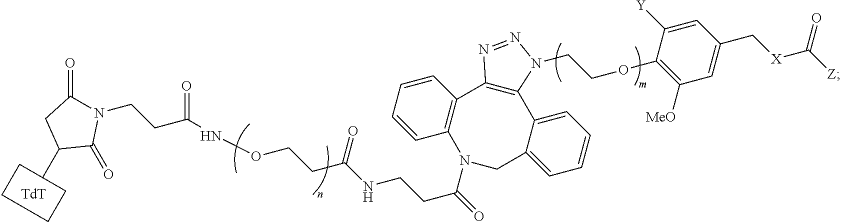

wherein X is O or NH;
wherein Y is H or OMe; and
wherein Z is any one of:

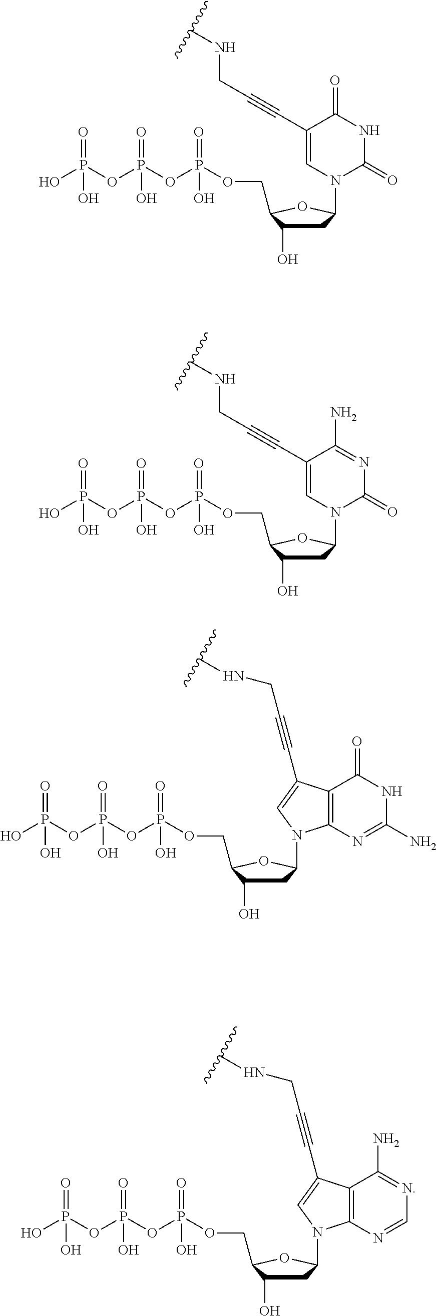

In some embodiments, the TdT, tether, and nucleotide molecule comprise any one of the following structures:

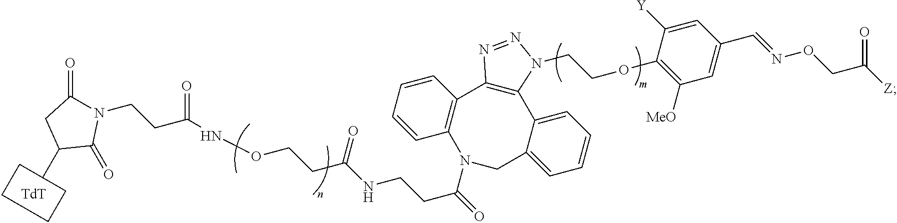

wherein Y is H or OMe; and
wherein Z is any one of:

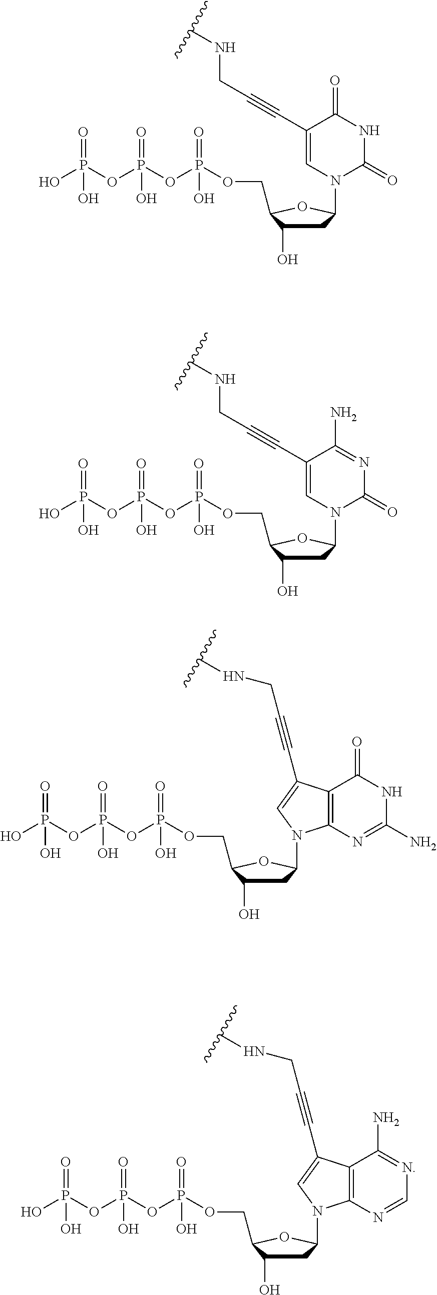

In some embodiments, the TdT, tether, and nucleotide molecule comprise any one of the following structures:

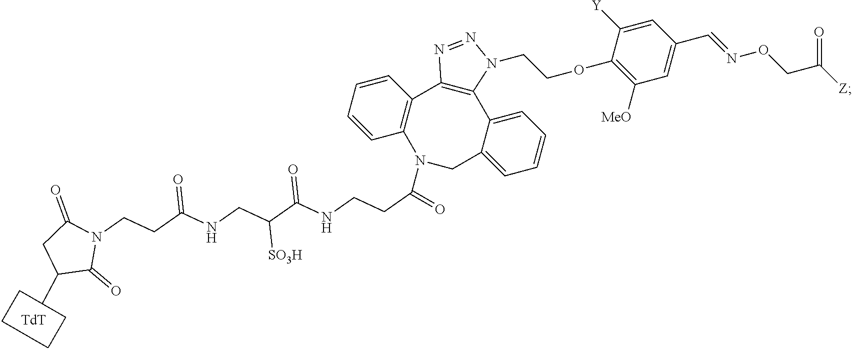

wherein Y is H or OMe; and
wherein Z is any one of:

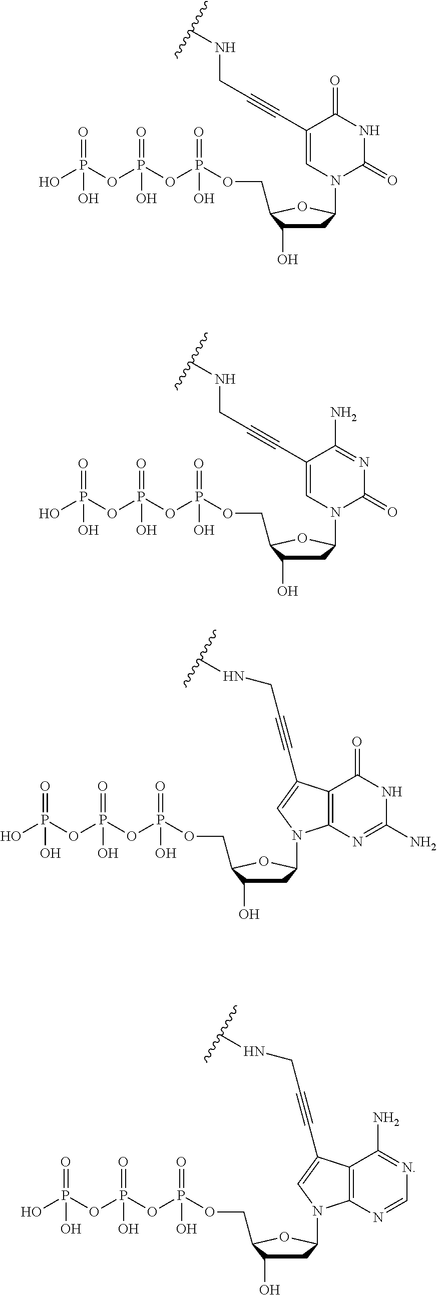

### IV. Kits

In some embodiments, a kit comprising an engineered TdT is provided that is capable of covalently binding to a nucleotide molecule at a conjugation residue of the engineered TdT via a tether. In some embodiments, the engineered TdT is covalently bound to a nucleotide molecule via a tether using a two-step conjugation. In some embodiments, in the first step, a bifunctional crosslinker (e.g., a maleimide-DBCO bifunctional crosslinker) is reacted with a cysteine residue on the engineered TdT; the maleimide reacts with the cysteine residue, leaving a free DBCO. In some embodiments, in the second step, an azide-redox-cleavable linker covalently bound to a nucleotide molecule is reacted with the free DBCO on the TdT-bifunctional crosslinker molecule, forming a triazole moiety and completing the tether binding the engineered TdT to the nucleotide molecule. An example of a two-step reaction described in these embodiments is shown in FIGS. 5 and 6.

In some embodiments, kits comprising molecules that can be directly conjugated to a cysteine residue on the TT enzyme via a tether using a one-step conjugation without a bifunctional crosslinker are provided.

All chemical structures showing protonated triphosphates and/or sulfonates should be interpreted to include the protonated and the ionized salt forms in different buffers.

In some embodiments, kits are provided comprising an engineered TdT, wherein the TdT is capable of covalently binding to a nucleotide molecule at a conjugation residue of the engineered TdT via a tether, wherein the kit further comprises: (i) a bifunctional crosslinker and/or a redox-cleavable linker comprising a functional group for covalent conjugation to a conjugation residue of the TdT; and (ii) a nucleotide molecule. In some embodiments, the kit further comprises a spacer.

In some embodiments, the functional group for covalent conjugation comprises a maleimide, N-hydroxysuccinimidyl group, or a dibenzylcyclooctyne (DBCO). In some embodiments, the functional group for covalent conjugation comprises a maleimide. In some embodiments, the maleimide is capable of attaching to a conjugation residue of the engineered TdT, wherein the conjugation residue is a cysteine residue in the TdT. In some embodiments, the functional group for covalent conjugation comprises a DBCO. In some embodiments, the DBCO is capable of attaching to a conjugation residue of the engineered TdT, wherein the conjugation residue is a non-canonical amino acid in the TdT. In some embodiments, the non-canonical amino acid is 4-azidophenylalanine.

In some embodiments, the redox-cleavable linker comprises a quinone oxime ether linker. In some embodiments, the redox-cleavable linker comprises a disulfide linker. In some embodiments, the redox-cleavable linker comprises a syringic acid-based linker or vanillin-based linker.

In some embodiments, the spacer comprises one or more polyethylene glycol molecules (PEG)ₙ, optionally wherein “n” is 1-12. In some embodiments, the spacer comprises a C1-C10 alkyl. In some embodiments, the spacer comprises a hydrocarbon chain wherein one or more of the carbons comprises a substituted sulfonate group. In some embodiments, the spacer comprises a 4-aminobenzyl alcohol or an aminopropanol spacer. In some embodiments, the spacer comprises an aminopropyl carbamate spacer. In some embodiments, the spacer comprises a hydrocarbon chain comprising 1-6 carbons wherein one or more of the carbons comprises a substituted sulfonate group. In some embodiments, the spacer comprises a C1-C6 alkyl.

In some embodiments, the kit comprises a bifunctional crosslinker capable of being covalently linked to the engineered TdT and comprising a DBCO, wherein the DBCO of the bifunctional crosslinker is capable of reacting with any one of the following

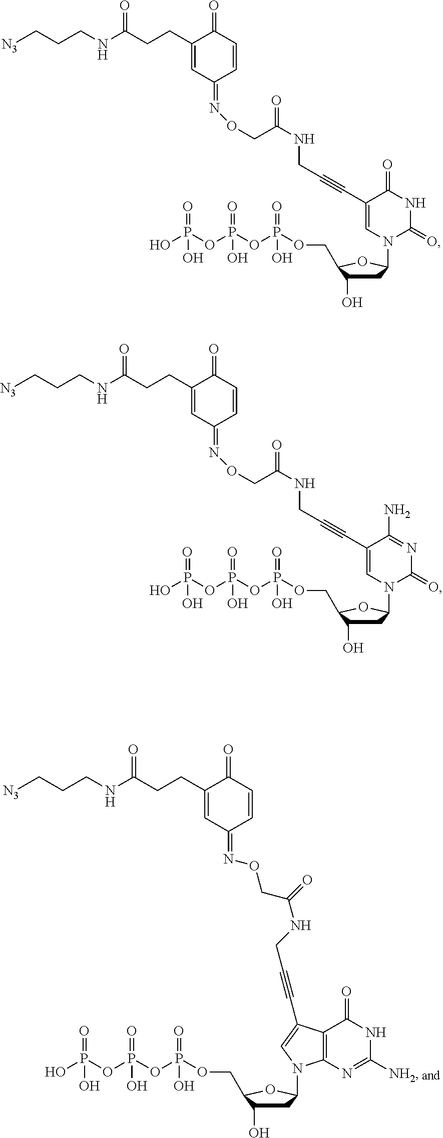

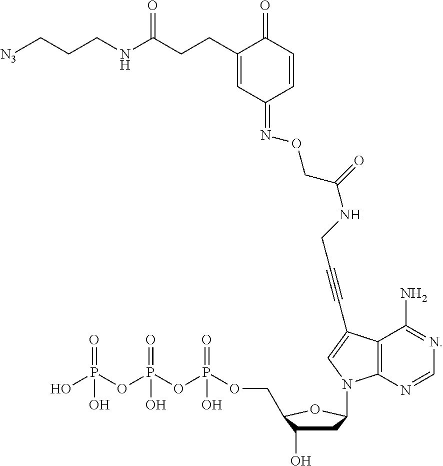

In some embodiments, the kit comprises a bifunctional crosslinker capable of being covalently linked to the engineered TdT and comprising a DBCO, wherein the DBCO of the bifunctional crosslinker is capable of reacting with any one of the following

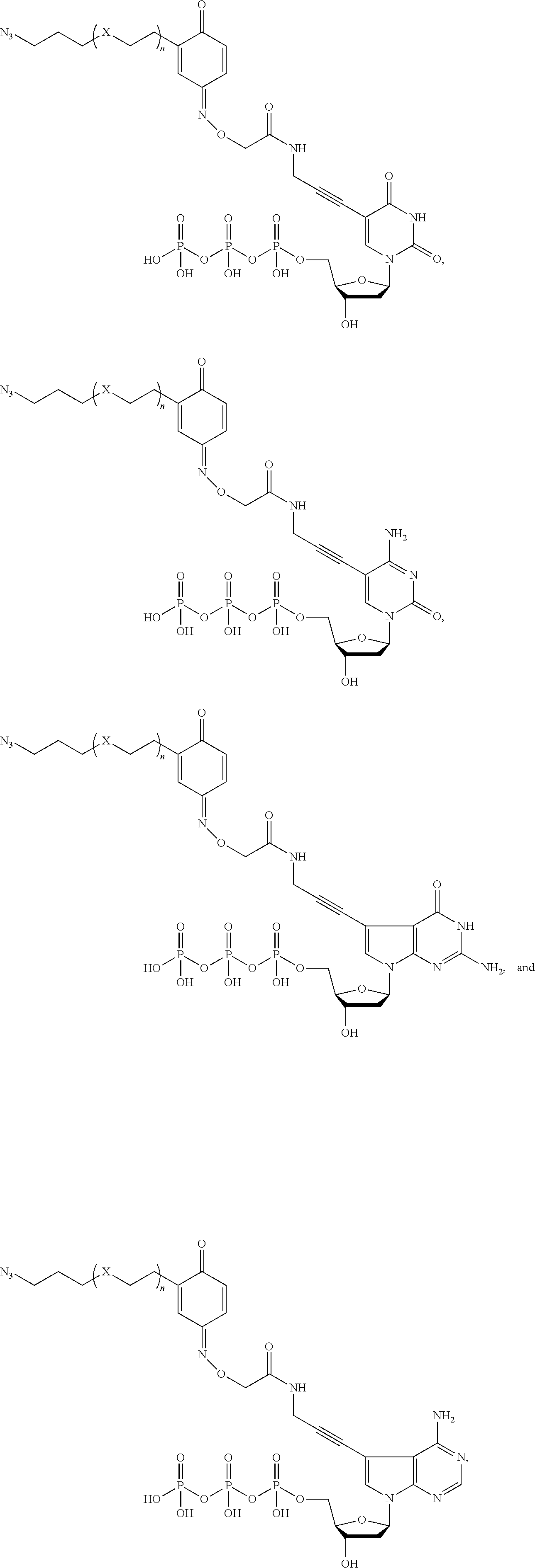

wherein X is C(O), C(O)NH, CH2, or O; and wherein n=1 for X═C(O), n=1 for C(O)NH, n=1 for CH2, and n=1-4 for X═O, wherein n is an integer.

In some embodiments, the kit comprises a bifunctional crosslinker capable of being covalently linked to the engineered TdT and comprising a DBCO, wherein the DBCO of the bifunctional crosslinker is capable of reacting with any one of the following

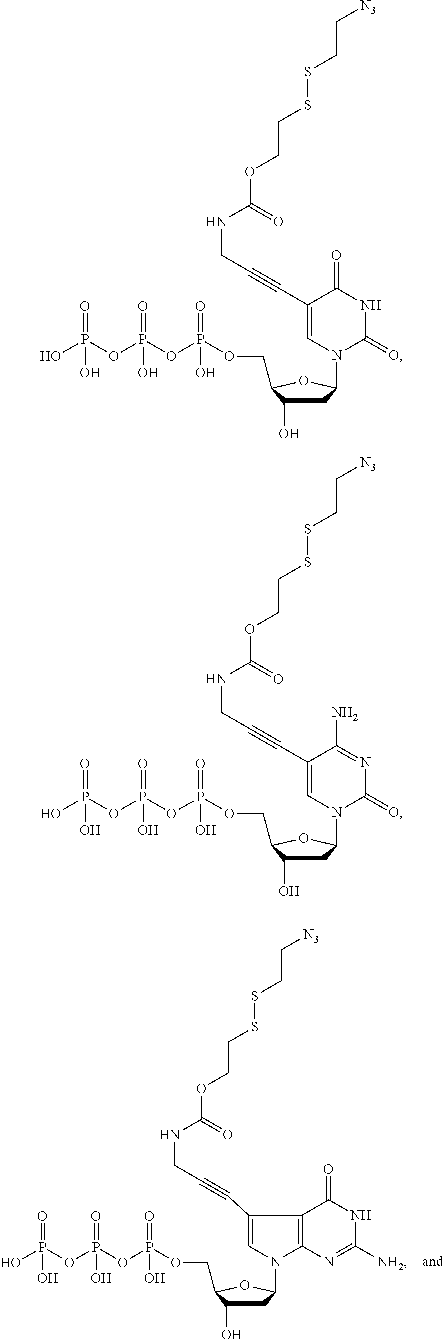

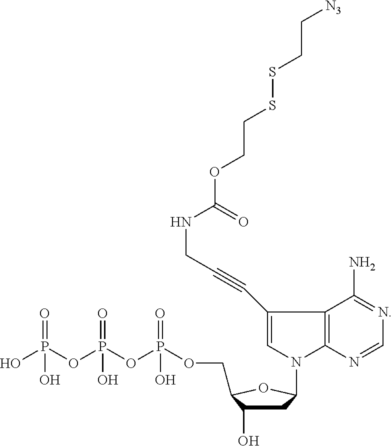

In some embodiments, the kit comprises a bifunctional crosslinker capable of being covalently linked to the engineered TdT and comprising a DBCO, wherein the DBCO of the bifunctional crosslinker is capable of reacting with any one of the following

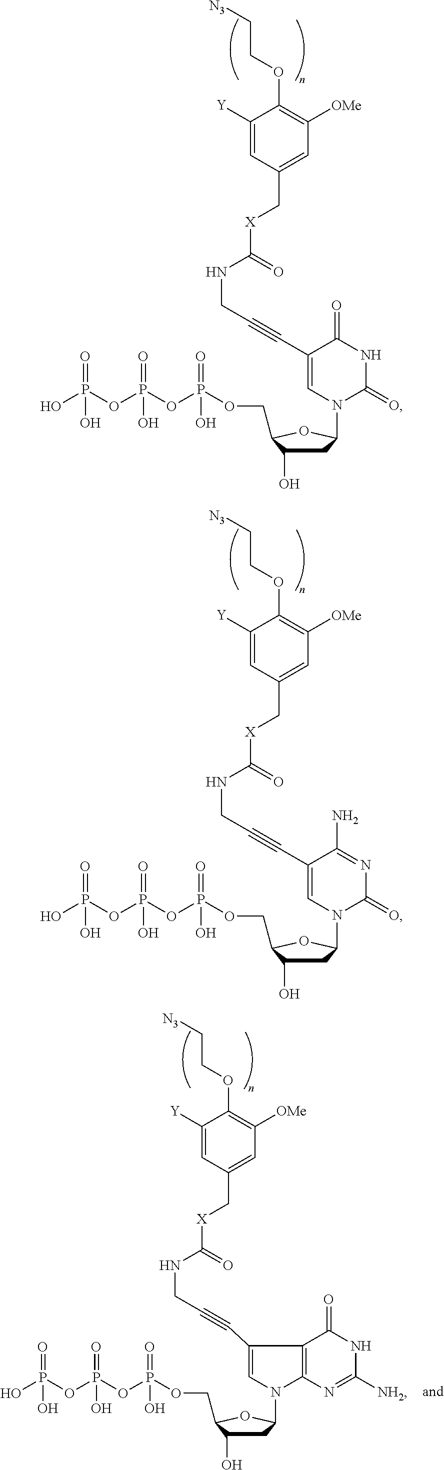

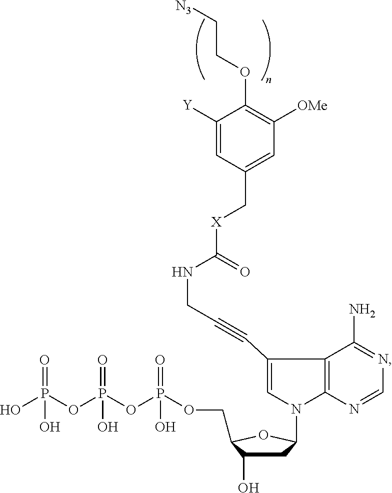

wherein X is O or NH, and wherein Y is H or OMe.

In some embodiments, the kit comprises one or more of the following molecules capable of being reacted with the engineered TdT, wherein the conjugation residue is a cysteine residue in the TdT:

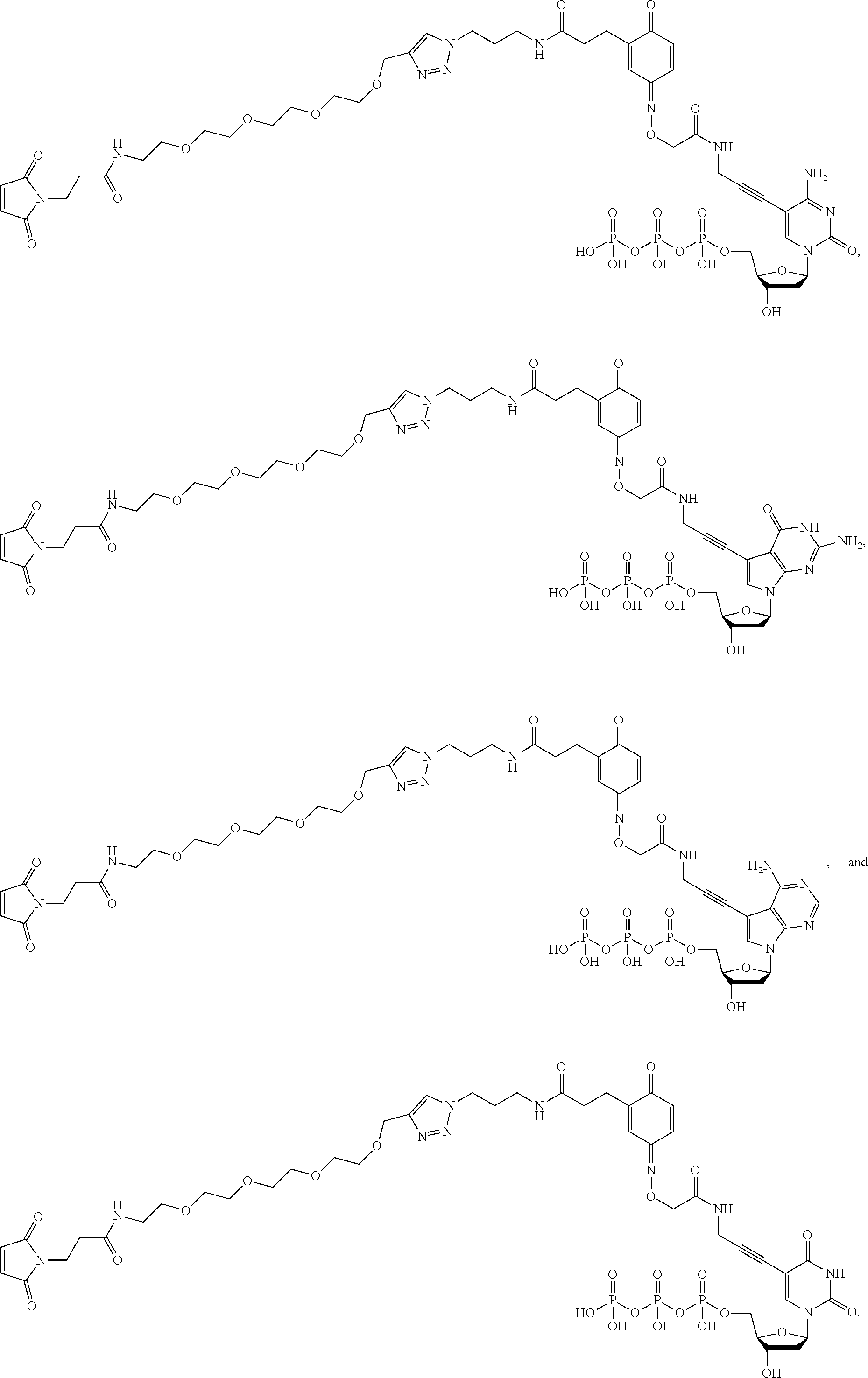

In some embodiments, the kit comprises one or more of the following molecules capable of being reacted with the engineered TdT, wherein the conjugation residue is a cysteine residue in the TdT:

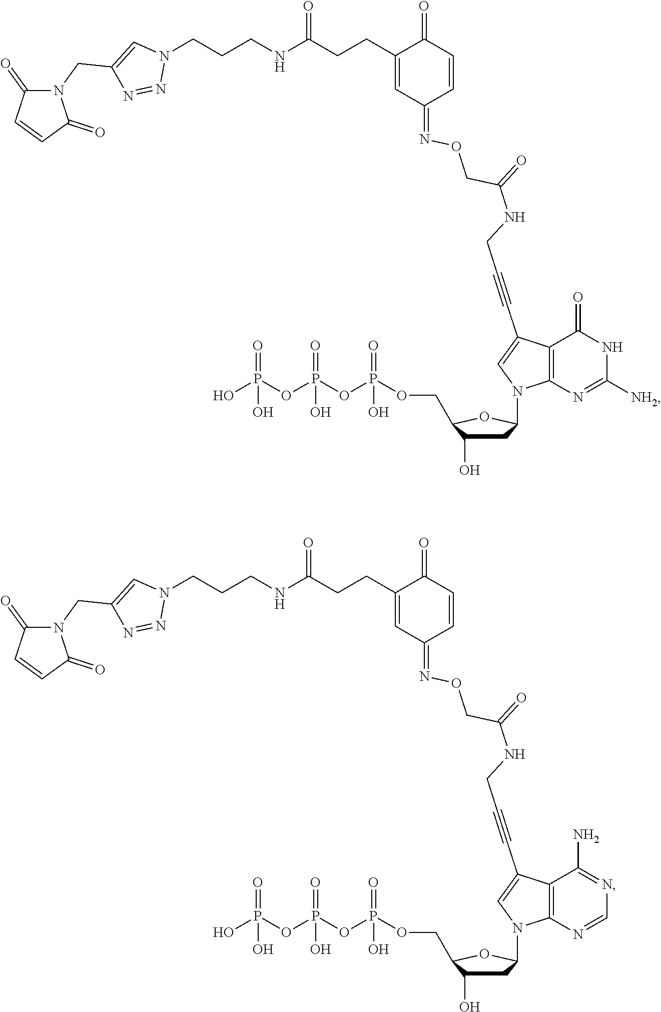

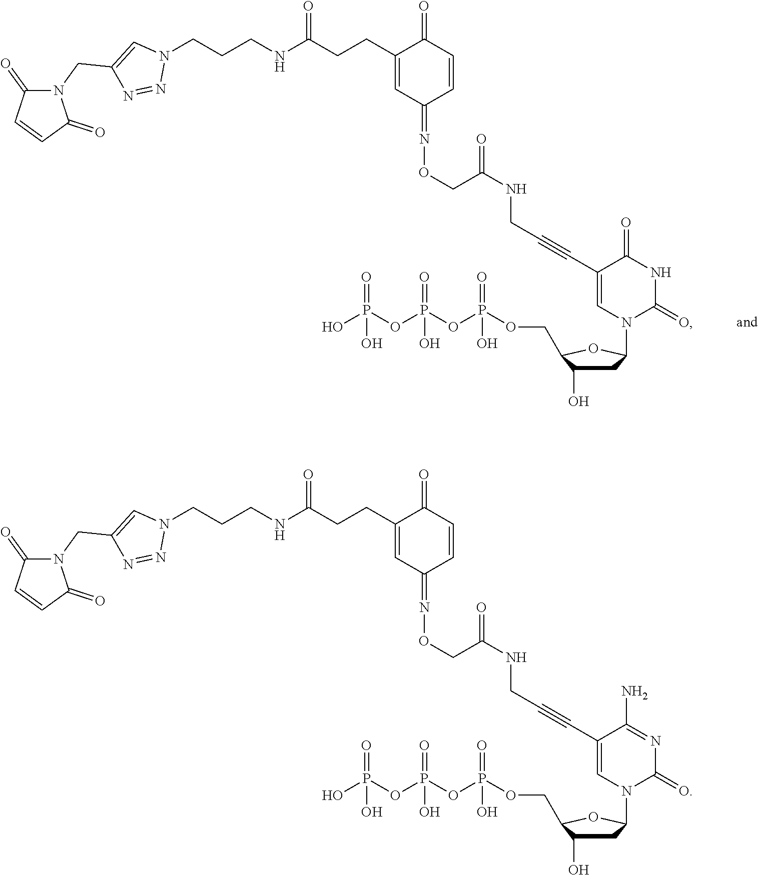

In some embodiments, the kit comprises one or more of the following molecules capable of being reacted with the engineered TdT, wherein the conjugation residue is a non-canonical amino acid residue in the TdT:

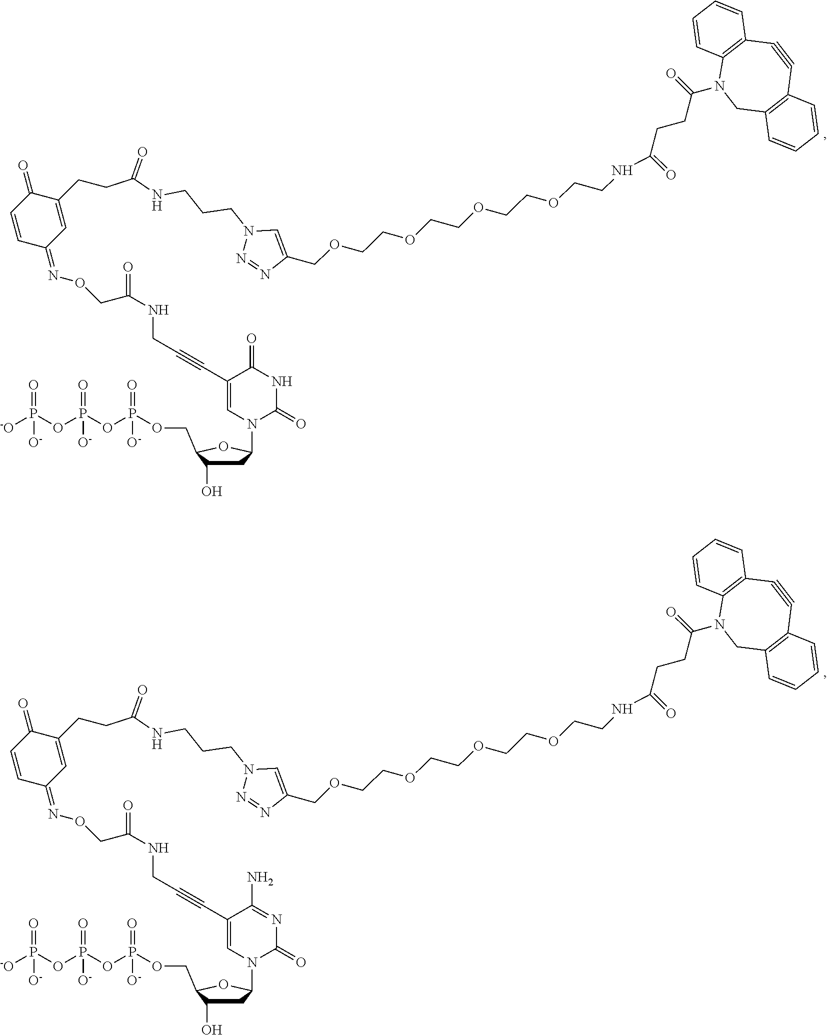

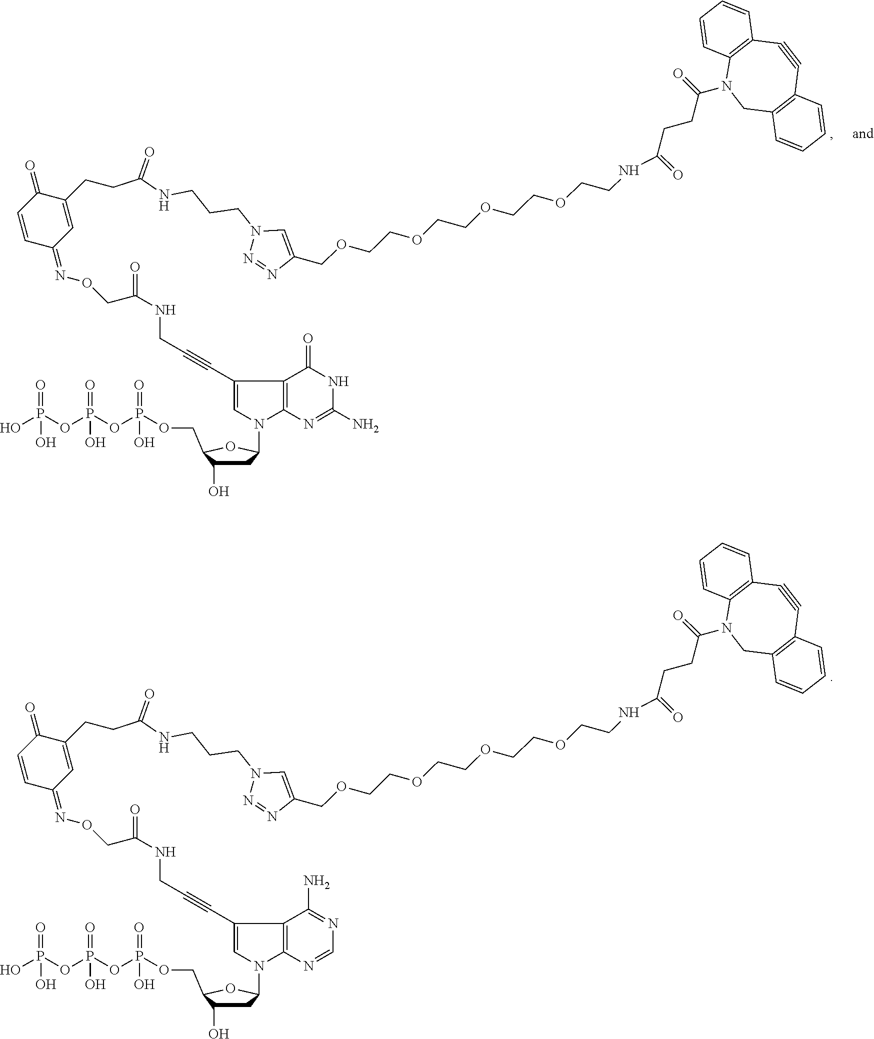

In some embodiments, the kit comprises one or more of the following molecules capable of being reacted with the engineered TdT, wherein the conjugation residue is a lysine residue or an arginine residue in the TdT:

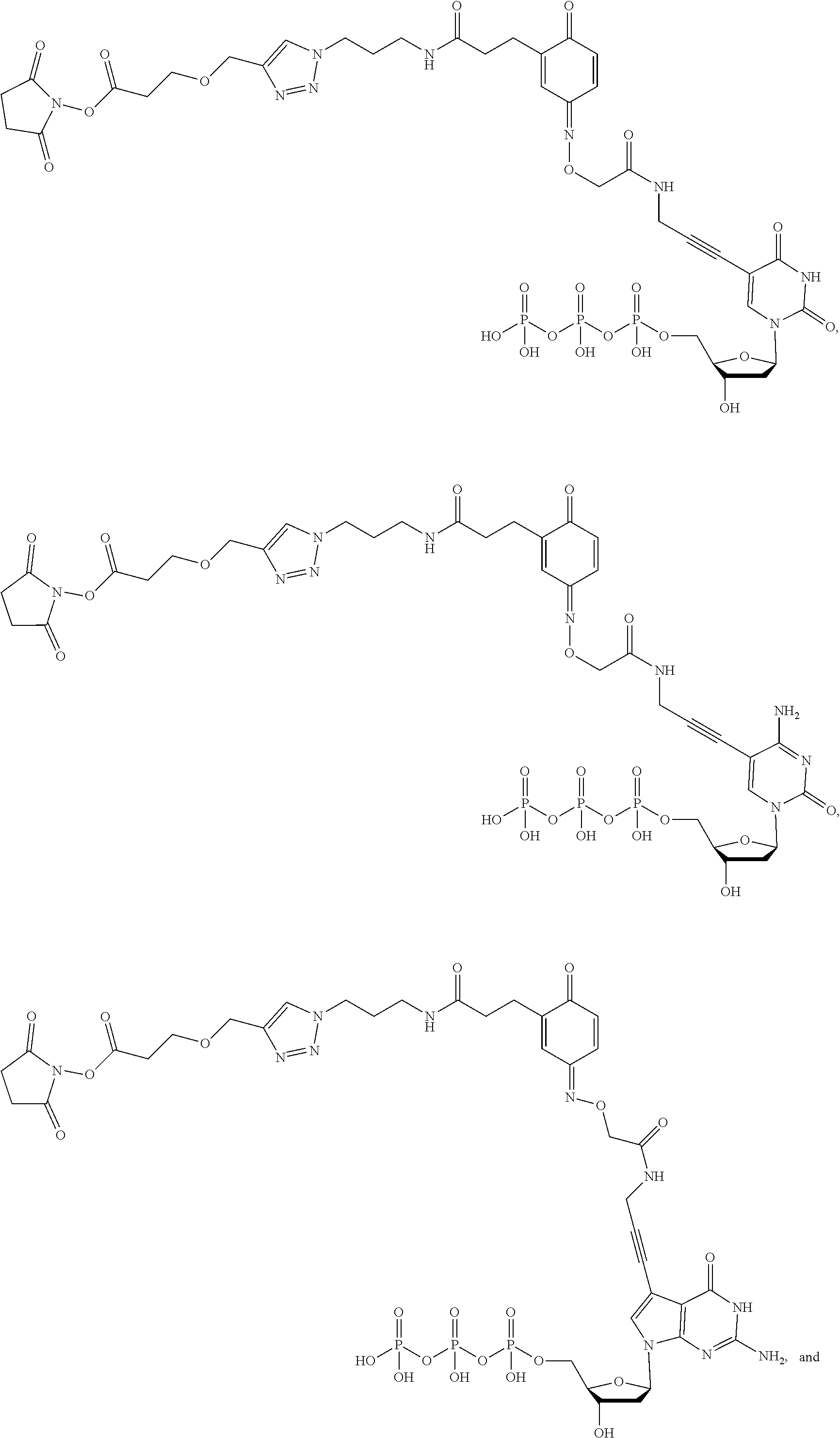

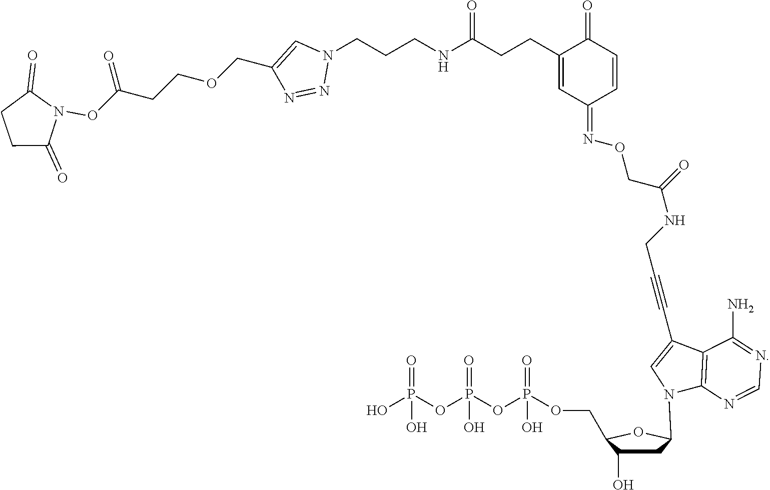

### V. Methods of Nucleic Acid Synthesis

Methods for nucleic acid molecule synthesis are described herein. In some embodiments, the method of nucleic acid molecule synthesis comprises the steps of: (a) providing an engineered terminal deoxynucleotidyl transferase (TdT), wherein the engineered TdT comprises a nucleotide molecule covalently attached to the TT via a redox-cleavable linker; (b) providing a single-stranded nucleic acid molecule comprising a 5′ and a 3′ end; (c) contacting the engineered TdT and the single-stranded nucleic acid molecule under conditions suitable for the TdT to bind to the 3′ end of the nucleic acid molecule and form a TdT-nucleic acid strand complex, thereby incorporating the nucleotide molecule covalently linked to the TdT into the nucleic acid strand, resulting in the nucleic acid molecule tethered to the TdT and thereby blocking subsequent additions of a nucleotide molecule to the nucleic acid strand; and (d) exposing the TdT-nucleic acid strand complex to suitable electrochemical conditions, wherein the redox-cleavable linker is cleaved thereby separating the TdT-redox-cleavable linker molecule from the nucleic acid strand with the newly incorporated nucleotide molecule, resulting in a nucleic acid strand with the newly incorporated nucleotide molecule; and (e) repeating steps (a) through (d) thereby synthesizing a nucleic acid molecule.

In some embodiments, the redox-cleavable linker is a quinone oxime ether linker. In some embodiments, the electrochemical conditions of step (d) comprise one or more suitable soluble redox shuttles in the presence of two or more electrodes, wherein the two or more electrodes control the oxidation state of the soluble redox shuttles. In some embodiments, the soluble redox shuttles comprise a thiazine, a viologen, dinucleotides, a flavin, a quinone, peroxide, bromide, vanadium and/or a metal complex. In some embodiments, the soluble redox shuttles comprise a thiazine. In some embodiments, the thiazine is methylene blue. In some embodiments, the soluble redox shuttles comprise a viologen. In some embodiments, the viologen is methyl viologen (MV) or bis-(trimethylammonio) propyl viologen (BTMAP-Vi). In some embodiments, the soluble redox shuttles comprise dinucleotides. In some embodiments, the dinucleotides comprise NAD/NADH, nicotinamide adenine dinucleotide, and/or reduced form. In some embodiments, the soluble redox shuttles comprise a flavin. In some embodiments, the flavin is riboflavin (RF), flavin mononucleotide (FMN), or flavin adenine dinucleotide (FAD). In some embodiments, the soluble redox shuttles comprise a quinone. In some embodiments, the quinone is 2,5-dihydroxy-1,4-benzoquinone (DHBQ), 9,10-anthraquinone-2,7-disulfonic acid (AQDS), 4,5-dihydroxybenzene-1,3-disulfonic acid (BQDS), or 2,3,5,6-tetrakis((dimethylamino)methyl)hydroquinone (FQH₂). In some embodiments, the soluble redox shuttles comprise peroxide. In some embodiments, the soluble redox shuttles comprise bromide. In some embodiments, the soluble redox shuttles comprise vanadium. In some embodiments, the soluble redox shuttles comprise a metal complex. In some embodiments, the metal complex is TEMPO (2,2,6,6-tetramethylpiperidin-1-yl)oxyl, (2,2,6,6-tetramethylpiperidin-1-yl) oxidanyl). In some embodiments, the metal complex is an organometallic coordination complex. In some embodiments, the organometallic coordination complex is a ferrocene, bis-(trimethylammonio) propyl ferrocene (BTMAP-Fc), ammonium ferricyanide ((NH₄)₄Fe(CN)₆), potassium ferricyanide (K₄Fe(CN)₆), hexaamineruthenium (III) chloride (RuHex), permanganate, or dichromate.

In some embodiments, the electrochemical conditions of step (d) further comprise a buffer, a supporting electrolyte, and/or a hydrotropic agent. In some embodiments, the buffer comprises sodium citrate, potassium hydrogen phosphate, and/or potassium dihydrogen phosphate. In some embodiments, the supporting electrolyte comprises sodium chloride, sodium salts, lithium salts, potassium salts, and/or magnesium salts. In some embodiments, the hydrotropic agent comprises caffeine, urea, and/or nicotinamide (NA). In some embodiments, the electrochemical conditions of step (d) comprise a redox shuttle solution comprising methylene blue, citrate-phosphate buffer, and sodium chloride. In some embodiments, the electrochemical conditions of step (d) comprise a redox shuttle solution comprising methylene blue, citrate-phosphate buffer, sodium chloride, and nicotinamide. In some embodiments, the electrochemical conditions of step (d) comprise a redox shuttle solution comprising flavin mononucleotide, citrate-phosphate buffer, sodium chloride, and nicotinamide. In some embodiments, the electrochemical conditions of step (d) comprise a redox shuttle solution comprising 10 mM methylene blue, 25 mM citrate-phosphate buffer at pH 3.4, and 100 mM sodium chloride. In some embodiments, the electrochemical conditions of step (d) comprise a redox shuttle solution comprising 10 mM methylene blue, 25 mM citrate-phosphate buffer at pH 3.4, 100 mM sodium chloride, and 1 M nicotinamide. In some embodiments, the electrochemical conditions of step (d) comprise a redox shuttle solution comprising 50 mM flavin mononucleotide, 25 mM citrate-phosphate buffer at pH 3.4, 1 M sodium chloride, and 1 M nicotinamide.

In some embodiments, the potential for cleavage is about +1.05 vs. SHE to −0.18V vs. SHE at 25 °C and pH 3; +0.84 V vs. SHE to −0.38 V vs. SHE at 25 °C and pH 6.5; or +0.64V vs. SHE to −0.59V vs. SHE at 25 °C and pH 10.

In some embodiments, the engineered TdT comprises one or more mutations to a wild-type TdT of SEQ ID NO: 1, wherein: (a) the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E, wherein position numbers are relative to SEQ ID NO: 1; or b) the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353E, wherein position numbers are relative to SEQ ID NO: 1; or c) the engineered TdT has at least 85, 90, 91, 92, 93, 94, 95, 96, 97, 98, 99, or 100% identity to SEQ ID NO: 2 or 3.

### VI. Systems for Enzymatic DNA Synthesis

Systems for enzymatic DNA systems are described herein. In some embodiments, the system for enzymatic DNA synthesis comprises: a) an engineered TdT described herein; b) a redox-cleavable linker; c) a redox shuttle solution; and d) two or more electrodes on a surface. In some embodiments, the electrodes enable an applied potential or galvanic field to be supplied locally to the device or system thus enabling generation of active redox shuttle via reduction or oxidation at one or more electrodes. In some embodiments, the electrode where the soluble redox shuttle is generated is comprised of a smaller surface area than the other electrode to which it is electrically coupled. In some embodiments, if the soluble redox shuttle is activated via cathodic process, then the cathode may be comprised of less active surface area than the anode by (a) having less electroactive area, or (b) being coupled to multiple anodes which in combination provide substantially more area than the cathode. In some embodiments, the surface, or electroactive, or area aspect ratios for the counter electrode relative to the working electrode, or C/E aspect ratio is at least: 3:1, 6:1, 10:1, 100:1, 1000:1, or higher. In some embodiments, all the electrodes lie in a single plane. In some embodiments, a combination of electrodes may lie in multiple planes. In some embodiments, the electrodes are oriented parallel to perpendicular with respect to one another during the current path. In some embodiments, the system has an inter-electrode gap (i.e., distance between electrodes included in the current path) of ≤100 μm, ≤10 μm, ≤1 μm, ≤100 μm, ≤10 nm, ≤1 nm, ≤100 μm, ≤10 μm, or ≤1 μm.

In some embodiments, the redox shuttle solution comprises at least one soluble redox shuttle in combination with a buffer, a supporting electrolyte, and/or a hydrotropic agent. In some embodiments, the buffer is sodium citrate, potassium hydrogen phosphate, or potassium dihydrogen phosphate. In some embodiments, the supporting electrolyte is sodium chloride, sodium salts, lithium salts, potassium salts, or magnesium salts. In some embodiments, the hydrotropic agent is caffeine, urea, and/or nicotinamide (NA). In some embodiments, the redox shuttle solution comprises methylene blue, citrate-phosphate buffer, and sodium chloride. In some embodiments, the redox shuttle solution comprises methylene blue, citrate-phosphate buffer, sodium chloride, and nicotinamide. In some embodiments, the redox shuttle solution comprises flavin mononucleotide, citrate-phosphate buffer, sodium chloride, and nicotinamide. In some embodiments, the redox shuttle solution comprises 10 mM methylene blue, 25 mM citrate-phosphate buffer at pH 3.4, and 100 mM sodium chloride. In some embodiments, the redox shuttle solution comprises 10 mM methylene blue, 25 mM citrate-phosphate buffer at pH 3.4, 100 mM sodium chloride, and 1 M nicotinamide. In some embodiments, the redox shuttle solution comprises 50 mM flavin mononucleotide, 25 mM citrate-phosphate buffer at pH 3.4, 1 M sodium chloride, and 1 M nicotinamide.

### VII. Redox Shuttles and Redox Shuttle Solutions

Table 2 shows non-limiting examples of viable soluble redox shuttles. Reduction potentials were experimentally determined or converted from literature values. SHE stands for standard hydrogen electrode.

TABLE 2
Exemplary Redox Shuttles

| | | E_{red} VS. |
|---|---|---|
| | Class of | SHE |
| Redox Shuttle | Molecule | (pH 6.5) |
| Methylene Blue (MB) | Thiazine | −0.27 V |
| Methyl Viologen (MV) | Viologen | −0.42 V |
| bis-(trimethylammonio) propyl viologen | Viologen | −0.34 V |
| (BTMAP-Vi) | | |
| Riboflavin (RF) | Flavin | −0.29 V |
| Flavin mononucleotide (FMN) | Flavin | −0.49 V |
| Flavin adenine dinucleotide (FAD) | Flavin | −0.47 V |
| 2,5-dihydroxy-1,4-benzoquinone (DHBQ) | Quinone | −0.28 V |
| 9,10-anthraquinone-2,7-disulfonic acid (AQDS) | Quinone | −0.57 V |
| Hexaamineruthenium (III) chloride (RuHex) | Metal | −0.20 V |
| | complex | |

Table 3 shows non-limiting examples of shuttle solution formulants/components/additives.

TABLE 3
Exemplary Redox Shuttle Solution Formulants

| Redox Shuttle | | |
|---|---|---|
| Solution Additives | Role | Reasoning |
| Sodium citrate | Buffer | Increase proton concentration; |
| | | maintain pH |
| Potassium hydrogen | Buffer | Increase proton concentration; |
| phosphate | | maintain pH |
| Potassium dihydrogen | Buffer | Increase proton concentration; |
| phosphate | | maintain pH |
| Sodium chloride | Supporting | Increase solution conductivity |
| | electrolyte | |
| Caffeine | Hydrotrope | Increase redox shuttle solubility; |
| | | stabilize redox shuttle |
| Urea | Hydrotrope | Increase redox shuttle solubility; |
| | | stabilize redox shuttle |
| Nicotinamide (NA) | Hydrotrope | Increase redox shuttle solubility; |
| | | stabilize redox shuttle |

In some embodiments, a redox shuttle that is activated via its reduction at an electrode is employed, so that it may migrate to the cleavage site and transfer its electron(s) to the cleavage site linker bond thus being oxidized in the process of breaking that bond, so as to enable subsequent nucleotide addition. In other embodiments, a redox shuttle that is activated via its oxidation at an electrode is employed, so that it may migrate to the cleavage site and harvest electron(s) from the cleavage site linker bond thus being reduced in the process of breaking that bond, so as to enable subsequent nucleotide addition. In some embodiments, the redox-cleavable linker requires the same number of electrons per molecule, or bonds to break, as the redox shuttle requires to be activated at an electrode. In other embodiments, the redox-cleavable linker requires a different number of electrons per molecule, or bonds to break, as the redox shuttle requires to be activated at an electrode. In some embodiments, the redox shuttle requires 1, 2, 3, or 4 electrons per molecule in order to be activated (i.e., reduced or oxidized to sufficient degree as to act upon the redox-cleavable linker).

Local generation of sufficient quantities of active redox shuttle molecules depends on the electrochemical kinetics at the electrode surface and reaction environment (e.g., concentration of the inactive shuttle, temperature, etc.). The composition, or formulation, of the redox shuttle solution is relevant to reproducible cleavage of the linker and highly efficient DNA synthesis (both within a given nucleotide addition step and within the overall sequence). Non-limiting example formulants are presented in Table 3, along with their role in the formulation. In some embodiments disclosed herein, electrochemical oxidation or reduction of the shuttle will involve protonation or deprotonation, however metal ions such as lithium, sodium, and magnesium and potassium may be utilized in addition to or instead of protons. In some embodiments, shuttle solutions relying upon protonation or deprotonation are sufficiently buffered to prevent local pH changes and sluggish electrochemical kinetics. Some examples of biological buffers disclosed herein include potassium phosphate buffer, citrate buffer, or citrate-phosphate buffer. Similarly, the electrochemical reaction at the working electrode may depend upon the use of an ionically conductive electrolyte between the working and counter electrodes. Supporting electrolytes, such as NaCl, may be included to enable efficient kinetics. Alternatively, other salts based upon sodium, lithium, potassium, magnesium may be employed as supporting electrolytes. Other formulants may be used to increase the solubility of the redox shuttle so as to increase the rate of generation. For example, some embodiments include amphiphilic hydrotropic agents, such as nicotinamide, caffeine, and urea, which are known to form weak molecular interactions with poorly soluble redox molecules, thereby increasing their stability and solubility. Orita, A. et al., “A biometric redox flow battery based on flavin mononucleotide,” 7 Nat. Commun. 7, 13230 (2016). Finally, specific formulants can play multiple roles. The pyridinic N group in nicotinamide, for example, has a pKₐ of 3.3, thereby enabling it to act as both a hydrotrope and a buffer when the solution pH is formulated close to its pKₐ value.

Some embodiments comprise a redox shuttle solution. In some embodiments, the redox shuttle solution comprises at least one soluble redox shuttle in combination with one or more of the following: supporting electrolyte, buffer, and hydrotrope. In some embodiments, the redox shuttle solution comprises: 10 mM methylene blue, 25 mM citrate-phosphate buffer at pH 3.4, and 100 mM sodium chloride. In some embodiments, the redox shuttle solution comprises 10 mM methylene blue, 25 mM citrate-phosphate buffer at pH 3.4, 100 mM sodium chloride, and 1 M nicotinamide. In some embodiments, the redox shuttle solution comprises: 50 mM flavin mononucleotide, 25 mM citrate-phosphate buffer at pH 3.4, 1 M sodium chloride, and 1 M nicotinamide.

The aforementioned compositions, or formulations, of redox shuttles solutions comprising, in some embodiments, at least one redox shuttle in combination with one or more of the following constituent formulants—supporting electrolyte, buffer, hydrotrope—are particularly useful for enabling enzymatic DNA synthesis in a device or system including two or more electrodes. Said electrodes enable an applied potential or galvanic field to be supplied locally to the device or system thus enabling generation or active redox shuttle via reduction or oxidation at one or more electrodes. In some embodiments, the electrode wherein the redox shuttle is generated is comprised of a substantially smaller surface area than the other electrode to which it is electrically coupled, so as to mitigate deleterious, unintended, or undesired electrochemical reactions from occurring. Other embodiments of the device or system may employ electrode(s) wherein the redox shuttle is generated to be comprised of a substantially smaller surface area by electrically coupling said electrode to more than one other electrode. For example, if the redox shuttle is activated via cathodic process, then the cathode may be comprised of less active surface area than the anode by (a) having less electroactive area, or (b) being coupled to multiple anodes which in combination provide substantially more area than the cathode so as to mitigate opportunity for deleterious, unintended, or undesired electrochemical reactions from occurring. Non-limiting examples of surface, or electroactive, area aspect ratios for the counter electrode (i.e., electrode wherein no redox chemistry should occur) relative to the working electrode (i.e., electrode wherein the redox shuttle is to be generated), or C/E aspect ratio, are at least: 3:1, 6:1, 10:1, 100:1, 1000:1, or higher. In some devices or systems, one or more reference electrodes may be employed to establish a potential difference between electrodes, however the aforementioned ratio relates to electroactive surface areas wherein the current path is involved. In some embodiments all the electrodes may lie in a single plane while in other embodiments a combination of electrodes may lie in multiple planes, or be oriented parallel to perpendicular with respect to one another during the current path. In some embodiments, it may be desirable to minimize the inter-electrode distance between the active electrodes as much as possible, so as to mitigate excess voltage from being required. Specific, non-limiting examples include an inter-electrode gap of any of the following distances (i.e., distance between electrodes included in the current path): ≤100 μm, ≤10 μm, ≤1 μm, ≤100 nm, ≤10 nm, ≤1 nm, ≤100 μm, ≤10 μm, ≤1 μm. Designing a device or system within these parameter ranges will likely enable a high degree of redox reversibility as exhibited by high Coulombic efficiency (i.e., charge transfer to the redox shuttle vs. other constituents) and high bond cleavage efficiency (i.e., migration of the active redox shuttle to the cleavage site followed by successful cleavage and subsequent nucleotide addition) over many cycles of redox shuttle activation, cleavage, and nucleotide addition. Exemplary values of high Coulombic efficiency include ≥75%, ≥85%, ≥95%, ≥99%, 99.9%, and ≥99.99% while exemplary values of high bond cleavage efficiency include ≥50%, ≥60%, ≥75%, ≥90%, ≥99%, ≥99.9%. Designing a device or system within these parameter ranges will likely enable the device or system to work over many cycles of redox shuttle activation, cleavage, and nucleotide addition on the order of ≥10×, ≥100×, ≥1,000×, ≥10,000×, ≥100,000×, ≥100,000,000×. In some embodiments, the electrodes are activated by applying intermittent, pulse, or continuous voltage or current at one or more values to enable many cycles of redox shuttle activation, cleavage, and nucleotide addition.

### VIII. Nucleotide Molecules

Some embodiments comprise a nucleotide molecule. In some embodiments, the molecule comprises any one of the following structures:

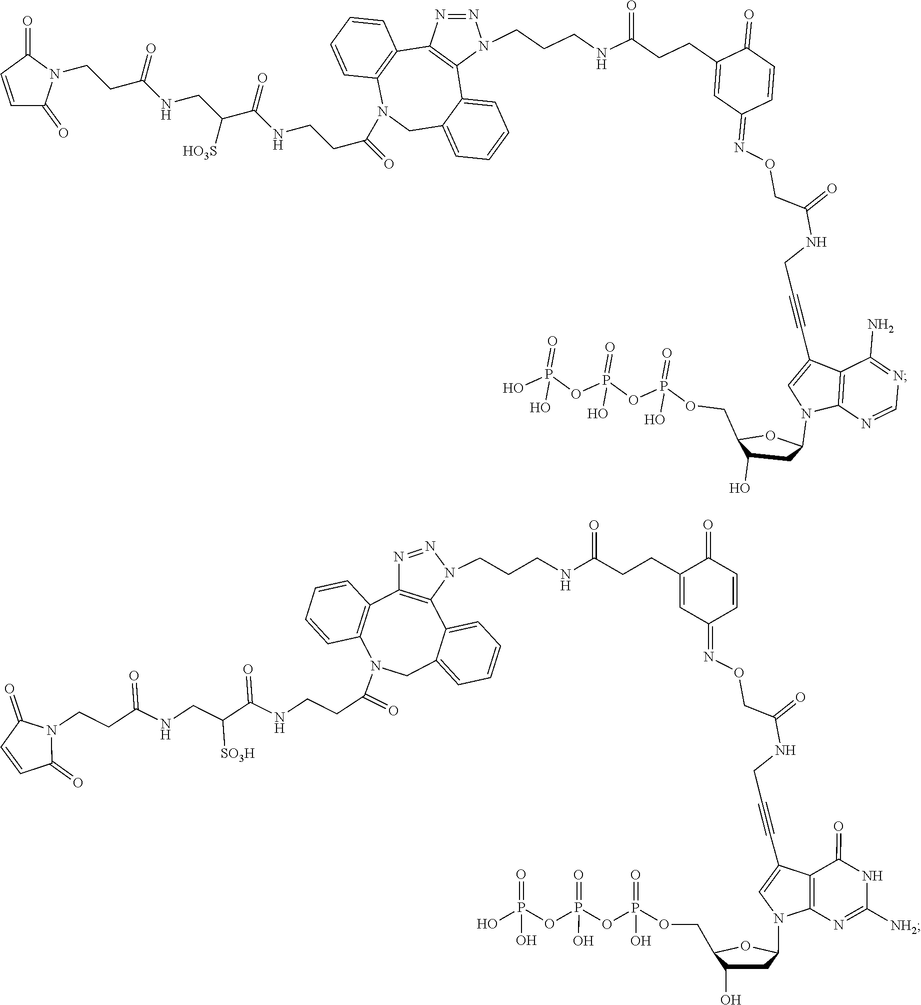

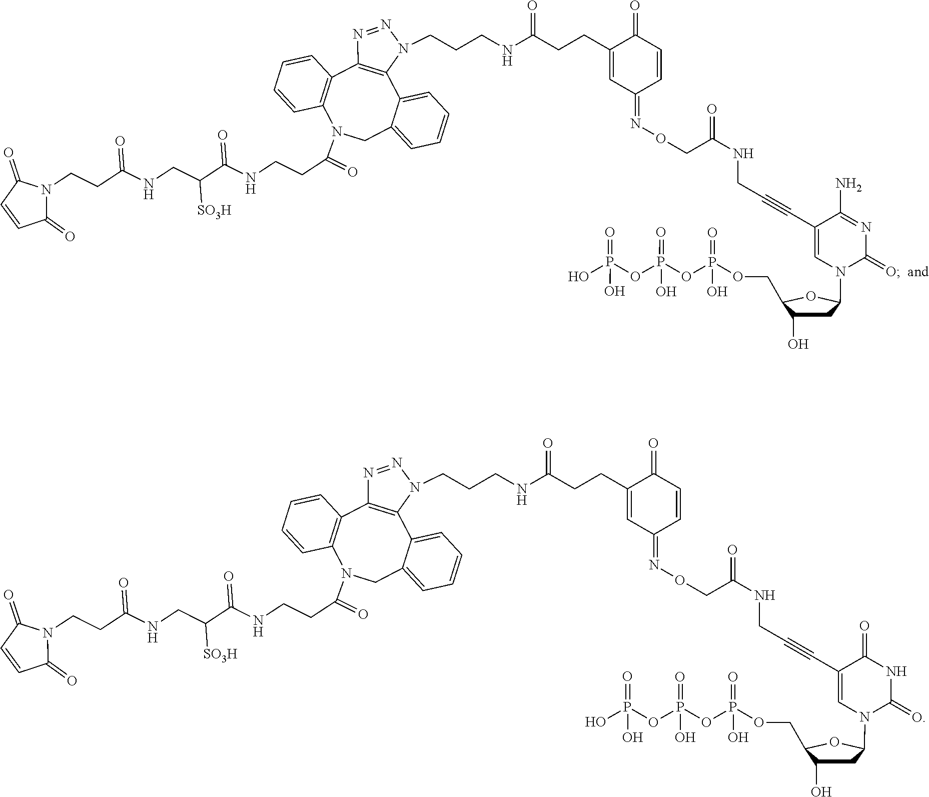

In some embodiments, the molecule comprises any one of the

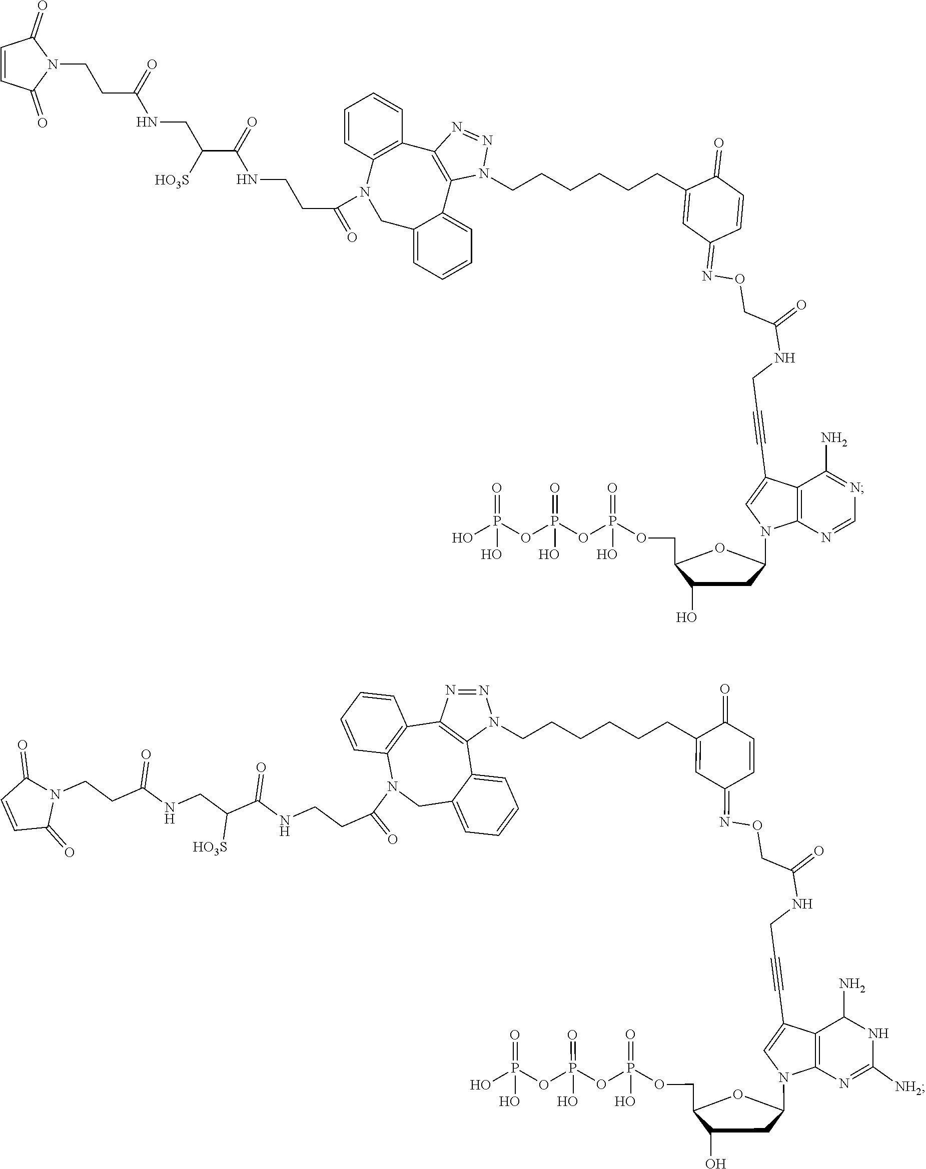 [SMILES: Nc1ncnc2c1c(C#CCNC(=O)CO/N=C1\C=CC(=O)C(CCCCCCn3nnc4c3-c3ccccc3CN(C(=O)CCNC(=O)C(CNC(=O)CCN3C(=O)C=CC3=O)S(=O)(=O)O)c3ccccc3-4)=C1)cn2[C@H]1C[C@@H](O)[C@@H](COP(=O)(O)OP(=O)(O)OP(=O)(O)O)O1 ; NC1=Nc2c(c(C#CCNC(=O)CO/N=C3\C=CC(=O)C(CCCCCCn4nnc5c4-c4ccccc4CN(C(=O)CCNC(=O)C(CNC(=O)CCN4C(=O)C=CC4=O)S(=O)(=O)O)c4ccccc4-5)=C3)cn2[C@H]2C[C@@H](O)[C@@H](COP(=O)(O)OP(=O)(O)OP(=O)(O)O)O2)C(N)N1]

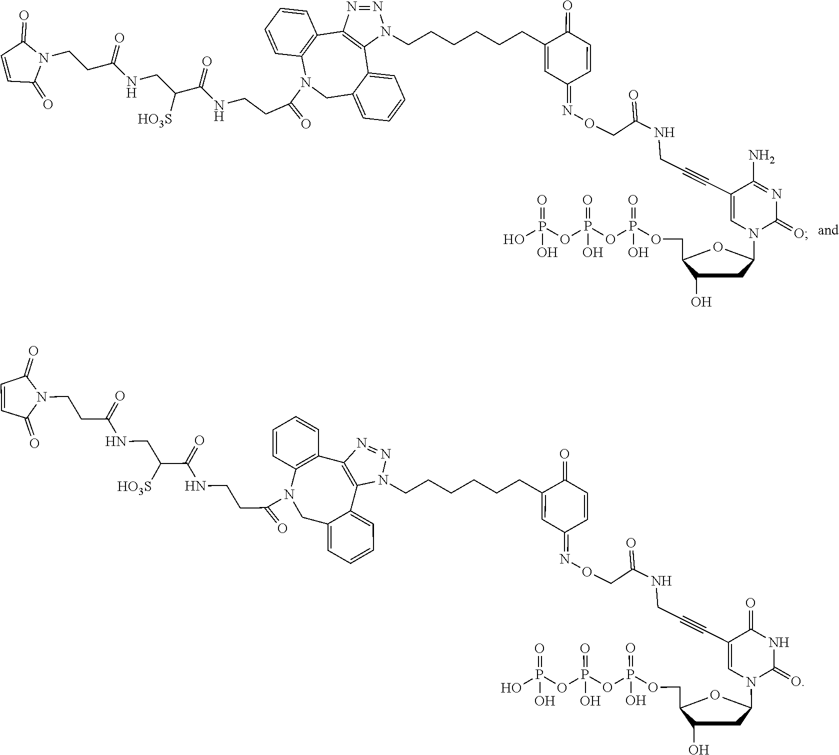 [SMILES: Nc1nc(=O)n([C@H]2C[C@@H](O)[C@@H](COP(=O)(O)OP(=O)(O)OP(=O)(O)O)O2)cc1C#CCNC(=O)CO/N=C1\C=CC(=O)C(CCCCCCn2nnc3c2-c2ccccc2CN(C(=O)CCNC(=O)C(CNC(=O)CCN2C(=O)C=CC2=O)S(=O)(=O)O)c2ccccc2-3)=C1 ; O=C(CCN1C(=O)C=CC1=O)NCC(C(=O)NCCC(=O)N1Cc2ccccc2-c2c(nnn2CCCCCCC2=C/C(=N/OCC(=O)NCC#Cc3cn([C@H]4C[C@@H](O)[C@@H](COP(=O)(O)OP(=O)(O)OP(=O)(O)O)O4)c(=O)[nH]c3=O)C=CC2=O)-c2ccccc21)S(=O)(=O)O]

In some embodiments, the molecule comprises the following structure:

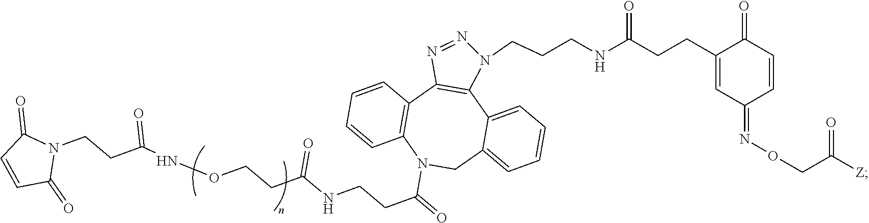

wherein Z is any one of the following:

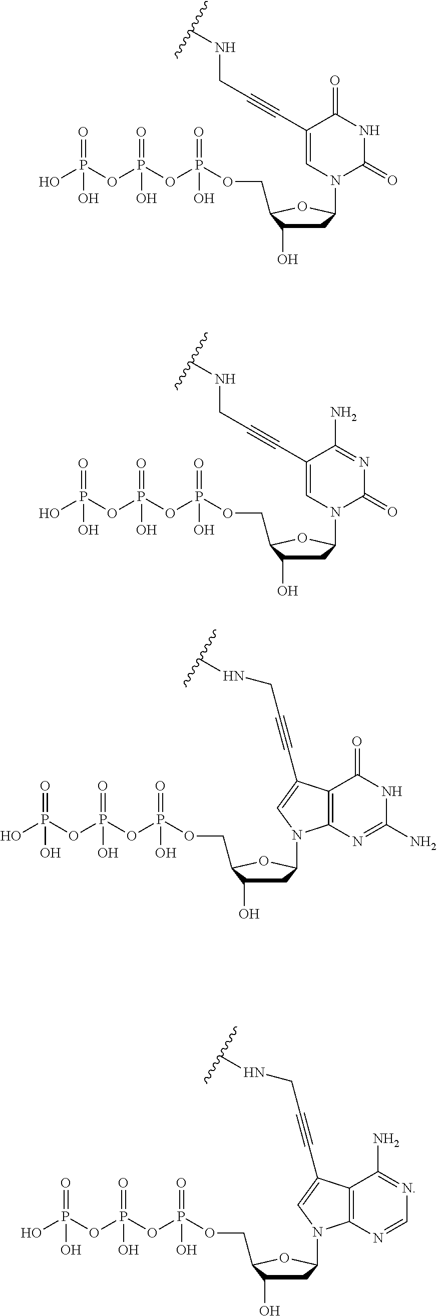

In some embodiments, the molecule comprises the following structure:

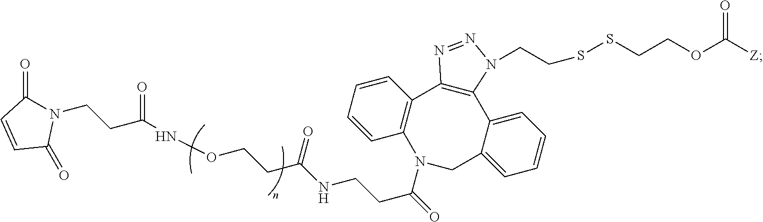

wherein Z is any one of the following:

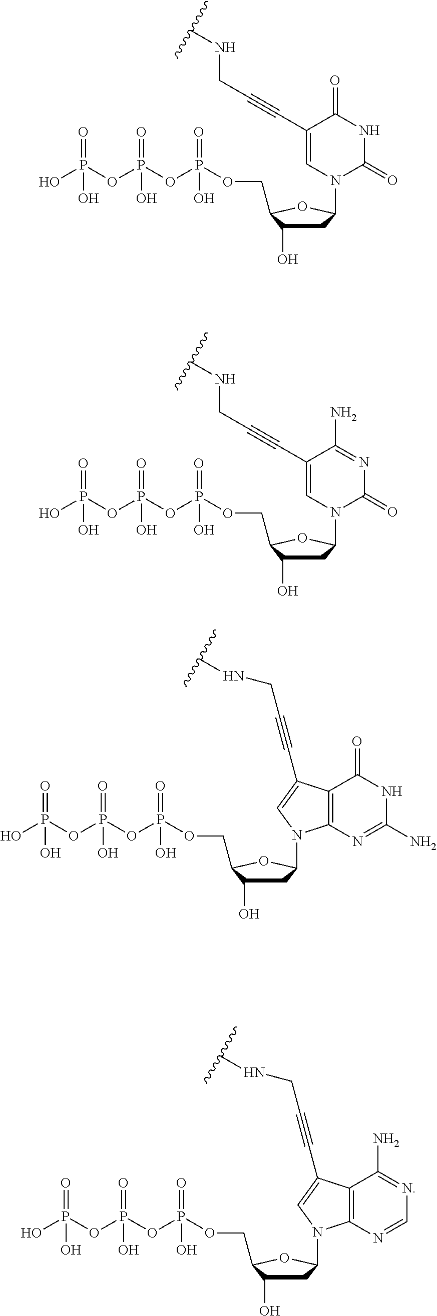

In some embodiments, the molecule comprises the following structure:

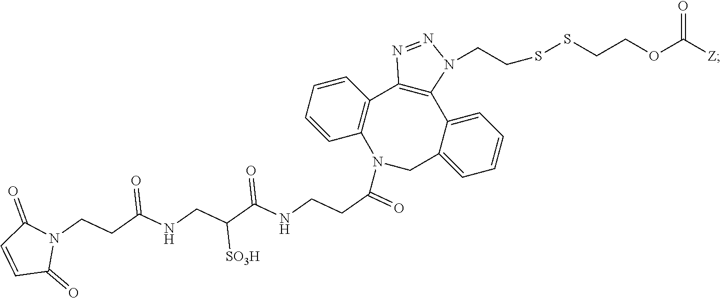

wherein Z is any one of the following:

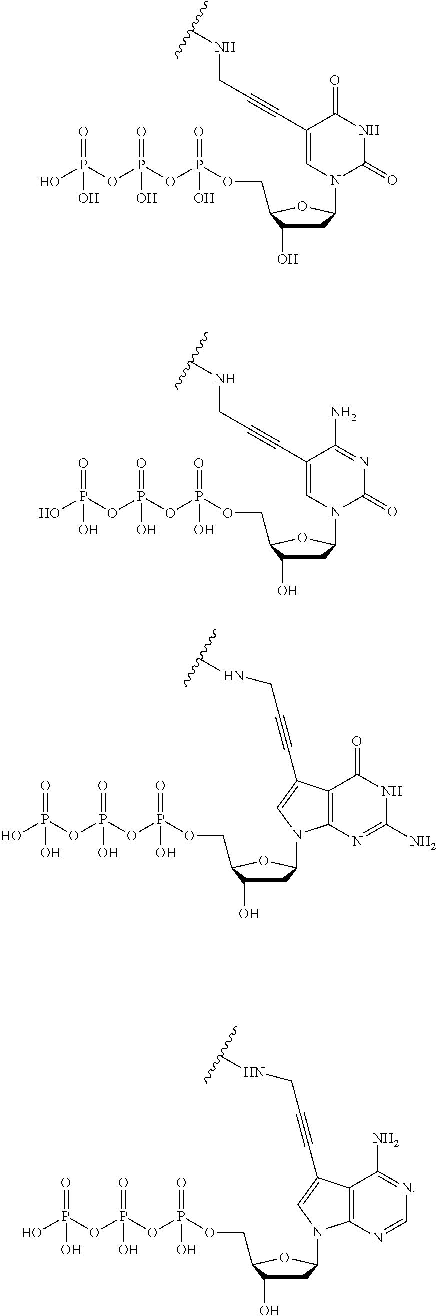

In some embodiments the molecule comprises the following structure:

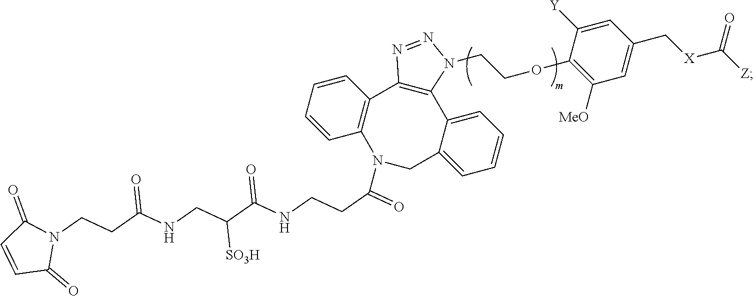

wherein X is O or NH;
wherein Y is H or OMe; and
wherein Z is any one of:

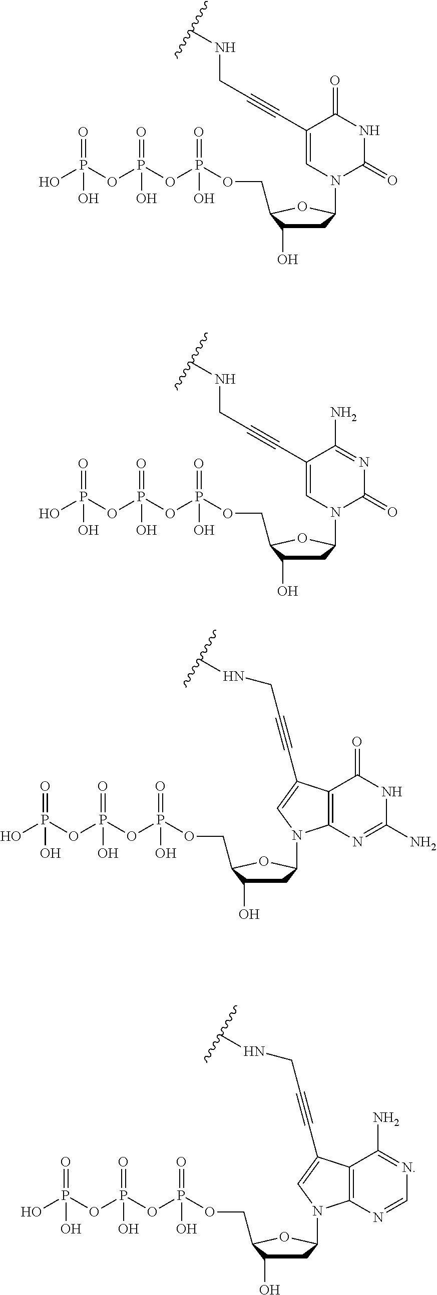

In some embodiments, the molecule comprises the following structure:

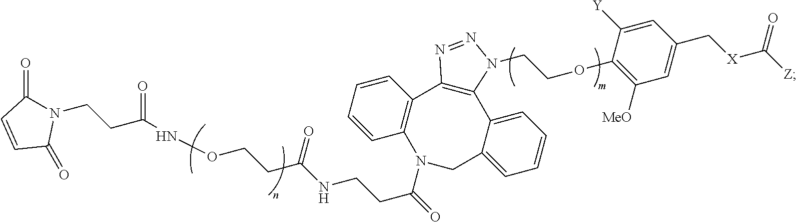

wherein X is O or NH;
wherein Y is H or OMe; and
wherein Z is any one of:

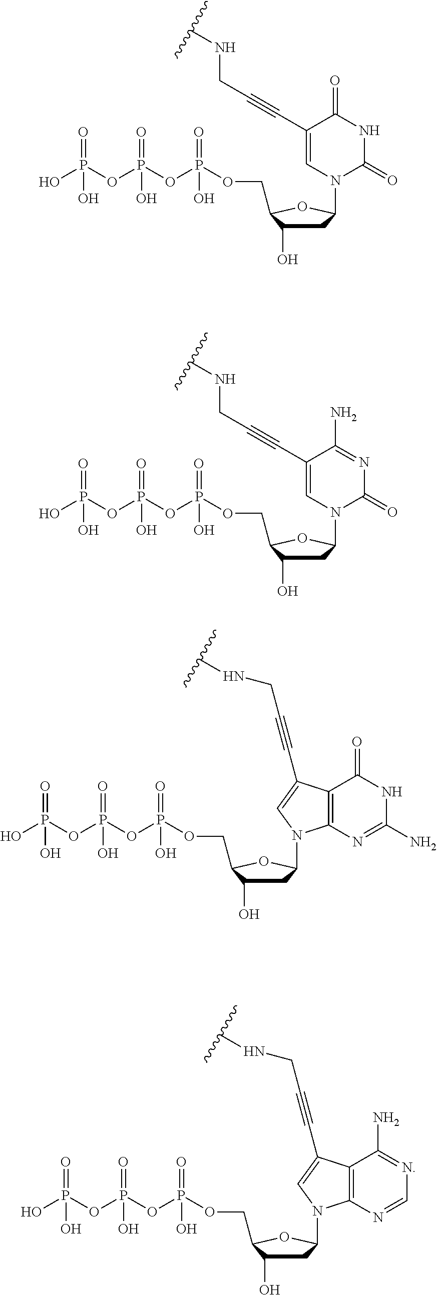

In some embodiments, the molecule comprises the following structure:

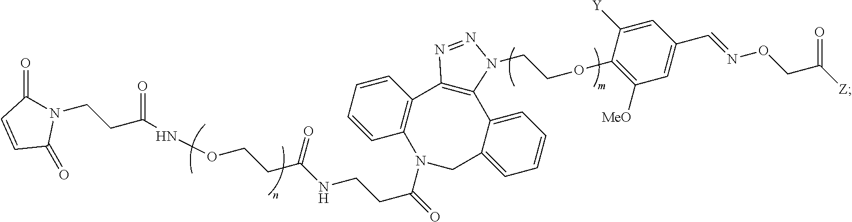

wherein Y is H or OMe; and
wherein Z is any one of:

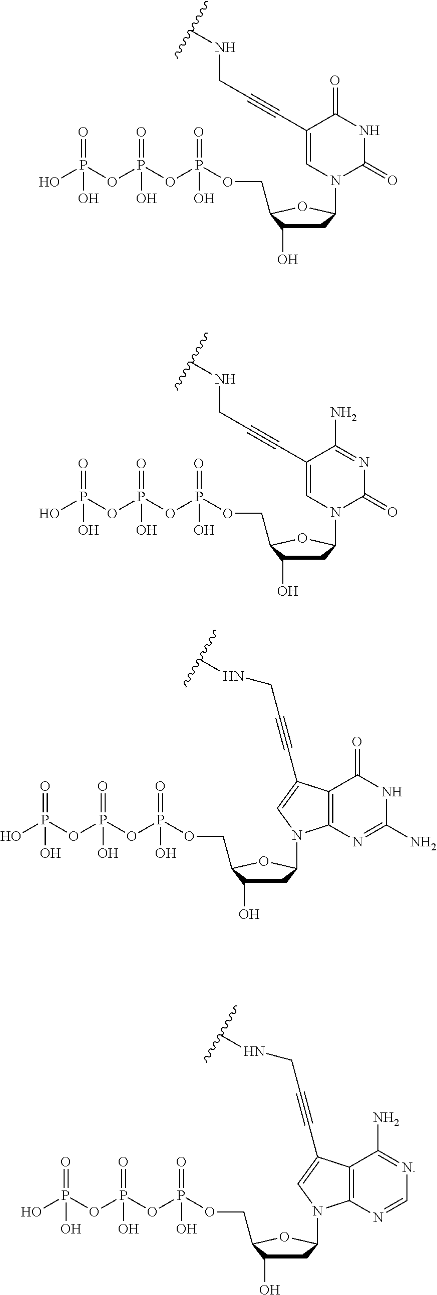

In some embodiments, the molecule is covalently linked to a tether, comprising the following structure:

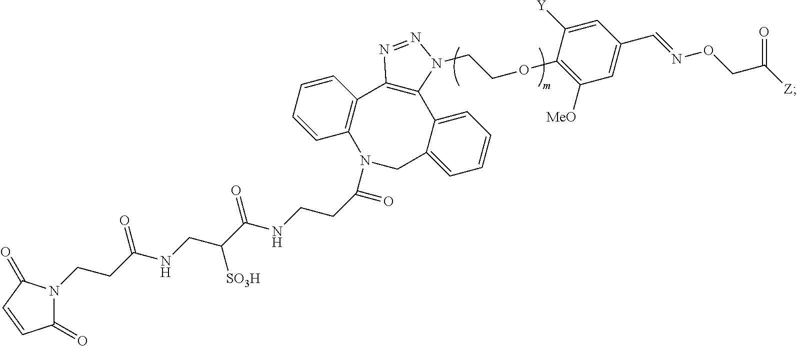

wherein Y is H or OMe; and
wherein Z is any one of:

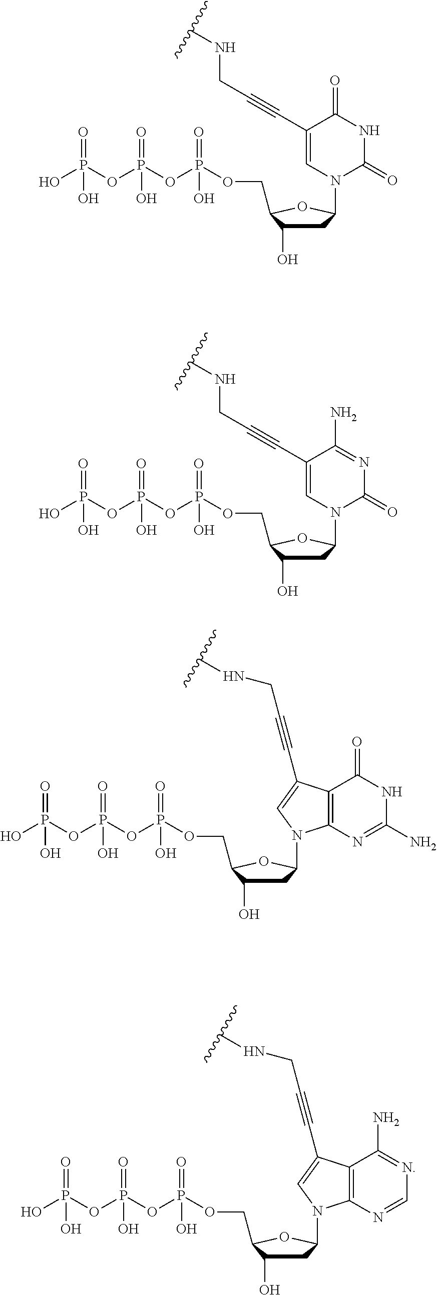

In some embodiments, the molecule comprises any one of the

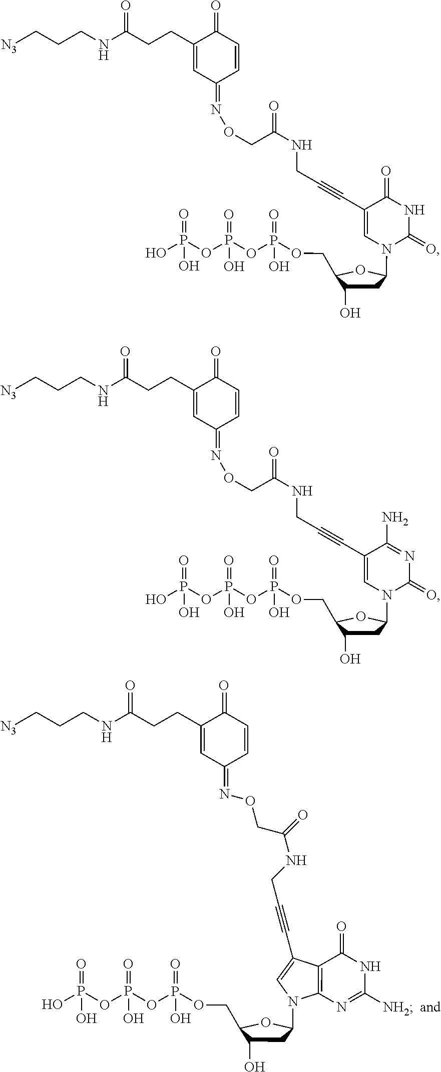

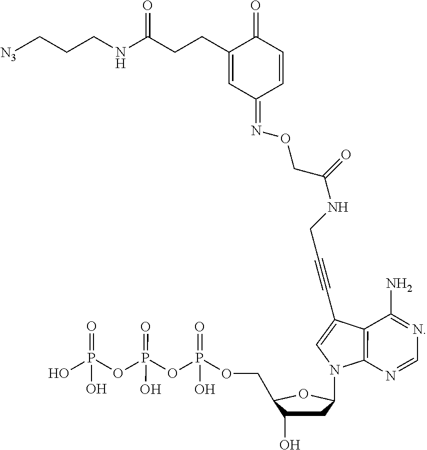

In some embodiments, the molecule comprises any one of the following structures: structures:

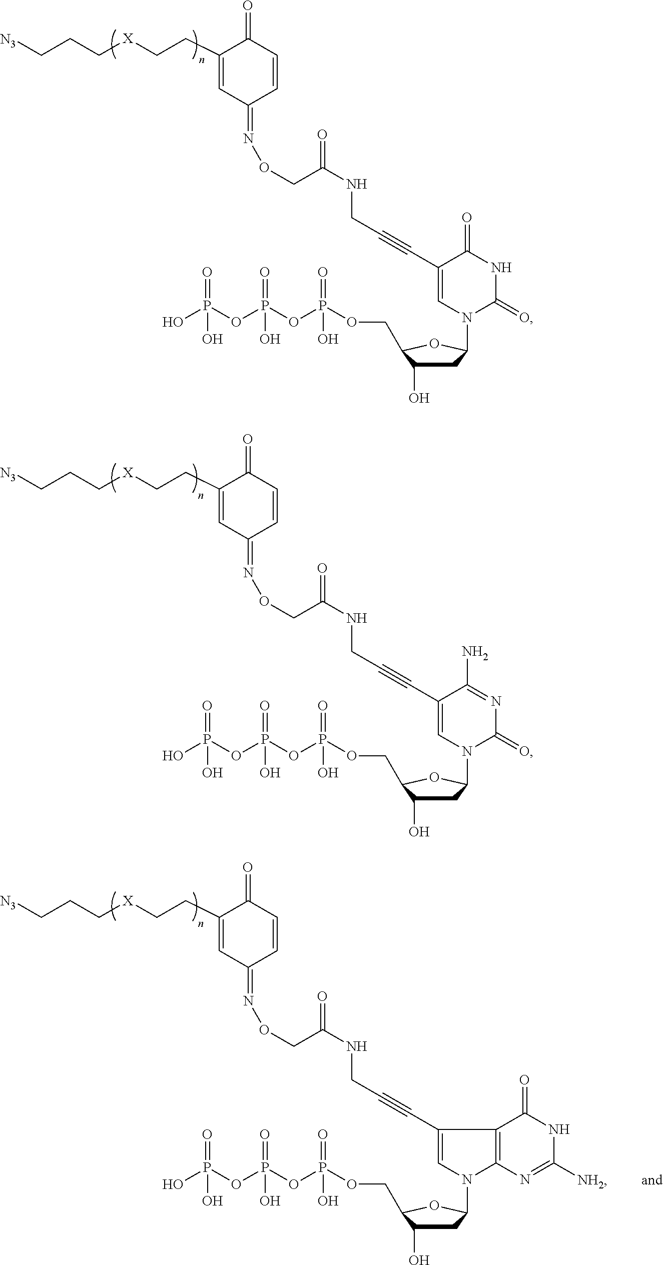

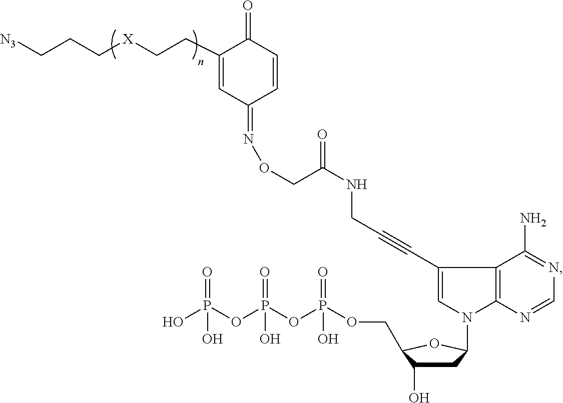

wherein X is C(O), C(O)NH, CH2, or O; and wherein n=1 for X═C(O), n=1 for C(O)NH, n=1 for CH2, and n=1-4 for X═O, wherein n is an integer.

In some embodiments, the molecule comprises any one of the following structures:

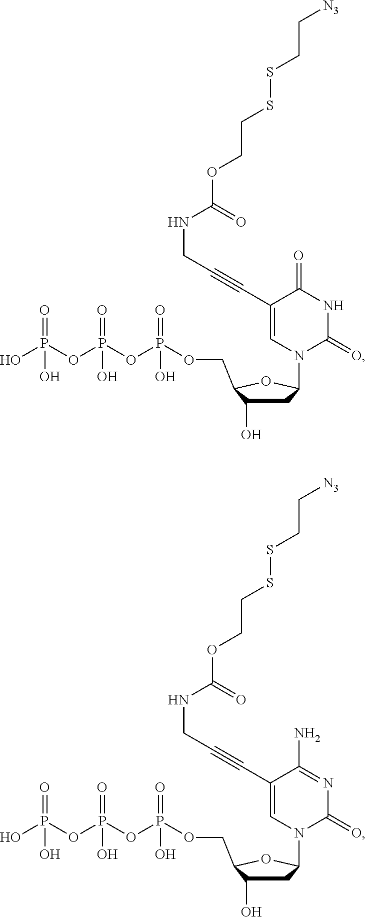

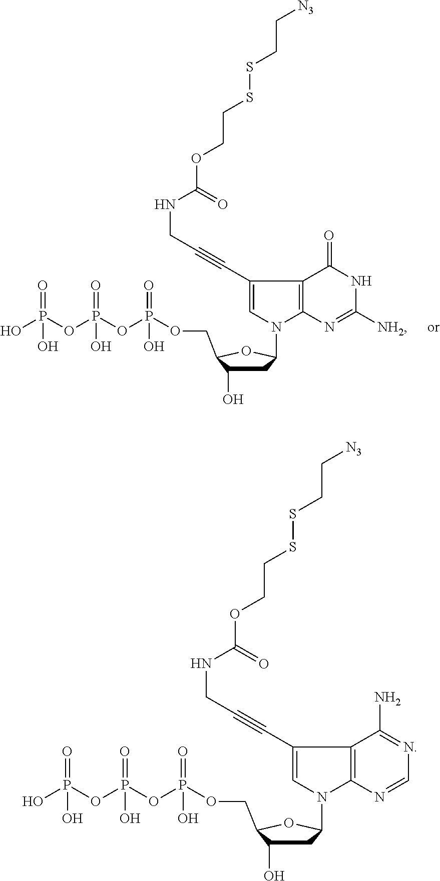

In some embodiments, the molecule comprises any one of the following structures:

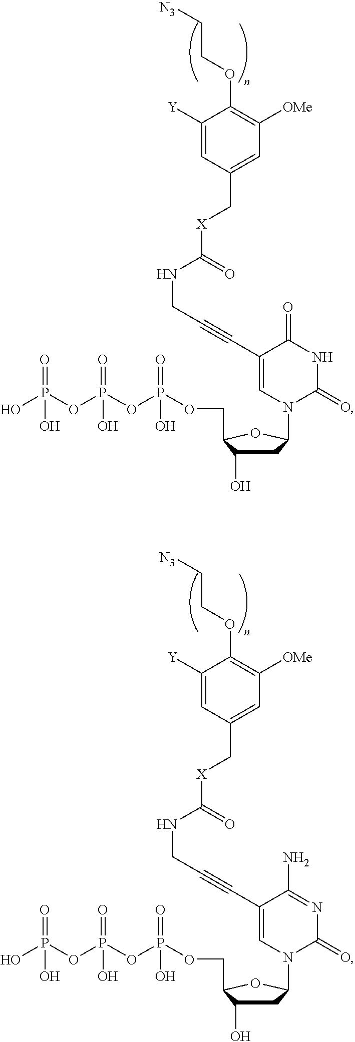

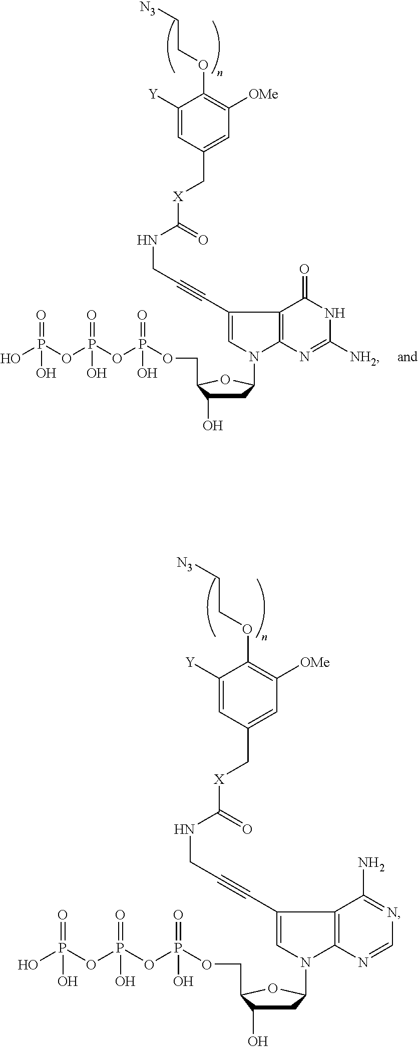

wherein X is O or NH, and wherein Y is H or OMe.

In some embodiments, the molecule comprises any one of the

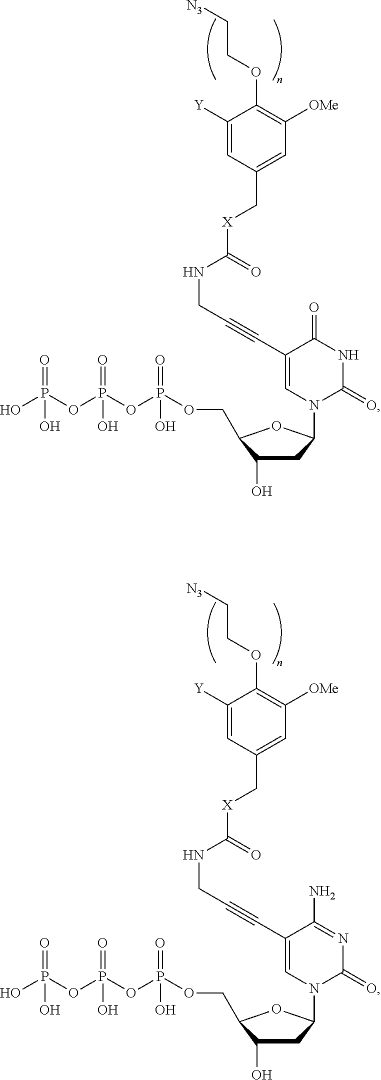

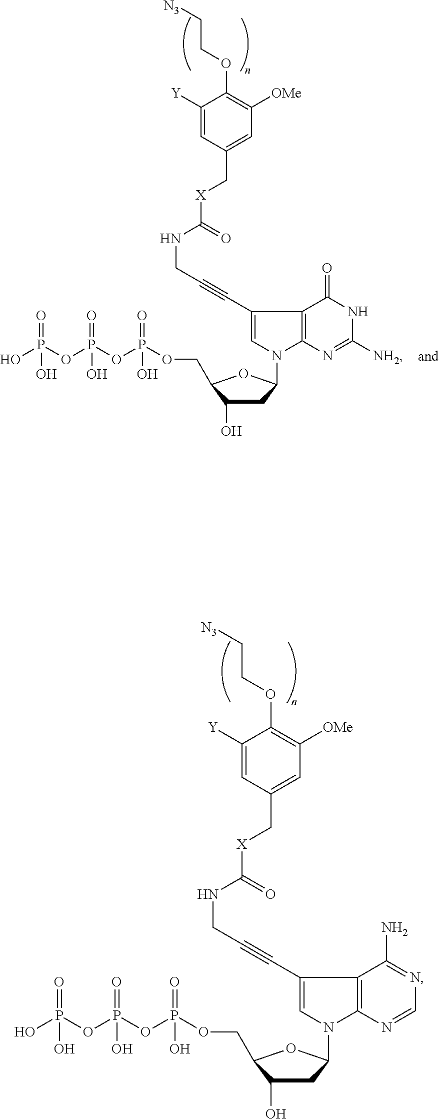

wherein X is O or NH, and wherein Y is H or OMe.

In some embodiments, the molecule comprises any one of the

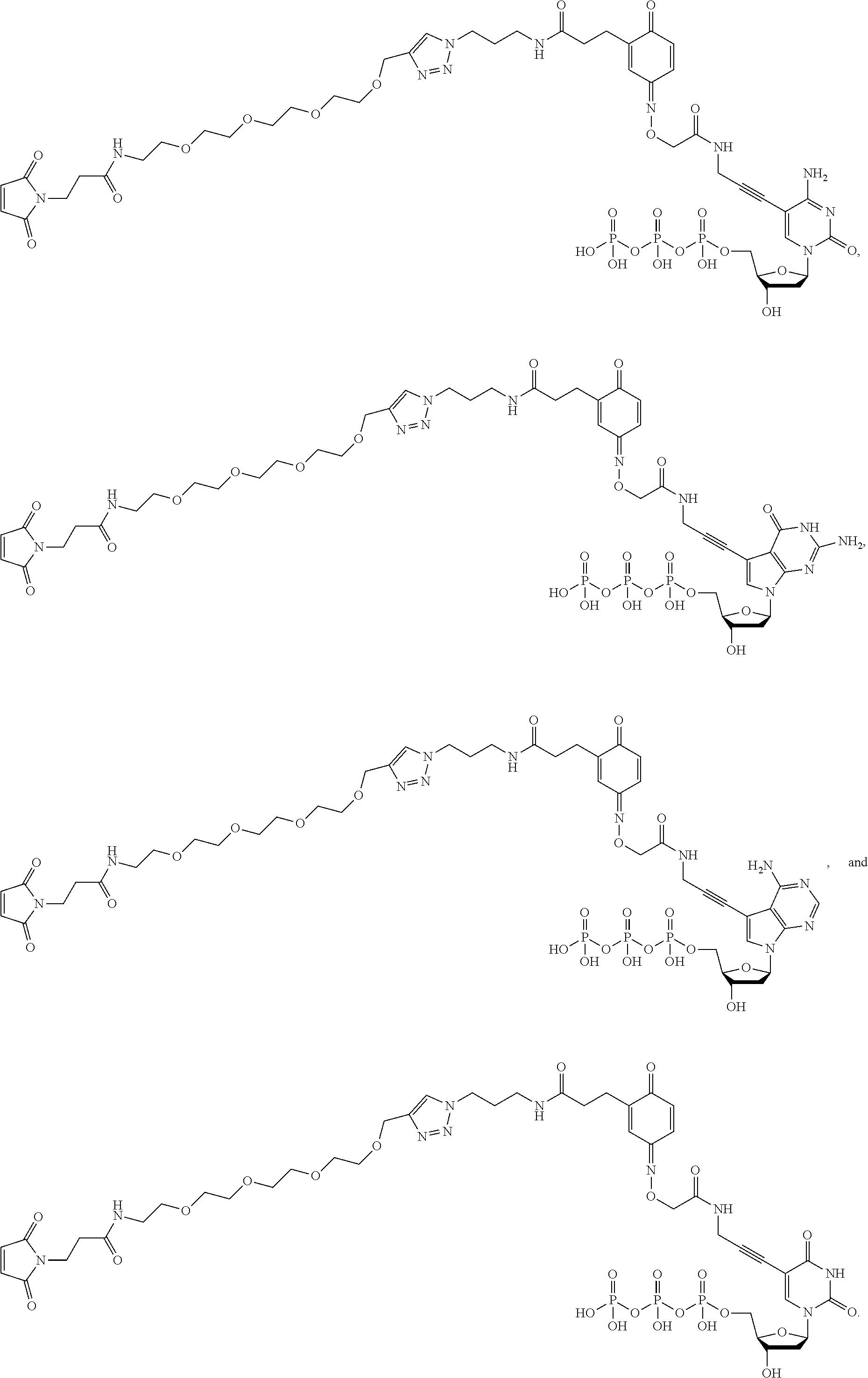

In some embodiments, the molecule comprises any one of the

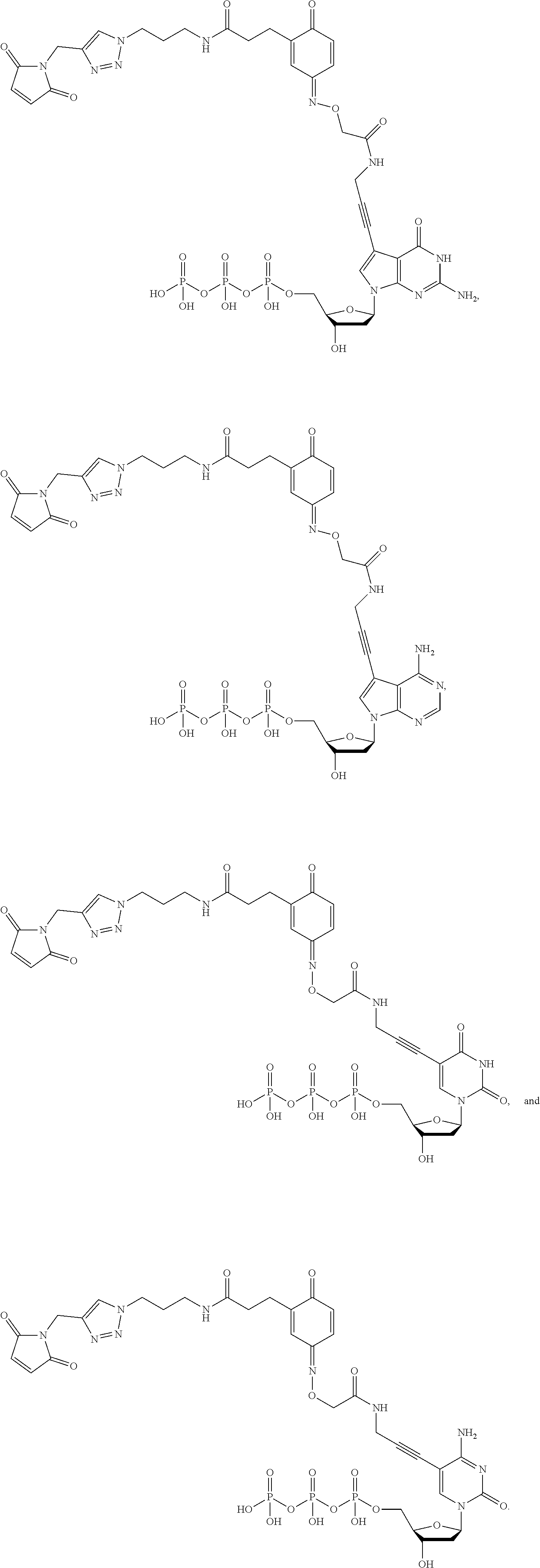

In some embodiments, the molecule comprises any one of the following structures:

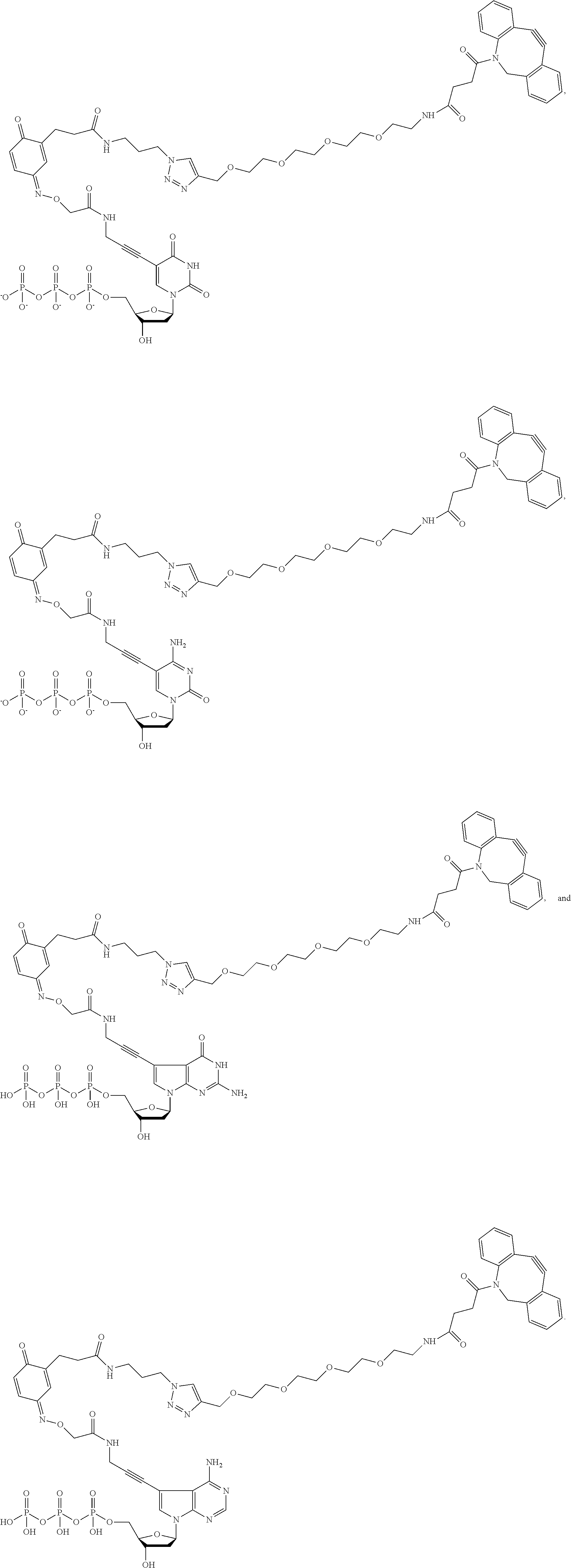

### EXAMPLES

### Example 1. Design of TdT Variants

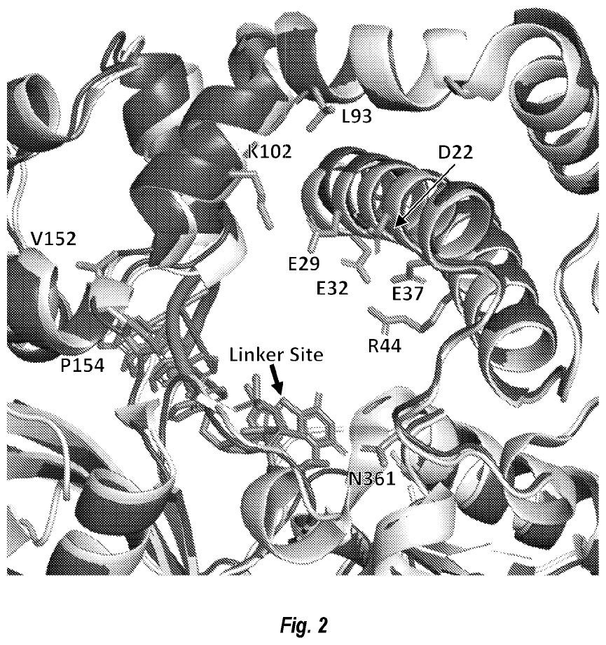

Variants of terminal deoxynucleotidyl tranferase (TdT) were generated starting from the amino acid sequence of wild-type murine TdT, and the software PROSS was used to design variants predicted to have increased stability (see for example amino acid sequences P1 and P2 of Table 1). Goldenzweig, A. et al. Molecular Cell “Automated Structure- and Sequence-Based Design of Proteins for High Bacterial Expression and Stability,” 63(2): 337-346 (2016). Sites for nucleotide conjugation were then identified, including E32, V152, and P154 (FIG. 2) by visualization of structural models of the stabilized variants in Pymol. In order to site-specifically attach a linker-nucleotide molecule via covalent chemistries, mutations were be made to introduce reactive amino acid residues at the selected attachment sites. In some cases, a cysteine residue (C) was installed. In other cases, non-canonical amino acids, such as p-azido-L-phenylalanine (AzF) were installed. A list of all variants studied/used is shown in Table 1.

Of note, a stabilized mTdT (called P1) was developed with 85.64% identity with wild-type mTdT.

P1 variant of Table 1:
(SEQ ID NO: 2)
KISQYAAQRRTTLNNYNKKFTDALDILAENAELRGNEGSALAF
RRAASVLKSLPFPITSMKDTEGIPNLGDKVKRIIQEILEDGESSEAKAVL
NDERYQAFKLFTSVFGVGPKTAEKWYRMGFRTLEDIRSDKTLRFTRMQKA
GFLYYEDLVSPVTRPEAEAIEQLVKEAVWQFLPGATVTMTGGFRRGKKTG
HDVDFLITSPEATEEEEKQLLHKVTDWWKKQGLLLYEDIQESTFEKFKLP
SRKVDALDHFQKAFLILKLHHQRVDSGKSGQQEGKGWKAIRVDLVMAPYE
RRAFALLGWTGSRQFERDLRRYARHERKMLLDNHALYDRTKNTFLRAESE
EEIFAHLGLEYIEPWERNA

Of interest, one variant (C7) was engineered to site-specifically install/insert a cysteine (i.e., substitute another amino acid in a specified position of the wild-type protein) for conjugation of molecules to the TdT. Other modifications were explored, but the C7 variant was the enzyme used in most studies.

C7 variant of P1 of Table 1:
(SEQ ID NO: 8)
KISQYAAQRRTTLNNYNKKFTDALDILAENAELRGNEGSALAF
RRAASVLKSLPFPITSMKDTEGIPNLGDKVKRIIQEILEDGESSEAKAVL
NDERYQAF{*}C{*}LFTSVFGVGPKTAEKWYRMGFRTLEDIRSDKTLRF
TRMQKAGFLYYEDLVSPVTRPEAEAIEQLVKEAVWQFLPGATVTMTGGFR
RGKKTGHDVDFLITSPEATEEEEKQLLHKVTDWWKKQGLLLYEDIQESTF
EKFKLPSRKVDALDHFQKAFLILKLHHQRVDSGKSGQQEGKGWKAIRVDL
VMAPYERRAFALLGWTGSRQFERDLRRYARHERKMLLDNHALYDRTKNTF
LRAESEEEIFAHLGLEYIEPWERN

### Example 2. Generation of TdT Variants

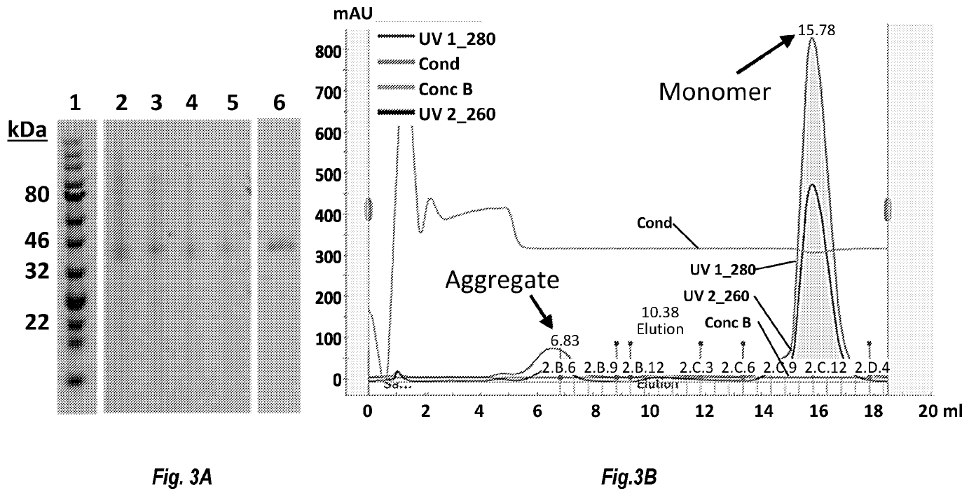

To generate the TdT variants, genes for all of the single conjugation site variants were cloned into the E. coli expression vector pET28b+. The cysteine variants yielded high soluble expression from BL21(DE3) cells via IPTG induction at 30 °C for 2 hours or overnight (16-20 hrs) at 15 °C. The p-AzF variants were co-transformed into BL21 (DE3) cells with pEvol-pAzFRS.1.t1 (Addgene 73547) containing genes for expression of the tRNA synthetase and tRNA necessary to incorporate p-AzF at the amber codon TAG. These variants also yielded a high level of soluble expression via IPTG and arabinose induction at 30 °C for 2 hours or overnight at 15 °C with the addition of 2 mM pAzF. All proteins were first purified over Ni-NTA via a His-tag that was incorporated into the expression construct. Size exclusion chromatography (SEC) using a Superdex200 column was then used to isolate the pure, monomeric proteins in TdT Storage Buffer (200 mM Potassium Phosphate pH 6.5, 100 mM NaCl). 7.4-18 mg of final, pure protein was obtained from IL culture for each protein and an example of one of the purifications (C1) is shown in FIG. 3A and FIG. 3B. Proteins were either stored at −80 C with 5% glycerol or lyophilized and stored at room temperature for conjugation and functional testing.

### Example 3. Test of Ability of TdT Variants to Add Nucleotides to ssDNA Chain

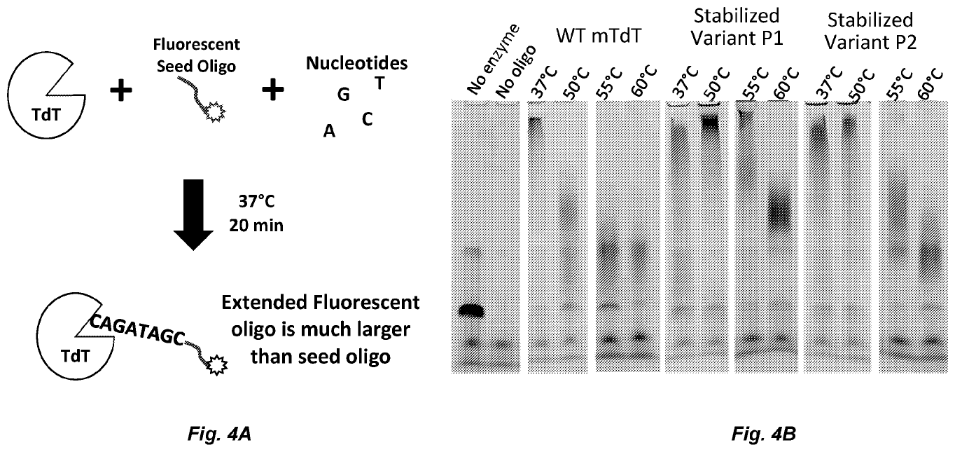

The ability of TdT variants to add nucleotides to a ssDNA chain, called a seed oligo, was tested via incubation of the TdT variant, a seed oligo with a fluorescent dye, and a mixture of 4 2′-deoxynucleotide triphosphates. The enzyme activity was tested at 37 °C, 50 °C, 55 °C, and 60 °C (FIG. 4B). Both P1 and P2 exhibited greater activity than WT mTdT at temperatures >50 °C, with P1 maintaining activity even at 55 °C.

### Example 4. Nucleotides with Redox-Cleavable Linkers

Nucleotides can be conjugated to the enzyme via redox-cleavable linkers. Structures of four azide-redox-linker-nucleotide molecules are depicted below, labeled A-D. The azide can be tethered to a nucleotide via redox-cleavable linker, such as a quinone oxime ether (A and B) or a quinone propionic acid linker with appropriate methyl group substitution, known as the trimethyl lock linker (C and D). Examples of molecules that contain a combination of an azide, a redox cleavable linker, and a nucleotide are shown below.

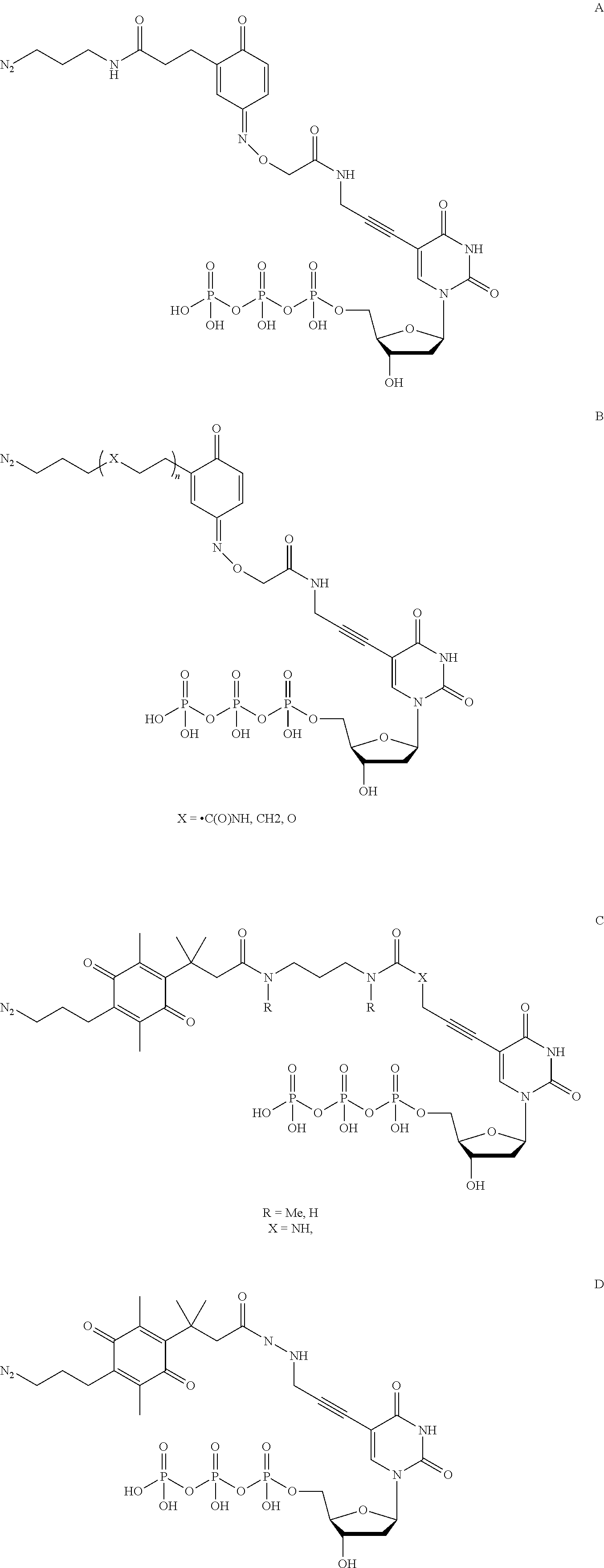

As shown above, A-D are structures of molecules containing an azide, redox cleavable linker (quinone) and nucleotide triphosphate. In this case, “T” (2′-deoxythymidine triphosphate) is shown as a representative example but non-limiting nucleotide triphosphate. A) Example of an azide-quinone-nucleotide wherein the quinone portion is installed as a quinone oxime ether. B) Example an azide-quinone-nucleotide wherein the quinone portion is installed as a quinone oxime ether, and the linker between the quinone and the azide consists of a methylene chain, a chain containing polyethylene oxide (PEG) groups, or a chain containing a ketone or amide functionality, wherein n=1 for X═C(O), n=1 for C(O)NH, n=1 for CH2, and n=1-4 for X═O, wherein n is an integer. C) Example of an azide-quinone-nucleotide wherein the quinone is tethered through a methyl-substituted propionic acid linker, and D) example of an azide-quinone-nucleotide wherein the quinone is tethered through a methyl-substituted propionic acid linker wherein “A” can be anywhere from 0 additional atoms to 6 additional atoms.

Additional examples of molecules containing the azide-redox-linker-nucleotide are depicted below. The quinone propionic acid linker with appropriate methyl group substitution, known as the trimethyl lock linker (A and B) can be attached to a nucleotide using suitable intermediate spacers, such as the 4-aminobenzyl alcohol or an aminopropanol spacer. Upon reduction of the quinone, both the redox-cleavable and the intermediate spacer could be cleaved from the nucleotide.

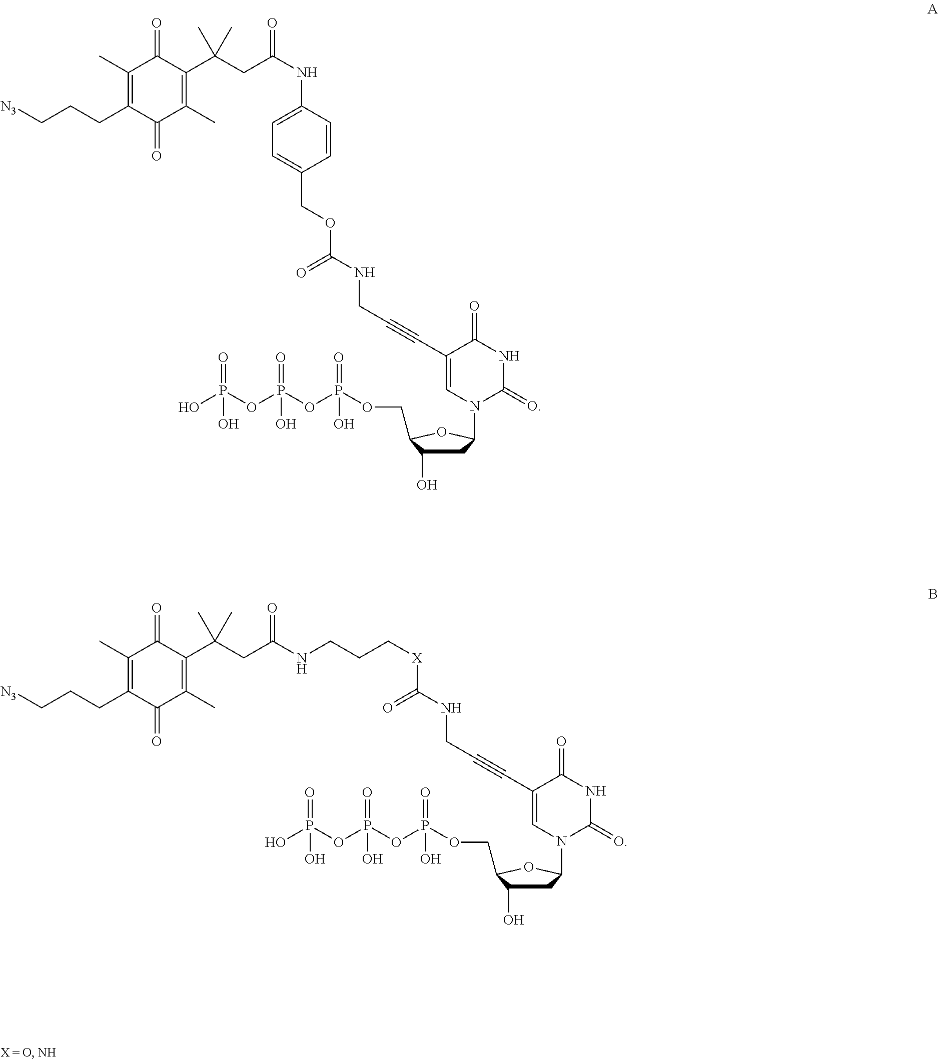

As shown above, A-B are structures of molecules containing an azide, redox cleavable linker (quinone) and nucleotide triphosphate. In this case, “T” (2′-deoxythymidine triphosphate) is shown as a representative nucleotide triphosphate. A) Example of an azide-quinone-nucleotide wherein the quinone is tethered through a methyl-substituted propionic acid linker, via an intermediate 4-aminobenzyl alcohol spacer and B) example of an azide-quinone-nucleotide wherein the quinone is tethered through a methyl-substituted propionic acid linker using a propylene diamine or aminopropanol spacer.

Schemes 1-3 below show the expected reductive cleavage mechanism for the quinone tethered through a methyl-substituted propionic acid linker. In the first case (Scheme 1, labeled A), there is no intermediate spacer; reduction of the quinone leads to cyclization and displacement of amine. In cases where an intermediate spacer is present, reduction of the quinone leads to cyclization, followed by a secondary cleavage of the intermediate spacer (Scheme 2, labeled B, and Scheme 3, labeled C). Use of methyl-substituted propionic acid linkers (also known as trimethyl lock linkers) are well described. Okoh et al. ChemBioChem “Trimethyl Lock: A Multifunctional Molecular Tool for Drug Delivery, Cellular Imaging, and Stimuli-Responsive Materials,” 19(7): 1668-1694 (2018).

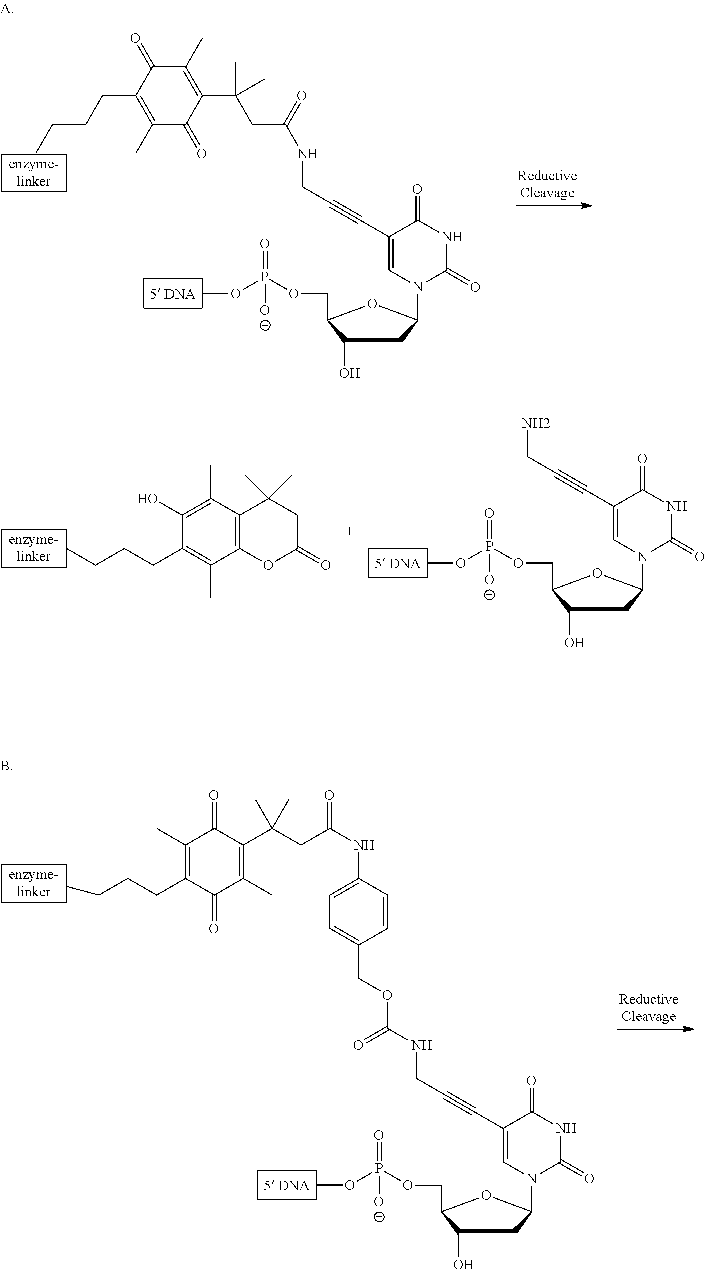 [SMILES: CCCCC1=C(C)C(=O)C(C(C)(C)CC(=O)NCC#Cc2cn([C@H]3C[C@@H](O)[C@@H](COP(=O)([O-])OC)O3)c(=O)[nH]c2=O)=C(C)C1=O ; CCCCc1c(C)c2c(c(C)c1O)C(C)(C)CC(=O)O2 ; COP(=O)([O-])OC[C@H]1O[C@@H](n2cc(C#CCN)c(=O)[nH]c2=O)C[C@H]1O ; CCCCC1=C(C)C(=O)C(C(C)(C)CC(=O)Nc2ccc(COC(=O)NCC#Cc3cn([C@H]4C[C@@H](O)[C@@H](COP(=O)([O-])OC)O4)c(=O)[nH]c3=O)cc2)=C(C)C1=O]

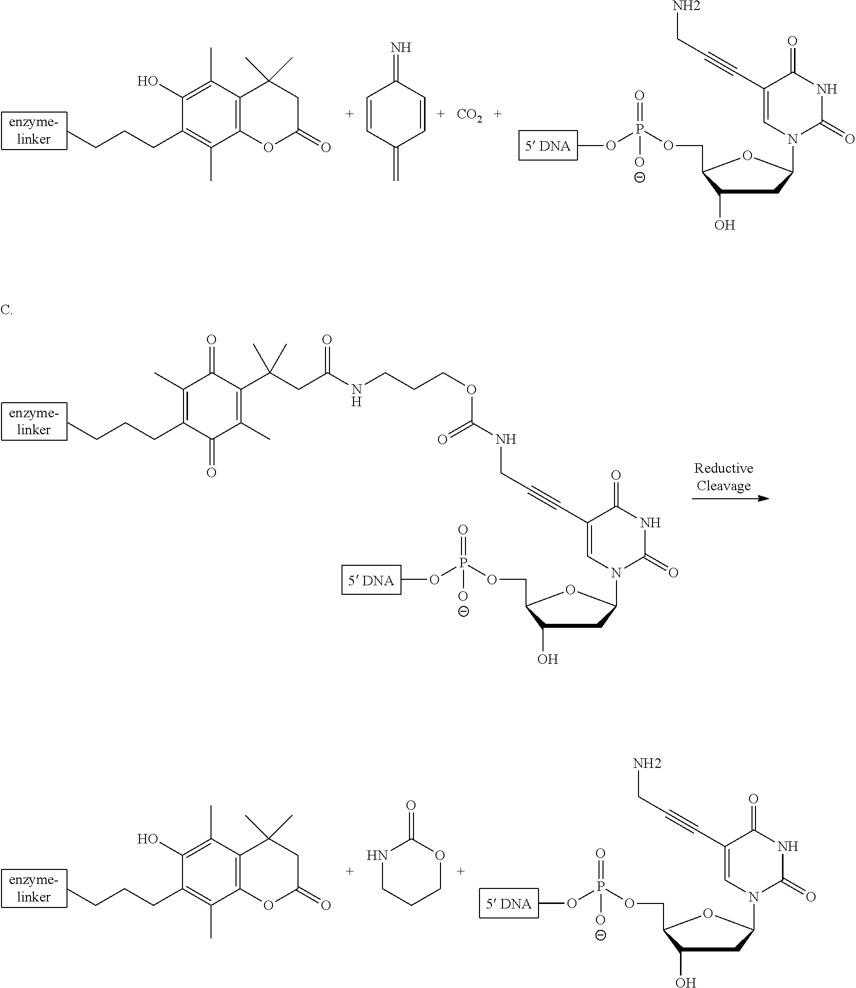

Shown above are expected products after the azide-quinone-nucleotide is tethered to the enzyme and the nucleotide is tethered to a growing strand of ssDNA. Scheme 1, A) Reduction of the quinone leads to cyclization and displacement of the amine. Scheme 2, B), and Scheme 3, C) Reduction of the quinone leads to cyclization, followed by a secondary cleavage of an intermediate spacer molecule.

However, when the compound shown as structure I below (with a type of trimethyl lock linker) was synthesized and cleavage was tested with reduced methylene blue (leucomethylene blue), it did not cleave as expected. The expected cleavage products are the propargyl-amine substituted nucleotide analog II and the cyclized hydroquinone III. As demonstrated by the HPLC traces shown in FIGS. 25A-25C, expected cleavage products were not observed. FIG. 25A shows an HPLC chromatogram of compound I. FIG. 25B shows an HPLC chromatogram of compound II (expected cleavage product). FIG. 25C shows an HPLC chromatogram of a crude reaction mixture when compound I was treated with leucomethylene blue (reducing agent). Reducing agents including dithiothreitol (DTT), sodium thiosulfate, sodium dithionite, and sodium bisulfite were also tested. Treatment with the reducing agents did not lead to observation of expected cleavage products.

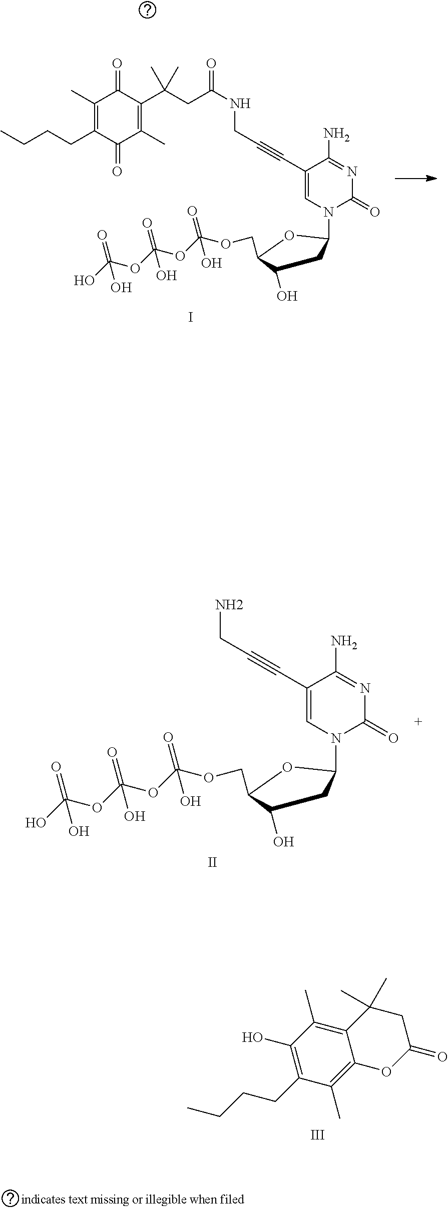

The redox-cleavable linker could also consist of a disulfide linkage. Disulfides have reduction potentials well within the range that will not split water or interfere with nucleic acid chemistry. Disulfides may be used in bioconjugation chemistries and are reduced under physiological conditions (for example, using glutathione). Common reducing agents such as dithiothreitol and (tris(2-carboxyethyl)phosphine) (TCEP) are used to cleave disulfide bonds. Below are examples of azide-disulfide-nucleotide constructs. These molecules could also provide a means to tether a nucleotide to the enzyme through a redox cleavable linker. Depending on the design, cleavage of the disulfide bond could result in an additional cyclization of the reduced thiol (shown in Scheme 4). It is likely desirable to generate nucleic acids free of thiol substitutions due to detrimental secondary structure effects that could arise from maintaining thiol substituents on the growing nucleic acid chains. Examples of nucleotide-disulfide conjugates have been reported. WO 2022/212408 A1.

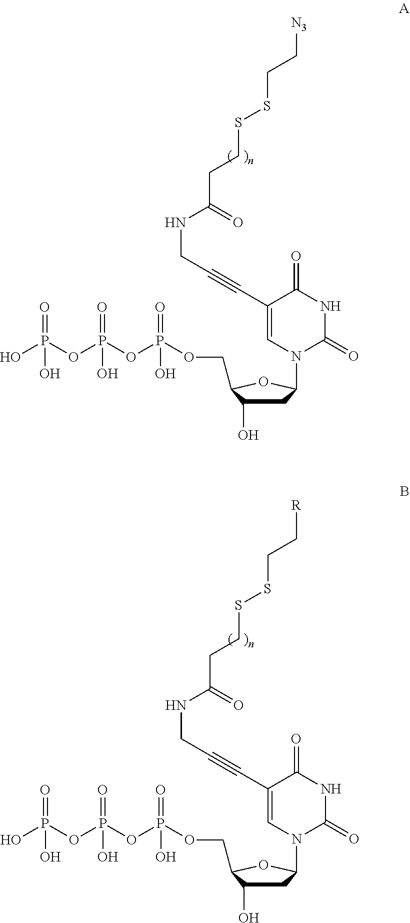

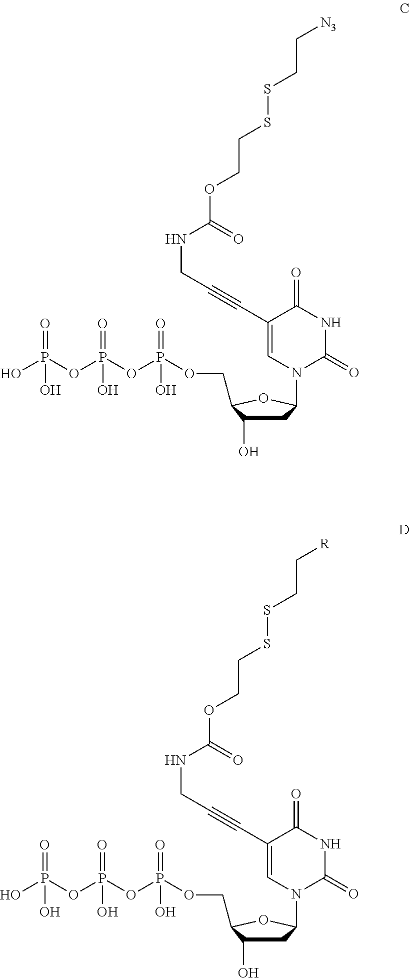

Shown above are representative structures consisting of A) azide-disulfide-nucleotides where the carbon chain length is variable (n=an integer from 1-3) B) disulfide-nucleotides wherein the R group is a functional group or linker-functional group capable of undergoing a bioconjugation reaction (and the carbon chain length is variable, n=an integer from 1-3) C) azide-disulfide nucleotides linked via a carbamate and D) disulfide-nucleotides tethered to a functional group, R, where R is a functional group or linker-functional group capable of undergoing a bioconjugation reaction. In this case, “T” (2′-deoxythymidine triphosphate) is shown as a representative nucleotide triphosphate.

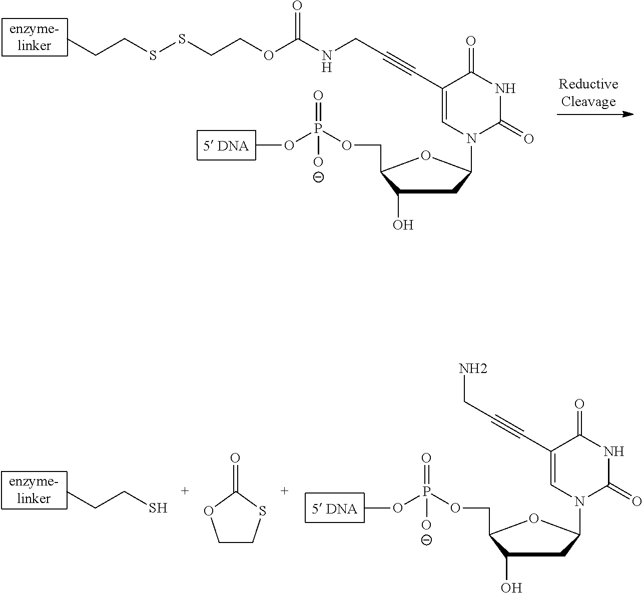

Scheme 4. Example of a nucleotide tethered to an enzyme via a disulfide linkage. Reductive cleavage of the disulfide bond leads to formation of free thiols. When configured appropriately, the thiol will cyclize into a carbonyl group to form a five-membered ring (1,3-oxathiolan-2-one), leaving a propargyl-amine as the pendant functional group on the incorporated nucleotide.

Another candidate for the redox-cleavable linker are substituted benzyl compounds, such as the syringic acid and vanillin family as depicted below. These linkers are cleaved through oxidative conditions that still fall within an acceptable potential. WO 2021/158412 A1; US 2022/0023820 A1. The proclivity toward cleavage can be altered via methoxy substitution on the ring (e.g., two meta-methoxy groups present in syringic acid analogs versus one meta-methoxy group for vanillin analogs). These linkers can be tethered to nucleotides via carbamate/urea linkages (A, B) or via an oxime linkage (C, D).

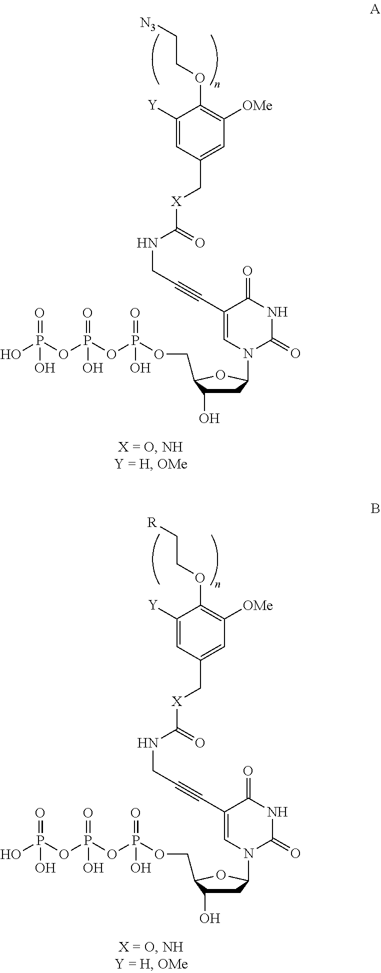

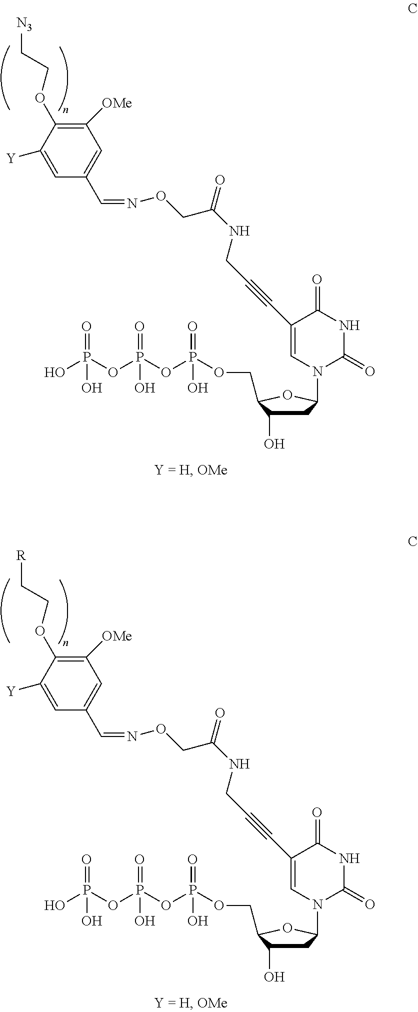

Shown above are representative structures consisting of A) azide-vanillin/syringic-nucleotides consisting of an azide for enzyme attachment, attached via a polyethylene glycol chain where n=1 or more units, and a vanillin or syringic acid-based linker tethered to the nucleotide via a urea or carbamate linkage B) analogs consisting of vanillin or syringic acid-based linkers tethered to the nucleotide via a urea or carbamate linkage wherein the R group is a functional group or linker-functional group capable of undergoing a bioconjugation reaction (and the carbon chain length is variable). C) azide-vanillin/syringic-nucleotides consisting of an azide for enzyme attachment, attached via a polyethylene glycol chain where n=1 or more units, and a vanillin or syringic acid-based linker tethered to the nucleotide via an oxime linkage D) analogs consisting of vanillin or syringic acid-based linker tethered to the nucleotide via an oxime linkage wherein the R group is a functional group or linker-functional group capable of undergoing a bioconjugation reaction (and the carbon chain length is variable). In the depicted cases, “T” (2′-deoxythymidine triphosphate) is shown as a representative but non-limiting nucleotide triphosphate.

### A. Synthesis of Nucleotides Tethered to Quinone Oxime Ether Linkers

### 1. Synthesis of Trifunctional Structures

Nucleotides tethered to a quinone oxime ether and azide linkage can be synthesized according to Schemes 5-7 below. Commercially available modified nucleotides with a propargyl amine substituent (e.g., 3) can be modified to contain an aminooxy substituent via reaction of the propargyl amine 3 with a Boc-protected aminooxyacetic acid 4 to provide compound 5. Mass spectral data for compound 5 is shown in FIG. 26. Removal of the Boc group using established protocols provides the aminooxy-substituted nucleotide 6. Mass spectral data for compound 6 is shown in FIG. 27. Hutter, D. et al. “Nucleosides Nucleotides Nucleic Acids,” Labeled nucleoside triphosphates with reversibly terminating aminoalkoxyl groups,” 29(11): (2010). Note that this synthesis to obtain 6 is an exemplary synthesis only. Compounds disclosed in Table 4 were prepared using aminooxy-substituted nucleotides obtained from suppliers. This example synthesis is demonstrated for 2-deoxy guanosine-5′-triphosphate, but the synthetic protocol can be adapted for preparing analogs of the other deoxynucleotide triphosphate analogs (analogs of 2′-deoxyadenosine triphosphate, 2′-deoxycytidine triphosphate, and 2′-deoxythymidine triphosphate).

A molecule containing both a quinone and an azide (deemed an azide-quinone, such as 7, Schemes 6 and 7) was condensed with the aminooxygroup at acidic pH. Purification using reversed-phase chromatography yielded molecules of type 8. These molecules contained an azide, a quinone oxime ether, and a nucleotide and are deemed “azide-quinone-nucleotides.” An example synthesis is demonstrated for an analog of 2-deoxyguanosine-5′-triphosphate, but all four DNA nucleotide analogs can be synthesized using the representative protocol. Starting material structures and mass spectral data are provided for the four nucleotide analogs generated (Table 4).

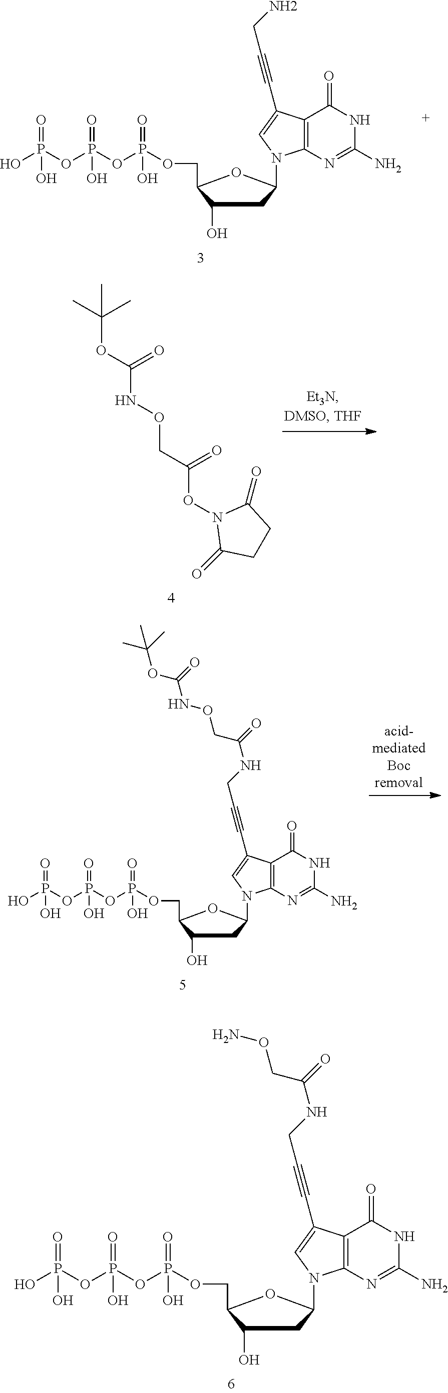 [SMILES: NCC#Cc1cn([C@H]2C[C@@H](O)[C@@H](COP(=O)(O)OP(=O)(O)OP(=O)(O)O)O2)c2nc(N)[nH]c(=O)c12 ; CC(C)(C)OC(=O)NOCC(=O)ON1C(=O)CCC1=O ; CC(C)(C)OC(=O)NOCC(=O)NCC#Cc1cn([C@H]2C[C@@H](O)[C@@H](COP(=O)(O)OP(=O)(O)OP(=O)(O)O)O2)c2nc(N)[nH]c(=O)c12 ; NOCC(=O)NCC#Cc1cn([C@H]2C[C@@H](O)[C@@H](COP(=O)(O)OP(=O)(O)OP(=O)(O)O)O2)c2nc(N)[nH]c(=O)c12]

Scheme 5. Synthesis of an aminooxy-substituted nucleotide.

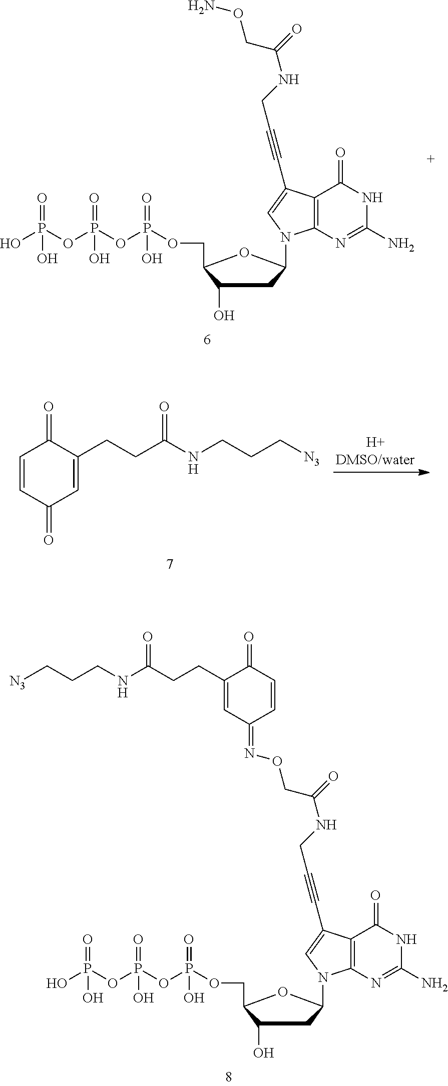 [SMILES: NOCC(=O)NCC#Cc1cn([C@H]2C[C@@H](O)[C@@H](COP(=O)(O)OP(=O)(O)OP(=O)(O)O)O2)c2nc(N)[nH]c(=O)c12 ; [N-]=[N+]=NCCCNC(=O)CCC1=CC(=O)C=CC1=O ; [N-]=[N+]=NCCCNC(=O)CCC1=C/C(=N/OCC(=O)NCC#Cc2cn([C@H]3C[C@@H](O)[C@@H](COP(=O)(O)OP(=O)(O)OP(=O)(O)O)O3)c3nc(N)[nH]c(=O)c23)C=CC1=O]

Scheme 6. Synthesis to generate compounds containing the azide, a quinone oxime ether, and a nucleotide. These structures are deemed azide-quinone-nucleotides.

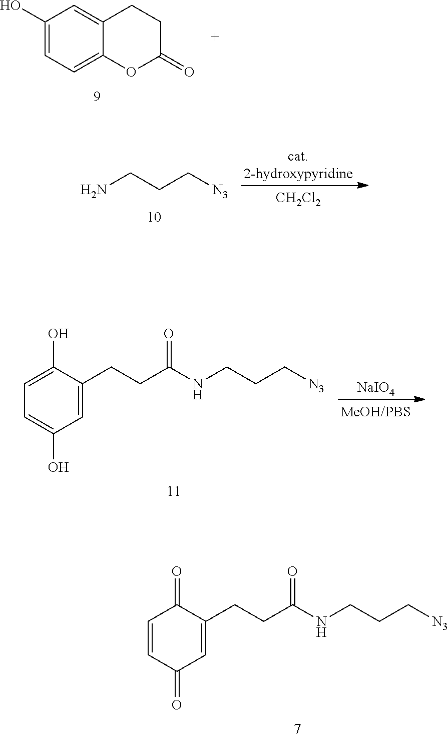

Scheme 7. Synthesis of azide-quinone compound 7.

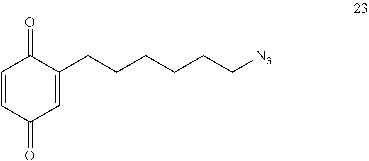 [SMILES: [N-]=[N+]=NCCCCCCC1=CC(=O)C=CC1=O]

Alternative example azide-quinone structure.

The synthesis of quinone 7 is shown in Scheme 7. The synthesis proceeded through aminolysis of a 6-hydroxychroman-2-one 9 with 3-azidopropylamine 10 followed by oxidation of the corresponding 1,4-hydroxyphenol with sodium periodate. Although one quinone oxime ether analog synthesis was demonstrated, other quinones can be condensed with the aminooxy-substituted deoxynucleotidetriphosphate analogs to generate azide-quinone-nucleotides with varying linker lengths. For example, a quinone with a hexamethylene linker (compound 23, shown above) can be synthesized using methods disclosed in Park, S.; Westcott, N. P.; Luo, W.; Dutto, D.; Yousaf, M. N. Bioconjugate Chemistry “General chemoselective and redox-responsive ligation and release strategy,” 25:543-551 (2014). Compound 23 was synthesized as shown in Scheme 8. from compounds reported in Park, et al., 25:543-551 (2014). Deprotection of the tetrahydropyranyl protecting groups of known compound 24 with pyridinium p-toluenesufonate in ethanol followed by oxidation with sodium periodate afforded compound 23. Quinone 23 was condensed with an aminooxy-substituted deoxynucleotidetriphosphate (in this case for an analog of 2-deoxyguanosine-5′-triphosphate) to generate compound 26.

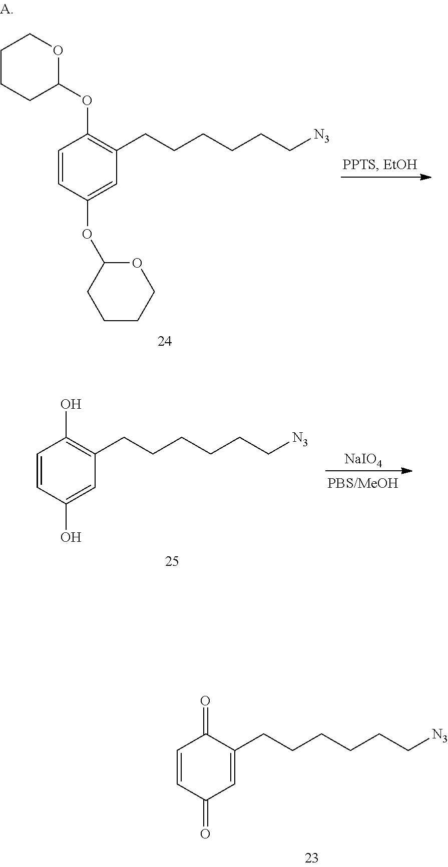

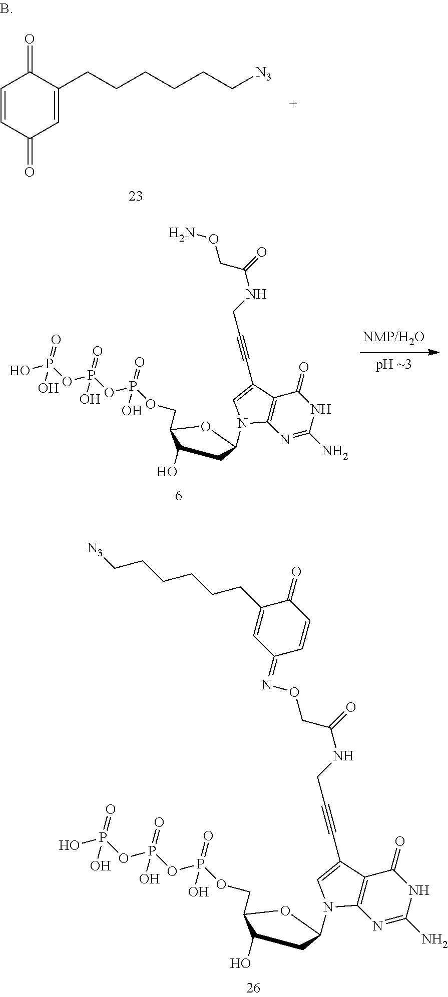 [SMILES: [N-]=[N+]=NCCCCCCC1=CC(=O)C=CC1=O ; NOCC(=O)NCC#Cc1cn([C@H]2C[C@@H](O)[C@@H](COP(=O)(O)OP(=O)(O)OP(=O)(O)O)O2)c2nc(N)[nH]c(=O)c12 ; [N-]=[N+]=NCCCCCCC1=C/C(=N/OCC(=O)NCC#Cc2cn([C@H]3C[C@@H](O)[C@@H](COP(=O)(O)OP(=O)(O)OP(=O)(O)O)O3)c3nc(N)[nH]c(=O)c23)C=CC1=O]

Scheme 8. Synthesis of azide-quinone-nucleotides with a hexamethylene spacer between the azide and the quinone. A) Synthesis of quinone 23 from known starting material 24. B) Synthesis of an azide-quinone-nucleotide using quinone 23.

Table 4 shows aminooxy-substituted nucleotide starting materials and tabulated mass spectral data for azide-quinone nucleotide products. The mass spectral data is shown in FIGS. 28, 29, 30, and 31.

TABLE 4
Aminooxy-substituted nucleotide starting materials and azide-quinone
nucleotide products with tabulated mass spectral data

| | | Product after Reaction with | Expected | Found |
|---|---|---|---|---|
| Nucleotide | Aminooxy-substituted | azide-quinone (azide- | mass | mass |
| analog | Nucleotide | quinone-nucleotide) | (m/z) | (m/z) |
| dATP analog | 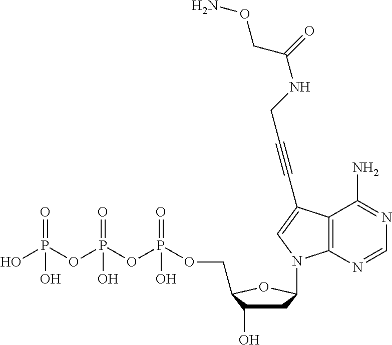 | 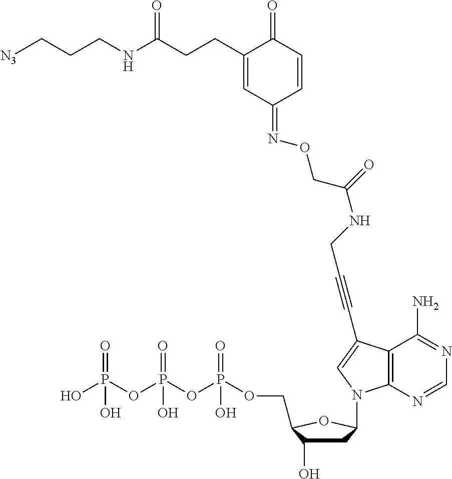 | 860.14 | 859.0 [M − H]⁻ (See FIG. 28) |
| | | 21 | | |
| dGTP analog | 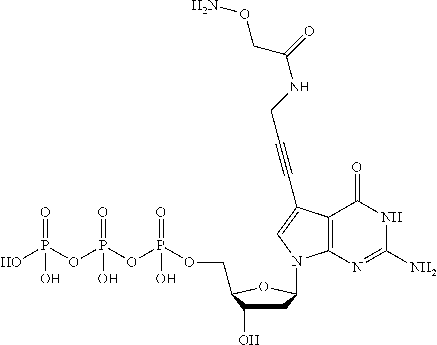 | 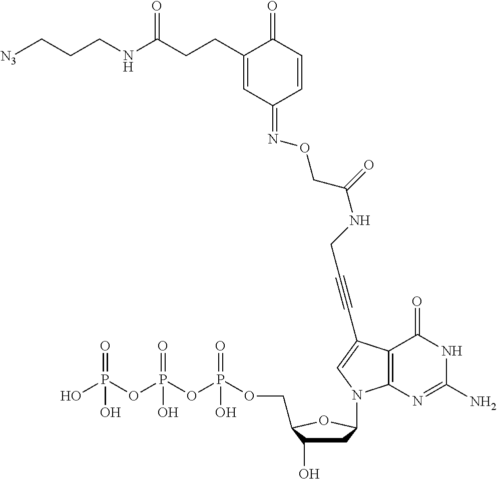 | 876.14 | 875.0 [M − H]⁻ (See FIG. 29) |
| | | 8 | | |
| dCTP analog | 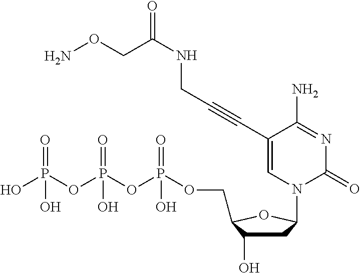 | 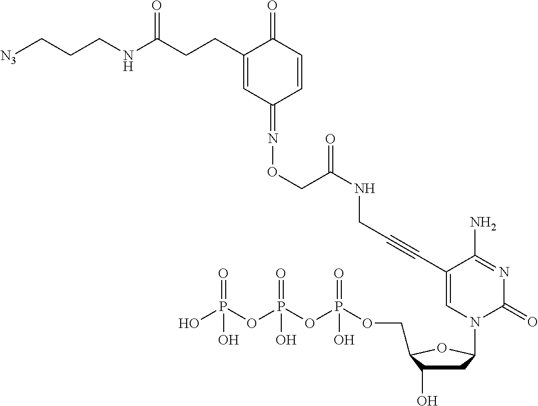 | 837.13 | 836.0 [M − H]⁻ (See FIG. 30) |
| | | 20 | | |
| dTTP analog (also dUTP analog) | 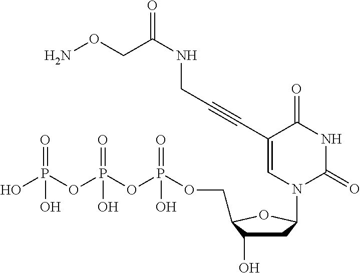 | 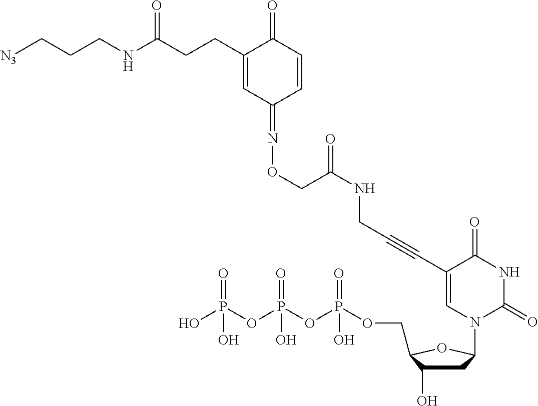 | 838.11 | 837.0 [M − H]⁻ (See FIG. 31) |
| | | 22 | | |

### 2. Protocols for Synthesis of Azide-Quinone Nucleotides

The compounds and/or intermediates were characterized by nuclear magnetic resonance spectroscopy (NMR) on 400 MHz NMR Spectrometer. Chemical shifts (8) are reported in ppm using the following convention: chemical shift, multiplicity (s=singlet, d=doublet, t=triplet, q=quartet, m=multiplet, br=broad), coupling constants, and integration. Spectra are referenced to residual dimethylsulfoxide (2.50 ppm). Mass spectrometric analysis was performed on an Agilent 1260 Infinity instrument with an Agilent 6120 Quadropole MS. Separations were performed using an Agilent Infinity Lab Poroshell EC-C18 column (4.6×100 mm, 2.7 μm) using the following solvent system at a flow rate of 0.5 mL/min: solvent A=0.05 M tricthylammonium acetate (TEAA); solvent B=20% MeCN/0.05M TEAA; gradient method: 90% A/10% B for 3 min; linear gradient form 90% A/10% B to 20% A/80% B from 3-5 min; linear gradient from 20% A/80% B from 5 min to 8 min; maintained at 100% B from 8 min to 20 min. For less polar compounds, an alternative gradient was used where solvent A=0.05 M TEAA and solvent B=80% MeCN/0.05 M TEAA; gradient method: 97.5% A/2.5% B for 3 min; linear gradient from 97.5% A/2.5% B to 90% A/10% B from 3-5 min; linear gradient from 90% A/10% B to 40% A/60% B from 5-10 min; linear gradient from 40% A/60% B to 100% B from 10-15 min; maintained at 100% B from 15 min to 25 min. Compounds were detected by UV absorption at 210 nm, 254 nm, 280 nm, or 320 nm. Molecular weight range 400-2000; capillary voltage 3750 (pos) and 3500 (neg). Analytical thin-layer chromatography (TLC) was performed on pre-coated silica gel 60 F-254 plates (particle size 0.040-0.050 mm, 230-400 mesh) and visualization was accomplished with UV, visual inspection, or potassium permanganate solutions.

### Synthesis of Azide-Quinone Molecule

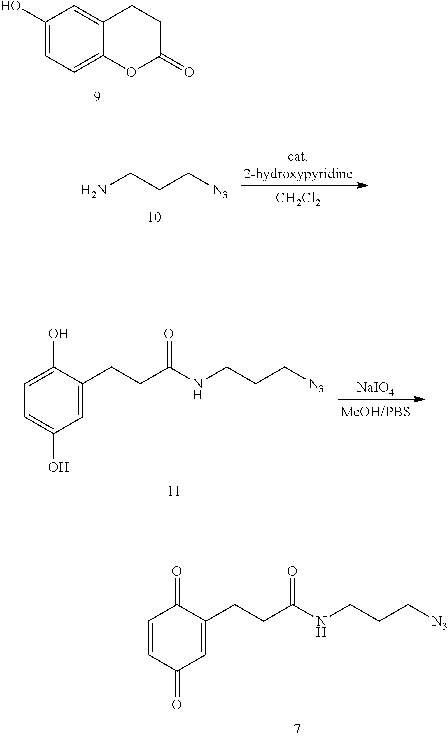

Scheme 7 (previously shown above). Protocol for synthesis of N-(3-azidopropyl)-3-(2,5-dihydroxyphenyl) propanamide 11.

To a solution of 6-hydroxy-3,4-dihydro-2H-1-benzopyran-2-one 9 (180 mg, 1.1 mmol) in CH₂Cl₂ (5.5 mL) was added 2-hydroxypyridine (21 mg, 0.22 mmol) and 3-azidopropylamine 10 (120 mg, 1.21 mmol). The reaction was stirred for 19 hours at room temperature. The solvent was removed in vacuo (rotary evaporation) and the residue was taken up in ethyl acetate and washed with 1 M aqueous HCl, water, and brine. The organic layer was dried using MgSO₄, filtered, and concentrated in vacuo. The crude residue was taken up in methanol (5.5 mL) and used directly in the next reaction.

### 3. Protocol for Synthesis of N-(3-azidopropyl)-3-(3,6-dioxocyclohexa-1,4-dien-1-yl) propanamide 7

To a solution of 1,4-hydroquinone 11 (1.1 mmol) in methanol (5.5 mL) was added a solution of NaIO4 (235 mg, 1.1 mmol) in water (2.25 mL). The reaction mixture was stirred at room temperature for 5 min, then additional water (2 mL) was added, and the reaction mixture was stirred for an additional 20 min. The mixture was diluted with ethyl acetate (40 mL) and washed with water (2×50 mL) and brine (30 mL), and dried using MgSO₄. The organic layer was concentrated in vacuo to afford quinone 7.

### 4. Alternative Protocol Starting from Neat 1,4-hydroquinone 11

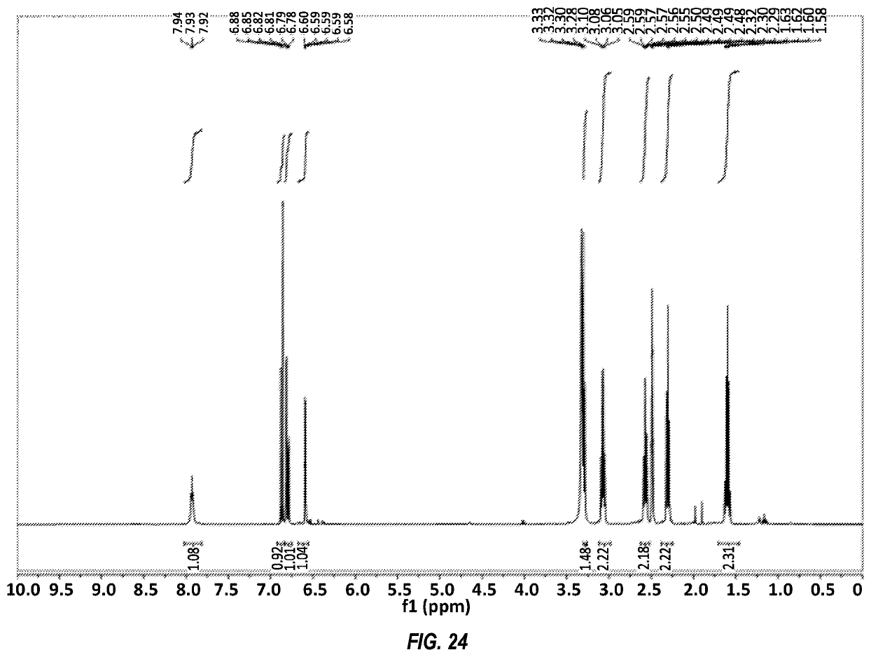

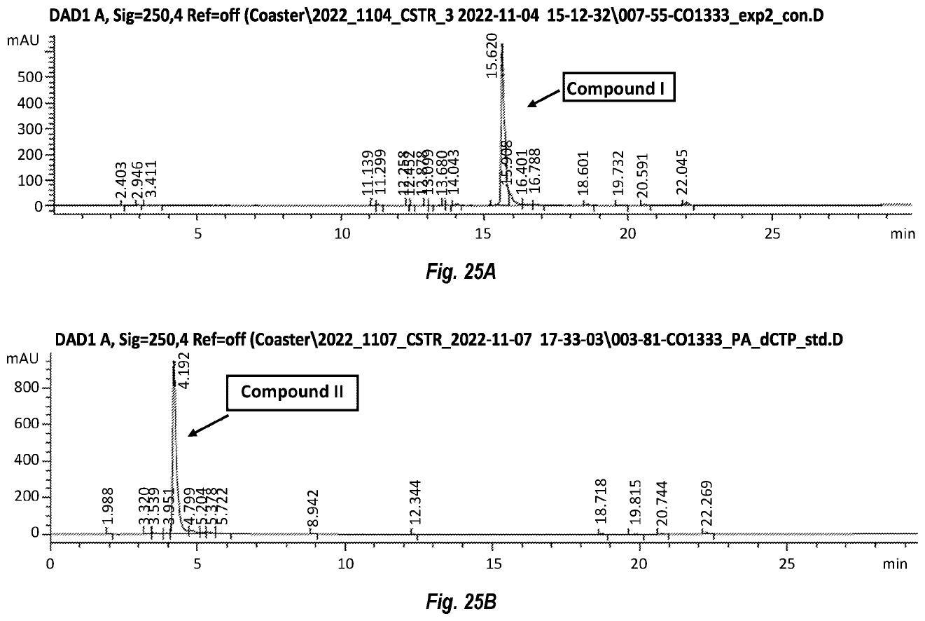

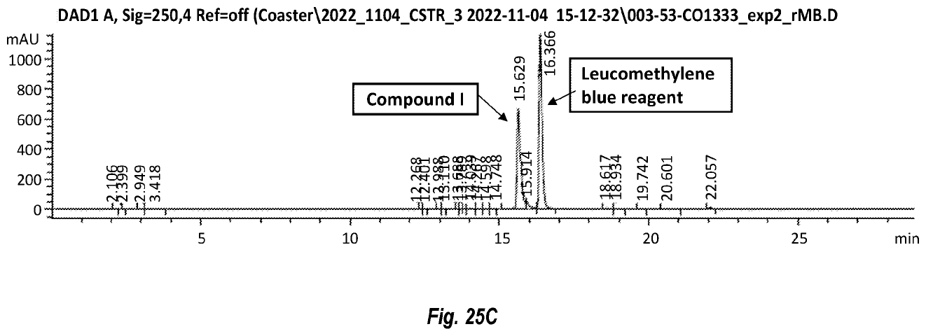

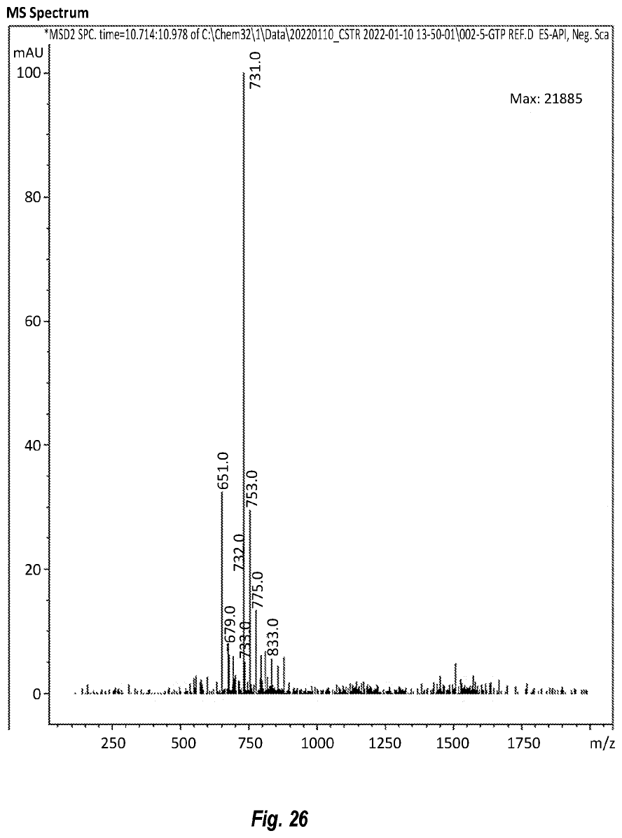

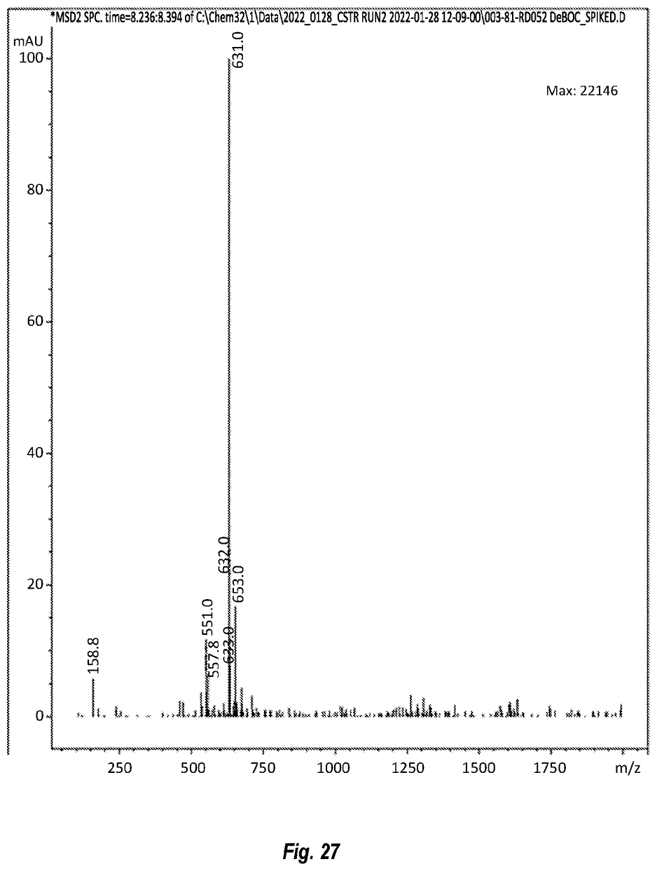

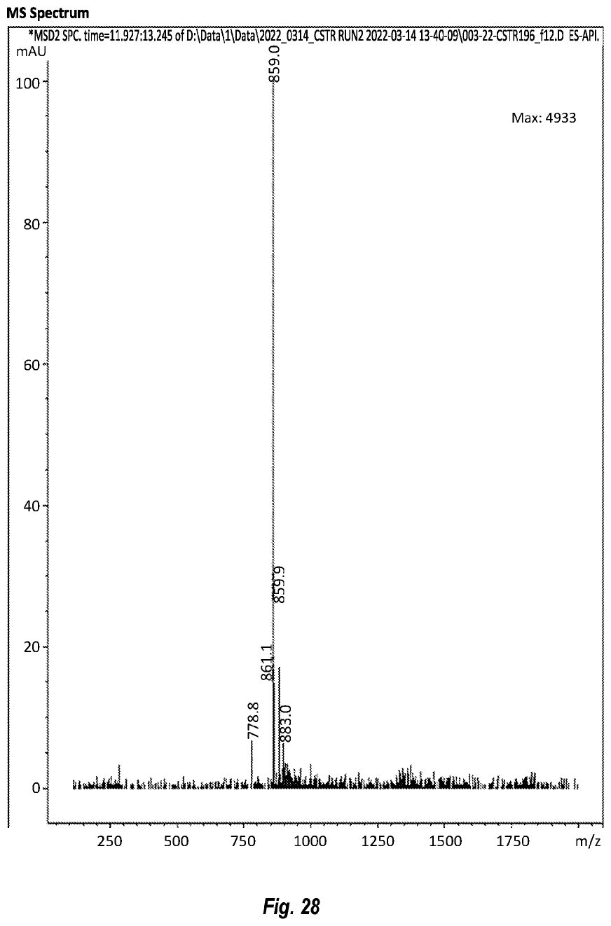

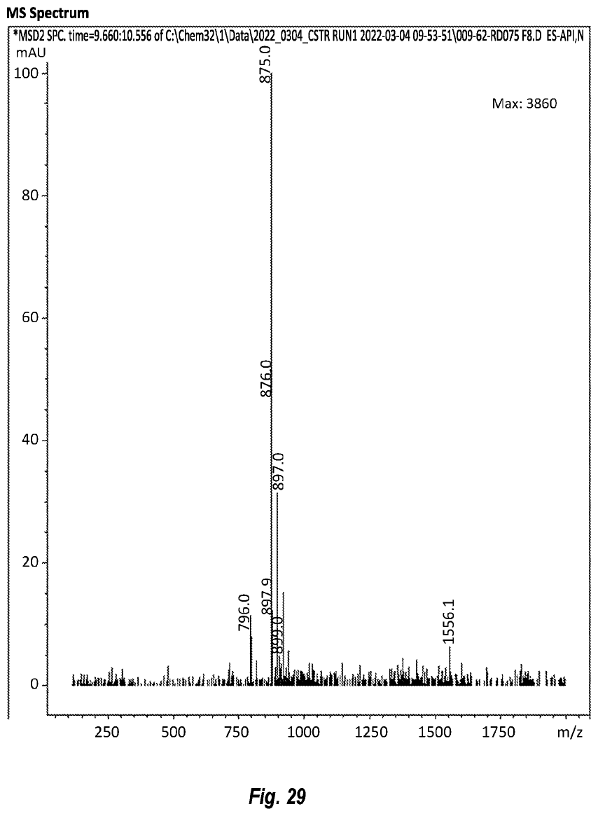

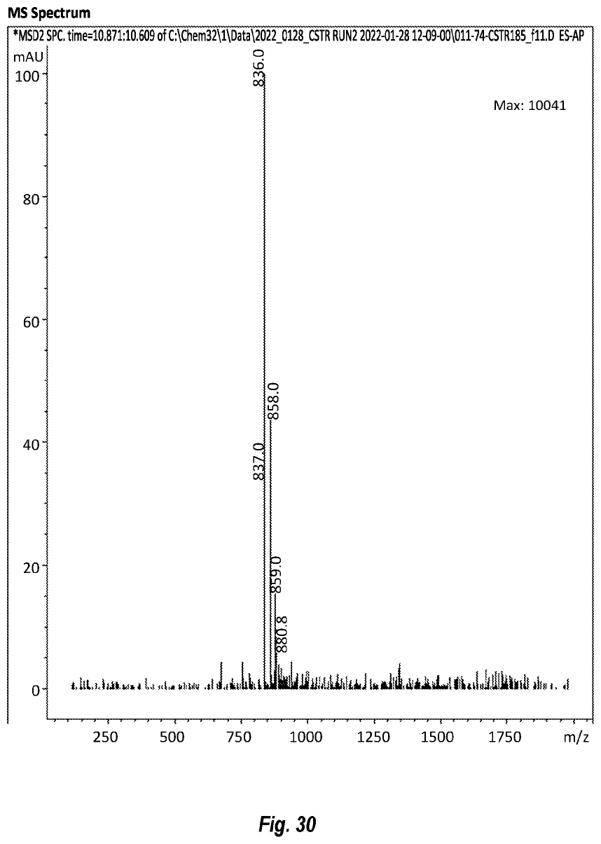

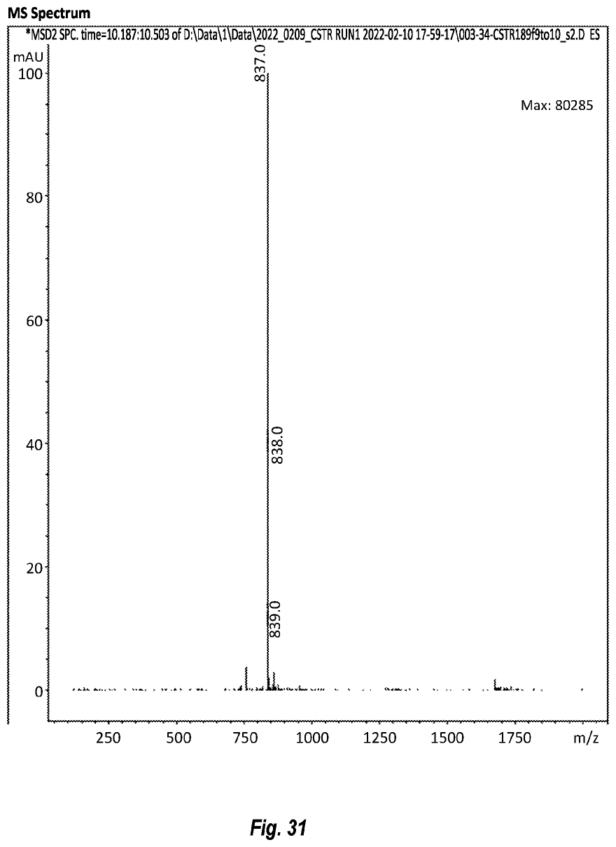

A solution of NaIO₄ (58 mg, 0.27 mmol) in 1×PBS (phosphate buffered saline) (1.0 mL) was generated and stirred for 15 minutes at room temperature. In a separate vial, compound 11 (57.6 mg, 0.22 mmol) was dissolved in methanol (2.0 mL). The solution of NaIO₄ was added to the solution of 11, and the reaction mixture was stirred at room temperature for 25 min. Additional methanol (1.0 mL) was added to help with dissolution. The mixture was diluted with water (30 mL) and extracted with ethyl acetate. The ethyl acetate layers were washed with water and brine, dried (Na₂SO₄) and concentrated in vacuo to afford 7 (49 mg, 85% yield) as a tan oil that solidifies upon standing. ¹H NMR data for compound 7 is shown in FIG. 24.

### 5. Synthesis of Modified Nucleotides

Example protocol for modifying a nucleotide to contain an aminooxy functionality:

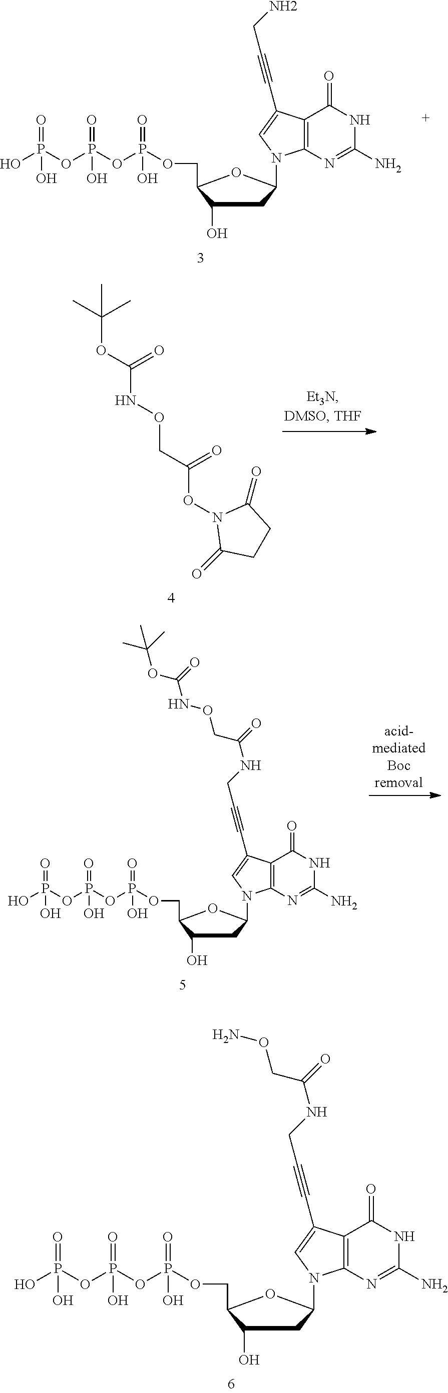 [SMILES: NCC#Cc1cn([C@H]2C[C@@H](O)[C@@H](COP(=O)(O)OP(=O)(O)OP(=O)(O)O)O2)c2nc(N)[nH]c(=O)c12 ; CC(C)(C)OC(=O)NOCC(=O)ON1C(=O)CCC1=O ; CC(C)(C)OC(=O)NOCC(=O)NCC#Cc1cn([C@H]2C[C@@H](O)[C@@H](COP(=O)(O)OP(=O)(O)OP(=O)(O)O)O2)c2nc(N)[nH]c(=O)c12 ; NOCC(=O)NCC#Cc1cn([C@H]2C[C@@H](O)[C@@H](COP(=O)(O)OP(=O)(O)OP(=O)(O)O)O2)c2nc(N)[nH]c(=O)c12]

Scheme 5 (previously shown above). Representative protocol for synthesis of aminooxysubstituted nucleotides

A stock solution of PA-dGTP 3 at 100 mM in H₂O was diluted to 25 mM using dimethylsulfoxide (DMSO)—5 μL of the 100 mM stock was diluted with 15 μL of DMSO. A separate solution of 2,5-dioxopyrrolidin-1-yl 2-(((tert-butoxycarbonyl)amino) oxy)acetate 4 was prepared by dissolving 13 mg 4 in tetrahydrofuran (THF) (1 mL). Another stock solution of trimethylamine was prepared by dissolving 27 mg of trimethylamine in THF (1 mL). The solution of 4 (12.2 μL) was added to the 25 mM PA-dGTP solution. Then, the solution of trimethylamine (5.5 μL) was added. The reaction mixture was incubated for no longer than 1 hour at room temperature. The material was purified by chromatography (Biotage, 6 g C18 column; linear gradient from 1-95% MeCN over 10 column volumes) to generate 5: MS (ESI-neg) m/z calc for C21H30N6O17P3 [M−H]⁻ 732.10, found 731.0. Concentrations of S for subsequent reactions can be determined by UV/VIS by generating calibration curves using starting materials at known stock concentrations (in this case, ε=11,000 cm⁻¹ M⁻¹ at 274 nm).

Removal of the tert-butoxycarbonyl (Boc) protecting group was performed following a protocol published in Hutter, D. et al. “Nucleosides Nucleotides Nucleic Acids” Labeled nucleoside triphosphates with reversibly terminating aminoalkoxyl groups,” 29(11) (2010) (see, in particular, the protocol for generation of compound 8m in the Supporting Information). The protocol uses trifluoroacetic acid (TFA) in methanol for a short time period (3 min), followed by precipitation with diethyl ether and dissolution in sodium bicarbonate, followed by neutralization with HCl and purification by ion-exchange chromatography. The material was prepared as proof-of-concept following the protocol, but without doing the final ion exchange chromatography: MS (ESI-neg) m/z calc for C16H22N6O15P3 632.04, found 631.0.

Note that this synthesis to obtain 6 is an exemplary synthesis only. Compounds disclosed in Table 4 were prepared using aminooxy-substituted nucleotides obtained from contracted suppliers.

### 6. Representative Protocol for Generating Azide-Quinone-Linked Nucleotide Triphosphate Analogs

Synthesis of azide-quinone-oxime ether dGTP analog 8

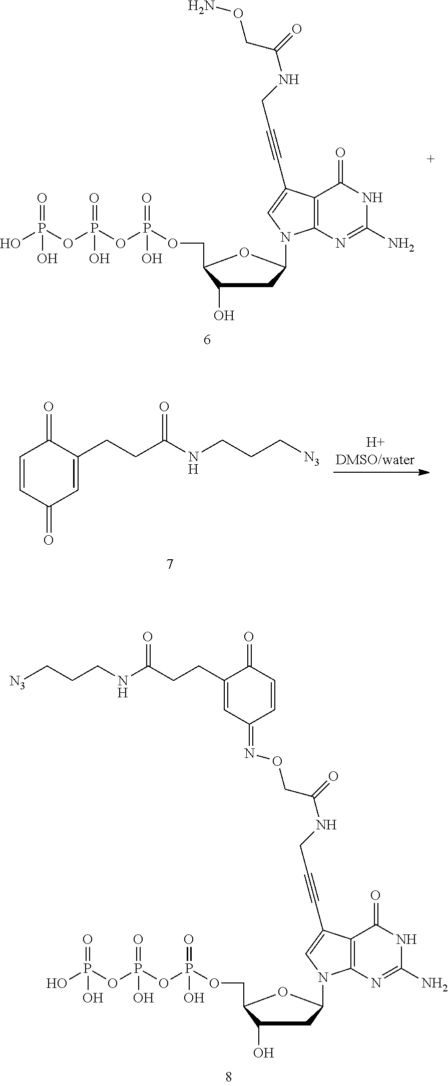 [SMILES: NOCC(=O)NCC#Cc1cn([C@H]2C[C@@H](O)[C@@H](COP(=O)(O)OP(=O)(O)OP(=O)(O)O)O2)c2nc(N)[nH]c(=O)c12 ; [N-]=[N+]=NCCCNC(=O)CCC1=CC(=O)C=CC1=O ; [N-]=[N+]=NCCCNC(=O)CCC1=C/C(=N/OCC(=O)NCC#Cc2cn([C@H]3C[C@@H](O)[C@@H](COP(=O)(O)OP(=O)(O)OP(=O)(O)O)O3)c3nc(N)[nH]c(=O)c23)C=CC1=O]

Scheme 6 (previously shown above). A 50 mM stock solution of azide-quinone 7 in DMSO was prepared (example: dissolving 7 (22.7 mg) in DMSO (1.73 mL). A 20 mM stock solution of 7 in DMSO was generated by diluting 200 μL of the 50 mM stock with 300 μL of DMSO.

A stock solution of NH2O-PA-dGTP 6 (800 μL of a 10 mM stock solution, 8 μmol) was aliquoted into a separate 2-mL tube. The solution was placed on ice, and the pH was adjusted by adding MES buffer (256 μL). Keeping the solution on ice, the DMSO solution of 7 (400 μL at 20 mM, 8 μmol) was slowly added. The solution was removed from ice and incubated at room temperature for 2.5 hours (protected from light). The solution was quenched with 1 M triethylammonium acetate (TEAA) and purified by chromatography (Biotage, 6 g C18 column, 2% MeCN/H2O for 2 CV, gradient from 2% MeCN to 5% MeCN for 3 CV; 5% to 15% MeCN for 3 CV; 15 to 50% MeCN for 5 CV; 50-95% MeCN for 4 CV. Fractions containing product were partially concentrated using a ThermoFisher Savant Speedvac, followed by lyophilization. Lyophilized materials were resuspended in water, and concentrations are determined using UV/VIS. Concentrations were estimated based on extinction coefficients of the corresponding starting aminooxy-substituted triphosphates. Products were analyzed by LC-MS. In this case: MS (ESI) m/z calc for C28H34N10O17P3 875.14 [M−H]⁻, found 875.0.

In other cases, HCl is used for lowering the pH of the solution. A representative protocol is provided below in Scheme 9:

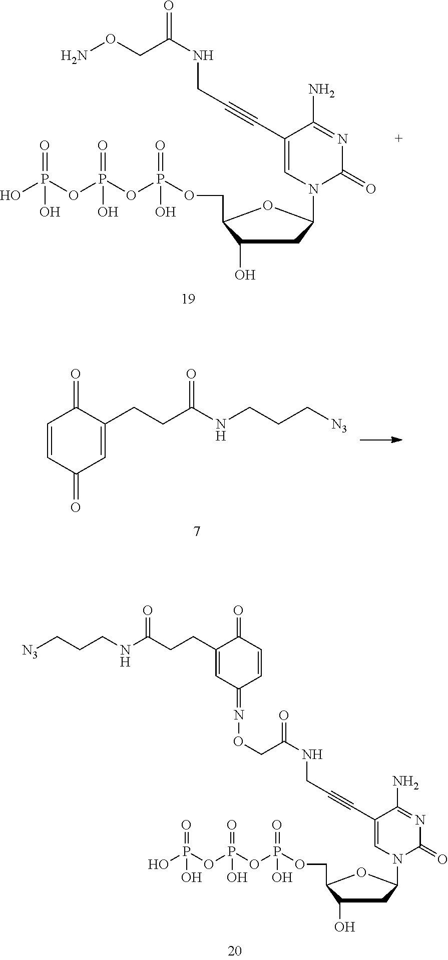 [SMILES: NOCC(=O)NCC#Cc1cn([C@H]2C[C@@H](O)[C@@H](COP(=O)(O)OP(=O)(O)OP(=O)(O)O)O2)c(=O)nc1N ; [N-]=[N+]=NCCCNC(=O)CCC1=CC(=O)C=CC1=O ; [N-]=[N+]=NCCCNC(=O)CCC1=CC(=NOCC(=O)NCC#Cc2cn([C@H]3C[C@@H](O)[C@@H](COP(=O)(O)OP(=O)(O)OP(=O)(O)O)O3)c(=O)nc2N)C=CC1=O]

### 7. Synthesis of Azide-Quinone-Oxime Ether dCTP Analog 20

An aliquot of NH2O-PA-dCTP 19 at 10 mM (800 μL, 8 μmol) was aliquoted into a 2-mL tube and placed on ice. The solution was acidified to pH ~2-3 with 1 M HCl (16 μL). To this solution (on ice), was added a solution of 7 in DMSO (400 μL of a 20 mM stock solution, 8 μmol). The solution was protected from light and incubated at room temperature for 2.5 hours. The reaction mixture was quenched with 1 M TEAA and purified by direct loading onto a column (Biotage, 6 g C18 column, 2% MeCN/H2O for 2 CV, gradient from 2% MeCN to 5% MecN for 3 CV; 5% to 15% MeCN for 3 CV; 15 to 50% MeCN for 5 CV; 50-95% MeCN for 4 CV. Fractions containing product were partially concentrated using a ThermoFisher Savant Speedvac, followed by lyophilization. Lyophilized materials were resuspended in water, and concentrations are determined using UV/VIS. Concentrations were estimated based on extinction coefficients of the corresponding starting aminooxy-substituted triphosphates. Products were analyzed by LC-MS. In this case: MS (ESI) m/z calc for C26H33N9O17P3 836.13 [M−H]⁻; found 836.0.

A protocol for synthesis of quinone oxime ether compound having the methylene linker is shown below in Scheme 10:

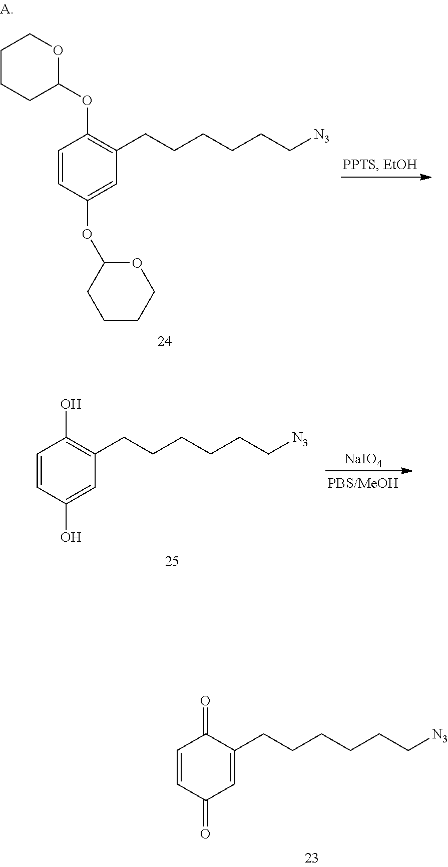

Scheme 10. A solution of 24 (298 mg, 0.74 mmol) in absolute ethanol (5 mL) was treated with pyridinium p-toluenesulfonate (PPTS) (296 mg, 1.18 mmol). The reaction mixture was stirred at room temperature for 22 hours. The mixture was diluted with brine (15 mL) and extracted into ethyl acetate (2×). The combined ethyl acetate layers were washed with water and brine, dried (Na₂SO₄), decanted, and concentrated under reduced pressure. The crude residue was purified on SiO₂, eluting with hexanes:ethyl acetate (3:1, v/v), providing 25 as a tan oil (116 mg, ~87% purity by ¹H NMR analysis—the impurity being residual ethyl acetate).

A solution of NaIO₄ (62 mg, 0.29 mmol) in 1×PBS (phosphate buffered saline) (0.5 mL) was generated and stirred for 5 minutes at room temperature. In a separate vial, compound 25 (54 mg, which translates to 47 mg based on 87% purity metric, 0.20 mmol) was dissolved in methanol (1.0 mL). The solution of NaIO₄ was added to the solution of 25, and the reaction mixture was stirred at room temperature for 15 min. Additional methanol and water (1.0 mL each) were added to help with dissolution. The mixture was diluted with water and extracted with ethyl acetate. The ethyl acetate layers were washed with water and brine, dried (Na₂SO₄) and concentrated in vacuo to afford 23 (33.5 mg, 72% yield) as a brown oil that reconstitutes bright yellow in solution.

A protocol for condensation reaction to form the quinone oxime ether is shown below in Scheme 11:

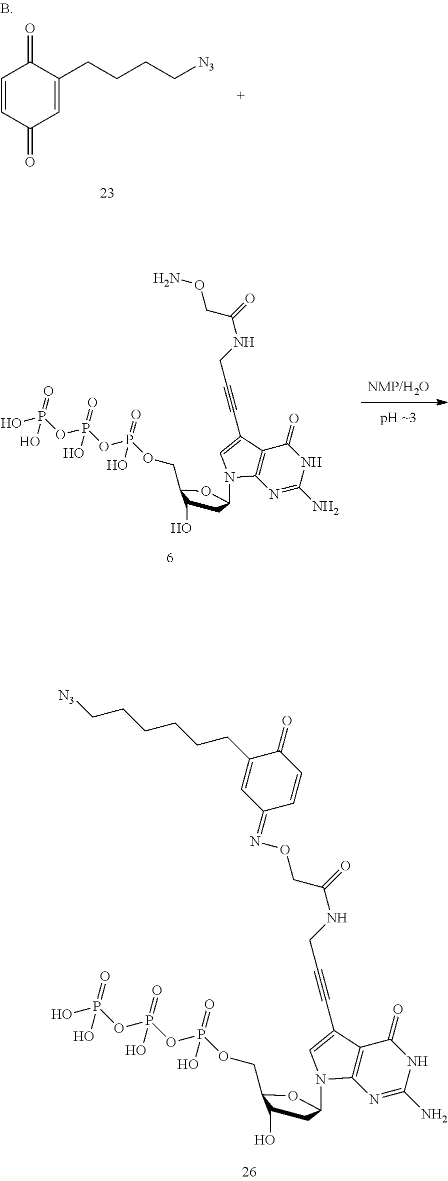

Scheme 11 A 50 mM stock solution of azide-quinone 23 in DMSO was prepared and diluted further to a final concentrated of 20 mM using NMP. A stock solution of NH2O-PA-dGTP 6 (100 μL of a 55 mM stock solution, 5.5 μmol) was aliquoted into a separate 2-mL tube. The solution was diluted with NMP 400 μL), placed on ice, and the pH was adjusted to ca. 3 by adding 1M HCl (aqueous). Keeping the solution on ice, the DMSO solution of 23 (100 ul) was slowly added. The solution was removed from ice and incubated at room temperature for 2.5 hours (protected from light). The solution was quenched with 1 M triethylammonium acetate (TEAA) and purified by chromatography (Biotage, 6 g C18 column, 2% MeCN/H2O for 2 CV, gradient from 2% MeCN to 5% MeCN for 3 CV; 5% to 15% MeCN for 3 CV; 15 to 50% MeCN for 5 CV; 50-95% MeCN for 4 CV. Fractions were concentrated via lyophilization. Lyophilized materials were resuspended in water, and concentrations were determined using UV/VIS. Concentrations are estimated based on extinction coefficients of the corresponding starting aminooxy-substituted triphosphates. Products were analyzed by LC-MS. In this case: MS (ESI) m/z calc for C28H35N9O16P3 846.14 [M−H]⁻, found 846.0.

### B. Synthesis of Nucleotides Tethered to Trimethyl Lock Linkers

### 1. Representative Synthesis of Compounds Containing the Methyl-Substituted Quinone Propionic Acid (Trimethyl Lock) Structure

Analogs wherein there is a quinone propionic acid (trimethyl lock) structure were generated using a starting material with reported synthesis and structure. Ciampi et al. J. Am. Chem. Soc. “Electrochemical “Switching” of Si (100) Molecular Assemblies,” 134:844-847 (2012). A representative synthesis that was carried out is depicted in Scheme 12. Oxidation of hydroquinone 30 with N-bromosuccinimide (NBS) affords acid 31. Acid 31 was converted to either the N-hydroxysuccinimidyl ester (NHS ester) or the water-soluble sulfo-N-hydroxysuccinimidyl ester (sulfo-NHS ester) 32 through reaction with 1-Ethyl-3-(3-dimethylaminopropyl) carbodiimide (EDCI) and the corresponding succinimide. The sulfo-NHS ester was then reacted with a propargylamino-substituted nucleotide in borate buffer (pH 8.5) to afford a compound containing an azide-quinone propionic acid (trimethyl lock) linker-nucleotide triphosphate construct.

Scheme 12. Representative synthesis that was carried out of a compound containing an azide, trimethyl lock functionality, and a nucleotide triphosphate.

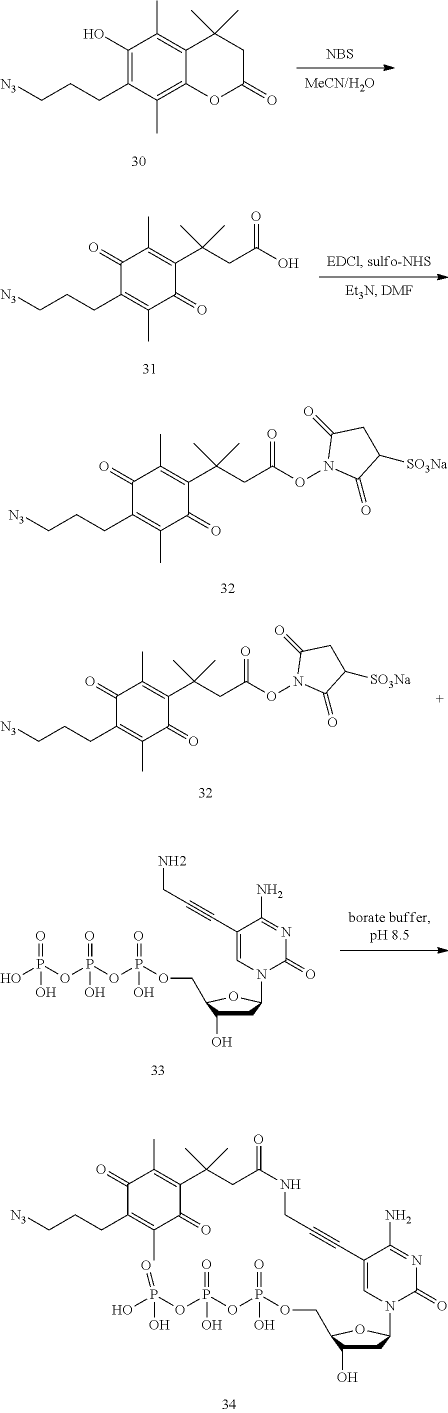

### 2. Protocols for Synthesis of Azide-Trimethyl Lock-dCTP (34)

Synthesis of compound 31. A solution of hydroquinone 30 (50 mg, 0.17 mmol) in acetonitrile (0.50 mL) and water (0.050 mL) was treated with N-bromosuccinimide (NBS) (32 mg, 0.18 mmol) and stirred at room temperature for 1 hour. The solvents were removed under reduced pressure, and the residue was either purified by chromatography on SiO₂ (elution with hexanes/ethyl acetate, 3:1) to afford 31, or the material carried on crude to the next reaction.

Formation of sulfo-NHS ester 32. A solution of acid 31 (20 mg, 0.060 mmol) in dichloromethane (0.6 mL) and N,N-dimethylformamide (0.6 mL) was treated with triethylamine (50 μL) followed by sulfo-N-hydroxysuccinimide, sodium salt (20 mg, 0.092 mmol) and EDCI-HCl (18 mg, 0.094 mmol). The reaction mixture was stirred at room temperature for 1 hour (overnight reaction times also yield product with no loss in yield/purity). Aliquots were quenched with water and used directly in the next reaction.

Addition of sulfo-NHS ester 32 to propargylamine-substituted nucleotide 33. An aliquot corresponding to approximately 0.63 mg sulfo-NHS ester 32 (0.001 mmol) was added to a solution of propargylamine-dCTP 33 (100 μL, 0.001 mmol, c=10 mM in water) and 200 mM borate buffer, pH 8.5 (50 μL). The reaction mixture was protected from light and incubated at room temperature for 2 hours and purified by direct loading onto a column (Biotage, 6 g C18 column, 2% MeCN/H2O for 2 CV, gradient from 2% MeCN to 5% MecN for 3 CV; 5% to 15% MeCN for 3 CV; 15 to 50% MeCN for 5 CV: 50-95% MeCN for 4 CV. Fractions containing product were concentrated via lyophilization. Lyophilized materials were resuspended in water, and concentrations were determined using UV/VIS. Concentrations were estimated based on extinction coefficients of the corresponding starting propargylamine-substituted triphosphates. The products were analyzed by LCMS. MS (ESI) m/z calc for C28H37N7016P3 820.15 [M−H]⁻, found 820.0.

### 3. Synthesis of Aminopropylcarbamate of the Quinone Propionic Acid (Trimethyl Lock)

Chemistry for attachment of the intermediate aminopropyl spacer was demonstrated using a model quinone propionic acid system (compound 35) that does not contain the azidopropyl substituent for enzyme conjugation. The synthesis of the model compound containing an aminopropyl carbamate spacer is shown in Scheme 13. Synthesis of compound 35 was achieved following established protocols. Ciampi et al. J. Am. Chem. Soc. “Electrochemical “Switching” of Si(100) Molecular Assemblies,” 134:844-847 (2012). Compound 37 was synthesized by addition of 3-aminopropan-1-ol 36 to 35 in the presence of diisopropylethylamine using N,N-dimethylformamide as a solvent. Resulting compound 37 was treated with N,N-disuccinimidyl carbonate (DSC) 38 in the presence of 4-dimethylaminopyridine (DMAP) to generate mixed carbonate 39, which was used crude. Crude mixed carbonate 39 was reacted with propargylamino-substituted nucleotides (in this case propargylamine-dCTP 33) in a mixture of N-methyl-2-pyrrolidone (NMP) and borate buffer (pH 8.5) to afford the resulting carbamate analog 40.

Scheme 13. Synthesis of a compound wherein a methyl-substituted quinone propionic acid is tethered to a nucleotide triphosphate via an intermediate aminopropanol spacer.

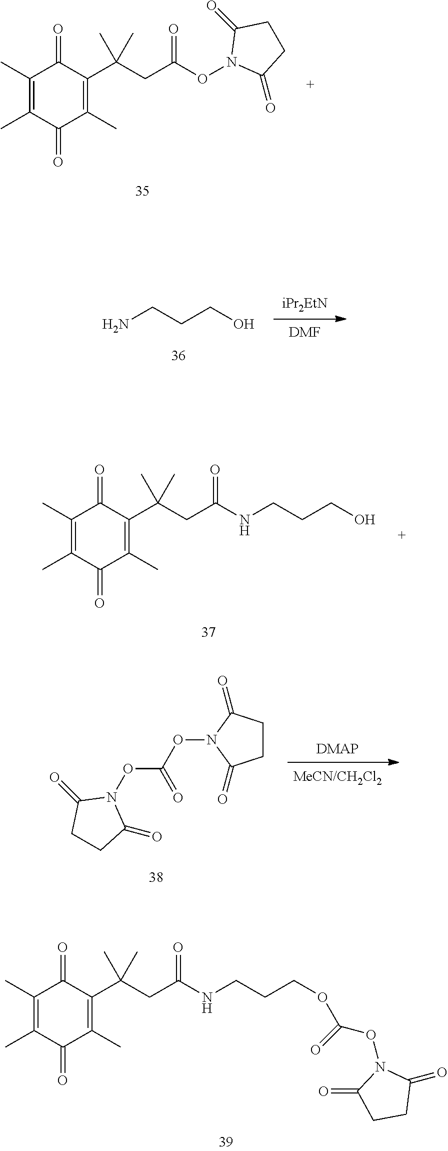

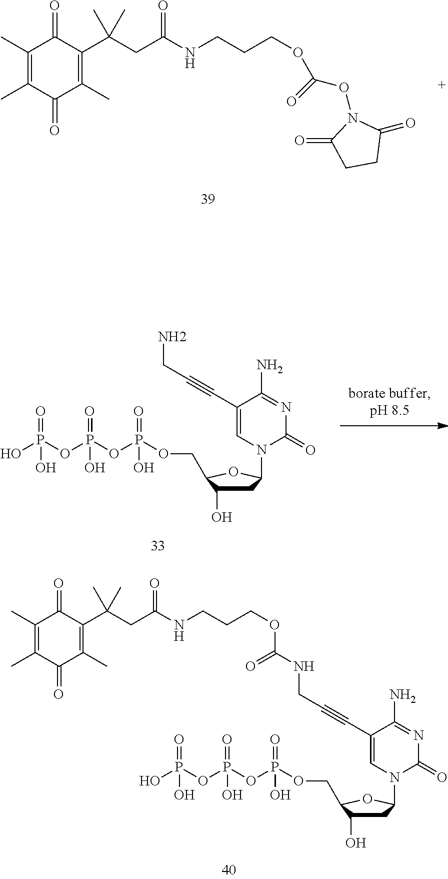

### 4. Protocol for Addition of 3-Amino-1-Propanol to the Trimethyl Lock NHS Ester Compound to Generate Compound 37

See Scheme 13 (above). To a solution of compound 35 (25 mg, 0.072 mmol) in N,N-dimethylformamide (DMF) (0.72 mL) was added a solution of 3-amino-1-propanol 36 (9 mg, 0.12 mol) and diisopropylethylamine (iPr₂NEt) (0.026 mL, 0.15 mmol). This reaction mixture was maintained at room temperature overnight and diluted with ethyl acetate. The diluted solution was washed with water and brine, dried (Na₂SO₄), and concentrated under reduced pressure. The crude material was purified by chromatography on SiO₂ (ethyl acetate as eluent) to afford compound 37 as a yellow solid (15.4 mg, 69% yield). This material was carried forward to the next reaction.

Formation of mixed NHS carbonate 39. A solution of 37 (12.8 mg, 0.042 mmol) and 4-dimethylaminopyridine (2.6 mg, 0.021 mmol) in acetonitrile/CH2Cl2 (0.4 mL, 1:1 volume/volume) was cooled to 0 °C and treated with N,N-disuccinimidyl carbonate (DSC) 38 (10.8 mg, 0.042 mmol). The reaction mixture was stirred overnight, gradually warming to room temperature. The mixture was diluted with CH2Cl2 and washed with water and brine, dried (MgSO4), and concentrated in vacuo. Compound 39 was used without further purification.

Reaction of Propargylamine-Substituted NT (dCTP) with Mixed NHS Carbonate 39.

A stock solution of mixed carbonate 39 was prepared at 100 mM concentration by dissolving 11.1 mg (0.0247 mmol) 39 into N-methyl-2-pyrrolidinone (NMP) (0.247 mL). In a separate tube, a solution of propargylamine-substituted-dCTP (supplied at 10 mM in water, 0.30 mL, 0.003 mmol) was diluted with 200 mM borate buffer, pH 8.5 (300 uL). To this solution was added NMP (0.45 mL) and the 100 mM solution of 39 (0.15 mL, 0.015 mmol, S equiv). The reaction mixture was protected from light and incubated at room temperature for 2 hours and purified by direct loading onto a column (Biotage, 6 g C18 column, 2% MeCN/H2O for 2 CV, gradient from 2% MeCN to 5% MecN for 3 CV; 5% to 15% MeCN for 3 CV; 15 to 50% MeCN for 5 CV; 50-95% MeCN for 4 CV. Fractions containing product 40 were concentrated via lyophilization. Lyophilized materials are resuspended in water, and concentrations were determined using UV/VIS. Concentrations were estimated based on extinction coefficients of the corresponding starting propargylamine-substituted triphosphates. The products were analyzed by LCMS. MS (ESI) m/z calc for C30H41N5018P3 852.17 [M−H]⁻, found 852.0.

### C. Synthesis of Nucleotides Tethered to Disulfide Linkers

Nucleotide analogs with a disulfide linker were synthesized by addition of either an N-hydroxysuccinimidyl ester 41 to an amine-substituted nucleotide (shown here as an analog of 2′-deoxythymidine triphosphate (Scheme 14) or by preparation of mixed N-hydroxysuccinimidyl carbonate 44 followed by addition to the corresponding propargylamine-substituted nucleotides 33 (Scheme 15) to generate carbamates such as compound 45.

Scheme 14. Addition of the N-hyroxysuccinimidyl ester of azidoethyldisulfidepropionic acid to a propargyl-amine-substituted nucleotide.

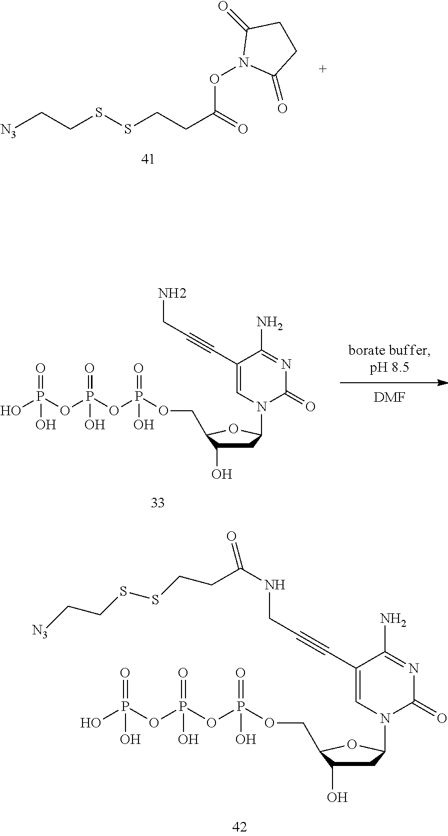

Scheme 15. Formation of and addition of a mixed carbonate of N-hydroxysuccinimide and an alcohol containing both a disulfide and an azide. The mixed carbonate is added to a propargylamine-substituted nucleotide to link the disulfide via a carbamate moiety.

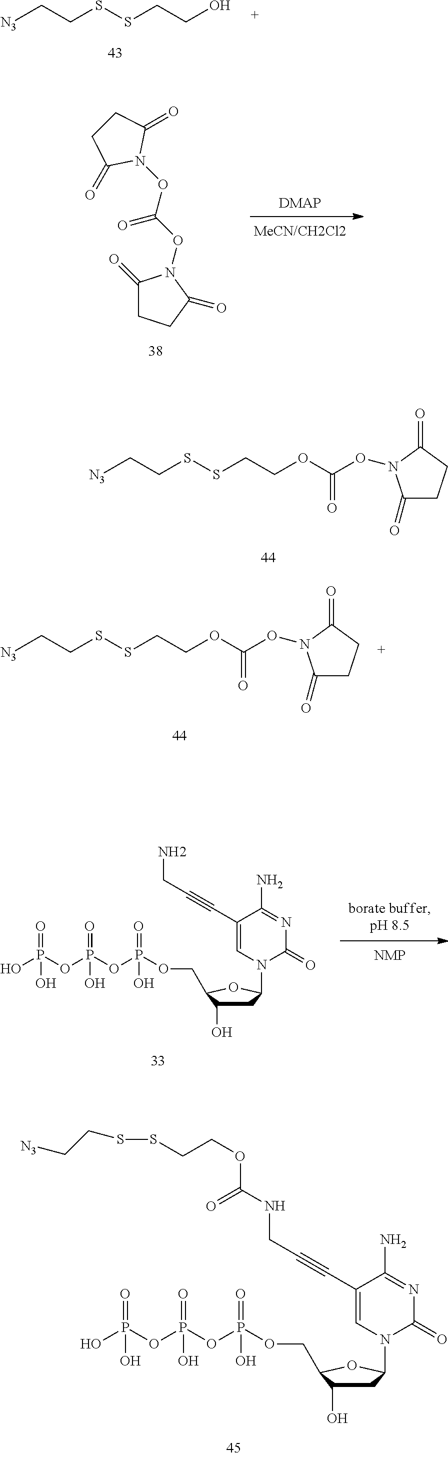

### Protocols:

Az-Disulfide-NHS Ester dCTP-PA Coupling Reaction (CSTR 390)

A 10 mM stock solution of azidoethyl-SS-propionic NHS ester 41 was prepared by dissolving 41 (3 mg, 0.01 mmol) into NMP (1 mL). In a separate tube, an aliquot of 10 mM of propargyl-amine-substituted dCTP (PA-dCTP) 33 (0.40 mL, 0.004 mmol) was diluted with 200 mM borate buffer, pH 8.5 (0.20 mL) and cooled in an ice bath for 10 minutes. The azidoethyl-SS-propionic NHS ester 41 stock solution (0.48 mL, 0.0048 mmol, 1.2 equivalents) was added slowly. Once fully added and mixed, the solution was incubated in the dark on a mixer for 3 hours. After 3 hours and confirming conversion by HPLC, the crude mixture was quenched with 1M triethylammonium acetate (aqueous) until the pH was approximately 7 and purified by direct loading onto a column (Biotage, 6 g C18 column) and eluted using an acetonitrile/water gradient. Fractions (at ~10% acetonitrile/water) containing product 42 were concentrated via lyophilization. Lyophilized materials were resuspended in water, and concentrations are determined using UV/VIS. Concentrations are estimated based on extinction coefficients of the corresponding starting propargylamino-substituted triphosphates. The products are analyzed by LCMS. MS (ESI) m/z calc for C17H25N7O14P3S2 708.01 [M−H]⁻, found 707.9.

Mixed carbonate of the azidoethyldisulfidealcohol 44. A solution of azidoetbyldisulfide-ethyl alcohol (53.8 mg, 0.30 mmol) and 4-dimethylaminopyridine (19 mg, 0.16 mmol) in acetonitrile/CH₂Cl₂ (1.6 mL, 1:1 volume/volume) was cooled to 0 °C and treated with N,N-disuccinimidyl carbonate (DSC) 38 (72 mg, 0.28 mmol). The reaction mixture was stirred overnight, gradually warming to room temperature. The mixture was diluted with CH₂Cl₂ and washed with water and brine, dried (MgSO₄), and concentrated in vacuo. Compound 44 (68 mg) was used in the following reaction without further purification.

Addition of Mixed NHS Carbonate to Propargylamine-Substituted Nucleotide (dCTP) for Preparation of Compound 45.

A stock solution of mixed carbonate 44 was prepared at 100 mM concentration by dissolving 44 63 mg (0.20 mmol) into N-methyl-2-pyrrolidinone (NMP) (2.0 mL). In a separate tube, a solution of propargylamine-substituted dCTP (supplied at 10 mM in water, 0.50 mL, 0.005 mmol) was diluted with 200 mM borate buffer, pH 8.5 (500 uL). To this solution was added NMP (0.75 mL), cooled to 0 °C on ice, and the 100 mM solution of 44 (0.25 mL, 0.025 mmol, 5 equiv) is added. The reaction mixture was protected from light and incubated at room temperature for 2 hours and purified by direct loading onto a column (Biotage, 6 g C18 column, 2% MeCN/H2O for 2 CV, gradient from 2% MeCN to 5% MecN for 3 CV; 5% to 15% MeCN for 3 CV; 15 to 50% MeCN for 5 CV; 50-95% MeCN for 4 CV. Fractions containing product were concentrated via lyophilization. Lyophilized materials were resuspended in water, and concentrations are determined using UV/VIS. Concentrations are estimated based on extinction coefficients of the corresponding starting propargylamino-substituted triphosphates. The products were analyzed by LCMS. MS (ESI) m/z calc for C17H25N7O15P3S2 724.01 [M−H]⁻, found 724.0.

### D. Synthesis of Nucleotides Tethered to Syringic Acid-Based Linkers or Vanillin-Based Linkers

### 1. Synthesis and Protocols for Vanillin/Syringic Analogs

Scheme 16. Synthesis of nucleotide analogs modified via a azide-vanillin-oxime linkage.

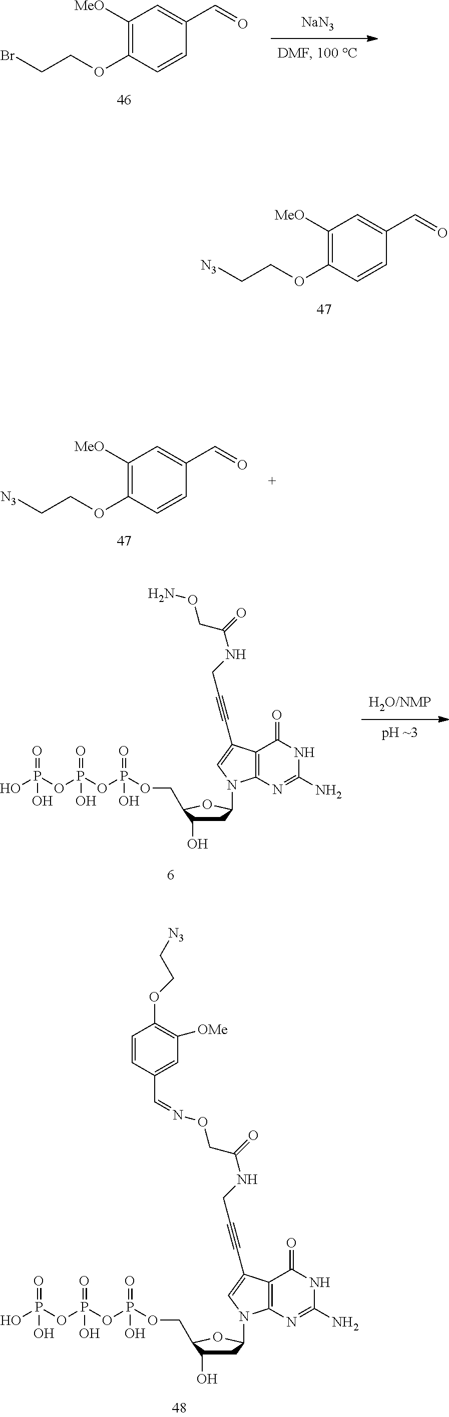

An azidoethyl substituted vanillin analog was prepared by reaction with 4-(2-bromoethoxy)-3-methoxybenzaldehyde 46 and sodium azide. The resulting aldehyde 47 was condensed with aminooxy-substituted nucleotide 6 to afford analog 48. (Scheme 16). In this case, analogs of 2-deoxyguanosine-5′-triphosphate are shown, but the synthesis would translate to other nucleotide analogs.

Analogs containing an additional meta-methoxy substituent were synthesized using similar protocols (Scheme 17). The aldehyde starting material was prepared in two steps; 4-hydroxy-3,5-dimethoxybenzaldehyde 49 was alkylated with 1,2-bromoethane 50 to generate precursor 51. The synthesis was then analogous to that in Scheme 9—bromide displacement with sodium azide yields compound 52 followed by condensation with aminooxy-substituted nucleotide 6 to generate analog 53.

Scheme 17. Synthesis of nucleotide analogs modified via a azide-syringic aldehyde-oxime linkage

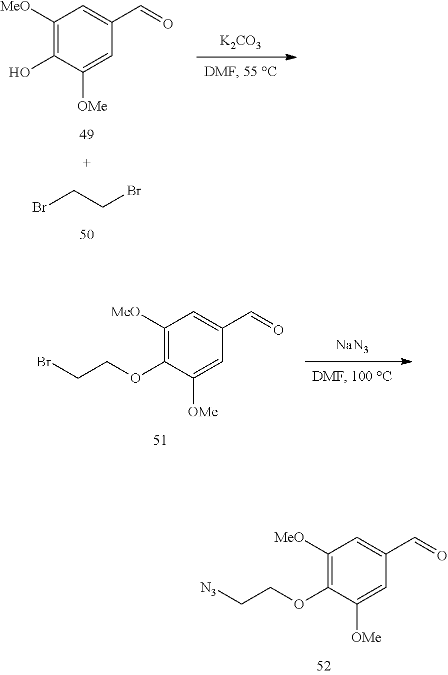

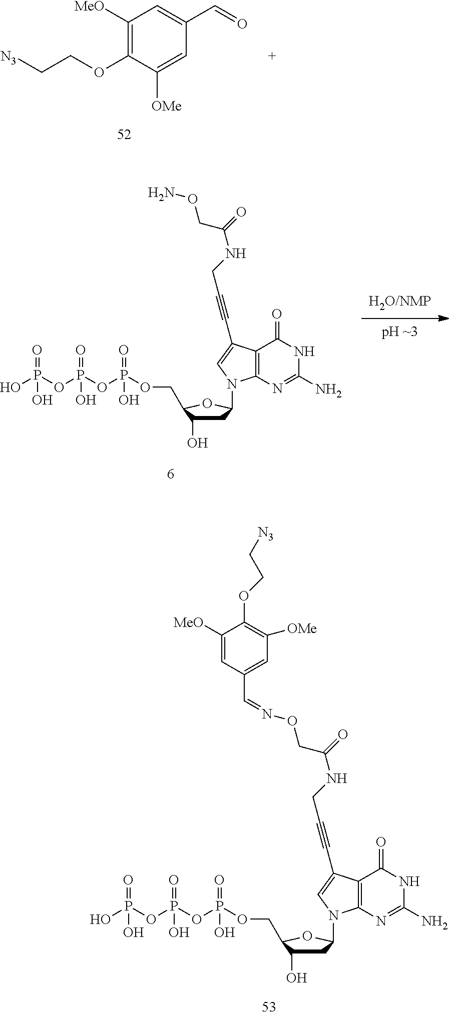

### Protocols:

Protocol for reaction to convert 4-(2-bromoethoxy)-3-methoxybenzaldehyde to the corresponding azide 47. Adapted from Kiran, K. et al., Russian Journal of General Chemistry “Synthesis, characterization, and antibacterial activity of some novel vanillin related hydrazone derivatives bearing 1,2,3-triazole ring,” 87:1288-1294 (2017). To a solution of 4-(2-bromoethoxy)-3-methoxybenzaldehyde 46 (0.00387 mol) in DMF (10 mL) was added sodium azide (0.00465 mol). The mixture was heated at 100 °C for 1 hour. The reaction mixture was cooled to room temperature before diluting the mixture with water (20 mL). The solution as then extracted twice with ethyl acetate (30 mL), combining the organic layers. The combined organic layers were washed with brine solution (25 mL) before drying the resulting mixture over anhydrous sodium sulfate and filtering. The crude mixture was concentrated under reduced pressure to give a light yellow oil. The residue was purified by flash column chromatography using ethyl acetate:hexane (v/v 1:9) to obtain 4-(2-azidoethoxy)-3-methoxybenzaldehyde 47.

Protocol for reaction to generate vanillin oxime 48. A 50 mM stock solution of 4-(2-azidoethoxy)-3-methoxybenzaldehyde 47 in NMP was prepared by dissolving aldehyde 47 (10 mg, 0.045 mmol into 900 μL of NMP). An aliquot of 55 mM aminoxy-substituted nucleotide (NH2O-PA-dGTP) 6 (91 μL, 0.005 mmol) was diluted with NMP (80 μL), and the pH was adjusted to between 2 and 3 using 1 M 4-Morpholineethanesulfonic acid (MES) (300 μL). The solution was chilled on an ice bath for 15 minutes before slowly adding the 4-(2-azidoethoxy)-3-methoxybenzaldehyde solution (100 μL of 50 mM stock, 0.005 mmol). Once fully added and mixed, the solution was incubated in the dark on a mixer for 3 hours. After 3 hours and confirming conversion by HPLC, the crude mixture was quenched with 1 M triethylammonium acetate until the pH was approximately 7. The reaction mixture was purified by direct loading onto a column (Biotage, 6 g C18 column and eluted using an acetonitrile/water gradient. Fractions containing product (eluted at ~10% acetonitrile/water) were concentrated via lyophilization. Lyophilized materials were resuspended in water, and concentrations were determined using UV/VIS. Concentrations were estimated based on extinction coefficients of the corresponding starting aminooxy-substituted triphosphates. The products were analyzed by LCMS. MS (ESI) m/z calc for C26H31N9O17P3 834.11 [M−H]⁻, found 834.0.

### Protocol for Reaction to Generate Syringic Oxime.

The starting materials (corresponding bromide and azide) were synthesized according to Chang, X. et al. Acta Pharm Sin. B “Design, synthesis, and biological evaluation of quinazolin-4(3H)-one derivatives co-targeting poly(ADP-ribose) polymerase-1 and bromodomain containing protein 4 for breast cancer therapy,” 11(1): 156-180 (2021) and Kiran, K. et al. Russian Journal of General Chemistry “Synthesis, characterization, and antibacterial activity of some novel vanillin related hydrazone derivatives bearing 1,2,3-triazole ring,” 87:1288-1294 (2017), respectively.

Synthesis of 4-(2-azidoethoxy)-3,5-dimethoxybenzaldehyde-NH2O-PA-dGTP oxime 53. A 50 mM stock solution of 4-(2-azidoethoxy)-3,5-dimethoxybenzaldehyde 52 in NMP was prepared by dissolving 12 mg (0.048 mmol) of 52 into 900 μL of NMP. An aliquot of 55 mM NH2O-PA-dGTP (91 μL, 0.005 mmol) 6 was diluted with NMP (80 μL), and the pH was adjusted to between 2 and 3 using 1 M 4-Morpholineethanesulfonic acid (MES) (300 μL). The solution was chilled on an ice bath for 15 minutes before slowly adding the 4-(2-azidoethoxy)-3,5-dimethoxybenzaldehyde solution (100 μL, 0.005 mmol). Once fully added and mixed, the solution was incubated in the dark on a mixer for 3 hours. After 3 hours and confirming conversion by HPLC, the crude mixture was quenched with 1M triethylammonium acetate until the pH was approximately 7. The reaction mixture was purified by direct loading onto a column (Biotage, 6 g C18 column and eluted using an acetonitrile/water gradient. Fractions containing product (eluted at ~10% acetonitrile/water) were concentrated via lyophilization. Lyophilized materials were resuspended in water, and concentrations were determined using UV/VIS. Concentrations are estimated based on extinction coefficients of the corresponding starting aminooxy-substituted triphosphates. The products were analyzed by LCMS. MS (ESI) m/z calc for C27H33N9O18P3 864.12 [M−H]−, found 864.0.

### Synthesis of Vanillin and Syringic Acid Nucleotide Analogs Via Carbamate Linkages

Scheme 18. Synthesis of nucleotide analogs with vanillin or syringic acid linkers joined via a carbamate linkage.

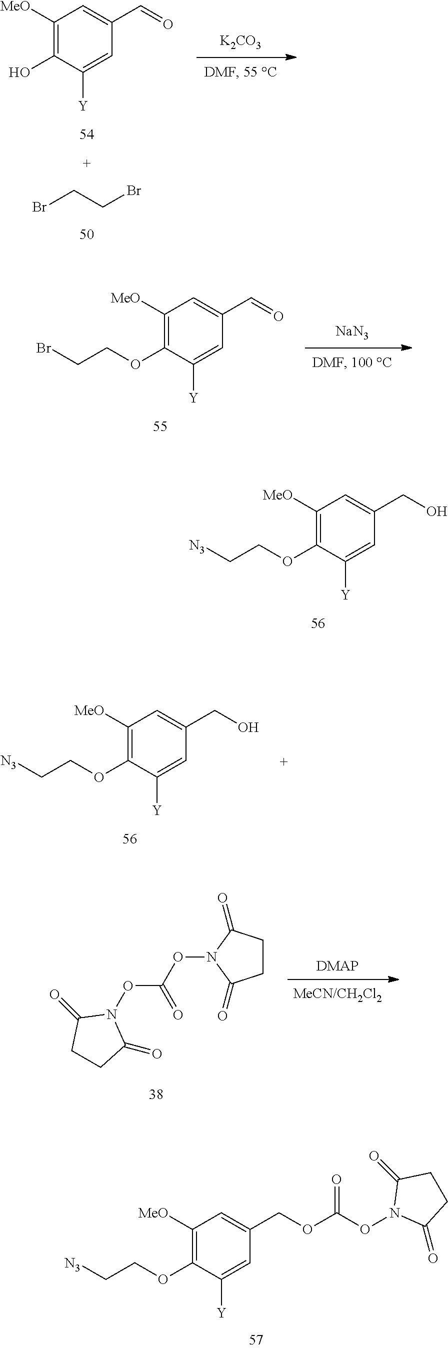

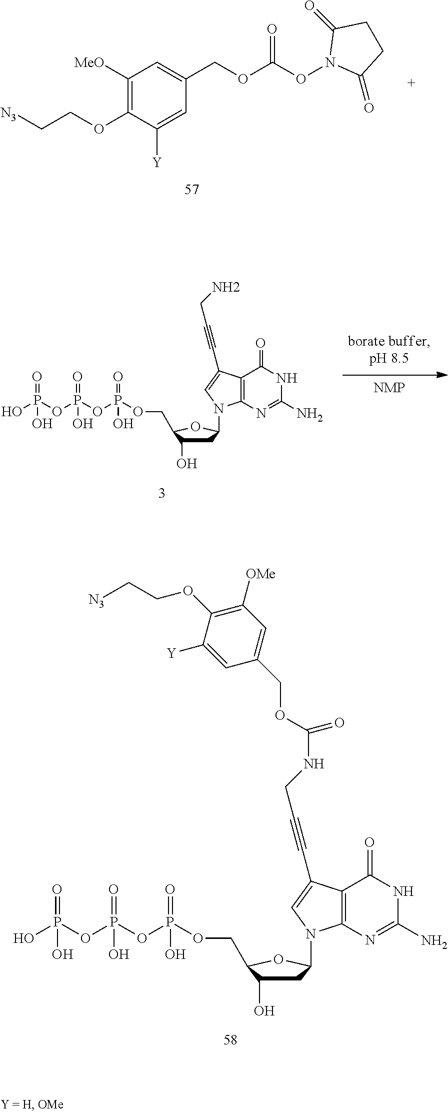

Analogs containing a vanillin or syringic acid linker joined to the nucleotide via a carbamate linkage can be synthesized as depicted in Scheme 18. Phenols of type 54 are alkylated with 1,2-dibromoethane 50, followed by substitution of the bromide with sodium azide to afford alcohols of type 55. The alcohol 55 is converted to the corresponding mixed N-hydroxysuccinimidyl carbonate via reaction with N,N-disuccinimidyl carbonate in the presence of 4-dimethylaminopyridine. The mixed carbonate of type 57 is reacted with propargylamine-substituted nucleotide 3 to generate the corresponding carbamates of type 58. In this case, analogs of 2-deoxyguanosine-5′-triphosphate are shown, but the synthesis would translate to other nucleotide analogs.

### Protocols for Synthesis of Vanillin/Syringic Carbamates

Synthesis of bromide and azide versions of the vanillin analog—note, protocols are written for the vanillin analog but can be translated to the syringic acid analog.

Synthesis of compound 55 (Y═H). To a mixture of potassium carbonate (0.44 g, 3.2 mmol), and 4-(hydroxymethyl)-2-methoxyphenol 54 (Y═H) (0.1 g, 0.65 mmol) in dry DMF (1.2 mL) was added 1,2-dibromoethane 50 (1.21 g. 6.5 mmol). The mixture was sealed and heated to 55 °C overnight. The reaction mixture was cooled to room temperature before diluting the mixture with water (5 mL). The solution was extracted twice with ethyl acetate (5 mL). The organic layers were combined and washed with 1 M NaOH, water, and brine solution (3 mL). The resulting organic solution was dried over anhydrous sodium sulfate and filtered. The crude mixture was concentrated under reduced pressure. The crude residue was purified by flash column chromatography using ethyl acetate:hexane (v/v 1:9) to obtain (4-(2-bromoethoxy)-3-methoxyphenyl) methanol 55 (Y═H).

Synthesis of compound 56 (Y═H). To a solution of bromide 55 (Y═H) (100 mg, 0.385 mmol) in DMF (1.2 mL) was added sodium azide (30 mg, 0.46 mmol). The mixture was heated at 100 °C for 1 hour. The reaction mixture was cooled to room temperature before diluting the mixture with water (20 mL). The solution was then extracted twice with ethyl acetate (30 mL), combining the organic layers. The combined organic layers were washed with brine solution (25 mL) before drying the resulting mixture over anhydrous sodium sulfate and filtering. The crude mixture was concentrated under reduced pressure to give a light yellow oil. The residue was purified by flash column chromatography using ethyl acetate:hexane (v/v 1:9) to obtain (4-(2-azidoethoxy)-3-methoxyphenyl) methanol 56 (Y═H).

Reaction to form mixed carbonate of type 57 (Y═H). A solution of 4-(2-azidoethoxy)-3-methoxyphenyl) methanol 56 (Y═H) (23.6 mg, 0.11 mmol) and 4-dimethylaminopyridine (6.7 mg, 0.055 mmol) in acetonitrile/CH2Cl2 (0.30 mL, 1:1 volume/volume) was cooled to 0 °C and treated with N,N-disuccinimidyl carbonate (DSC) 38 (28 mg, 0.11 mmol). The reaction mixture was stirred overnight, gradually warming to room temperature. The mixture was diluted with CH2Cl2 and washed with water and brine, dried (MgSO4), and concentrated under reduced pressure. Compound 57 (Y═H) (68 mg) was used in the following reaction without further purification.

Addition of mixed NHS carbonate 57 (Y═H) to propargylamine-substituted nucleotide (dGTP). A stock solution of mixed carbonate 57 (Y═H) was prepared at 100 mM concentration by dissolving crude 57 (Y═H) 12 mg (0.033 mmol) into N-methyl-2-pyrrolidinone (NMP) (0.33 mL). In a separate tube, a solution of propargylamine-substituted dGTP (supplied at 10 mM in water, 0.30 mL, 0.003 mmol) was diluted with 200 mM borate buffer, pH 8.5 (300 μL). To this solution was added NMP (0.45 mL) and the 100 mM solution of 57 (Y═H) (0.15 mL, 0.015 mmol, 5 equiv). The reaction mixture was protected from light and incubated at room temperature for 2 hours. Analysis by LCMS indicated product formation of compound 58 (Y═H). MS (ESI) m/z calc for C25H30N8O17P3 807.09 [M−H]−, found 807.0. Note, in this case, the product was a minor constituent, but optimization of reaction conditions and mixed carbonate formation would lead to larger amounts of product. Additionally, the reaction was accomplished on a model system 59 (shown below) (wherein the azidoethyl substituent is replaced with a methyl group). The model system was purified using reversed-phase chromatography (C18 column, acetonitrile/water gradient) followed by lyophilization. These conditions would be readily translated to the system with the azidocthyl substituent described herein. (For model system 59, MS (ESI) m/z calc for C23H30N4O18P3 743.08 [M−H]−, found 743.0.

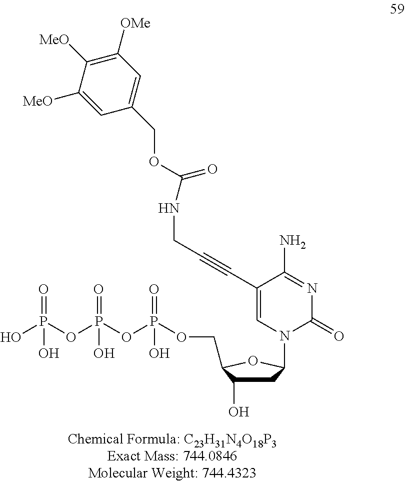

Model compound wherein a carbamate linkage connects a syringic acid analog to a nucleotide.

### Approaches for Small Molecules Attachment to TdT

The tethered small molecule can be a nucleotide triphosphate, and the small molecule can be tethered via a cleavable linker. The linker can be cleaved via electrochemical (redox) means. Synthesis and preparation of the small molecules (modified nucleotide triphosphates) that are covalently attached to the protein are described herein.

Two approaches were taken to install a redox-cleavable linker that is tethered to both TdT and a pendant nucleotide. In approach 1, trifunctional systems were synthesized with three key elements: 1) a functional group handle for covalent conjugation to the TdT enzyme 2) a redox-cleavable linker (e.g., a quinone oxime ether) and 3) the nucleotide to be incorporated (see approaches 1 and 2 below). The synthetic strategy facilitates changes to the enzyme attachment chemistry. In one case, a maleimide was introduced for attachment to cysteine residues. In another case, a DBCO (dibenzylcyclooctyne) was introduced for attachment to azides, such as a 4-azidophenylalanine (non-canonical amino acid) residue. A synthetic scheme for synthesis of these molecules is described herein.

Approaches 1 and 2. The trifunctional structures below each contain a functional group for enzyme attachment; structure 1 has a maleimide for attachment to cysteine; structure 2 has a dibenzylcyclooctyne (DBCO) for attachment to a non-canonical amino acid. The structures have a redox cleavable linker (quinone oxime ether) and a modified nucleotide.

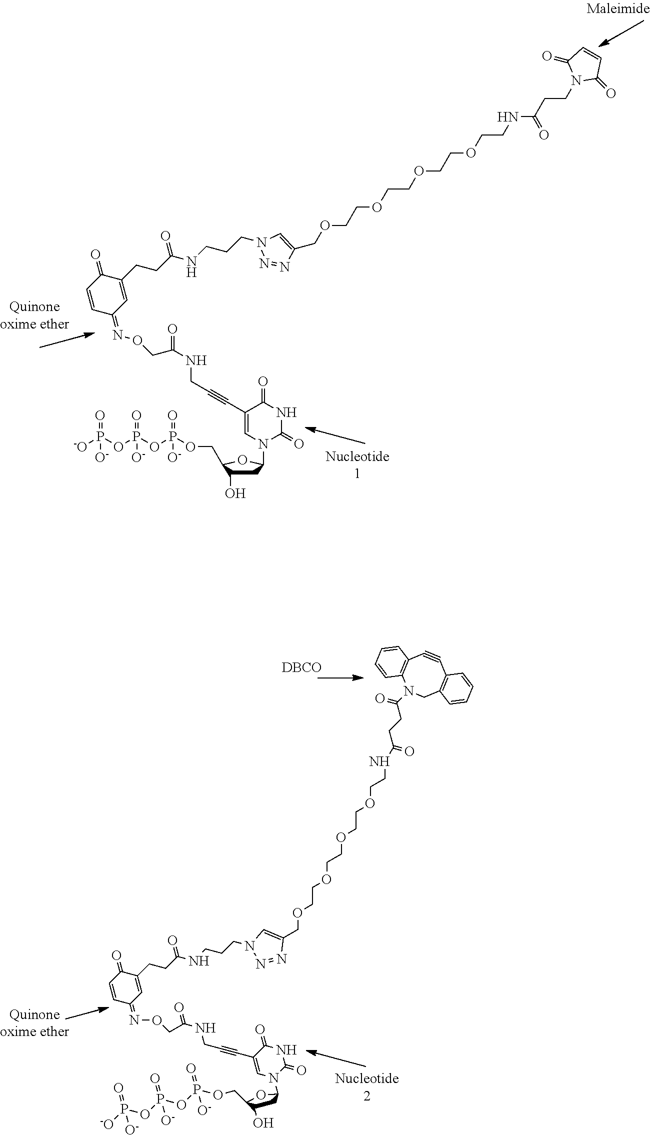

The synthetic routes for obtaining structures 1 and 2 (above) are depicted below (Scheme 19 and Scheme 20).

A common intermediate hydroquinone (reduced form of quinone) was synthesized by aminolysis of commercially available lactone 9 with 3-azidopropylamine 10, in the presence of catalytic 2-hydroxypyridine. The 1,4-hydroquinone 11 is oxidized with sodium periodate to form quinone 7.

The synthesis of the trifunctional system for conjugation to cysteine is shown in Scheme 19.

Copper-catalyzed click chemistry to react the azide of compound 7 with the alkyne of compound 12 provided the quinone 13. Compound 13 was purified using reversed phase chromatography (C18 column with an acetonitrile/water gradient). The purified azide-quinone 13 was condensed with the hydroxylamine of the modified nucleotide triphosphate 14 by mixing 13 and 14 in a 1:1 ratio in acidic media. The final product 1 was used for enzyme conjugations without further purification.

Scheme 19. Synthesis of the trifunctional system for covalently attaching nucleotides to cysteine residues through a redox-cleavable linker. (THF=tetrahydrofuran, THPTA=tris-hydroxypropyltriazolylmethylamine (a Cu-ligand for accelerating click chemistry reactions; DMSO=dimethylsulfoxide).

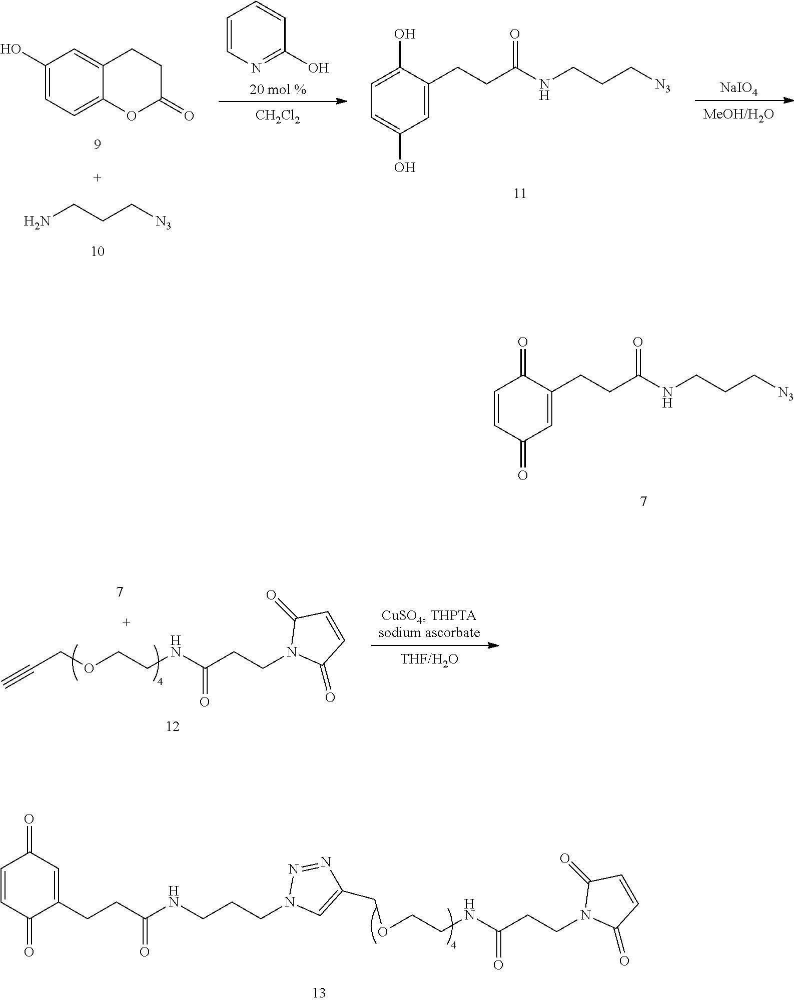

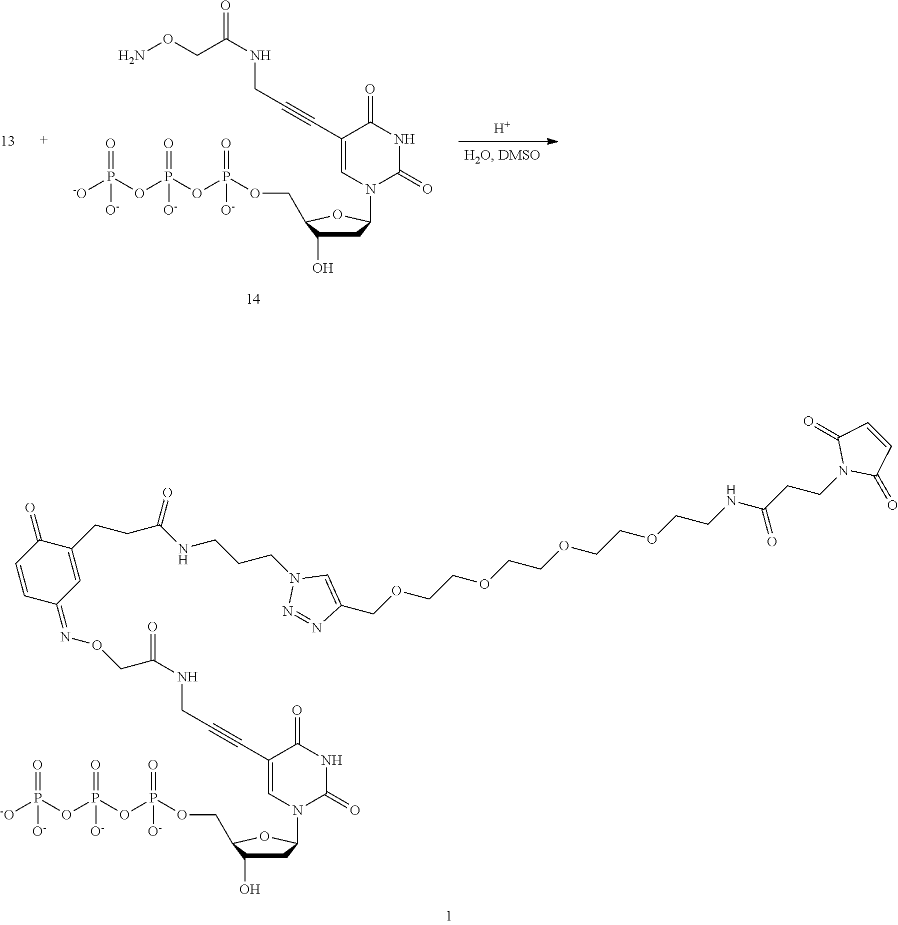

The synthesis of the trifunctional system for conjugation to 4-azidophenylalanine (a non-canonical amino acid) is depicted in Scheme 20. The same azide-quinone 7 described in Scheme 1 is used. Quinone 7 can be reacted with amine-functionalized alkyne 15 using copper-catalyzed click chemistry. Once this reaction proceeds for 1 hour, dibenzocyclooctyne-N-hydroxysuccinimidyl ester (DBCO-NHS) 17 can be added in the same pot to afford compound 18. Compound 18 can be purified using reverse-phase chromatography (C18 column with an acetonitrile/water gradient); purified compound 18 can be condensed with modified nucleotide triphosphate 14 as described above.

Scheme 20. Synthesis of the trifunctional system for covalently attaching nucleotides to 4-azidophenylalanine (non-canonical amino acid) residues through a redox-cleavable linker. (MeCN=acetonitrile; THPTA=tris-hydroxypropyltriazolylmethylamine (a Cu-ligand for accelerating click chemistry reactions).

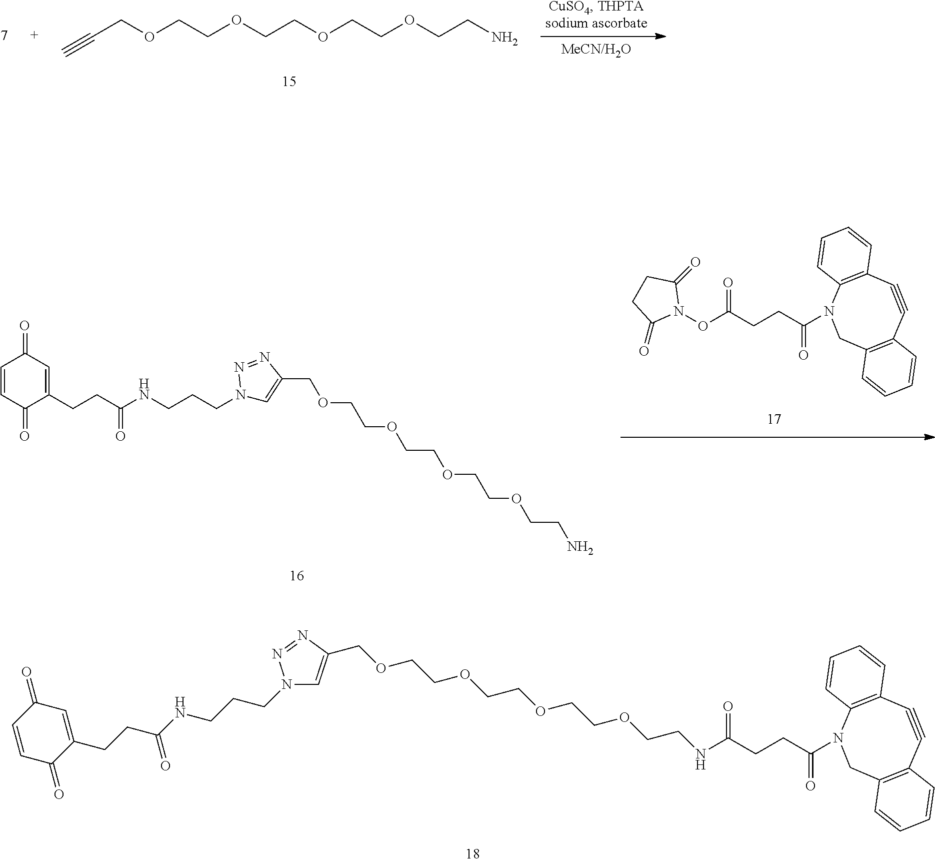

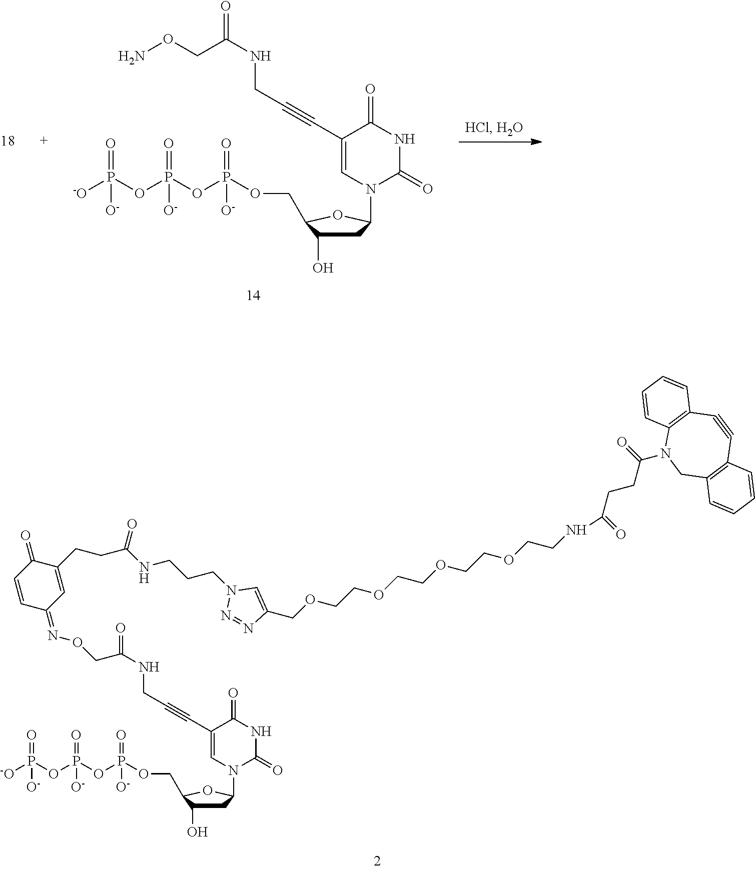

Trifunctional molecules are synthesized by copper-catalyzed click reactions between formed azide-quinone-nucleotides (tabulated in Table 4) and alkynes. Propargyl maleimides of different lengths were reacted with the azide-quinone-nucleotides (Scheme 21 and Scheme 22).

Scheme 21. Synthesis of a trifunctional molecule containing a maleimide, cleavable linker (quinone oxime ether) and a nucleotide triphosphate. Synthesis is accomplished by copper-catalyzed click chemistry of pre-formed azide-quinone-nucleotides and an alkyne.

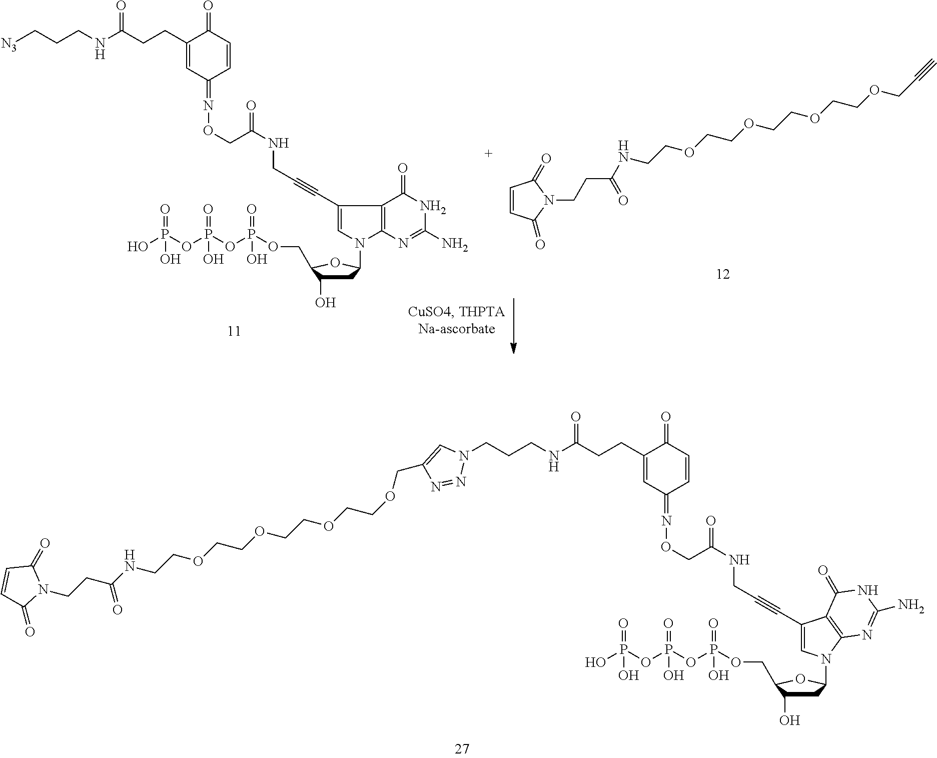

Scheme 22. Synthesis of a trifunctional molecule containing a maleimide, cleavable linker (quinone oxime ether) and a nucleotide triphosphate. Synthesis is accomplished by copper-catalyzed click chemistry of pre-formed azide-quinone-nucleotides and an alkyne.

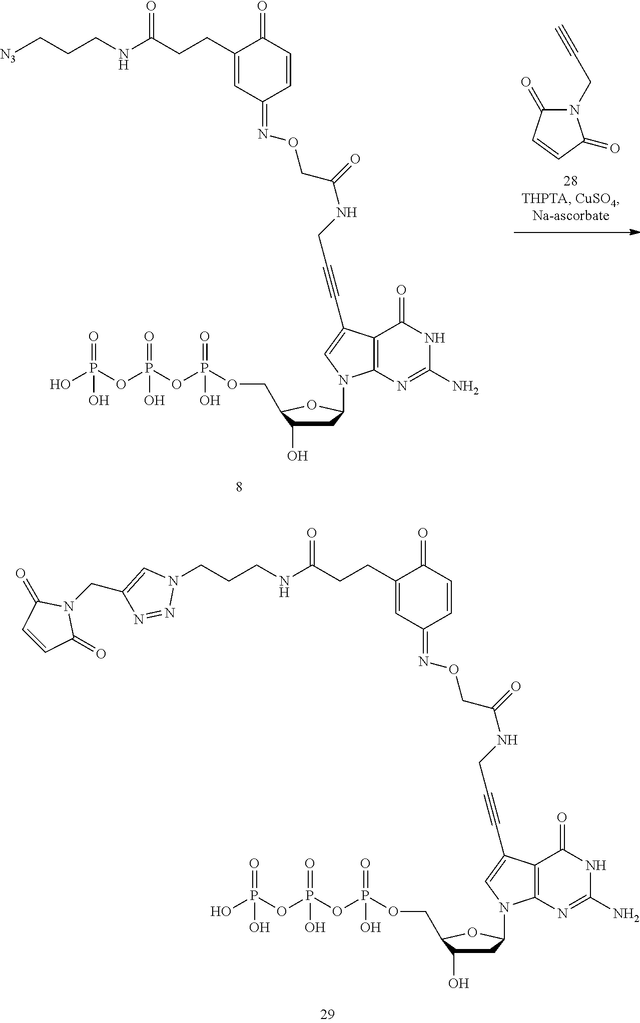

Protocol for click reaction to generate compound 27. Note that the protocol is written for the 2-deoxyguanosine-5′-triphosphate analog but would translate to other nucleotide triphosphates. A solution of azide-quinone-nucleotide 8 at 10 mM in H2O (100 μL, 0.001 mmol) was aliquoted into a tube, followed by a solution of Maleimide-PEG4-alkyne 12 (10 mM stock in water, 100 μL, 0.001 mmol). In a separate tube, a 200 mM THPTA solution in water was mixed with a 100 mM CuSO₄ solution in water (10 μL each). The THPTA/CuSO₄ solution (8 μL, 0.0004 mmol CuSO₄ and 0.0008 mmol THPTA) was added to the tube containing azide-quinone-nucleotide 8 and the maleimide-PEG4-alkyne 12. A solution of 100 mM sodium ascorbate in water (16 μL, 0.0016 mmol) was added to the tube, and the mixture was incubated at room temperature for 1.5 hours. The reaction mixture was purified by chromatography (Biotage, 6 g C18 column, 2% MeCN/H2O for 2 CV, gradient from 2% MeCN to 5% MeCN for 3 CV; 5% to 15% MeCN for 3 CV; 15 to 50% MeCN for 5 CV; 50-95% MeCN for 4 CV. Fractions were concentrated via lyophilization. Lyophilized materials are resuspended in water, and concentrations were determined using UV/VIS. Concentrations are estimated based on extinction coefficients of the corresponding starting aminooxy-substituted triphosphates. Products are analyzed by LC-MS. In this case: MS (ESI) m/z calc for C46H60N12024P3 1257.31 [M−H]⁻, found 1257.1. Note that lower equivalents of Cu and THPTA and shorter reaction times (as reflected in the protocol for synthesis of 29) are recommended.

Protocol for click reaction to generate compound 29. Note that the protocol is written for the 2-deoxyguanosine-5′-triphosphate analog but would translate to other nucleotide triphosphates. A solution of azide-quinone-nucleotide 8 at 10 mM in H2O (100 μL, 0.001 mmol) was aliquoted into a tube, followed by a solution of N-propargyl maleimide 28 (50 mM stock in DMSO, 20 μL, 0.001 mmol). In a separate tube, a 200 mM THPTA solution in water was mixed with a 100 mM CuSO₄ solution in water (5 μL each). The THPTA/CuSO₄ solution (5 μL, 0.00025 mmol CuSO₄ and 0.0005 mmol THPTA) was added to the tube containing azide-quinone-nucleotide 8 and N-propargyl maleimide 28. A solution 100 mM sodium ascorbate in water (5 μL, 0.0005 mmol) was added to the tube, and the mixture was incubated at room temperature for 30 min. The reaction mixture was quenched with 100 mM triethylammonium acetate buffer, then purified by chromatography (Biotage, 6 g C18 column, 2% MeCN/H2O for 2 CV, gradient from 2% MeCN to 5% MeCN for 3 CV; 5% to 15% MeCN for 3 CV; 15 to 50% MeCN for 5 CV; 50-95% MeCN for 4 CV. Fractions were concentrated via lyophilization. Lyophilized materials was resuspended in water, and concentrations were determined using UV/VIS. Concentrations were estimated based on extinction coefficients of the corresponding starting aminooxy-substituted triphosphates. Products were analyzed by LC-MS. In this case: MS (ESI) m/z calc for C35H39N11O19P3 1010.16 [M−H]⁻, found 1010.0.

In another approach, a bifunctional crosslinker is first introduced to a naturally occurring amino acid with specific reactivity, for example, a cysteine residue. Bifunctional molecules containing a maleimide, a spacer section, and a functional group for click chemistry can be used, for example, maleimide-PEG(n)-DBCO compounds (where DBCO stands for dibenzylcycooctyne). The maleimide reacts selectively with the cysteine residue, leaving a reactive DBCO moiety (FIG. 5). Other bifunctional maleimide-alkynes include maleimide-PEG(n)-BCN compounds, where BCN stands for bicyclononyne. Other linking molecules can also be used to react with either naturally occurring or non-natural amino acids; for example, a crosslinker containing a N-hydroxysuccinimidyl group on one end and a clickable moiety (such as an azide or alkyne) at another site. The N-hydroxysuccinimidyl group could react with primary amine functionalities, including lysine residues.

Using the above-mentioned crosslinker chemistries converts an amino acid residue to a reactive functional group. In some instances, this reactive functional group is a bioorthogonal functional group, meaning that the introduced functional group can be reacted exclusively without side reactions from other portions of the biomolecule. In FIGS. 5 and 6, the method is demonstrated using maleimide-alkyne bifunctional crosslinkers. Incubation of the enzyme with a maleimide-alkyne crosslinker installs an alkyne—the alkyne can be a DBCO group. The enzyme is then purified using a desalting or size-exclusion column to remove unconjugated small molecule. The enzyme can then be incubated with a second small molecule containing an azide; the azide reacts with DBCO functional groups without use of additional reagents (FIG. 6).

Conjugation of the azide-quinone-nucleotides to TdT variants was accomplished using the methods portrayed in FIGS. 5 and 6. A general protocol for addition of a bifunctional maleimide-DBCO crosslinker (sulfo-DBCO-maleimide): a solution of 200 μM TdT with a site-specific cysteine residue was incubated with a 10-fold excess of sulfo-DBCO maleimide (Click Chemistry Tools) in TdT Storage Buffer (200 mM Potassium Phosphate pH 6.5, 100 mM NaCl) at room temperature overnight (or incubated at room temperature for 4 hours, then moved to 4 °C for ~24 hours). Excess sulfo-DBCO-maleimide was removed using a desalting column (Zeba™ spin) equilibrated with TdT Storage Buffer. The concentration of recovered protein was determined by measuring UV/VIS (Nanodrop) and calculating using ε=54890 M⁻¹ cm⁻¹.

Once the TdT was modified with a pendant DBCO, the azide-quinone-nucleotide was added in 3-fold molar excess of the TdT-DBCO (e.g., 140 μM TdT-DBCO and 420 M azide-quinone-nucleotide) in TdT Storage Buffer. The mixture was incubated at room temperature overnight (or incubated at room temperature for 4 hours, then moved to 4 °C if used >24 hours after the initial addition). Excess azide-quinone-nucleotide was removed using a desalting column (Zeba™ spin) equilibrated with 20 mM potassium phosphate pH 6.5, 100 mM NaCl. The eluent was further purified using a Zeba™ Dye and Biotin Removal Column equilibrated with 20 mM potassium phosphate pH 6.5, 100 mM NaCl. Samples were lyophilized and stored at room temperature until reconstituted.

### Example 5. Verification of Nucleotide Addition to TdT

The conjugation of small molecules to the modified TdT was verified by LC-mass spectrometry. LCMS analysis is done using a Waters Acquity UPLC coupled to a Waters Xevo QTof. The typical method used was as follows: Buffer A 0.05% TFA in water and Buffer B 0.05% acetonitrile. The gradient was 10-20% B in 1 min, 20-50% B in 9 min, 0.5 mL/min, post-column split, using the following column: 2.1×50 mm Halo Diphenyl 2.7 μm.

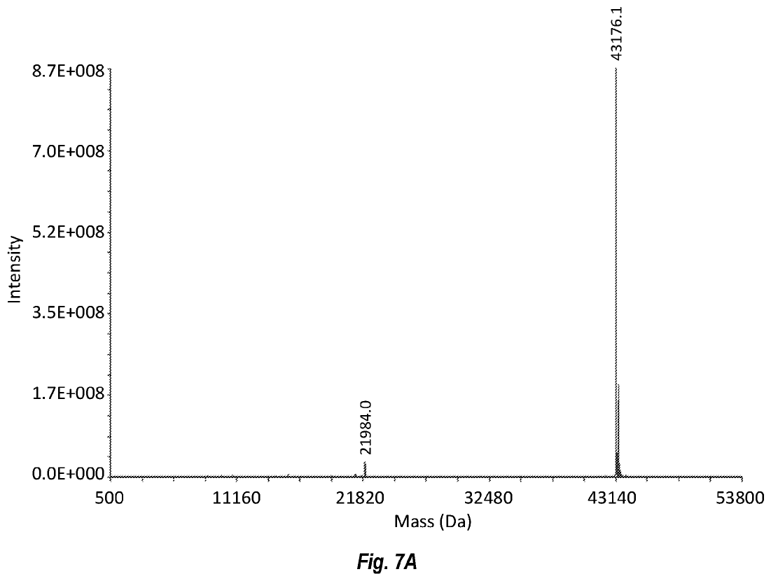

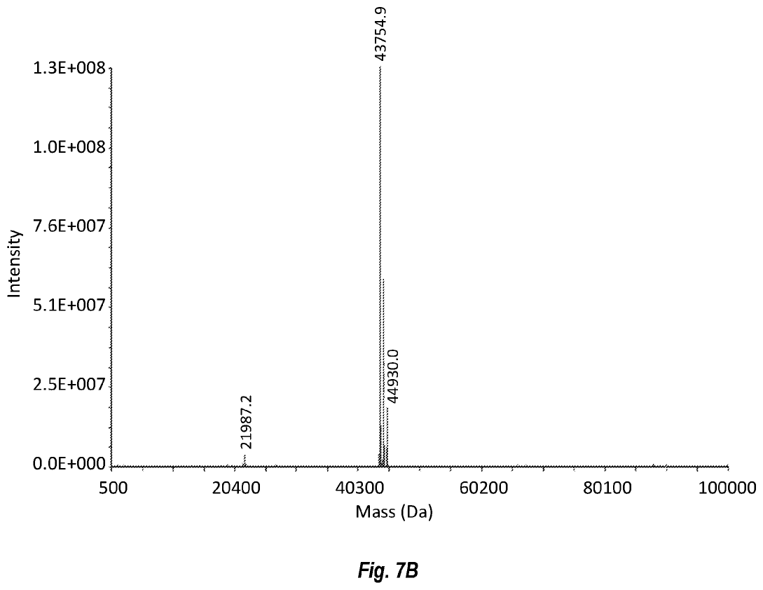

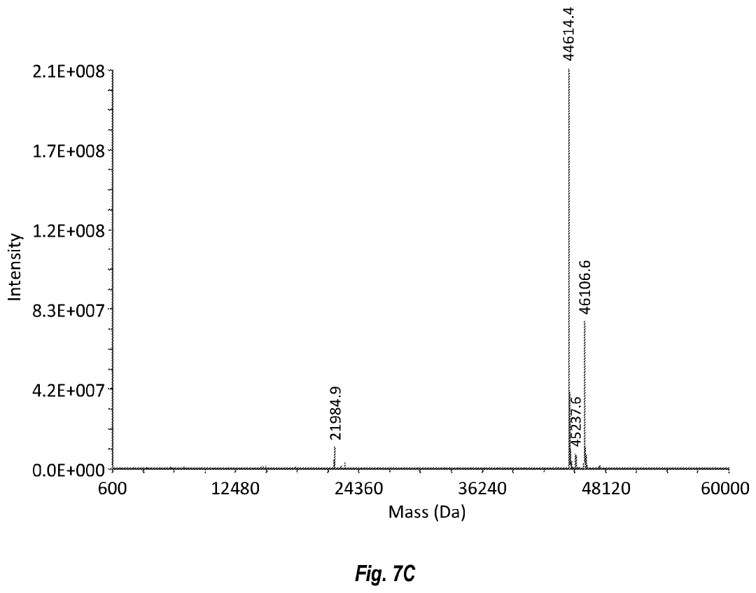

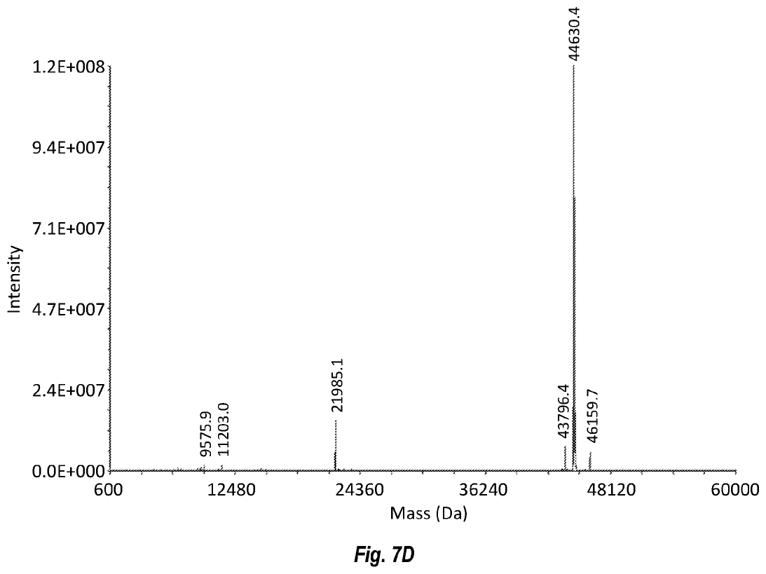

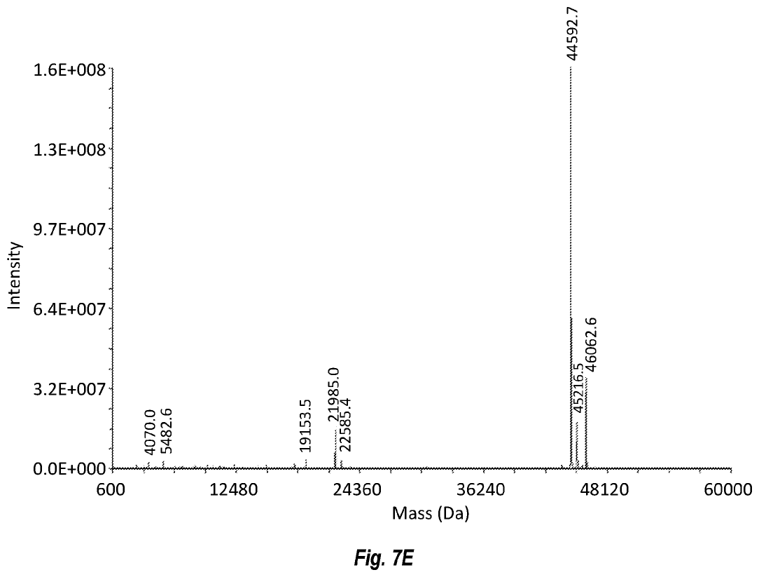

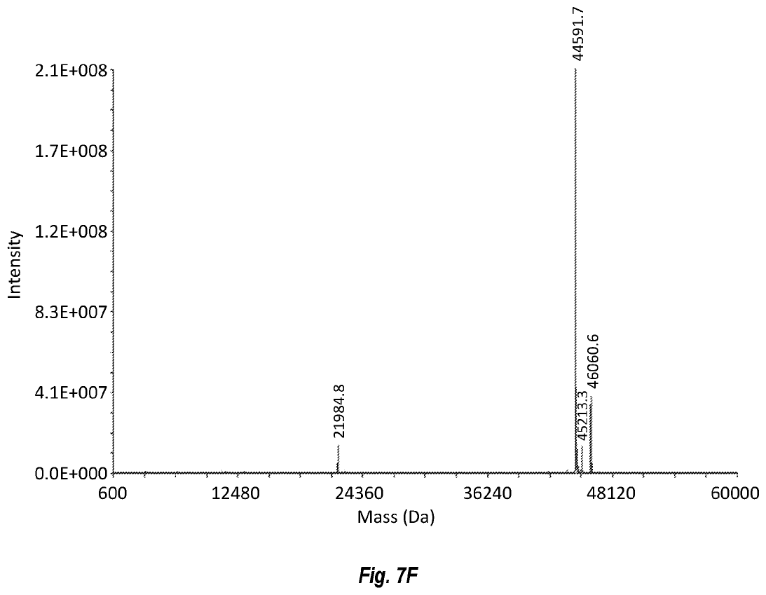

Deconvoluted mass spectral data was tabulated in Table 5 and FIG. 7A, FIG. 7B, FIG. 7C, FIG. 7D, FIG. 7E, and FIG. 7F. The C7 variant (unconjugated) mass was detected without the N-terminal methionine; proteins typically lose the N-terminal methionine when recombinantly expressed in bacteria. The structures and expected masses of the small molecule conjugates added to the modified TdT are shown below.

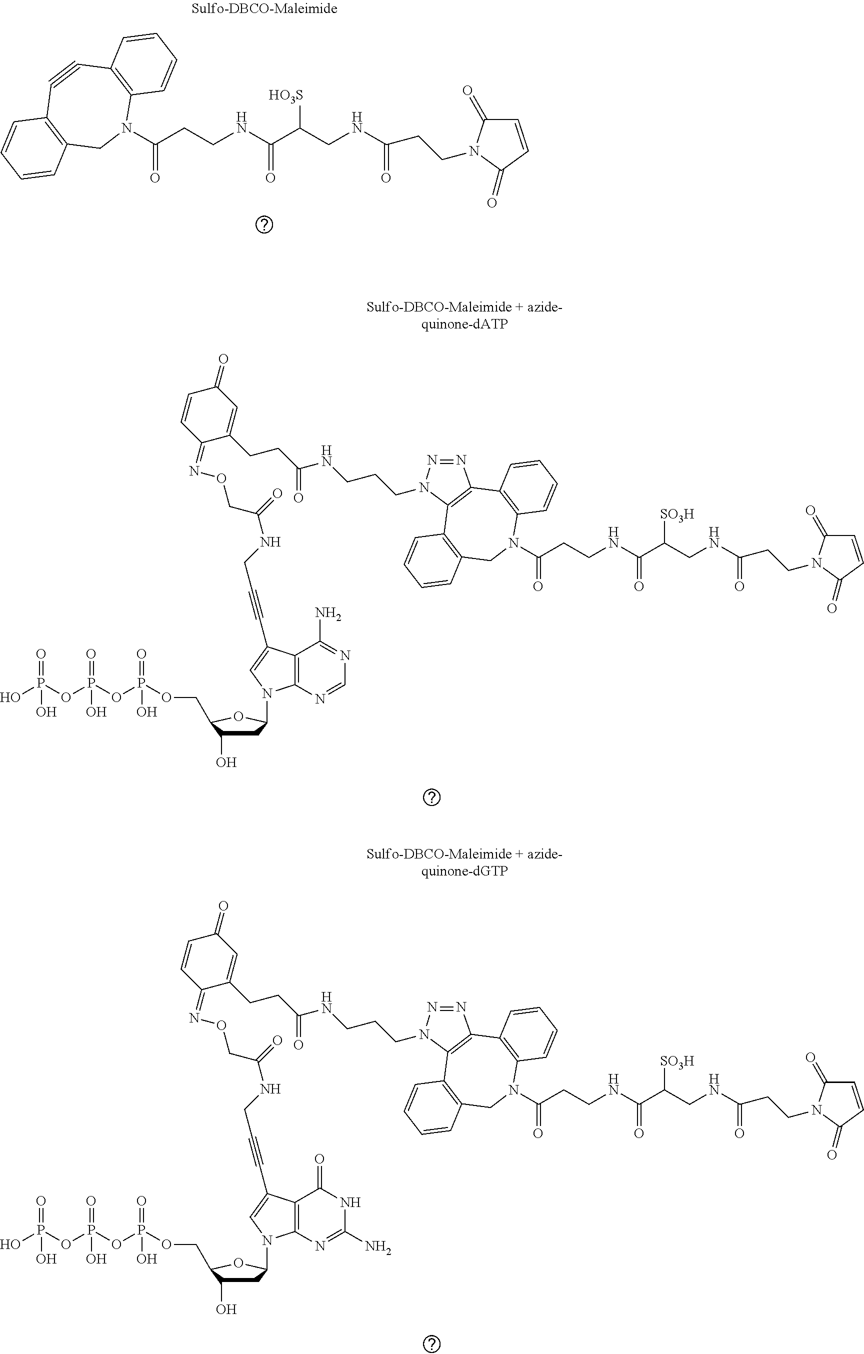

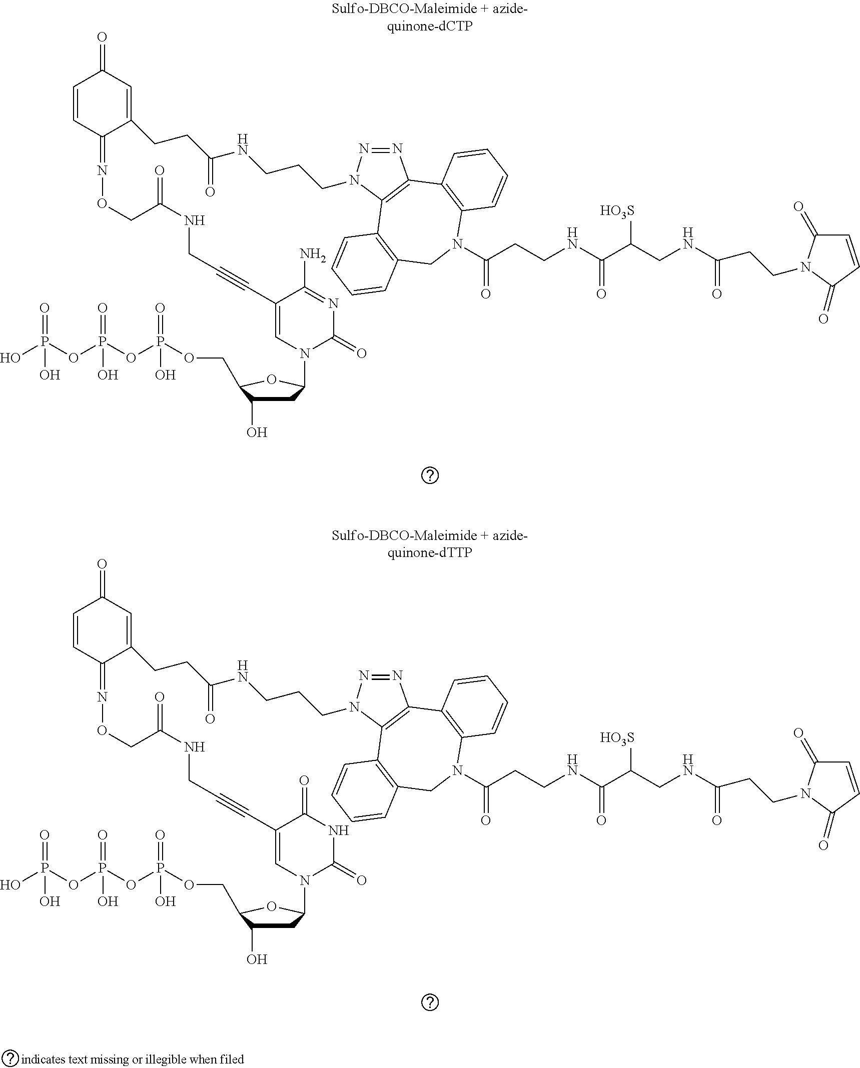

Note that the maleimide is drawn in its unreacted form for simplicity. The calculated mass of this segment attached to protein would not change. Deconvoluted mass spectral data for the unconjugated C7 variant, the C7 variant with the sulfo-DBCO-maleimide bifunctional crosslinker added, and the C7 variant reacted first with sulfo-DBCO-maleimide, followed by the azide-quinone-nucleotides are provided (FIG. 7A, FIG. 7B, FIG. 7C, FIG. 7D, FIG. 7E, FIG. 7F).

TABLE 5
Tabulated expected masses and masses obtained from deconvolution
of mass spectral data for the C7 protein before and after
conjugation with a bifunctional small molecule crosslinker
(sulfo-DBCO-maleimide) and azide-quinone-nucleotides

| | | | | Found mass |
|---|---|---|---|---|
| | | | Expected mass of | (deconvoluted |
| | | | protein-small | m/z) of protein |
| | | Small molecule | molecule | small molecule |
| Entry | Protein | added | conjugate (Da) | conjugate |
| 1 | C7 only | None | 43,177.01 | 43176.1 |
| 2 | C7- | Sulfo-DBCO- | 43,177.01 + 578.14 = | 43754.9 |
| | DBCO | maleimide | 43,755.15 | |
| 3 | C7- | Sulfo-DBCO- | 43,177.01 + 1438.29 = | 44614.4 |
| | DBCO- | maleimide + | 44,615.30 | |
| | dATP | azide-quinone- | | |
| | | dATP | | |
| 4 | C7- | Sulfo-DBCO- | 43,177.01 + 1454.29 = | 44630.4 |
| | DBCO- | maleimide + | 44,631.30 | |
| | dGTP | azide-quinone- | | |
| | | dGTP | | |
| 5 | C7- | Sulfo-DBCO- | 43,177.01 + 1416.26 = | 44592.7 |
| | DBCO- | maleimide + | 44593.27 | |
| | dTTP | azide-quinone- | | |
| | | dTTP | | |
| 6 | C7- | Sulfo-DBCO- | 43.177.01 + 1415.28 = | 44591.7 |
| | DBCO- | maleimide + | 44592.29 | |
| | dCTP | azide-quinone- | | |
| | | dCTP | | |

### Example 6. Cleavage of Nucleotides and Synthetic Control Using Soluble Redox Agents

The protein-linker-small molecule complex can be controlled via soluble electron (redox) shuttles when placed in an environment with electrodes; the electrodes can be used to control the oxidation state of the soluble redox shuttle. Examples of soluble redox shuttles include methylene blue, quinones, NAD/NADH (nicotinamide adenine dinucleotide, and reduced form), flavins (e.g., riboflavin), viologens, TEMPO (2,2,6,6-tetramethylpiperidin-1-yl)oxyl or (2,2,6,6-tetramethylpiperidin-1-yl) oxidanyl)/related compounds and certain organometallic coordination complexes, including ferrocenes, (NH₄)₄Fe(CN)₆, and K₄Fe(CN)₆. When the soluble redox shuttle is in a particular oxidation state, it will react with the redox-cleavable linker, facilitating cleavage of the small molecule from the protein. The user can thus control cleavage events via the applied voltage at the electrodes.

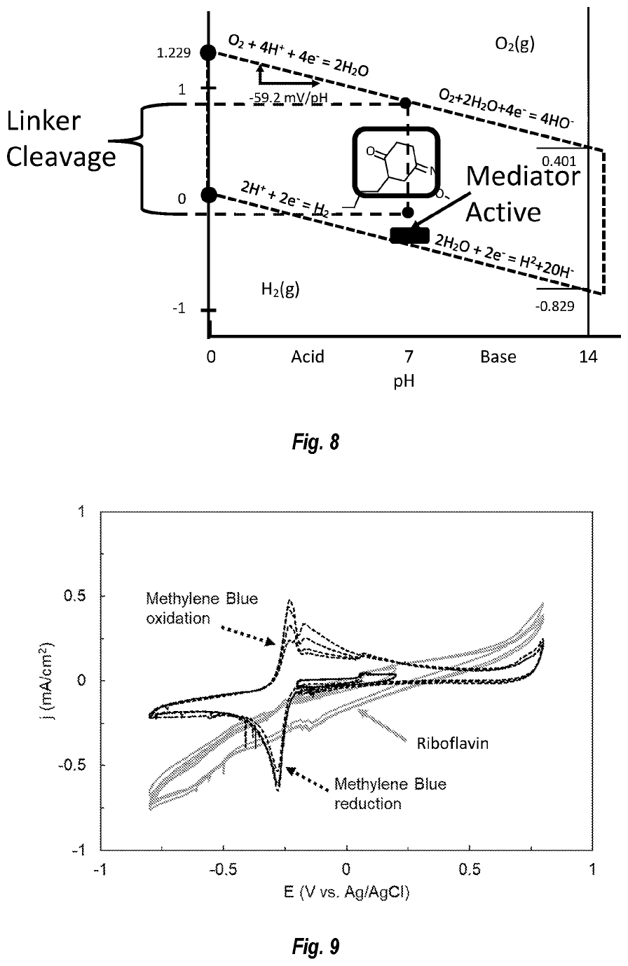

A soluble redox shuttle can be selected based on knowledge of the redox-cleavable linker used and desired operating pH. For example, an operating window for cleavage of quinone oxime ethers at pH 6.5 is estimated to be between +0.85 V and −0.38 V at 25 °C (FIG. 8). This estimate is based on the fact that DTT (dithiothreitol) (with potential of −0.27 V at pH 6.5) has been shown to cleave quinone oxime ethers. Park, S.; Westcott, N. P.; Luo, W.; Dutto, D.; Yousaf, M. N. Bioconjugate Chemistry “General chemoselective and redox-responsive ligation and release strategy,” 25:543-551 (2014). Comparative cyclic voltammetry data for a series of soluble redox shuttles is presented in FIG. 9.

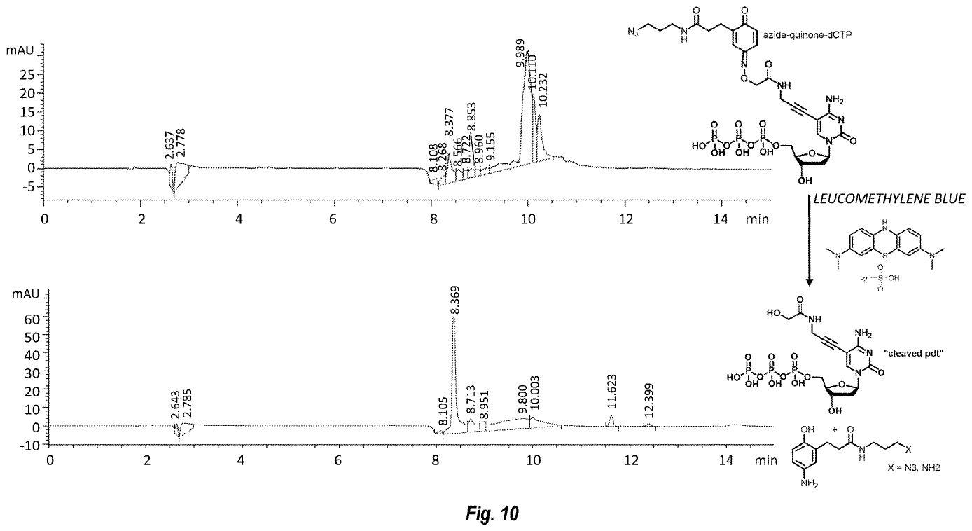

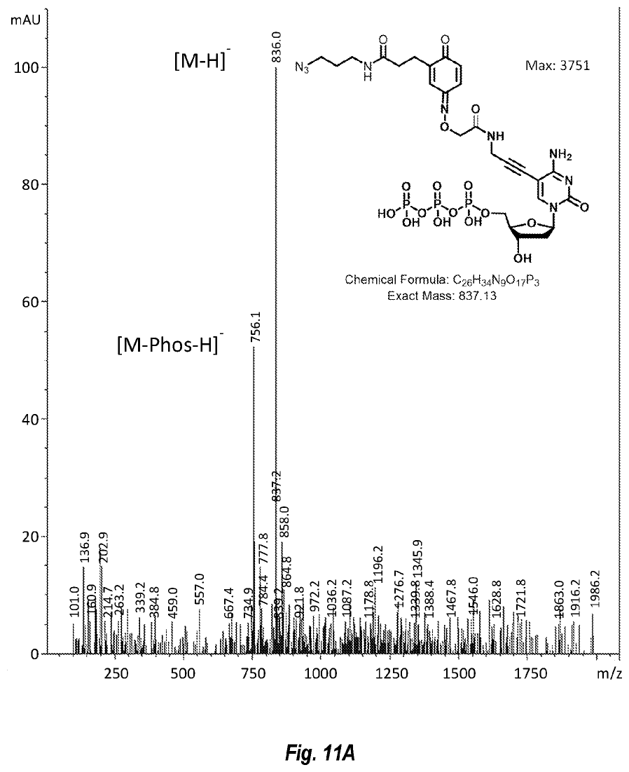

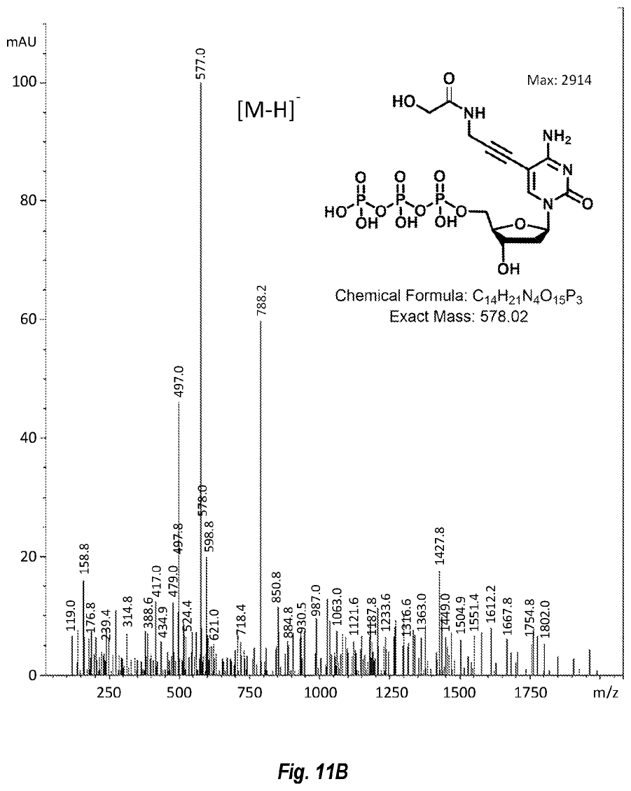

The quinone oxime ether bond (redox-cleavable linker) was cleaved by soluble reducing agents, including leucomethylene blue (the reduced form of methylene blue). Cleavage was demonstrated both using the precursor small molecule azide-quinone nucleotide and on the full conjugate with TdT. An azide-quinone-nucleotide (azide-quinone-dCTP) in 200 mM phosphate, pH 6.5 with 100 mM NaCl was treated with leucomethylene blue (at 10-fold excess) at room temperature. After dilution with 20% MeCN/0.01 M TEAA (triethylammonium acetate), the mixture was analyzed by LCMS (FIG. 10, FIG. 11A and FIG. 11B). The azide-quinone nucleotide disappeared, and the resulting product (labeled “cleaved pdt”) was detected by mass spectrometry.

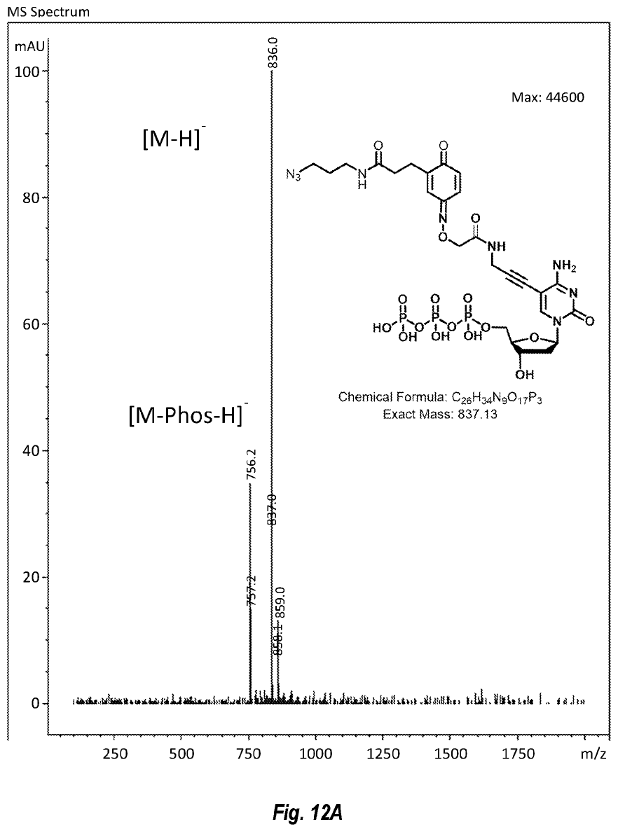

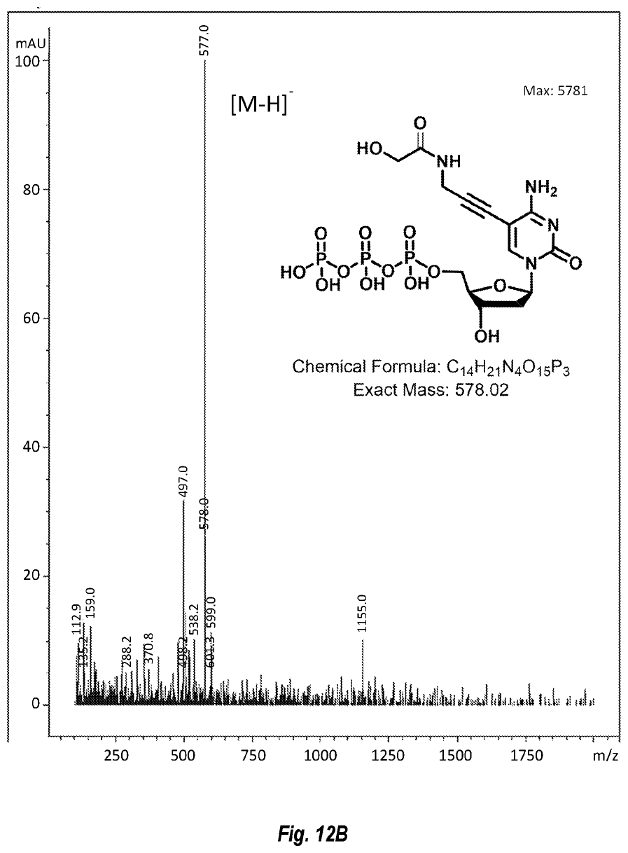

A second batch of leucomethylene blue reactions was performed on starting material that had fewer impurities in the mixture. MS data for the starting material and converted product are shown in FIG. 12A and FIG. 12B.

### Example 7. Redox Shuttle Solutions

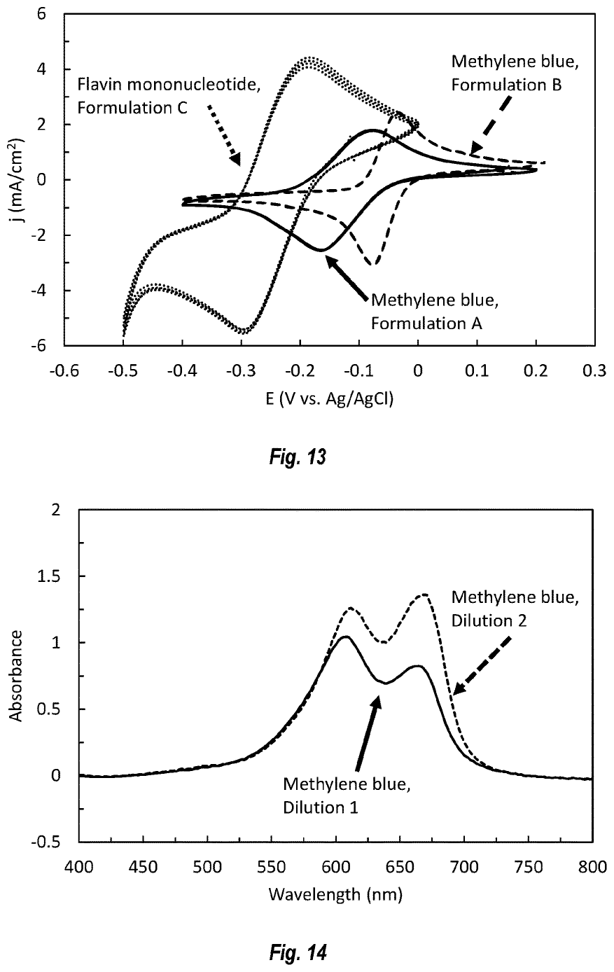

Three non-limiting examples of exemplary redox shuttle solution compositions, or formulations, are presented in Table 6 and comprise a redox shuttle (methylene blue or flavin mononucleotide) with a buffer, a supporting electrolyte, and a hydrotrope constituent(s). Analogous cyclic voltammograms for Formulations A, B, and C are shown in FIG. 13. These examples demonstrate the range of reduction potentials and current densities accessible in the generation of active soluble redox shuttles, based on their identity and solution formulation. Methylene blue shows a distinct improvement in electrochemical kinetics between Formulations A and B, due to the addition of 1 M nicotinamide. The peak separation (ΔEₚ) decreases from 105 to 49 mV, and its corresponding standard rate constant (k₀) increases from 4.5×10⁻⁴ to 2.6×10⁻³ cm/s. This improvement suggests both greater stability of methylene blue in solutions containing formulants such as nicotinamide due to its buffering effect. Nicotinamide also induces an anodic shift in the reduction potential of methylene blue despite a constant nominal pH, either from its increased buffer capacity or by acting as an additional proton source/sink during reduction/oxidation. Formulation C shows a higher current response than Formulation B, despite slower kinetics (ΔEₚ=103 mV, k₀=1.9×10⁻⁴ cm/s), due to the greater concentrations of both the redox shuttle (flavin mononucleotide) and supporting electrolyte (NaCl).

TABLE 6
Non-limiting exemplary examples of
redox shuttle solution formulations

| Example | |
|---|---|
| formulation | Formulation |
| Formulation A | Redox Shuttle: 10 mM methylene blue |
| | Buffer: 25 mM citrate-phosphate buffer, pH 3.4 |
| | Supporting electrolyte: 100 mM sodium chloride |
| | Hydrotrope: none |
| Formulation B | Redox Shuttle: 10 mM methylene blue |
| | Buffer: 25 mM citrate-phosphate buffer, pH 3.4 |
| | Supporting electrolyte: 100 mM sodium chloride |
| | Hydrotrope: 1M nicotinamide |
| Formulation C | Redox Shuttle: 50 mM flavin mononucleotide |
| | Buffer: 25 mM citrate-phosphate buffer, pH 3.4 |
| | Supporting electrolyte: 1M sodium chloride |
| | Hydrotrope: 1M nicotinamide |

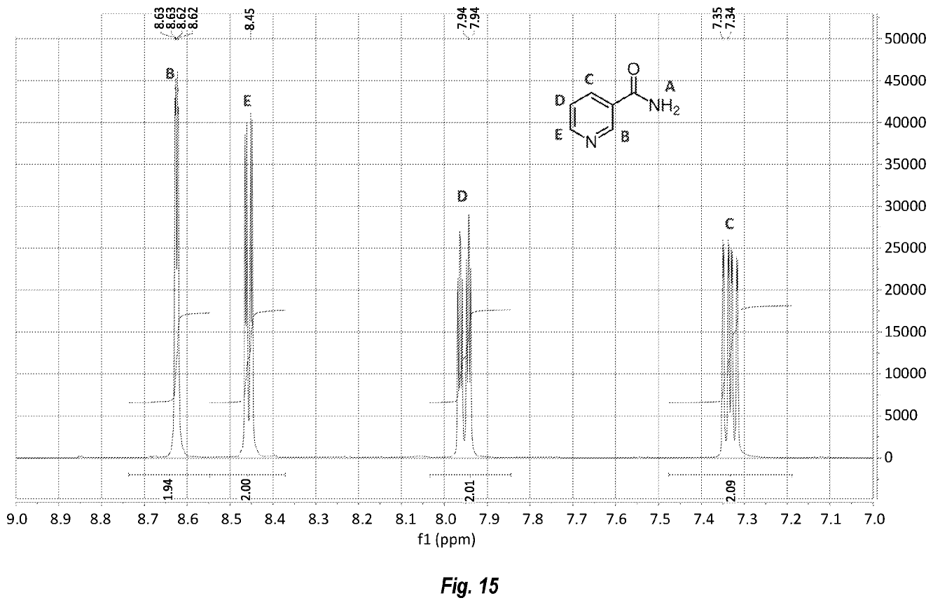

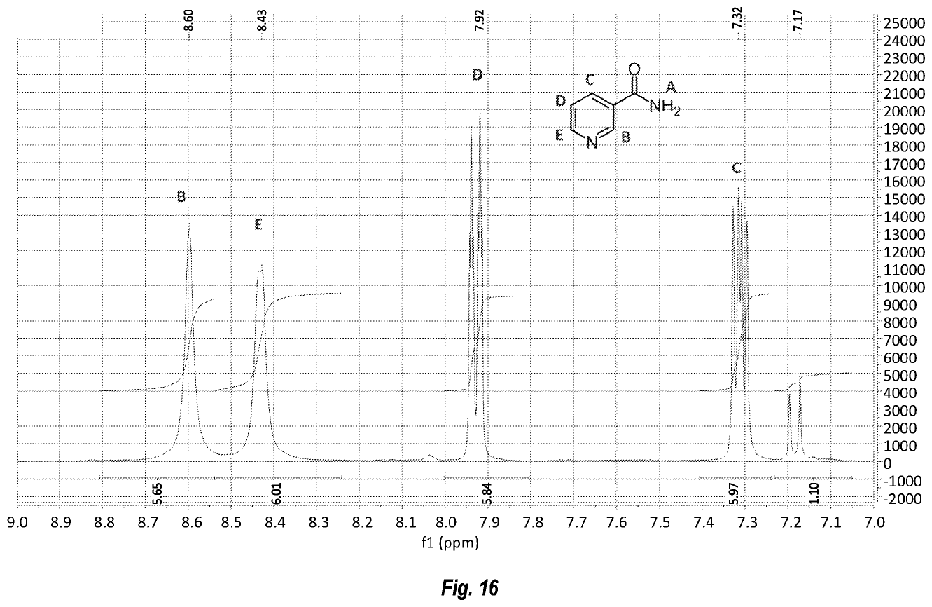

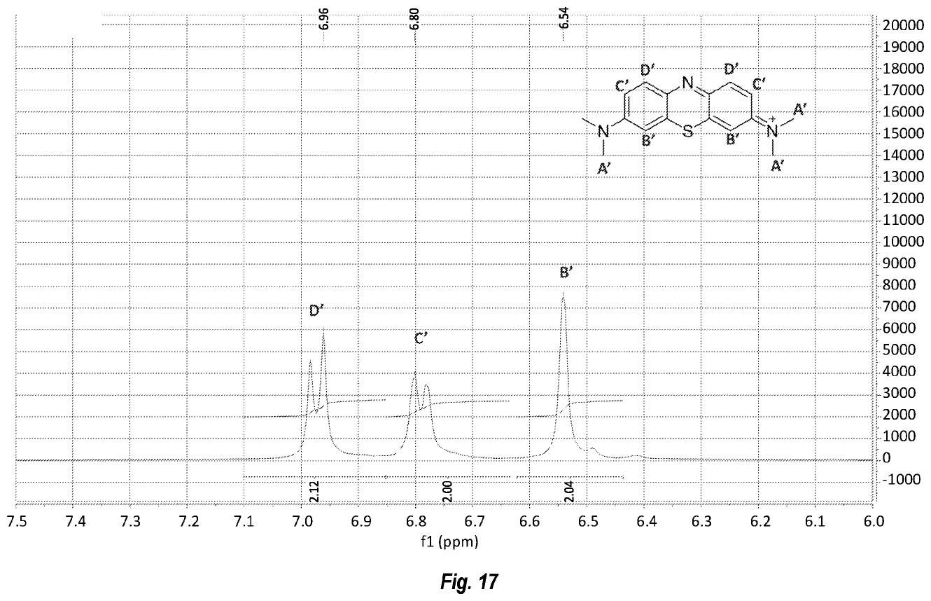

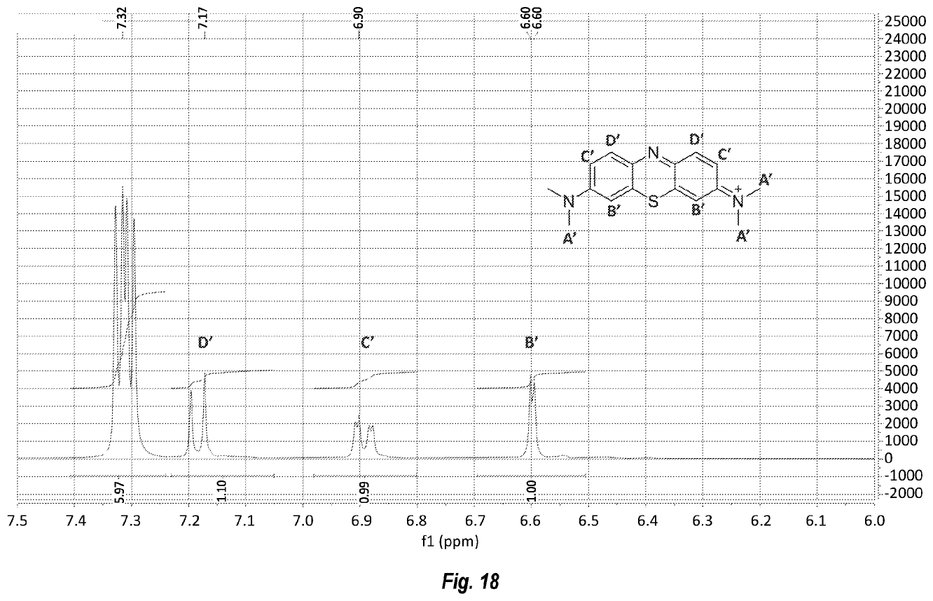

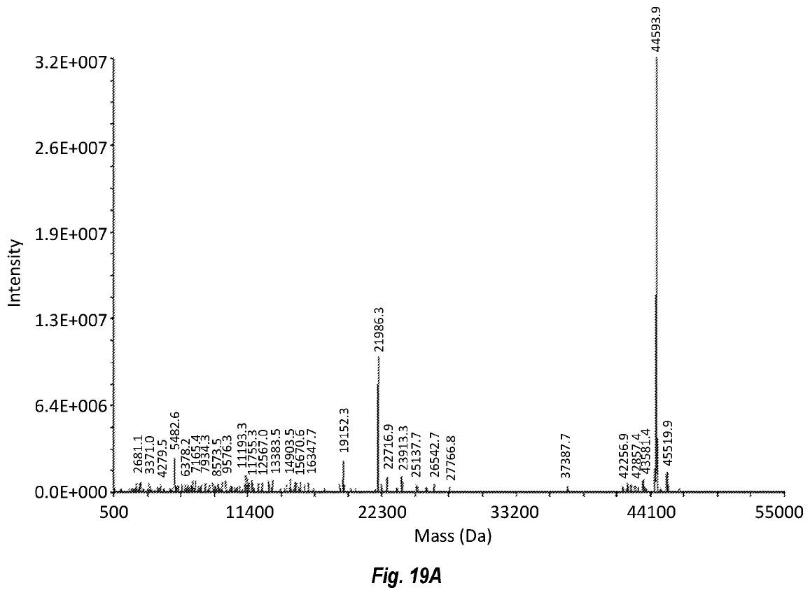

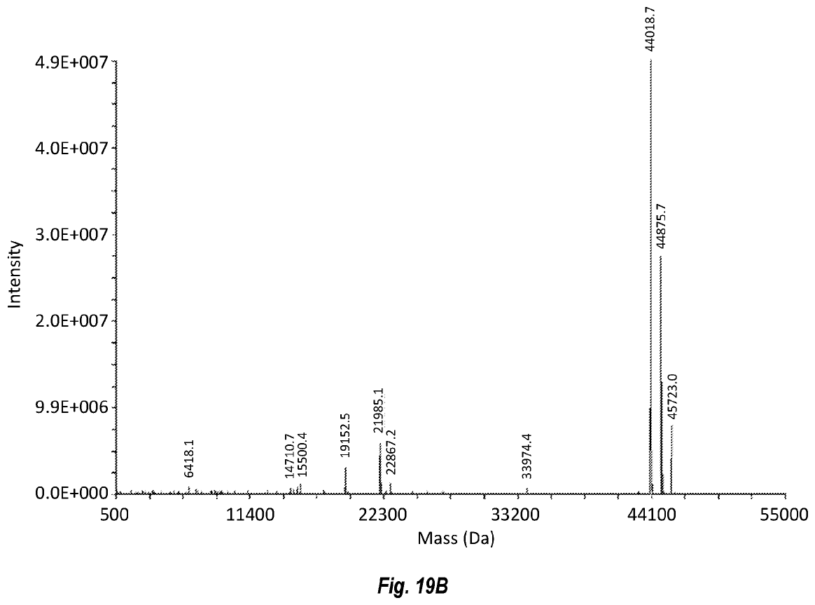

Further chemical analysis of Formulation B supports the role nicotinamide plays in stabilizing methylene blue during the generation of the active redox shuttle. FIG. 14 shows ultraviolet-visible light (UV-Vis) absorbance spectra of Formulation B dilutions (Dilution 1:0.25 mM methylene blue and 2.5 mM nicotinamide; Dilution 2:0.25 mM methylene blue and 100 mM nicotinamide). The peak at 605 nm is associated with the dimeric or tetrameric form, and the peak at 670 nm is attributed to the monomer. Fernández-Pérez, A. & Marbán, G., “Visible Light Spectroscopic Analysis of Methylene Blue in Water; What Comes after Dimer?” ACS Omega 5:29801-29815 (2020). Large excess of nicotinamide induces a clear absorbance shift from oligomeric to monomeric methylene blue species in solution, providing evidence for solution stabilization through the formation of nicotinamide-methylene blue associations. Coordination is further supported by 1H nuclear resonance spectroscopy (1H-NMR, FIGS. 15-18). 1H peaks assigned to hydrogens B and E of nicotinamide undergo broadening in the presence of methylene blue, as do the 1H peaks assigned to hydrogens B′, C′, and D′ of methylene blue in the presence of nicotinamide. Moreover, the j-coupling constants for hydrogens B′ and E are identical at 2.56 Hz, indicating close physical proximity between the two species. Suitable formulation design, such as inclusion of buffering hydrotropic agents like nicotinamide, improves the stability of the soluble redox shuttle, supports rapid generation kinetics (i.e. electrochemical reversibility), and maximizes the efficiency of breaking the redox-cleavable linker.

### Example 8. Cleavage of Linker Attaching C7 Variant to Nucleotide

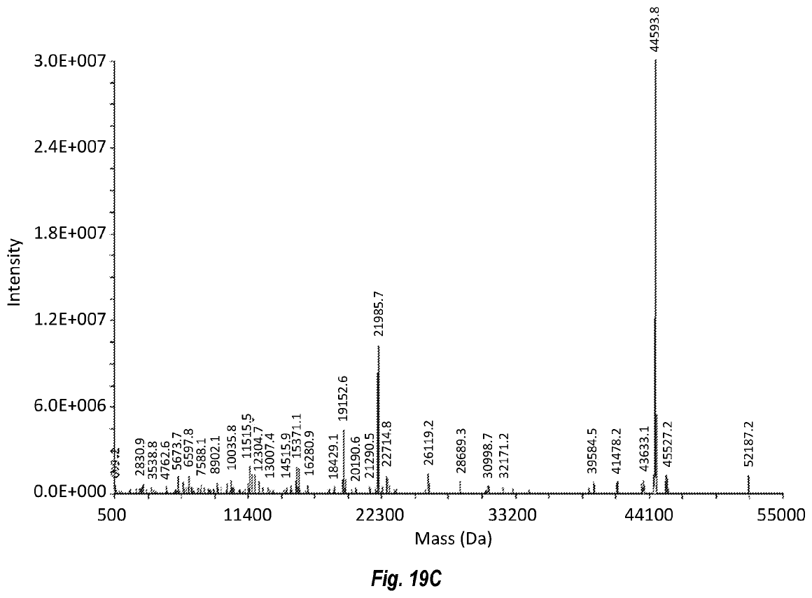

The C7 variant conjugated to sulfo-DBCO-maleimide followed by the azide-quinone-dTTP was treated with leucomethylene blue. Another sample was treated with methylene blue (not yet reduced to the active form). The expected mass loss from reductive cleavage of the quinone oxime ether bond was observed when the protein conjugate was treated with leucomethylene blue; no change in mass was observed when the protein conjugated was treated with methylene blue (in the inactive form). Mass spectral data are tabulated in Table 7, and deconvoluted mass spectra are shown in FIG. 19A, FIG. 19B, and FIG. 19C. The expected cleavage products and expected masses for the small molecules that were conjugated and/or cleaved from the enzyme are shown below. The small molecule is drawn as the unconjugated maleimide for simplicity, but the maleimide would be bonded to the protein as a thioether (via a reaction of the cysteine residue with the maleimide)

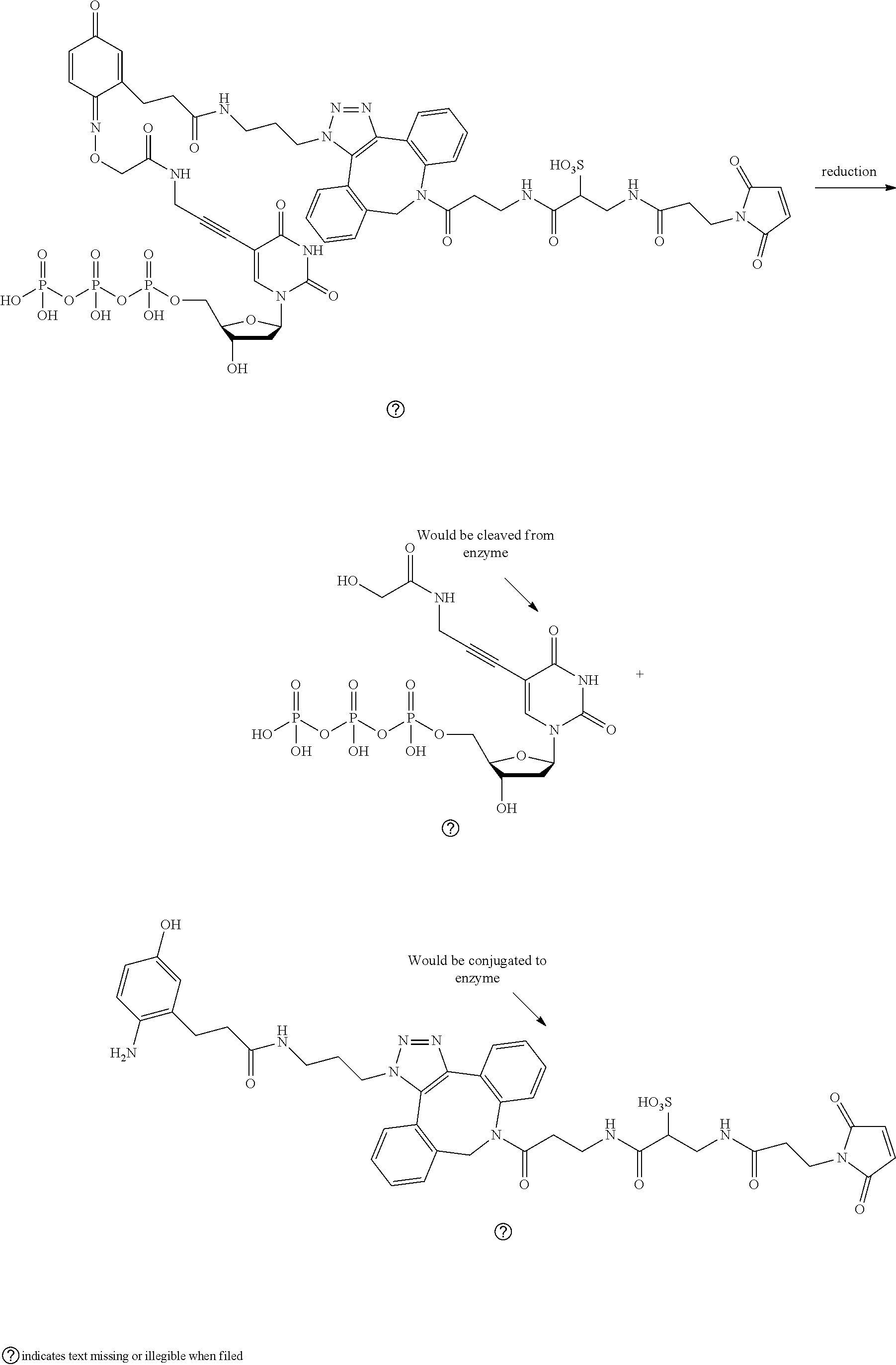

The conjugated protein was split into batches. One batch was treated with leucomethylene blue (soluble reducing agent); reduction was observed. One batch was treated with methylene blue (not in the active, reduced form); no change in protein mass was observed.

TABLE 7
Tabulated protein masses and deconvoluted mass spectral data for a C7 variant
that was conjugated with sulfo-DBCO-maleimide followed by azide-quinone-dTTP

| | | | | Found mass |
|---|---|---|---|---|
| | | | Expected mass of | (deconvolutated |
| | | | protein-small | m/z) of protein |
| | | | molecule conjugate | small molecule |
| Entry | Protein | Small molecule added | (Da) | conjugate |
| 1 | C7-DBCO-dTTP | Sulfo-DBCO- | 43,177.01 + 1416.26 = | 44593.9 |
| | | maleimide + | 44593.27 | |
| | | azide-quinone-dTTP | | |
| 2 | C7-DBCO-dTTP; | Sulfo-DBCO- | 43,177.01 + 841.29 = | 44018.7 |
| | treated with | maleimide + | 44018.3 | |
| | leucomethylene | azide-quinone-dTTP | | |
| | blue | | | |
| 4 | C7-DBCO-dTTP; | Sulfo-DBCO- | 43,177.01 + 1416.26 = | 44593.8 |
| | treated with | maleimide + | 44593.27 | |
| | methylene blue | azide-quinone-dTTP | | |

### Example 9. TdT Attached to Nucleotide Via Quinone Oxime Ether Linker Bound to ssDNA

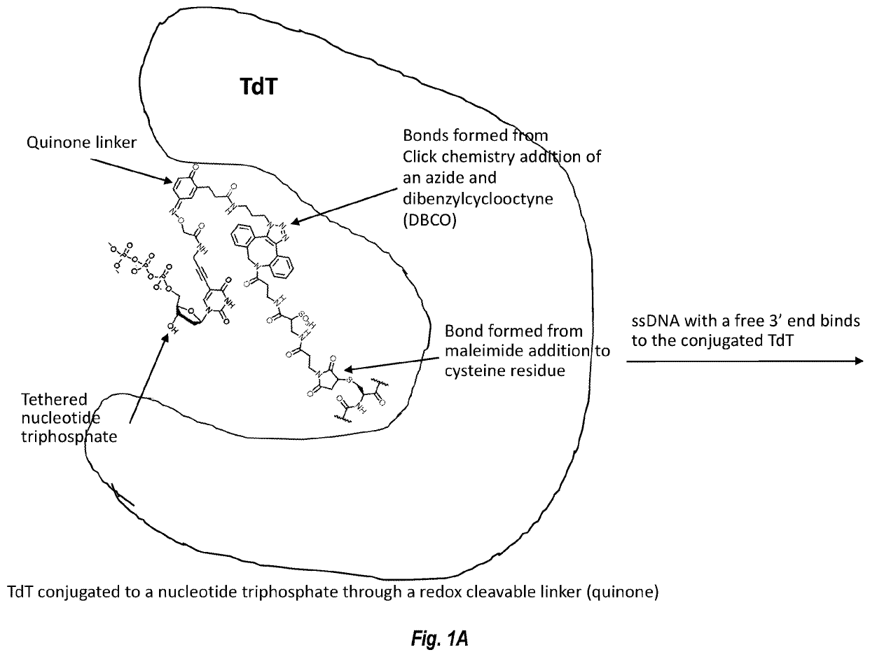

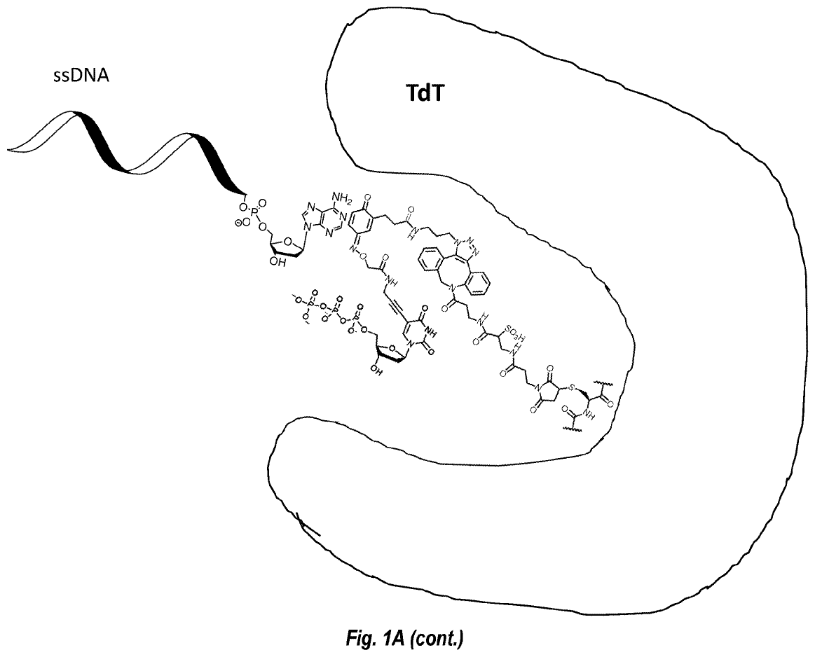

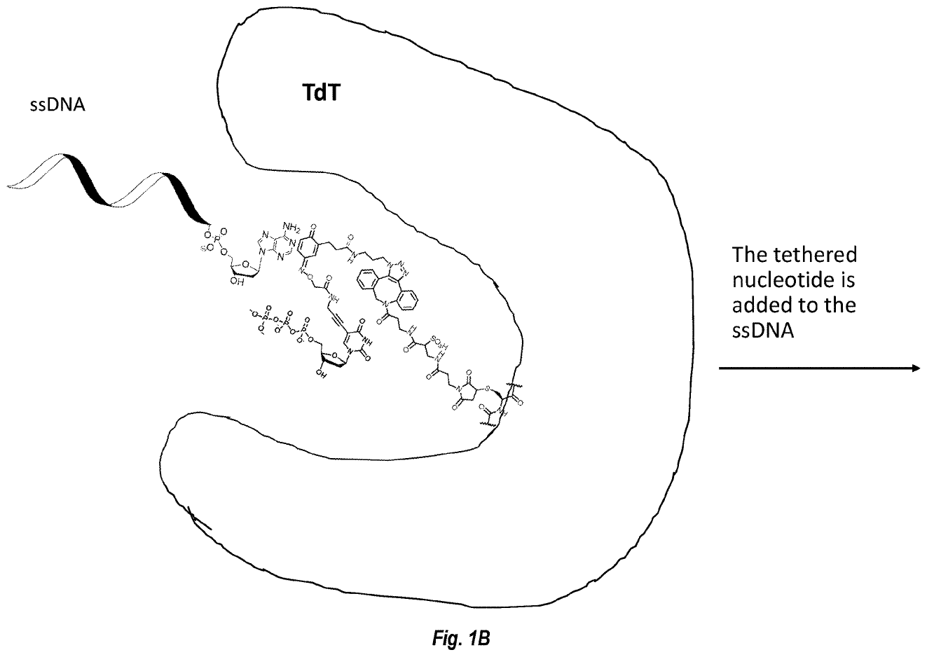

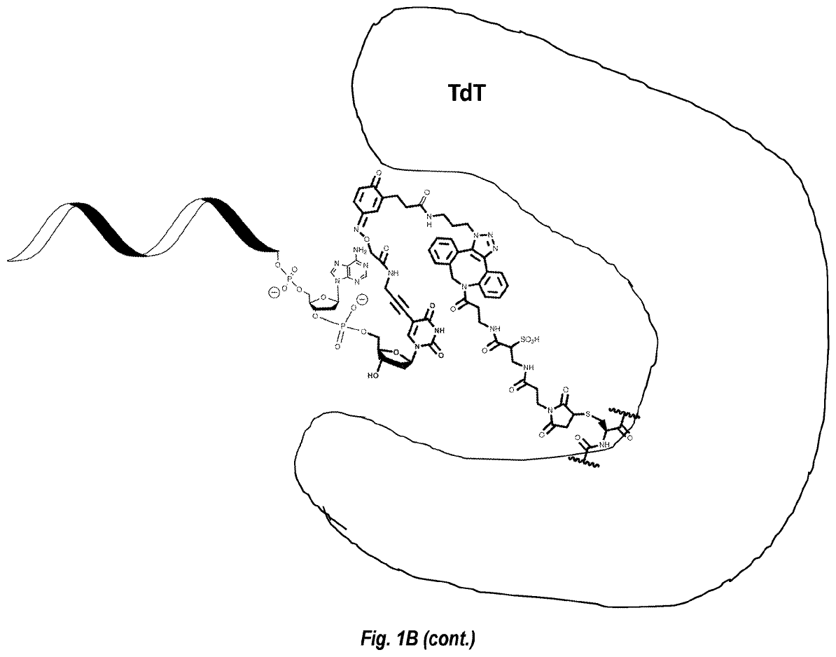

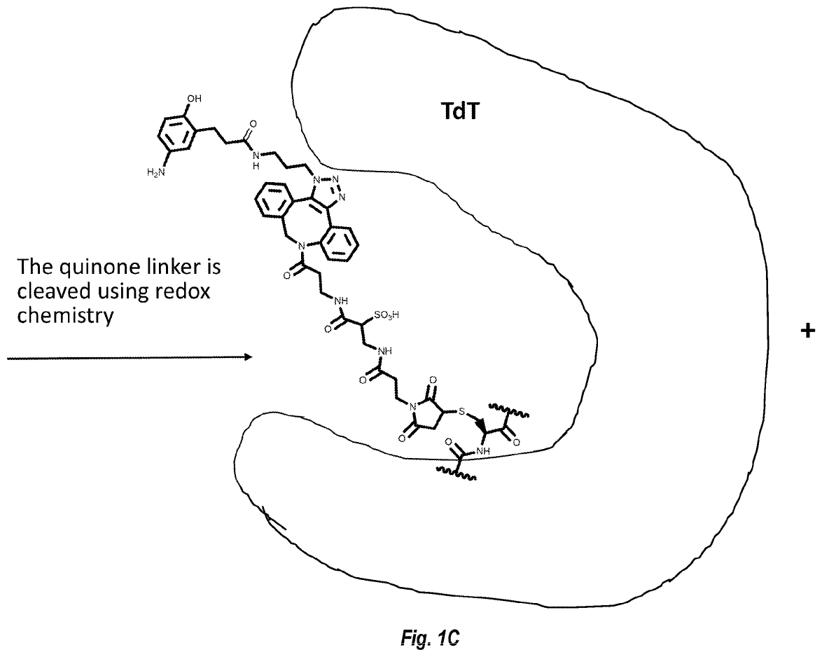

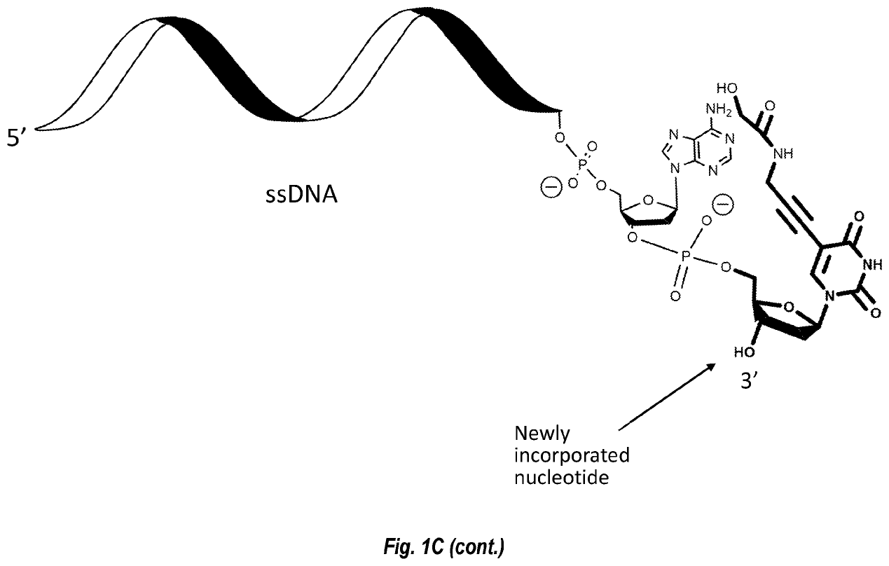

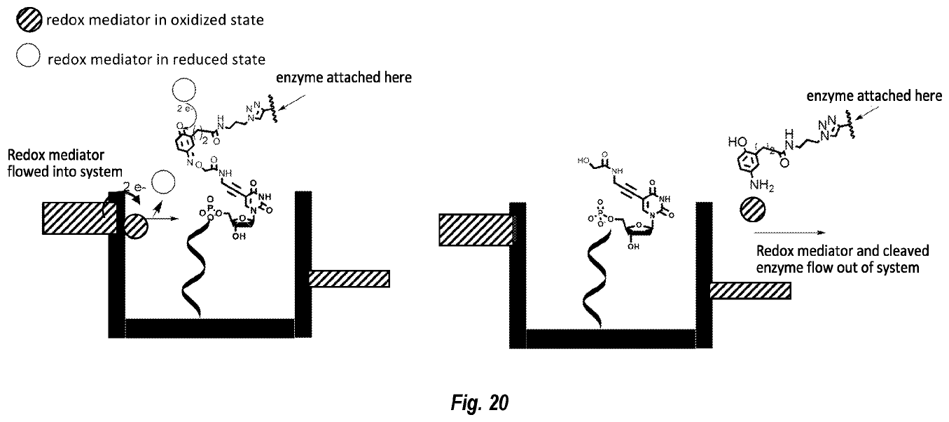

One application of the technology is nucleic acid synthesis. In this application, engineered TdT is covalently attached to a nucleotide triphosphate via a redox cleavable linker. The attachment and linker chemistry is designed so that the nucleotide triphosphate is accessible to the active site of the enzyme. In the presence of a single stranded nucleic acid (ssDNA), the TdT will bind the ssDNA and incorporate the covalently attached linker to the 3′ end of the ssDNA, effectively tethering the ssDNA to the TdT protein by means of the newly incorporated nucleotide. This mechanism is also depicted in FIG. 1. The TdT protein attached to the ssDNA would effectively block subsequent additions of nucleotide to the ssDNA strand. The ssDNA-TdT complex, when in the presence of electrodes and a suitable redox shuttle (FIG. 20) would be electrochemically/redox-cleaved to generate ssDNA with the newly incorporated nucleotide, now separated from the TdT/linker. A fresh TdT covalently bound to nucleotide triphosphate would be introduced, allowing the next nucleotide in the sequence to be added to the growing chain.

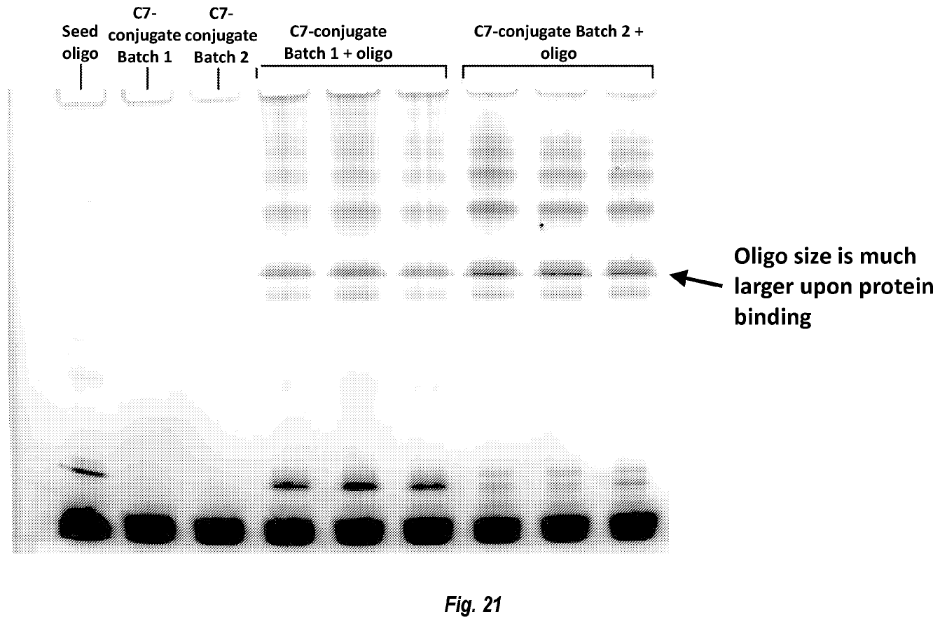

It was demonstrated that TdT proteins conjugated to a nucleotide via a quinone oxime ether linker do indeed bind ssDNA. A Cy2-labeled seed oligo was incubated with C7 variants conjugated to an azide-quinone-dTTP molecule for 30 minutes at 37 °C. This reaction was then analyzed via SDS-PAGE on a denaturing Tris-Glycine gel that separated the unbound seed oligo from the seed oligo covalently bound to TdT based on size. Results in FIG. 21 show the presence of additional higher molecular weight species shifted upwards on the gel following incubation with conjugated C7 compared to seed oligo alone confirming covalent binding of the conjugated enzyme to seed oligo.

### Example 10. Verification that Oligonucleotides Extended by C7 Variant

TdT proteins covalently conjugated to nucleotides can be used to incorporate multiple nucleotides onto the end of single-stranded DNA, using leucomethylene blue as a soluble reducing agent to cleave the protein from the ssDNA complex after nucleotide incorporation at each step (see FIG. 1 for an illustration of this process).

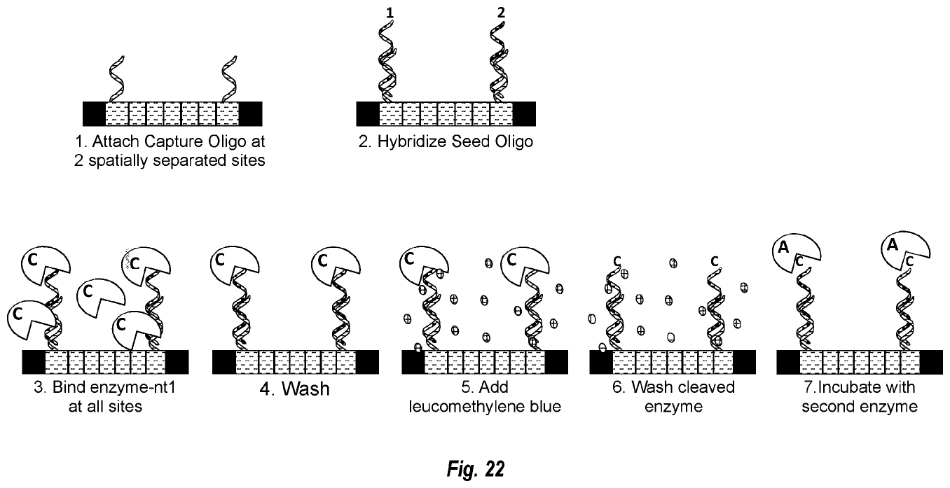

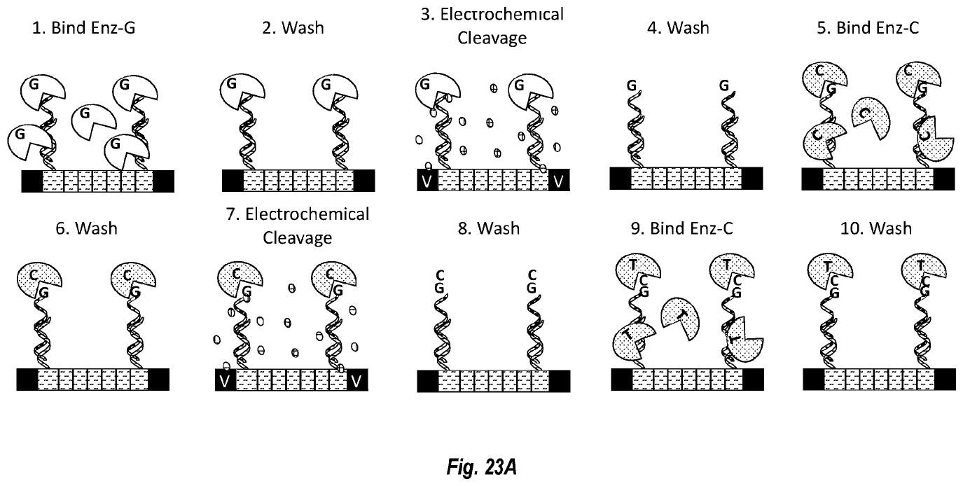

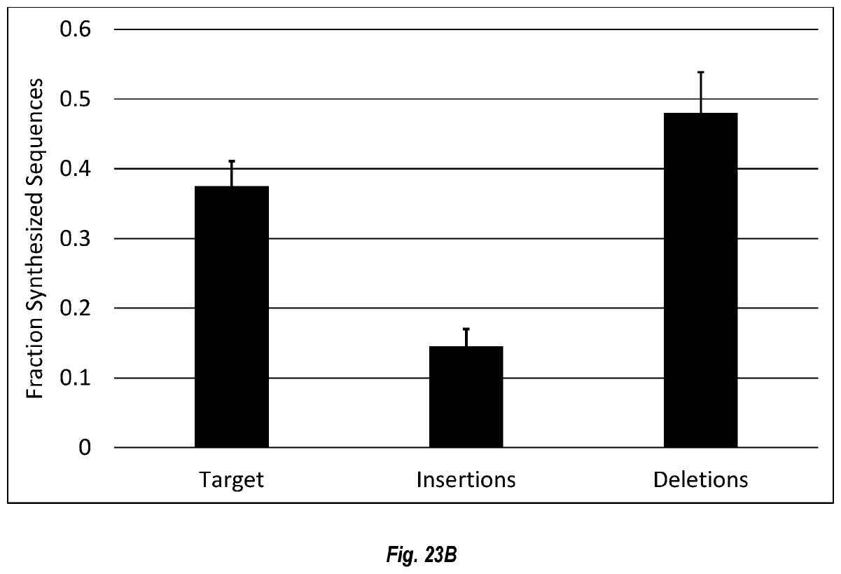

To verify that oligonucleotides could be extended by the covalently-tethered C7 conjugates, an oligonucleotide was anchored to a surface at the 3′ end; a second oligonucleotide was hybridized to that strand to generate a 3′ end available for extension via addition of nucleotides (FIG. 22). The surface was outfitted with a gasket to allow for introduction of reagents. In this case, a C7-nucleotide conjugate was introduced, incubated on the surface, then washed from the surface. After cleavage with leucomethylene blue, a second C7-nucleotide conjugate was added (with a different nucleotide). The starting C7-nucleotide conjugate was a result of reaction with azide-quinone-CTP in some cases or azide-quinone-dATP in other cases. In both cases, the expected addition of the first nucleotide (either A or C) followed by the second nucleotide (either A or C) was observed (FIG. 22, Table 8). Addition of the appropriate nucleotides was determined via sequencing.

A single cycle of extension with either C7-C or C7-A resulted in 85% and 64% intended single addition respectively. Two combinations of sequences were tried (adding either C or A first, followed by either A or C), and the expected two-nucleotide addition was observed 32% of the time regardless of order of nucleotide addition.

TABLE 8
Sequencing results from additions of nucleotides to a seed oligonucleotide
after incubation with a TdT-nucleotide conjugate and cleavage
with a soluble reducing agent (leucomethylene blue)

| | 0 | | | | |
|---|---|---|---|---|---|
| | Extensions | +A | +C | +AC | +CA |
| C7-C + | 6.82 | 0.13 | 85.65 | 0.01 | 0.06 |
| Buffer | | | | | |
| C7-A + | 7.28 | 63.73 | 0.02 | 0.01 | 0.00 |
| Buffer | | | | | |
| C7-C + | 5.87 | 1.79 | 11.61 | 0.00 | 32.77 |
| C7-A | | | | | |
| C7-A + | 5.95 | 4.37 | 0.80 | 31.97 | 0.00 |
| C7-C | | | | | |

### Example 11. Electrochemically Controlled DNA Synthesis

Electrochemically controlled DNA synthesis using a nucleotide-conjugated enzyme was also tested in a 3-cycle synthesis run. As depicted in FIG. 22, in each cycle of synthesis the enzyme conjugated to the nucleotide to be added was incubated with seed DNA attached to a surface surrounded by electrodes. Unbound enzyme was washed away and then a methylene blue solution was introduced. Upon applying a voltage to the electrodes, the methylene blue was reduced and cleaved the enzyme from the extended DNA. The cleaved enzyme was washed away and then the next enzyme conjugated to the next nucleotide to be added was introduced. Following this method, enzymes conjugated to guanine (G), cytosine (C), and then thymine (T) were added over three cycles with electrochemical cleavages in between. The DNA was then recovered from the surface using sodium hydroxide denaturation and then the sample was sequenced. The distribution of synthesized sequences is shown in FIG. 23B. 37% target was achieved with the remaining sequences containing largely deletions (48%) and a minor population containing insertions (25%).

### Example 12. Protocol for Sequencing Products

The addition of nucleotides to the oligonucleotide was determined using sequencing techniques described here. The hybridized seed oligonucleotide was eluted from the surface via denaturation from the capture oligo using 0.1M NaOH and then subjected to the following protocol.

Sequencing sample preparation began with polyadenylation (for sequences expected to end in thymine, cytosine, or guanine) or the addition of a poly(T) tail (for sequences expected to end in adenine) to the 3′ end of each DNA sample using commercial TdT. The samples were then amplified using primers that bind to a conserved 5′ sequence and the complementary 3′ tail while incorporating Illumina sequencing priming sites into the synthesized amplicons. This step was performed using qPCR in order to achieve optimal amplification of all samples regardless of input DNA concentration. A portion of each PCR product was analyzed by gel electrophoresis to confirm the size and relative abundance of the amplicons. The products of all samples were then normalized based on relative abundance and used as template for a second PCR reaction, which incorporated Illumina flow cell adaptors and a variable index into each sample. The products of the final PCR reaction were characterized by gel electrophoresis and all indexed samples were pooled together proportionally based on relative abundance. Size selection and purification of the pooled library was performed by DNA gel extraction and the final library was analyzed by Qubit dsDNA HS assay and Tapestation D1000 screentape to determine the final molar concentration. The library was then diluted, denatured and sequenced following standard Illumina protocols.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A method of nucleic acid molecule synthesis comprising the steps of:
(a) providing an engineered terminal deoxynucleotidyl transferase (TdT), wherein the engineered TdT comprises a nucleotide molecule covalently attached to the TdT via a redox-cleavable linker;
(b) providing a single-stranded nucleic acid molecule comprising a 5′ and a 3′ end;
contacting the engineered TdT and the single-stranded nucleic acid molecule under conditions suitable for the TdT to bind to the 3′ end of the nucleic acid molecule and form a TdT-nucleic acid strand complex, thereby incorporating the nucleotide molecule covalently linked to the TdT into the nucleic acid strand, resulting in the nucleic acid molecule tethered to the TdT and thereby blocking subsequent additions of a nucleotide molecule to the nucleic acid strand; and
(d) exposing the TdT-nucleic acid strand complex to suitable electrochemical conditions, wherein the redox-cleavable linker is cleaved thereby separating the TdT-redox-cleavable linker molecule from the nucleic acid strand with the newly incorporated nucleotide molecule, resulting in a nucleic acid strand with the newly incorporated nucleotide molecule; and
(e) repeating steps (a) through (d) thereby synthesizing a nucleic acid molecule.

2. The method of claim 1, wherein the redox-cleavable linker is a quinone oxime ether linker.

3. The method of claim 1 or claim 2, wherein the electrochemical conditions of step (d) comprise one or more suitable soluble redox shuttles in the presence of two or more electrodes, wherein the two or more electrodes control the oxidation state of the soluble redox shuttles.

4. The method of any one of claims 1-3, wherein the soluble redox shuttles comprise a thiazine, a viologen, dinucleotides, a flavin, a quinone, peroxide, bromide, vanadium and/or a metal complex.

5. The method of claim 4, wherein the soluble redox shuttles comprise a thiazine.

6. The method of claim 4, wherein the soluble redox shuttles comprise a viologen.

7. The method of claim any one of claims 1-3, wherein the soluble redox shuttles comprise dinucleotides.

8. The method of claim 7, wherein the dinucleotides comprise NAD/NADH, nicotinamide adenine dinucleotide, and/or reduced form.

9. The method of claim 4, wherein the soluble redox shuttles comprise a flavin.

10. The method of claim 4, wherein the soluble redox shuttles comprise a quinone.

11. The method of claim 4, wherein the soluble redox shuttles comprise peroxide.

12. The method of claim 4 wherein the soluble redox shuttles comprise bromide.

13. The method of claim 4, wherein the soluble redox shuttles comprise vanadium.

14. The method of claim 4, wherein the soluble redox shuttles comprise a metal complex.

15. The method of claim 5, wherein the thiazine is methylene blue.

16. The method of claim 4, wherein the quinone is 2,5-dihydroxy-1,4-benzoquinone (DHBQ), 9,10-anthraquinone-2,7-disulfonic acid (AQDS), 4,5-dihydroxybenzene-1,3-disulfonic acid (BQDS), or 2,3,5,6-tetrakis((dimethylamino)methyl)hydroquinone (FQH₂).

17. The method of claim 9, wherein the flavin is riboflavin (RF), flavin mononucleotide (FMN), or flavin adenine dinucleotide (FAD).

18. The method of claim 6, wherein the viologen is methyl viologen (MV) or bis-(trimethylammonio) propyl viologen (BTMAP-Vi).

19. The method of claim 14, wherein the metal complex is TEMPO (2,2,6,6-tetramethylpiperidin-1-yl)oxyl, (2,2,6,6-tetramethylpiperidin-1-yl) oxidanyl).

20. The method of claim 14, wherein the metal complex is an organometallic coordination complex.

21. The method of claim 20, wherein the organometallic coordination complex is a ferrocene, bis-(trimethylammonio) propyl ferrocene (BTMAP-Fc), ammonium ferricyanide ((NH₄)₄Fe(CN)₆), potassium ferricyanide (K₄Fe(CN)₆), hexaamineruthenium (III) chloride (RuHex), permanganate, or dichromate.

22. The method of any one of claims 1-21, wherein the electrochemical conditions of step (d) further comprise a buffer, a supporting electrolyte, and/or a hydrotropic agent.

23. The method of claim 22, wherein the buffer comprises sodium citrate, potassium hydrogen phosphate, and/or potassium dihydrogen phosphate.

24. The method of claim 22, wherein the supporting electrolyte comprises sodium chloride, sodium salts, lithium salts, potassium salts, and/or magnesium salts.

25. The method of claim 22, wherein the hydrotropic agent comprises caffeine, urea, and/or nicotinamide (NA).

26. The method of any one of claims 1-3, wherein the electrochemical conditions of step (d) comprise a redox shuttle solution comprising methylene blue, citrate-phosphate buffer, and sodium chloride.

27. The method of any one of claims 1-3, wherein the electrochemical conditions of step (d) comprise a redox shuttle solution comprising methylene blue, citrate-phosphate buffer, sodium chloride, and nicotinamide.

28. The method of any one of claims 1-3, wherein the electrochemical conditions of step (d) comprise a redox shuttle solution comprising flavin mononucleotide, citrate-phosphate buffer, sodium chloride, and nicotinamide.

29. The method of any one of claims 1-3, wherein the electrochemical conditions of step (d) comprise a redox shuttle solution comprising 10 mM methylene blue, 25 mM citrate-phosphate buffer at pH 3.4, and 100 mM sodium chloride.

30. The method of any one of claims 1-3, wherein the electrochemical conditions of step (d) comprise a redox shuttle solution comprising 10 mM methylene blue, 25 mM citrate-phosphate buffer at pH 3.4, 100 mM sodium chloride, and 1 M nicotinamide.

31. The method of any one of claims 1-3, wherein the electrochemical conditions of step (d) comprise a redox shuttle solution comprising 50 mM flavin mononucleotide, 25 mM citrate-phosphate buffer at pH 3.4, 1 M sodium chloride, and 1 M nicotinamide.

32. The method of any one of claims 1-31, wherein the potential for cleavage is about +1.05 vs. SHE to −0.18V vs. SHE at 25 °C and pH 3; +0.84 V vs. SHE to −0.38 V vs. SHE at 25 °C and pH 6.5; or +0.64V vs. SHE to −0.59V vs. SHE at 25 °C and pH 10.

33. The method of any one of claims 1-32, wherein the engineered TdT comprises one or more mutations to a wild-type TdT of SEQ ID NO: 1, wherein:
a. the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E, wherein position numbers are relative to SEQ ID NO: 1; or
b. the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353E, wherein position numbers are relative to SEQ ID NO: 1; or
c. the engineered TdT has at least 85, 90, 91, 92, 93, 94, 95, 96, 97, 98, 99, or 100% identity to SEQ ID NO: 2 or 3.

34. An engineered terminal deoxynucleotidyl transferase (TdT) comprising one or more mutations to a wild-type TdT of SEQ ID NO: 1, wherein:
a. the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E, wherein position numbers are relative to SEQ ID NO: 1; or
b. the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353E, wherein position numbers are relative to SEQ ID NO: 1; or
c. the engineered TdT has at least 85, 90, 91, 92, 93, 94, 95, 96, 97, 98, 99, or 100% identity to SEQ ID NO: 2 or 3.

35. The engineered TdT of claim 34, wherein the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E.

36. The engineered TdT of claim 34 or claim 35, wherein the one or more mutations comprise 10, 15, 20, 25, 30, 35, 40, 45, 50, or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E.

37. The engineered TdT of any one of claims 34-36, wherein the one or more mutations comprise 50 or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E.

38. The engineered TdT of any one of claims 34-37, wherein the one or more mutations comprise all of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E.

39. The engineered TdT of any one of claims 34-38, wherein the one or more mutations consist of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, D263H, G265Q, H268D, E270G, C290A, D293E, T317R, M323L, R335N, V336T, E339R, D353E.

40. The engineered TdT of any one of claims 34-39, wherein the engineered TdT comprises an amino acid sequence with 85, 90, 91, 92, 93, 94, 95, 96, 97, 98, 99, or 100% identity to SEQ ID NO: 2.

41. The engineered TdT of any one of claims 34-40, wherein the engineered TdT comprises the amino acid sequence of SEQ ID NO: 2.

42. The engineered TdT of any one of claims 34-41, wherein the amino acid sequence of the engineered TdT consists of the amino acid sequence of SEQ ID NO: 2.

43. The engineered TdT of any one of claims 34-41, wherein the amino acid sequence of the engineered TdT consists of the amino acid sequence of SEQ ID NO: 2 and a fluorescent protein label, optionally a green fluorescent protein (GFP) label.

44. The engineered TdT of any one of claims 34-41, wherein the amino acid sequence of the engineered TdT consists of the amino acid sequence of SEQ ID NO: 2 and an affinity tag, optionally a His-tag.

45. The engineered TdT of any one of claims 34-41, wherein the engineered TdT further comprises a fluorescent protein label, optionally a GFP label.

46. The engineered TdT of any one of claims 34-41, wherein the engineered TdT further comprises an affinity tag, optionally a His-tag.

47. The engineered TdT of any one of claims 34-46, wherein the TdT further comprises the conjugation mutation E32C.

48. The engineered TdT of any one of claims 34-46, wherein the TdT further comprises the conjugation mutation E29C.

49. The engineered TdT of any one of claims 34-46, wherein the TdT further comprises the conjugation mutation E37C.

50. The engineered TdT of any one of claims 34-46, wherein the TT further comprises the conjugation mutation V152C.

51. The engineered TdT of any one of claims 34-46, wherein the TdT further comprises the conjugation mutation K102C.

52. The engineered TdT of any one of claims 34-46, wherein the TdT further comprises the conjugation mutation D22C.

53. The engineered TdT of any one of claims 34-46, wherein the TdT further comprises the conjugation mutation R44C.

54. The engineered TdT of any one of claims 34-46, wherein the TdT further comprises the conjugation mutation L93C.

55. The engineered TdT of any one of claims 34-46, wherein the TdT further comprises the conjugation mutation P154C.

56. The engineered TdT of any one of claims 34-46, wherein the TdT further comprises the conjugation mutation N361C.

57. The engineered TdT of any one of claims 34-38, wherein the TdT comprises the amino acid sequence of any one of SEQ ID NOs: 11, 12, 13, and/or 15.

58. The engineered TdT of any one of claims 34-38, wherein the TdT comprises the amino acid sequence of SEQ ID NO: 15.

59. The engineered TdT of any one of claims 34-38, wherein the TdT comprises the amino acid sequence of any one of SEQ ID NOs: 25, 26, 27, 28, and 29.

60. An engineered TdT comprising the amino acid sequence of any one of SEQ ID NOs: 20, 21, 22, 23, and 24.

61. An engineered terminal deoxynucleotidyl transferase (TdT) consisting of the amino acid sequence of SEQ ID NO: 15.

62. The engineered TdT of claim 34, wherein the one or more mutations comprise one or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353E, wherein position numbers are relative to SEQ ID NO: 1.

63. The engineered TdT of claim 34 or 62, wherein the one or more mutations comprise 10, 15, 20, 25, 30, 35, 40, 45, 50, or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353E, wherein position numbers are relative to SEQ ID NO: 1.

64. The engineered TdT of claim 34, 62 or 63, wherein the one or more mutations comprise 50 or more of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353E, wherein position numbers are relative to SEQ ID NO: 1.

65. The engineered TdT of any one of claim 34 or 62-64, wherein the one or more mutations comprise the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353E, wherein position numbers are relative to SEQ ID NO: 1.

66. The engineered TdT of any one of claim 34 or 62-65, wherein the one or more mutations consist of the following mutations: C7A, Q18K, L19K, D31A, E35G, C40A, M44R, S47A, C68N, S75R, E78Q, G79E, I81L, K99Q, S100A, L112P, F119Y, S127E, K128D, Q130R, S134T, Q139R, C154P, N156T, V163I, S164E, M165Q, V172W, T173Q, D177G, L179T, M191K, D208E, Q211K, F220W, Q223K, C230E, L233Q, Q242L, C256A, a deletion of D263, a deletion of H264, a deletion of G265, a deletion of R266, a deletion of V267, a deletion of H268, a deletion of S269, a deletion of E270, a deletion of K271, a deletion of S272, Q274P, Q275S, E276P, a deletion of G279, a deletion of W280, a deletion of K281, C290A, D293E, T317R, M323L, R335N, V336T, E339R, D353B, wherein position numbers are relative to SEQ ID NO: 1.

67. The engineered TdT of any one of claim 34 or 62-66, wherein the engineered TdT comprises an amino acid sequence with 85, 90, 91, 92, 93, 94, 95, 96, 97, 98, 99, or 100% identity to SEQ ID NO: 3.

68. The engineered TdT of any one of claim 34 or 62-67, wherein the engineered TdT comprises the amino acid sequence of SEQ ID NO: 3.

69. The engineered TdT of any one of claim 34 or 62-68, wherein the amino acid sequence of the engineered TdT consists of the amino acid sequence of SEQ ID NO: 3.

70. The engineered TdT of any one of claim 34 or 62-68, wherein the amino acid sequence of the engineered TdT consists of the amino acid sequence of SEQ ID NO: 3 and a fluorescent protein label, optionally a GFP label.

71. The engineered TdT of any one of claim 34 or 62-68, wherein the amino acid sequence of the engineered TdT consists of the amino acid sequence of SEQ ID NO: 3 and an affinity tag, optionally a His-tag.

72. The engineered TdT of any one of claim 34 or 62-68, wherein the engineered TdT further comprises a fluorescent protein label, optionally a green fluorescent protein (GFP) label.

73. The engineered TdT of any one of claim 34 or 62-68, wherein the engineered TdT further comprises an affinity tag, optionally a His-tag.

74. The engineered TdT of any one of claim 34 or 62-73, wherein the TdT further comprises the conjugation mutation E32nc, wherein ne is a non-canonical amino acid.

75. The engineered TdT of any one of claim 34 or 62-73, wherein the TdT further comprises the conjugation mutation V152nc, wherein nc is a non-canonical amino acid.

76. The engineered TdT of any one of claim 34 or 62-73, wherein the TdT further comprises the conjugation mutation P154nc, wherein ne is a non-canonical amino acid.

77. The engineered TdT of any one of claim 34 or 62-73, wherein the TdT further comprises the conjugation mutation E32C.

78. The engineered TdT of any one of claim 34 or 62-73, wherein the TdT further comprises the conjugation mutation V152C.

79. The engineered TdT of any one of claim 34 or 62-73, wherein the TdT further comprises the conjugation mutation P154C.

80. The engineered TdT of any one of claim 34 or 62-73, wherein the TdT further comprises the conjugation mutation E29C.

81. The engineered TdT of any one of claim 34 or 62-73, wherein the TdT further comprises the conjugation mutation E37C.

82. The engineered TdT of any one of claim 34 or 62-73, wherein the TdT further comprises the conjugation mutation R44C.

83. The engineered TdT of any one of claim 34 or 62-73, wherein the TdT further comprises the conjugation mutation K102C.

84. The engineered TdT of any one of claim 34 or 62-64, wherein the TdT comprises the amino acid sequence of SEQ ID NO: 4.

85. The engineered TdT of any one of claim 34 or 62-64, wherein the TdT comprises the amino acid sequence of SEQ ID NO: 5.

86. The engineered TdT of any one of claim 34 or 62-64, wherein the TdT comprises the amino acid sequence of SEQ ID NO: 6.

87. The engineered TdT of any one of claim 34 or 62-64, wherein the TdT comprises the amino acid sequence of SEQ ID NO: 7.

88. The engineered TdT of any one of claim 34 or 62-64, wherein the TdT comprises the amino acid sequence of SEQ ID NO: 9.

89. The engineered TdT of any one of claim 34 or 62-64, wherein the TdT comprises the amino acid sequence of SEQ ID NO: 10.

90. The engineered TdT of any one of claim 34 or 62-64, wherein the TdT comprises the amino acid sequence of SEQ ID NO: 16.

91. The engineered TdT of any one of claim 34 or 62-64, wherein the TdT comprises the amino acid sequence of SEQ ID NO: 17.

92. The engineered TdT of any one of claim 34 or 62-64, wherein the TdT comprises the amino acid sequence of SEQ ID NO: 18

93. The engineered TdT of any one of claim 34 or 62-64, wherein the TdT comprises the amino acid sequence of SEQ ID NO: 19.

94. The engineered TdT of any of claims 34-93, wherein the TdT is capable of covalently binding to a nucleotide molecule at a conjugation residue of the engineered TdT via a tether, wherein:
(i) the conjugation residue comprises a cysteine residue, a non-canonical amino acid residue, or a lysine residue capable of reacting with a bifunctional crosslinker or a redox-cleavable linker; and
(ii) wherein the nucleotide molecule comprises a nucleotide, nucleotide triphosphate, or a nucleotide analog.

95. The engineered TdT of claim 94, wherein the TdT, tether, and nucleotide molecule comprise any one of the following structures:

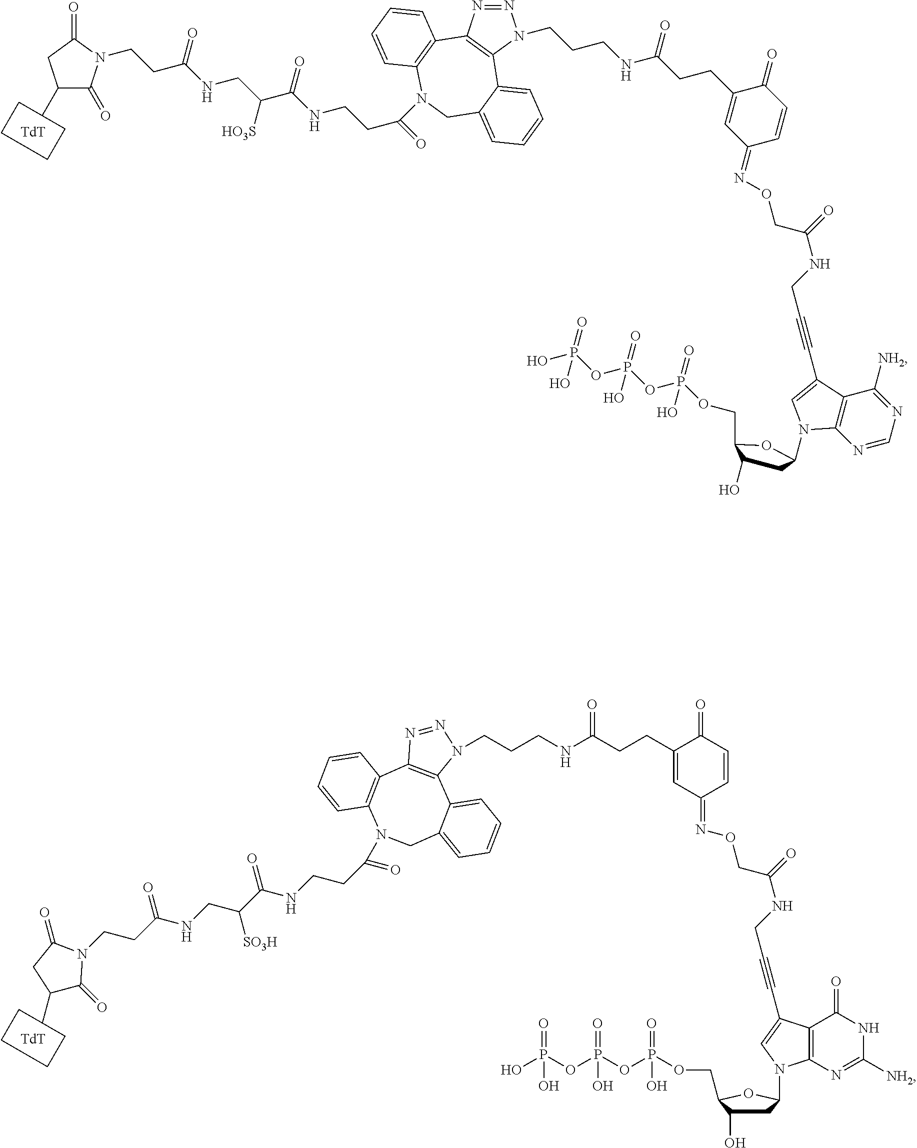

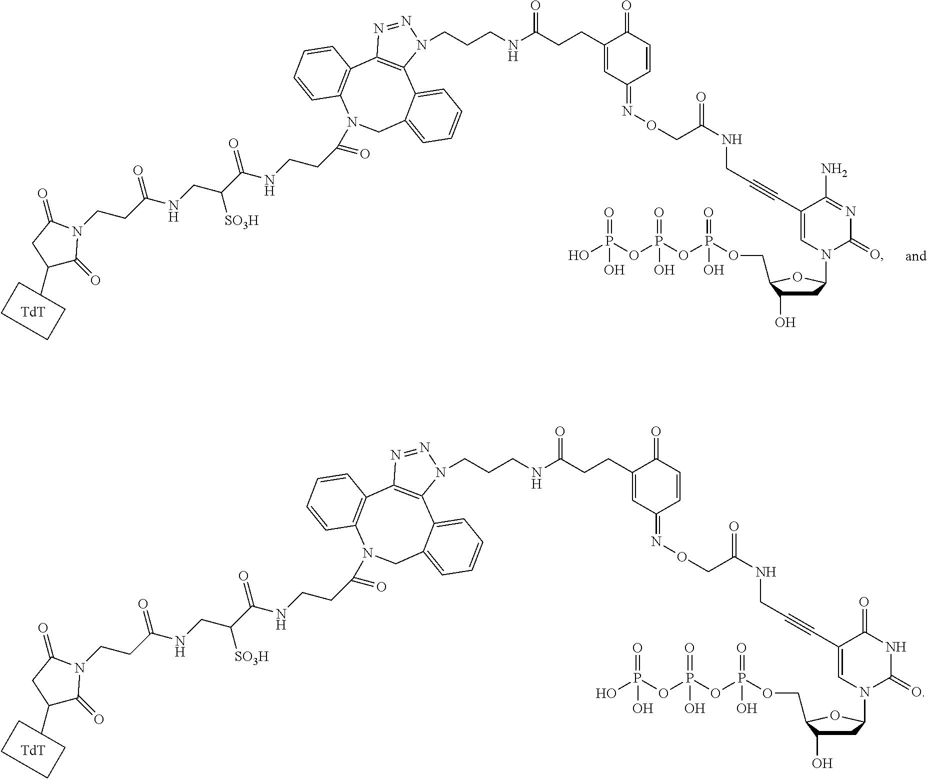

96. The engineered TdT of claim 94, wherein the TdT, tether, and nucleotide molecule comprise any one of the following structures:

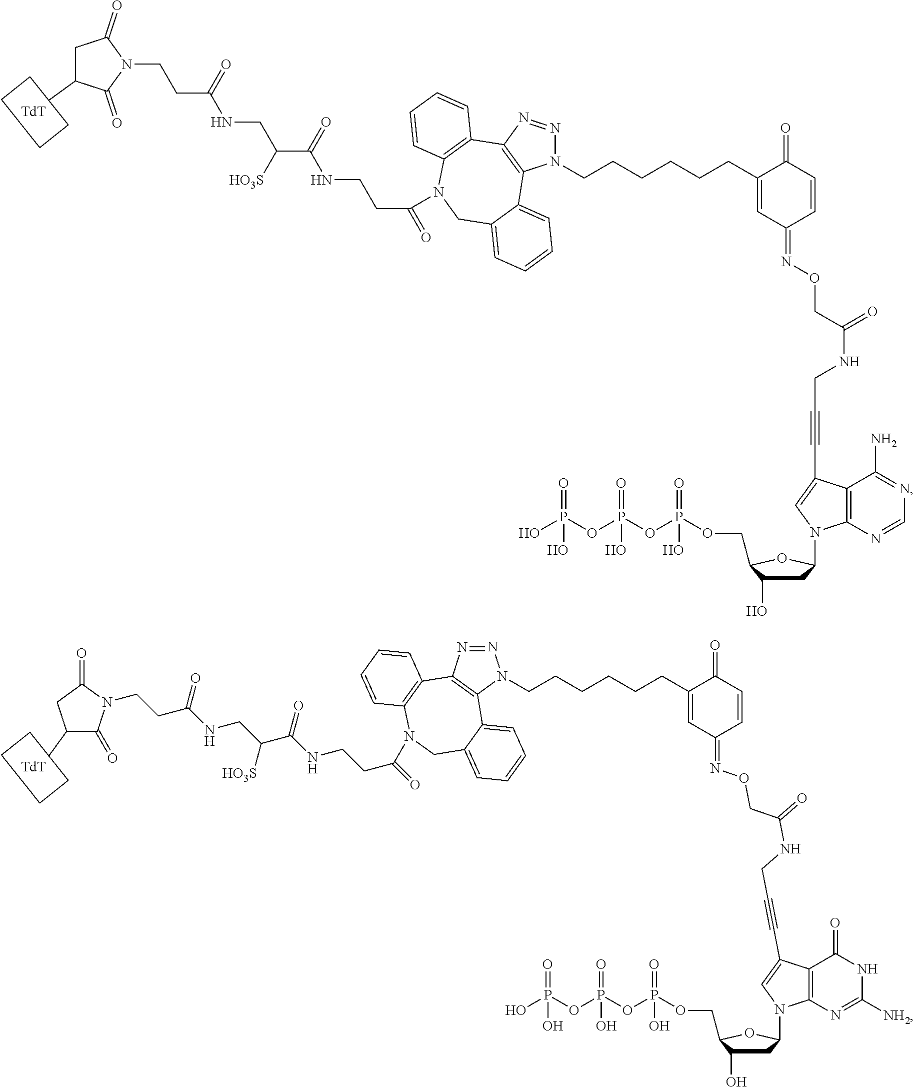

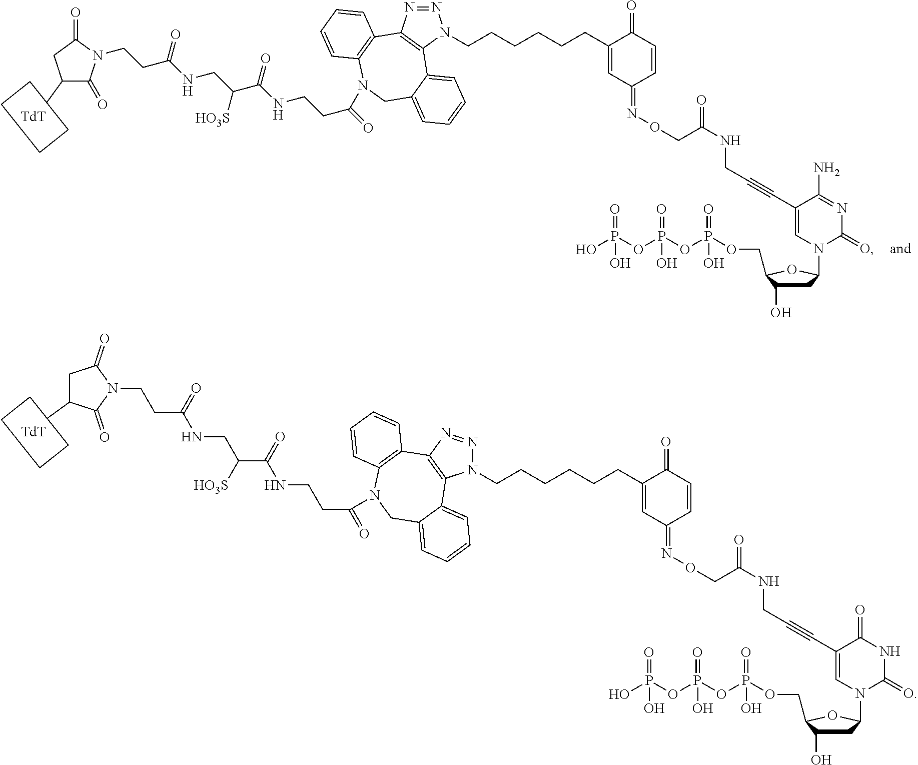

97. The engineered TdT of claim 94, wherein the TdT, tether, and nucleotide molecule comprise any one of the following structures: 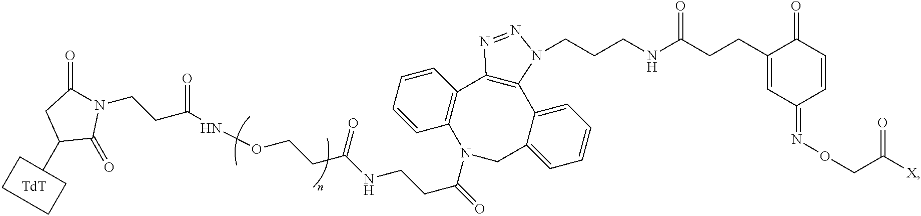
wherein X is any one of:

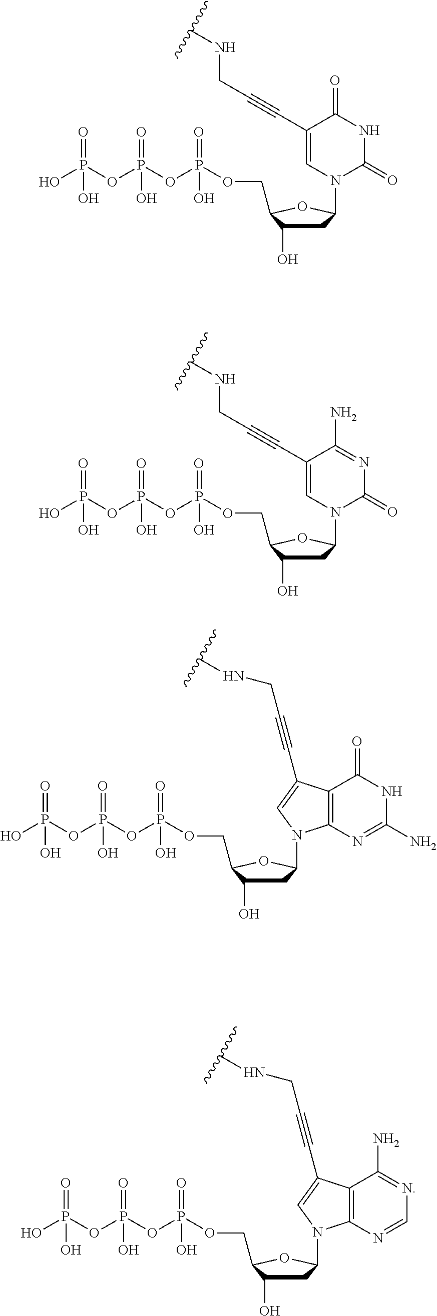

98. The engineered TdT of claim 94, wherein the TdT, tether, and nucleotide molecule comprise any one of the following structures: 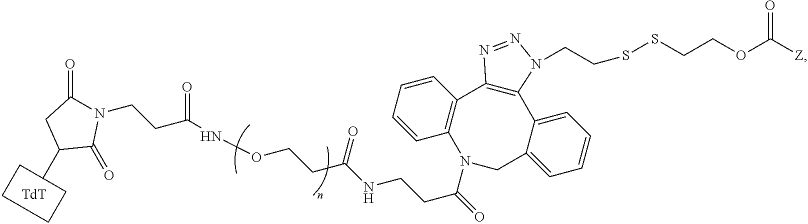
wherein Z is any one of:

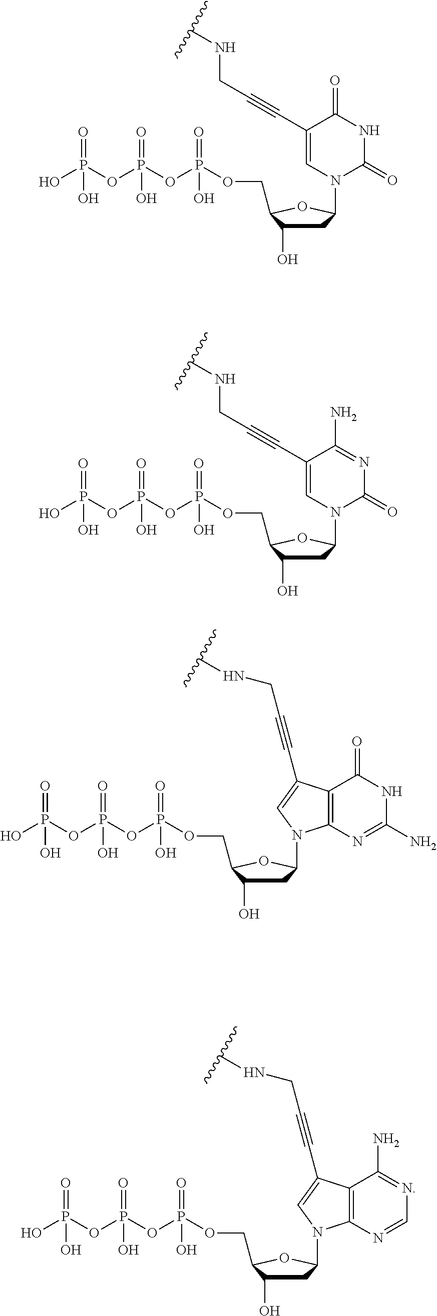

99. The engineered TdT of claim 94, wherein the TdT, tether, and nucleotide molecule comprise any one of the following structures: 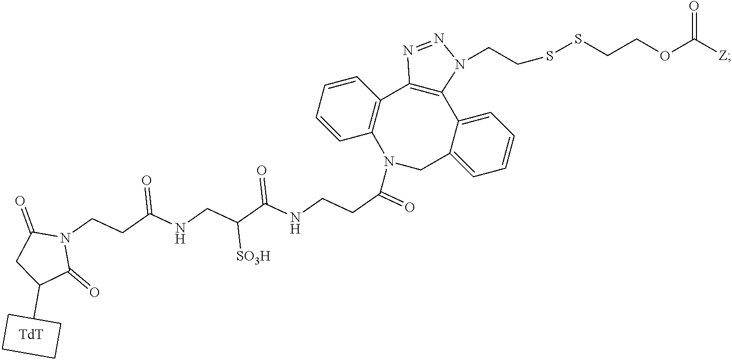
wherein Z is any one of:

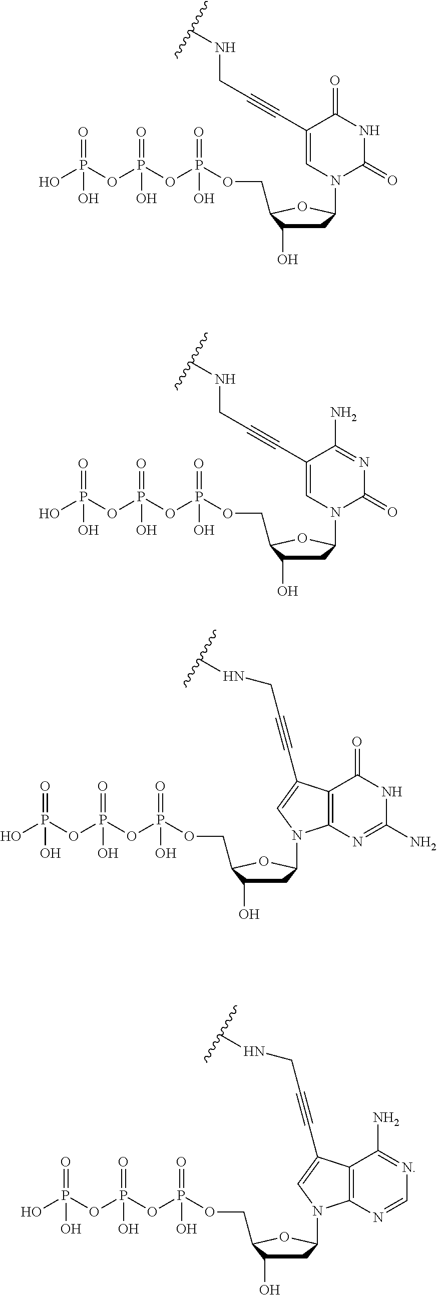

100. The engineered TdT of claim 94, wherein the TdT, tether, and nucleotide molecule comprise any one of the following structures: 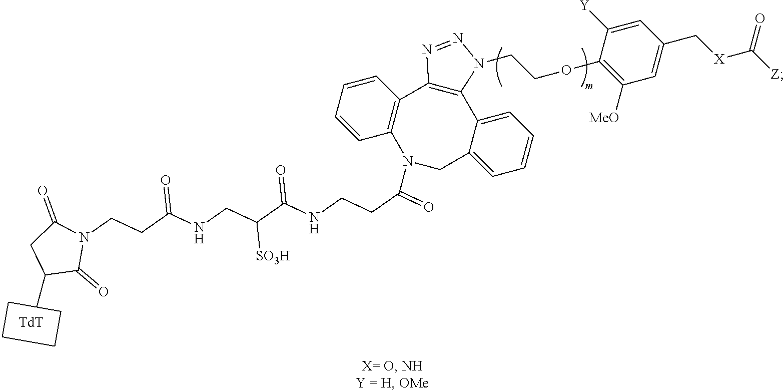
wherein X is O or NH;
wherein Y is H or OMe; and
wherein Z is any one of:

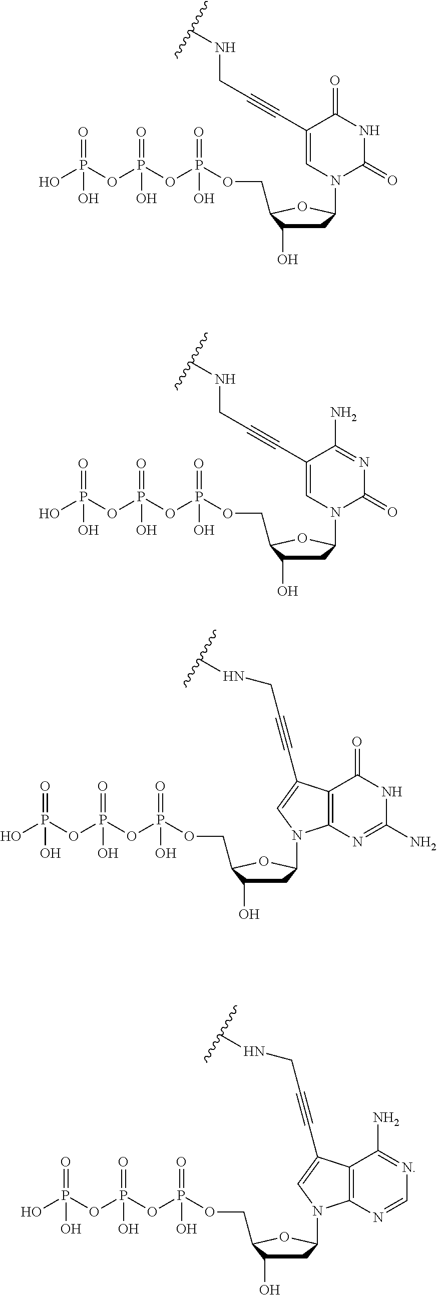

101. The engineered TdT of claim 94, wherein the TdT, tether, and nucleotide molecule comprise any one of the following structures: 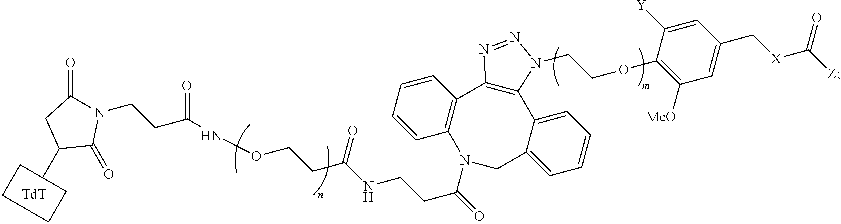
wherein X is O or NH;
wherein Y is H or OMe; and
wherein Z is any one of:

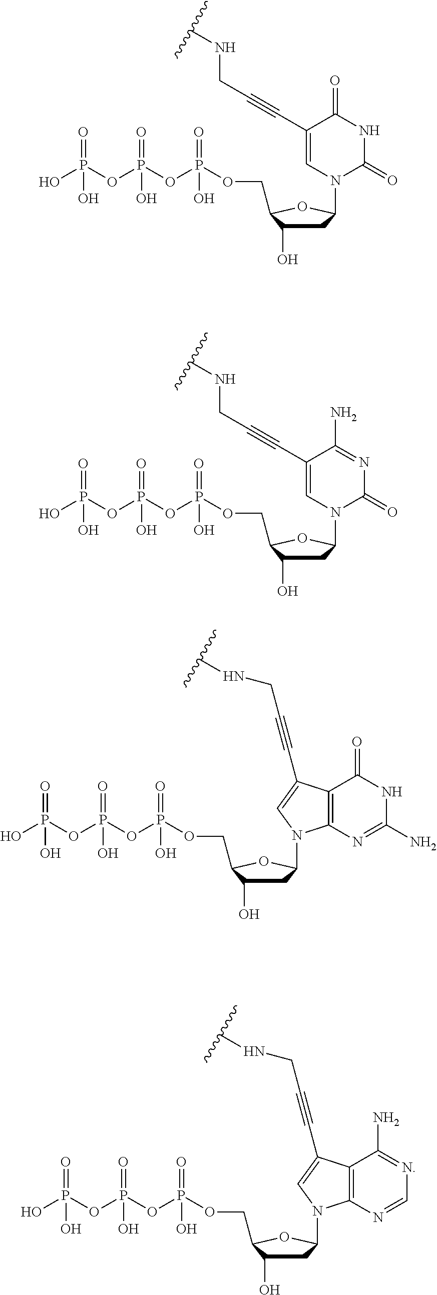

102. The engineered TdT of claim 94, wherein the TdT, tether, and nucleotide molecule comprise any one of the following structures: 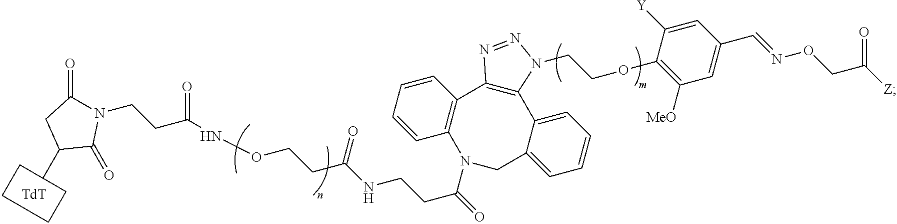
wherein Y is H or OMe; and
wherein Z is any one of:

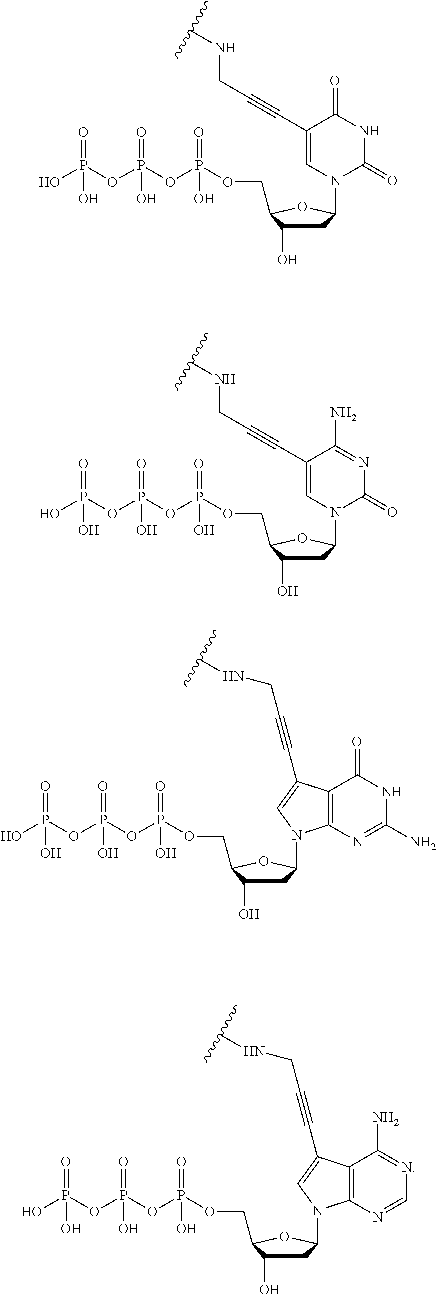

103. The engineered TdT of claim 94, wherein the TdT, tether, and nucleotide molecule comprise any one of the following structures: 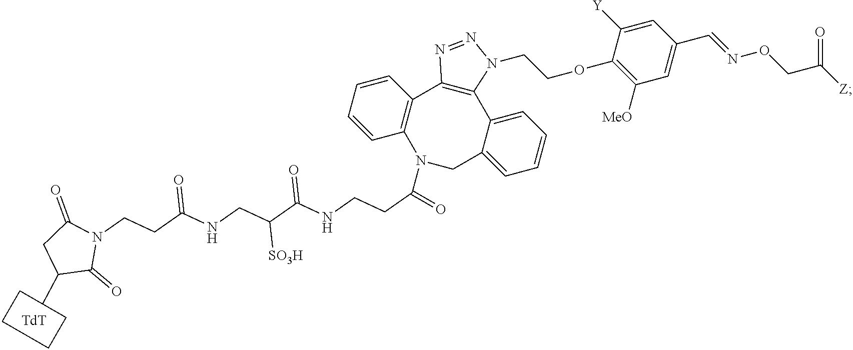
wherein Y is H or OMe; and
wherein Z is any one of:

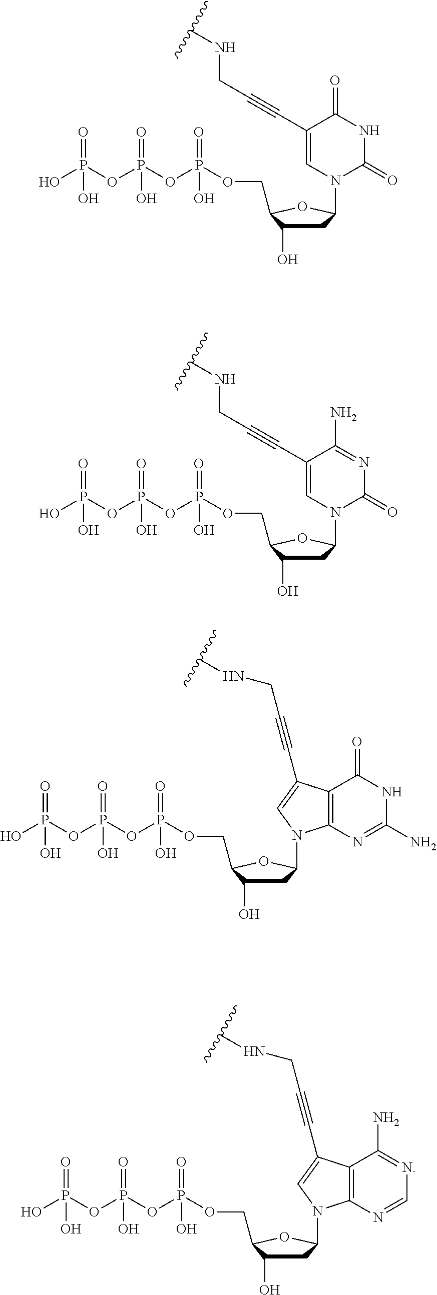

104. A kit comprising the engineered TdT of any one of claims 34-93, wherein the TdT is capable of covalently binding to a nucleotide molecule at a conjugation residue of the engineered TdT via a tether, wherein the kit further comprises:
(i) a bifunctional crosslinker and/or a redox-cleavable linker comprising a functional group for covalent conjugation to a conjugation residue of the TdT; and
(ii) a nucleotide molecule.

105. The kit of claim 104, wherein the kit further comprises a spacer.

106. The kit of any one of claims 104-106, wherein the functional group for covalent conjugation comprises a maleimide, N-hydroxysuccinimidyl group, or a dibenzylcyclooctyne (DBCO).

107. The kit of any one of claims 104-106, wherein the functional group for covalent conjugation comprises a maleimide.

108. The kit of claim 107, wherein the maleimide is capable of attaching to a conjugation residue of the engineered TdT, wherein the conjugation residue is a cysteine residue in the TdT.

109. The kit of any one of claims 104-106, wherein the functional group for covalent conjugation comprises a DBCO.

110. The kit of claim 109, wherein the DBCO is capable of attaching to a conjugation residue of the engineered TdT, and wherein the conjugation residue is a non-canonical amino acid in the TdT.

111. The kit of claim 110, wherein the non-canonical amino acid is 4-azidophenylalanine.

112. The kit of any one of claims 104-111, wherein the redox-cleavable linker comprises a quinone oxime ether linker.

113. The kit of any one of claims 104-111, wherein the redox-cleavable linker comprises a disulfide linker.

114. The kit of any one of claims 104-111, wherein the redox-cleavable linker comprises a syringic acid-based linker or vanillin-based linker.

115. The kit of any one of claims 105-114, wherein the spacer comprises one or more polyethylene glycol molecules (PEG)ₙ, optionally wherein “n” is 1-12.

116. The kit of any one of claims 105-114, wherein the spacer comprises a C1-C10 alkyl.

117. The kit of any one of claims 105-114, wherein the spacer comprises a hydrocarbon chain wherein one or more of the carbons comprises a substituted sulfonate group.

118. The kit of any one of claims 105-114, wherein the spacer comprises a hydrocarbon chain comprising 1-6 carbons wherein one or more of the carbons comprises a substituted sulfonate group.

119. The kit of any one of claims 105-114, wherein the spacer comprises a C1-C6 alkyl.

120. The kit of claim 104, wherein the kit comprises a bifunctional crosslinker capable of being covalently linked to the engineered TdT and comprising a DBCO, wherein the DBCO of the bifunctional crosslinker is capable of reacting with any one of the following

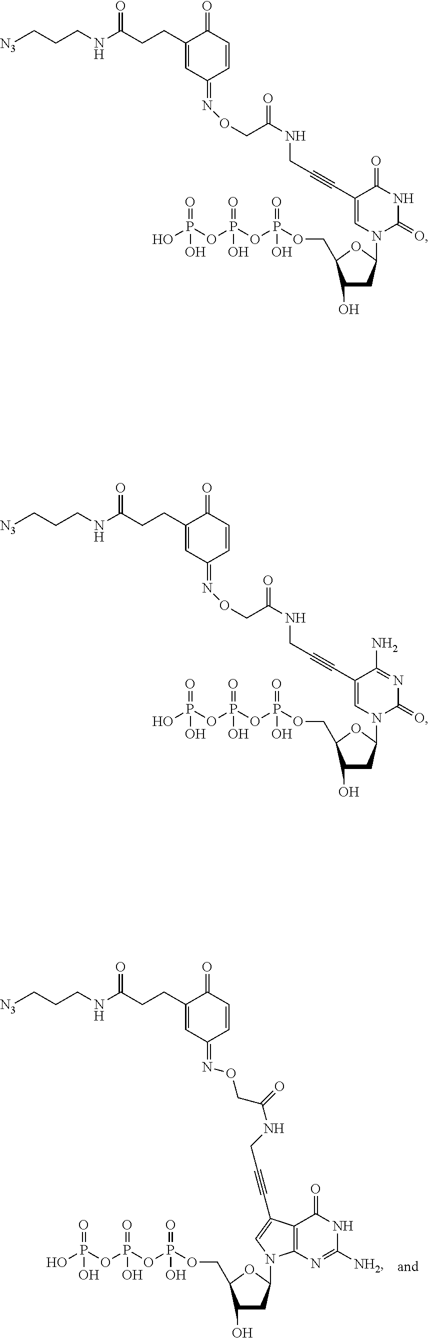 [SMILES: [N-]=[N+]=NCCCNC(=O)CCC1=C/C(=N/OCC(=O)NCC#Cc2cn([C@H]3C[C@@H](O)[C@@H](COP(=O)(O)OP(=O)(O)OP(=O)(O)O)O3)c(=O)[nH]c2=O)C=CC1=O ; [N-]=[N+]=NCCCNC(=O)CCC1=C/C(=N/OCC(=O)NCC#Cc2cn([C@H]3C[C@@H](O)[C@@H](COP(=O)(O)OP(=O)(O)OP(=O)(O)O)O3)c(=O)nc2N)C=CC1=O ; [N-]=[N+]=NCCCNC(=O)CCC1=C/C(=N/OCC(=O)NCC#Cc2cn([C@H]3C[C@@H](O)[C@@H](COP(=O)(O)OP(=O)(O)OP(=O)(O)O)O3)c3nc(N)[nH]c(=O)c23)C=CC1=O]

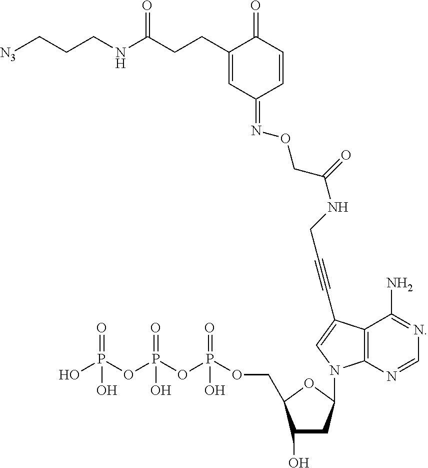 [SMILES: [N-]=[N+]=NCCCNC(=O)CCC1=C/C(=N/OCC(=O)NCC#Cc2cn([C@H]3C[C@@H](O)[C@@H](COP(=O)(O)OP(=O)(O)OP(=O)(O)O)O3)c3ncnc(N)c23)C=CC1=O]

121. The kit of claim 104, wherein the kit comprises a bifunctional crosslinker capable of being covalently linked to the engineered TdT and comprising a DBCO, wherein the DBCO of the bifunctional crosslinker is capable of reacting with any one of the following 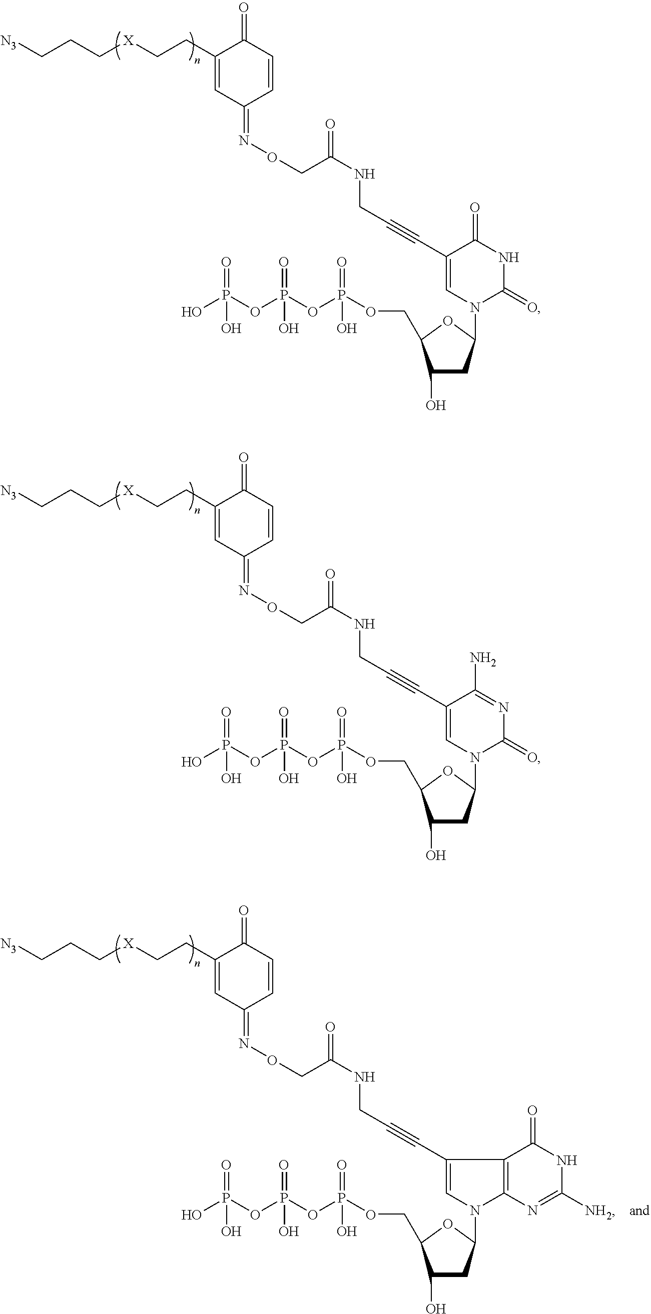 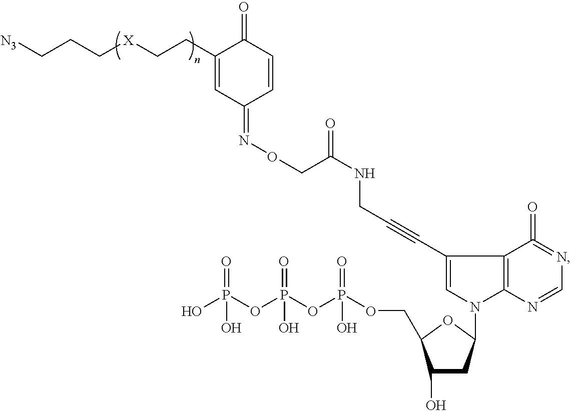
wherein X is C(O), C(O)NH, CH2, or O; and wherein n=1 for X═C(O), n=1 for C(O)NH, n=1 for CH2, and n=1-4 for X═O, wherein n is an integer.

122. The kit of claim 104, wherein the kit comprises a bifunctional crosslinker capable of being covalently linked to the engineered TdT and comprising a DBCO, wherein the DBCO of the bifunctional crosslinker is capable of reacting with any one of the following

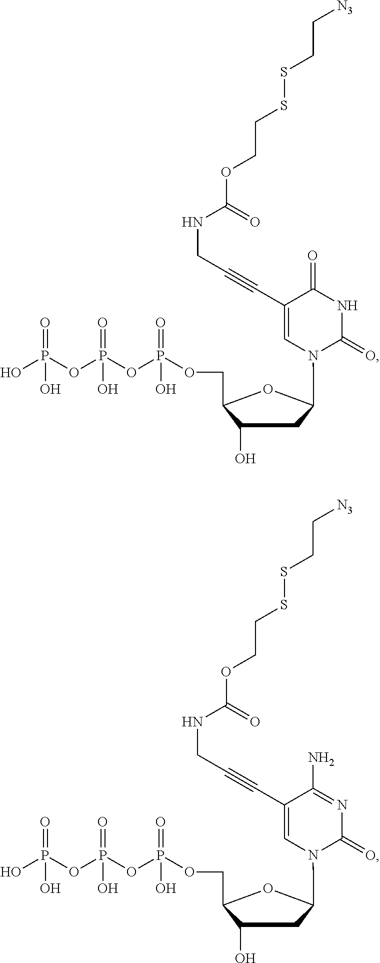 [SMILES: [N-]=[N+]=NCCSSCCOC(=O)NCC#Cc1cn([C@H]2C[C@@H](O)[C@@H](COP(=O)(O)OP(=O)(O)OP(=O)(O)O)O2)c(=O)[nH]c1=O ; [N-]=[N+]=NCCSSCCOC(=O)NCC#Cc1cn([C@H]2C[C@@H](O)[C@@H](COP(=O)(O)OP(=O)(O)OP(=O)(O)O)O2)c(=O)nc1N]

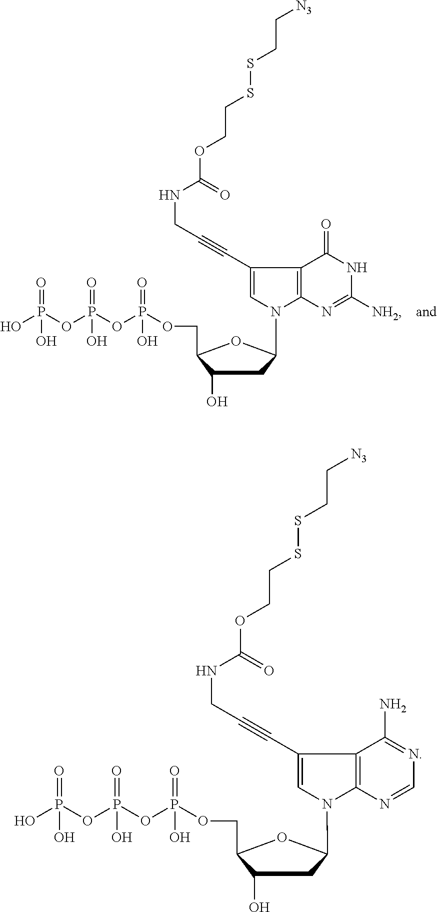 [SMILES: [N-]=[N+]=NCCSSCCOC(=O)NCC#Cc1cn([C@H]2C[C@@H](O)[C@@H](COP(=O)(O)OP(=O)(O)OP(=O)(O)O)O2)c2nc(N)[nH]c(=O)c12 ; [N-]=[N+]=NCCSSCCOC(=O)NCC#Cc1cn([C@H]2C[C@@H](O)[C@@H](COP(=O)(O)OP(=O)(O)OP(=O)(O)O)O2)c2ncnc(N)c12]

123. The kit of claim 104, wherein the kit comprises a bifunctional crosslinker capable of being covalently linked to the engineered TdT and comprising a DBCO, wherein the DBCO of the bifunctional crosslinker is capable of reacting with any one of the following 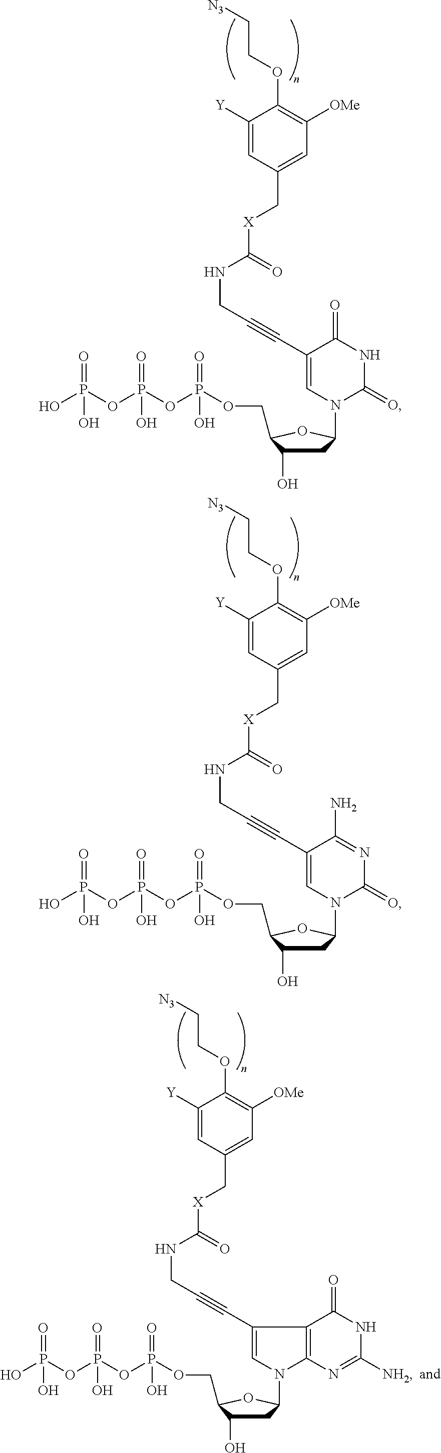 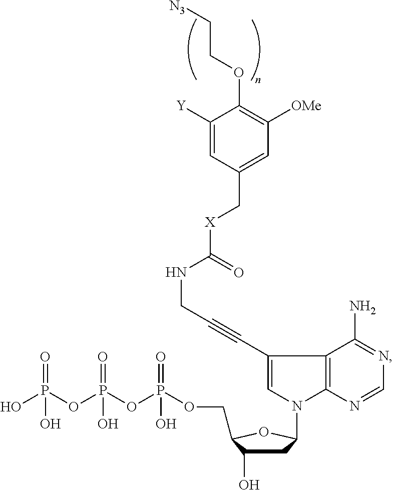
wherein X is O or NH, and wherein Y is H or OMe.

124. The kit of claim 104, wherein the kit comprises one or more of the following molecules capable of being reacted with the engineered TdT, wherein the conjugation residue is a cysteine residue in the TdT:

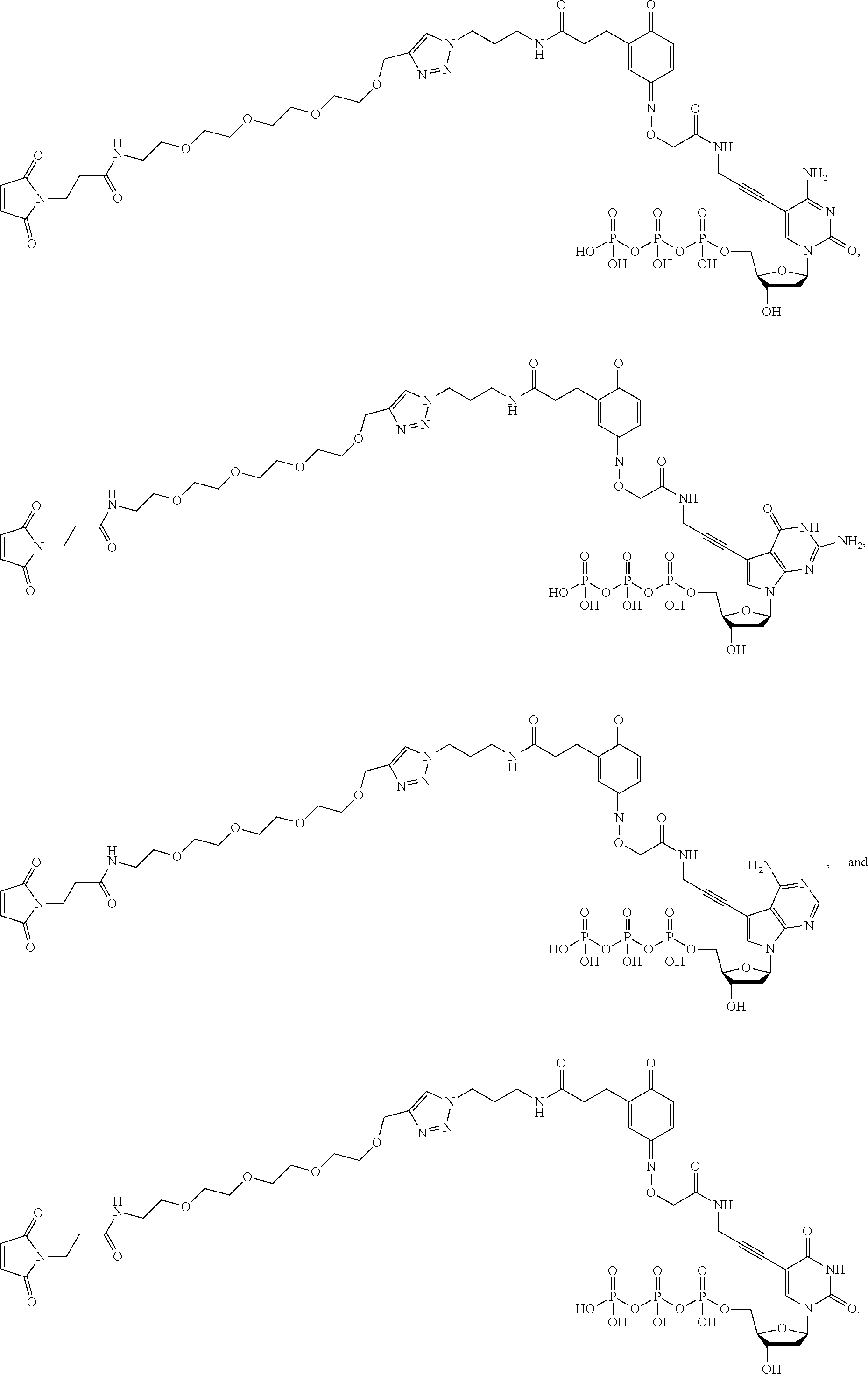

125. The kit of claim 104, wherein the kit comprises one or more of the following molecules capable of being reacted with the engineered TdT, wherein the conjugation residue is a cysteine residue in the TdT:

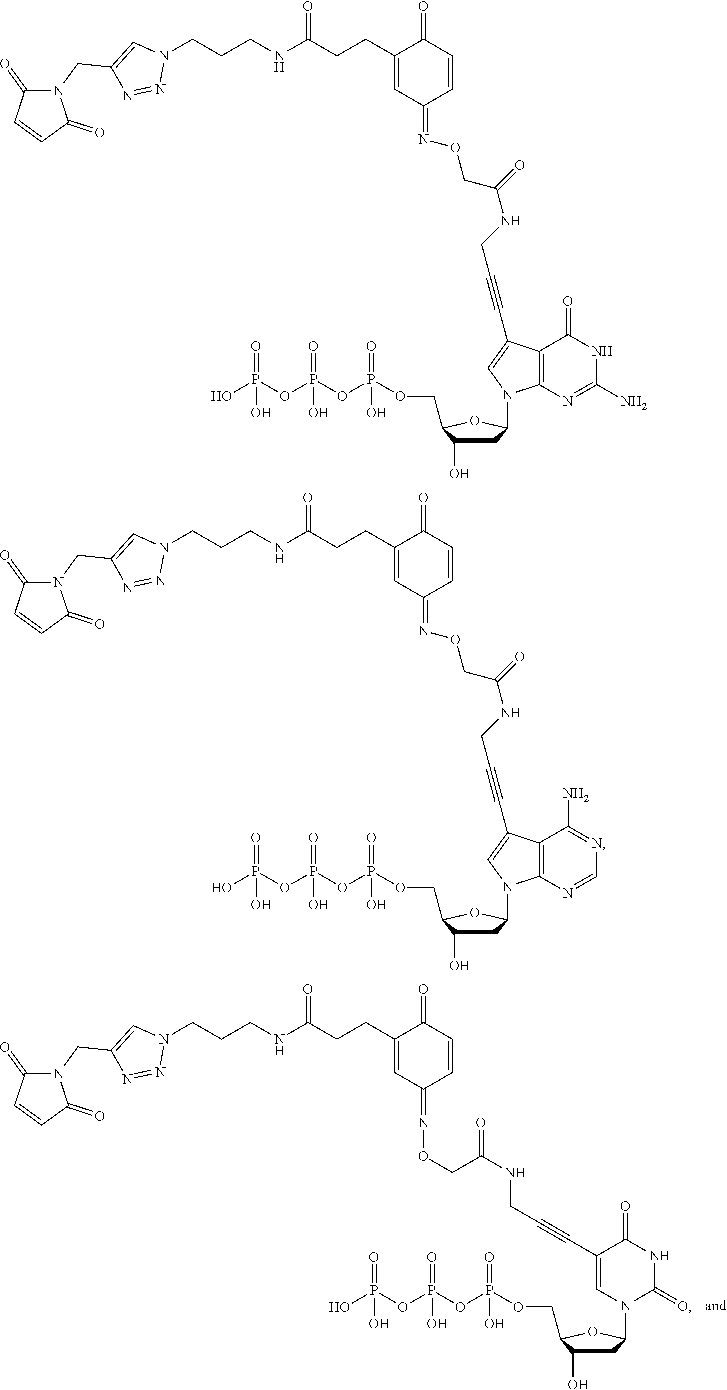

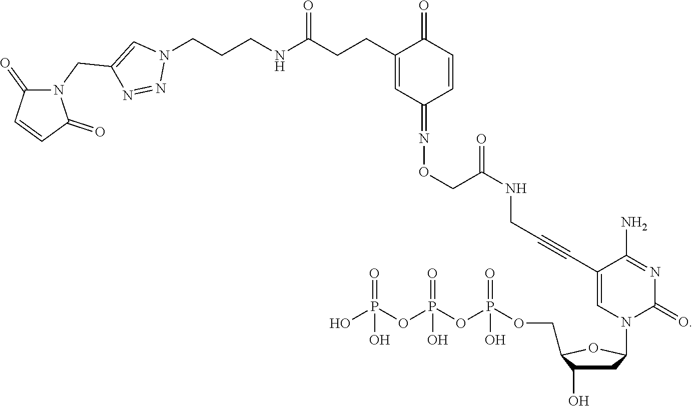

126. The kit of claim 104, wherein the kit comprises one or more of the following molecules capable of being reacted with the engineered TdT, wherein the conjugation residue is a non-canonical amino acid residue in the TdT:

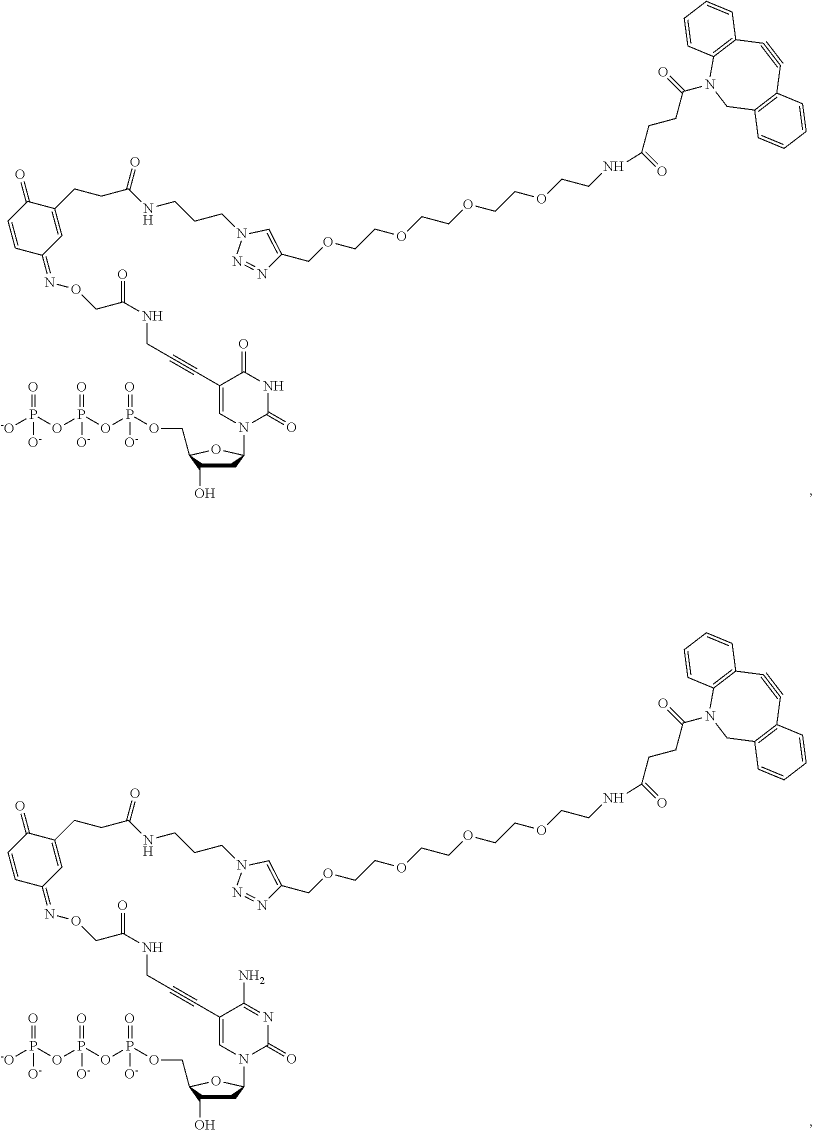

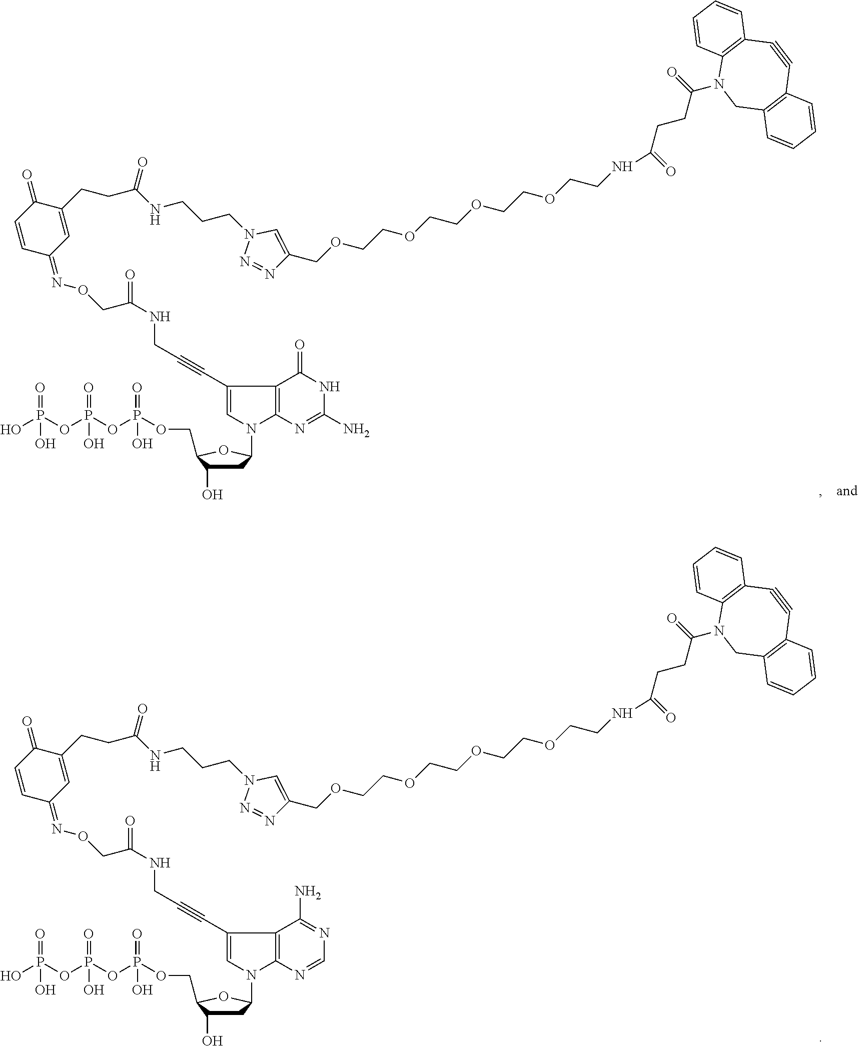

127. The kit of claim 104, wherein the kit comprises one or more of the following molecules capable of being reacted with the engineered TdT, wherein the conjugation residue is a lysine residue in the TdT:

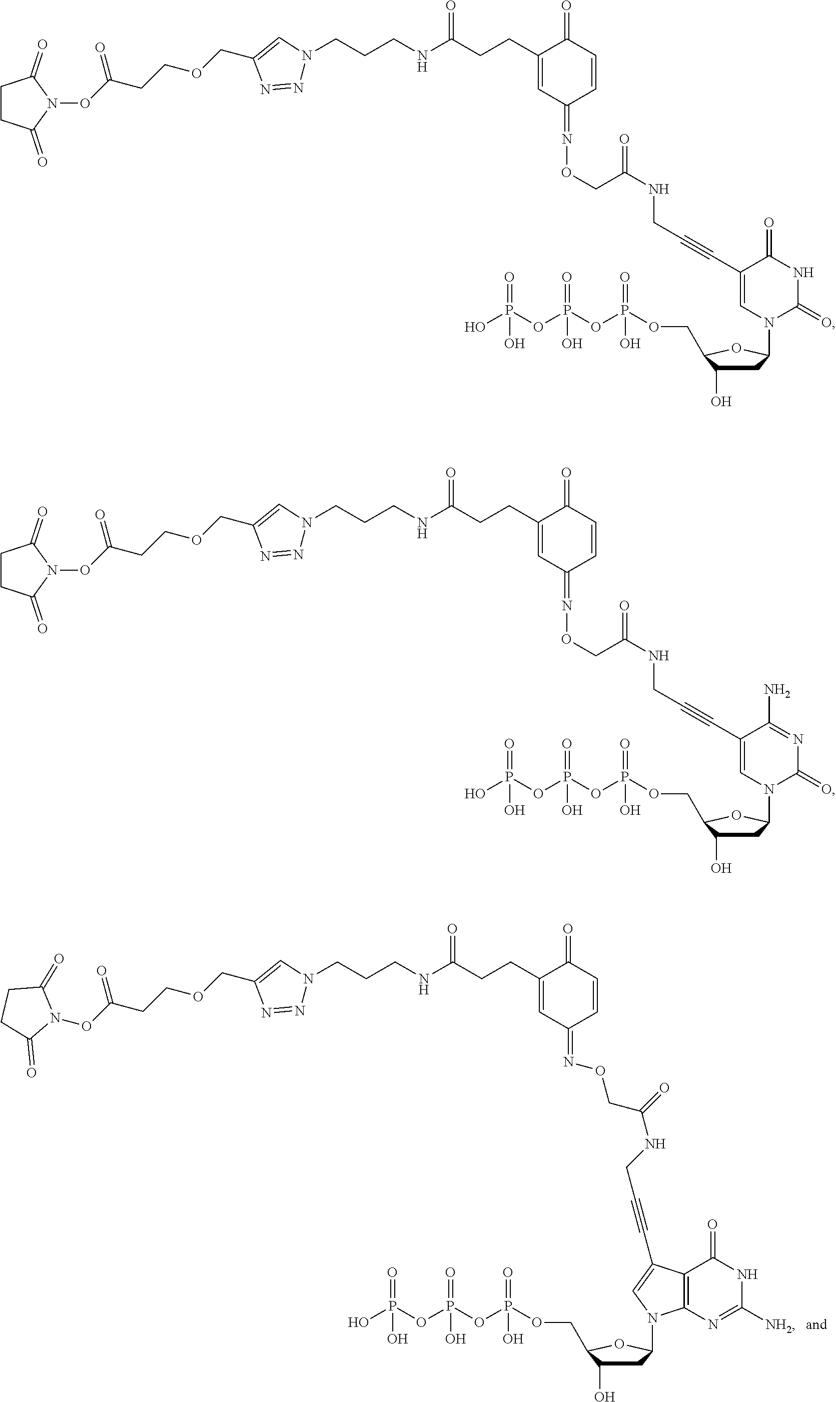

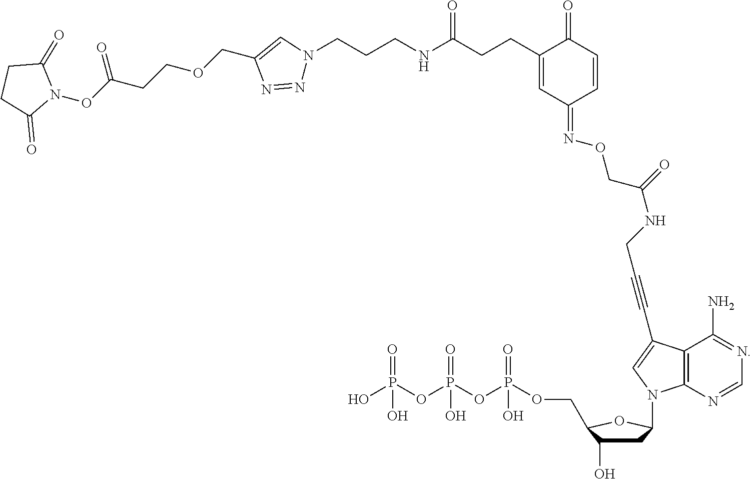

128. A system for enzymatic DNA synthesis comprising:
a. the engineered TdT of any one of claims 34-93;
b. a redox-cleavable linker;
c. a redox shuttle solution; and
d. two or more electrodes on a surface.

129. The system of claim 128, wherein the electrodes enable an applied potential or galvanic field to be supplied locally to the device or system thus enabling generation of active redox shuttle via reduction or oxidation at one or more electrodes.

130. The system of claim 128 or claim 129, wherein the electrode where the soluble redox shuttle is generated is comprised of a smaller surface area than the other electrode to which it is electrically coupled.

131. The system of any one of claims 128-130, wherein if the soluble redox shuttle is activated via cathodic process, then the cathode may be comprised of less active surface area than the anode by (a) having less electroactive area, or (b) being coupled to multiple anodes which in combination provide substantially more area than the cathode.

132. The system of any one of claims 128-131, wherein the surface, or electroactive, or area aspect ratios for the counter electrode relative to the working electrode, or C/E aspect ratio is at least: 3:1, 6:1, 10:1, 100:1, 1000:1, or higher.

133. The system of any one of claims 128-132, wherein all the electrodes lie in a single plane.

134. The system of any one of claims 128-132, wherein a combination of electrodes may lie in multiple planes.

135. The system of any one of claims 128-132, wherein electrodes are oriented parallel to perpendicular with respect to one another during the current path.

136. The system of any one of claims 128-135, wherein the system has an inter-electrode gap (i.e., distance between electrodes included in the current path) of ≤100 μm, ≤10 μm, ≤1 μm, ≤100 μm, ≤10 nm, ≤1 nm, ≤100 μm, ≤10 μm, or ≤1 μm.

137. The system of any one of claims 128-136, wherein the redox shuttle solution comprises at least one soluble redox shuttle in combination with a buffer, a supporting electrolyte, and/or a hydrotropic agent.

138. The system of claim 137, wherein the buffer is sodium citrate, potassium hydrogen phosphate, or potassium dihydrogen phosphate.

139. The system of claim 137 or 138, wherein the supporting electrolyte is sodium chloride, sodium salts, lithium salts, potassium salts, or magnesium salts.

140. The system of any one of claims 137-139, wherein the hydrotropic agent is caffeine, urea, and/or nicotinamide (NA).

141. The system of any one of claims 128-136, wherein the redox shuttle solution comprises methylene blue, citrate-phosphate buffer, and sodium chloride.

142. The system of any one of claims 128-136, wherein the redox shuttle solution comprises methylene blue, citrate-phosphate buffer, sodium chloride, and nicotinamide.

143. The system of any one of claims 128-136, wherein the redox shuttle solution comprises flavin mononucleotide, citrate-phosphate buffer, sodium chloride, and nicotinamide.

144. The system of any one of claims 128-136, wherein the redox shuttle solution comprises 10 mM methylene blue, 25 mM citrate-phosphate buffer at pH 3.4, and 100 mM sodium chloride.

145. The system of any one of claims 128-136, wherein the redox shuttle solution comprises 10 mM methylene blue, 25 mM citrate-phosphate buffer at pH 3.4, 100 mM sodium chloride, and 1 M nicotinamide.

146. The system of any one of claims 128-136, wherein the redox shuttle solution comprises 50 mM flavin mononucleotide, 25 mM citrate-phosphate buffer at pH 3.4, 1 M sodium chloride, and 1 M nicotinamide.

147. A redox shuttle solution comprising at least one soluble redox shuttle in combination with one or more of the following: supporting electrolyte, buffer, and hydrotrope.

148. The redox shuttle solution of claim 147, comprising: 10 mM methylene blue, 25 mM citrate-phosphate buffer at pH 3.4, and 100 mM sodium chloride.

149. The redox shuttle solution of claim 147, comprising: 10 mM methylene blue, 25 mM citrate-phosphate buffer at pH 3.4, 100 mM sodium chloride, and 1 M nicotinamide.

150. The redox shuttle solution of claim 147, comprising: 50 mM flavin mononucleotide, 25 mM citrate-phosphate buffer at pH 3.4, 1 M sodium chloride, and 1 M nicotinamide.

151. A molecule comprising any one of the following structures:

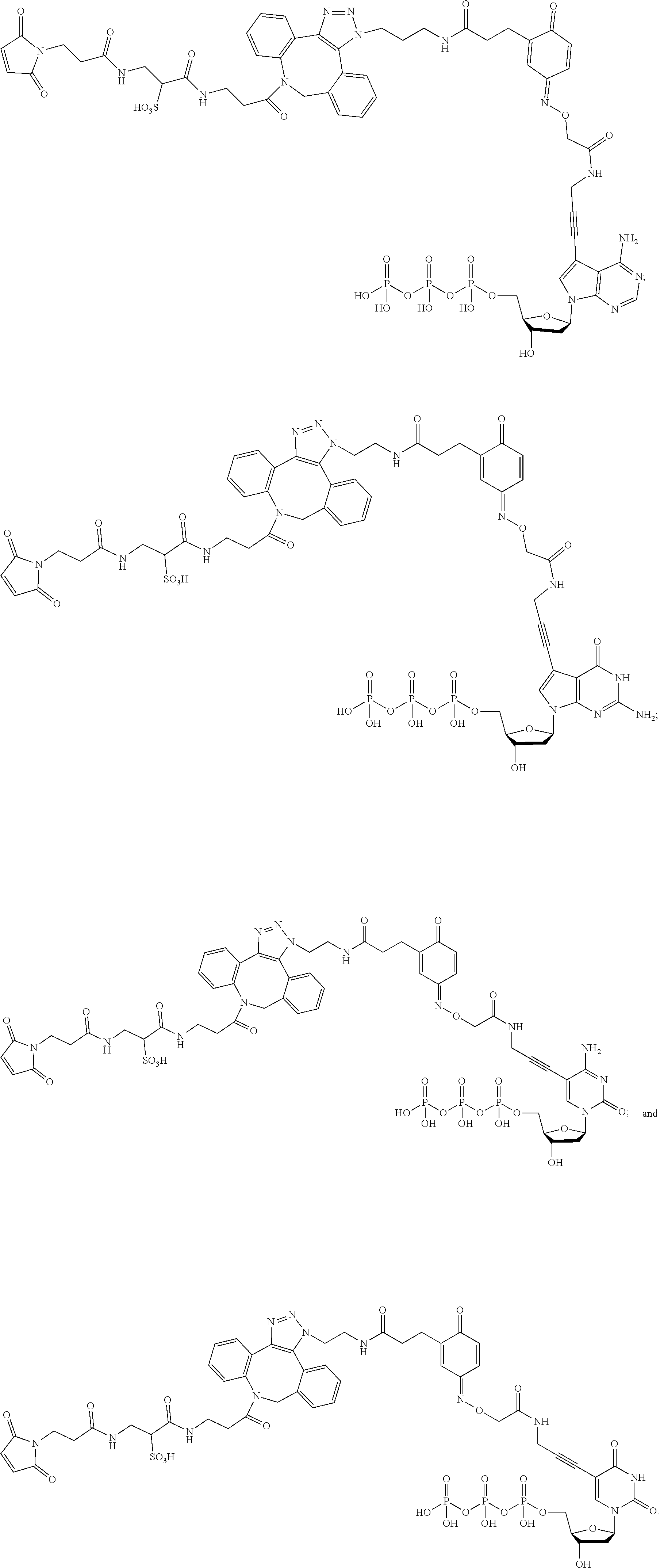

152. A molecule comprising any one of the following structures:

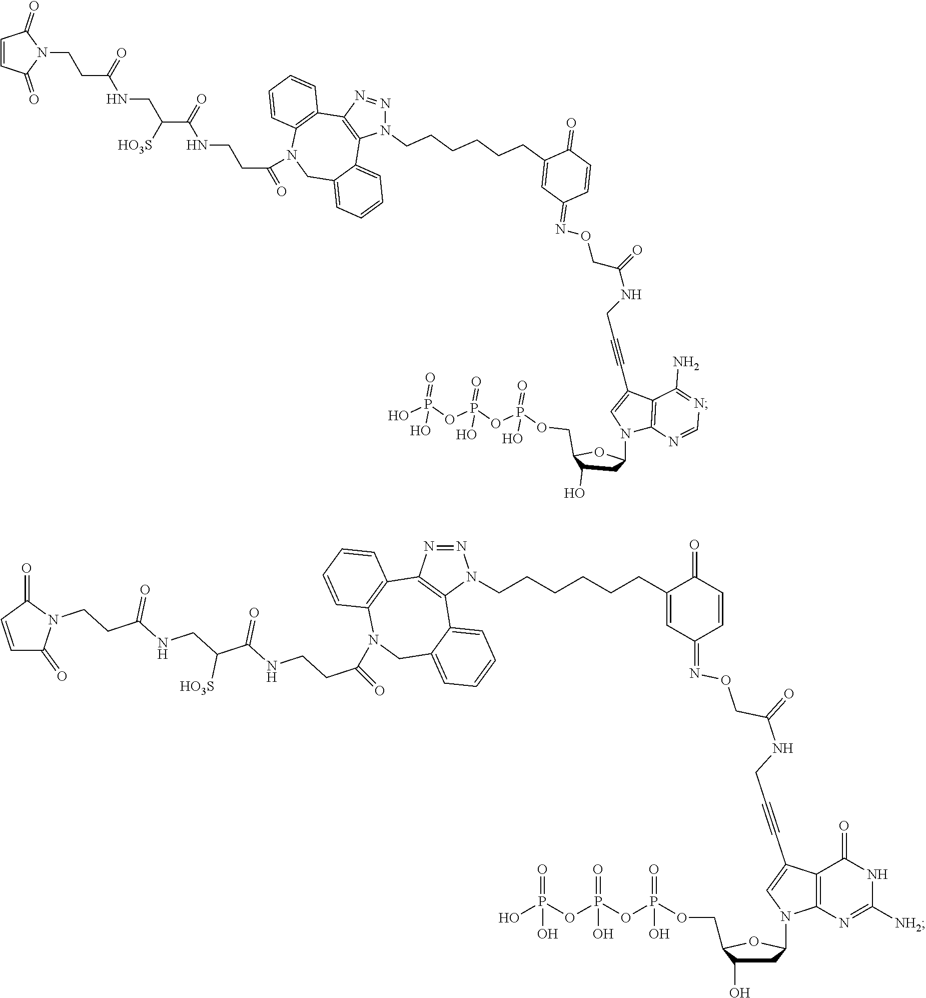

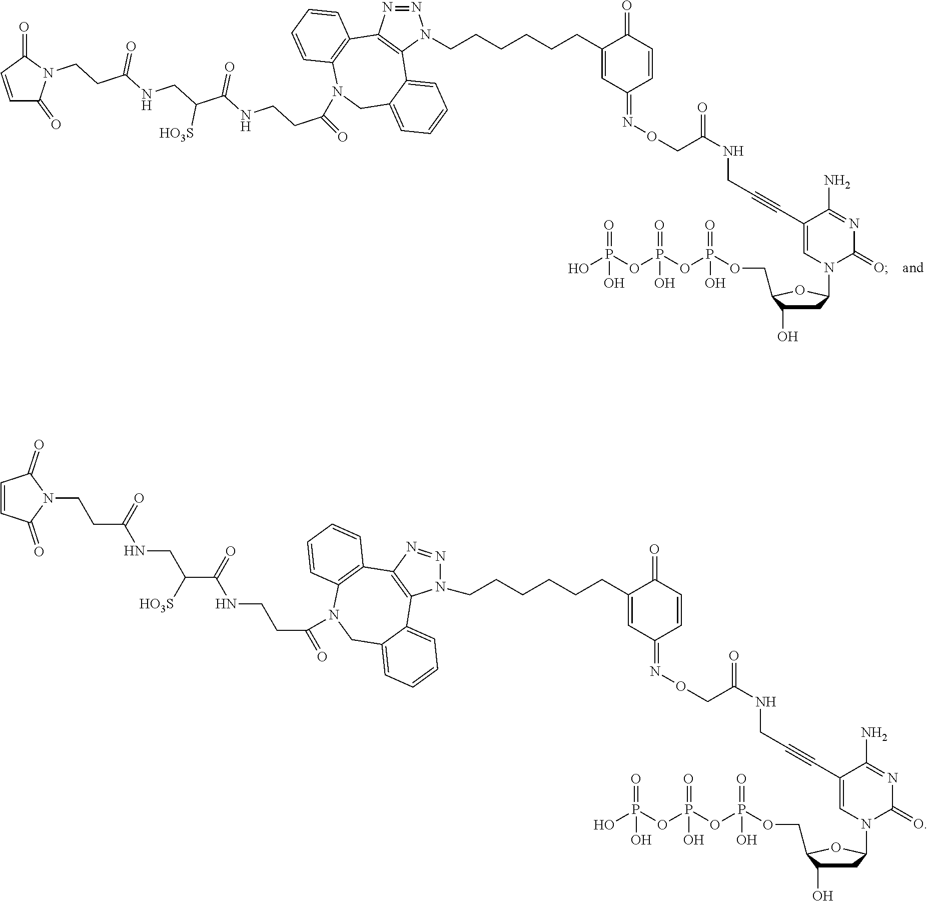

153. A molecule comprising the following structure: 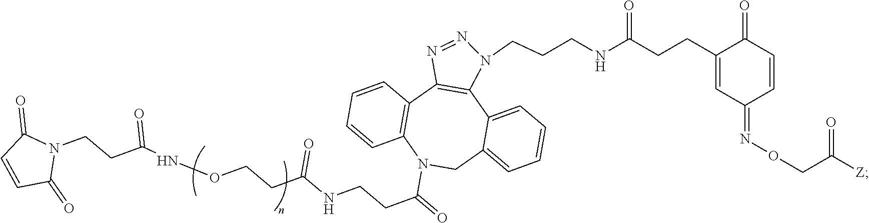
wherein Z is any one of the following:

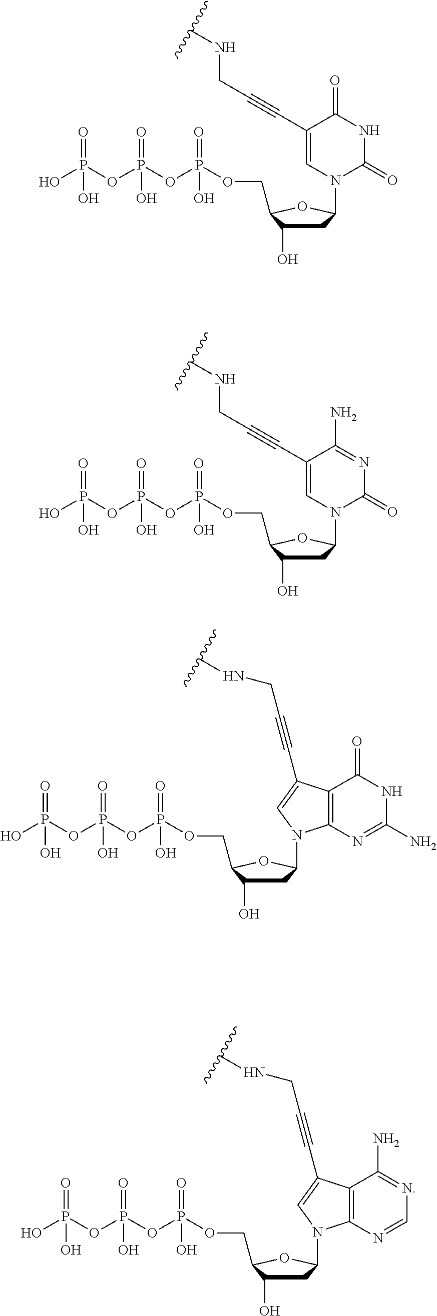

154. A molecule comprising the following structure: 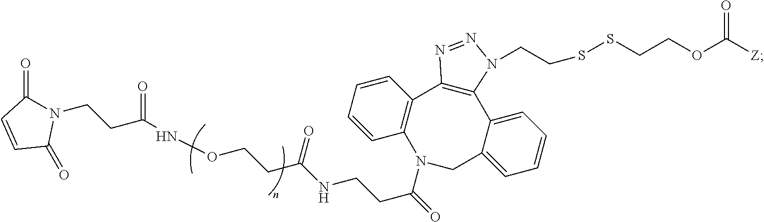
wherein Z is any one of the following:

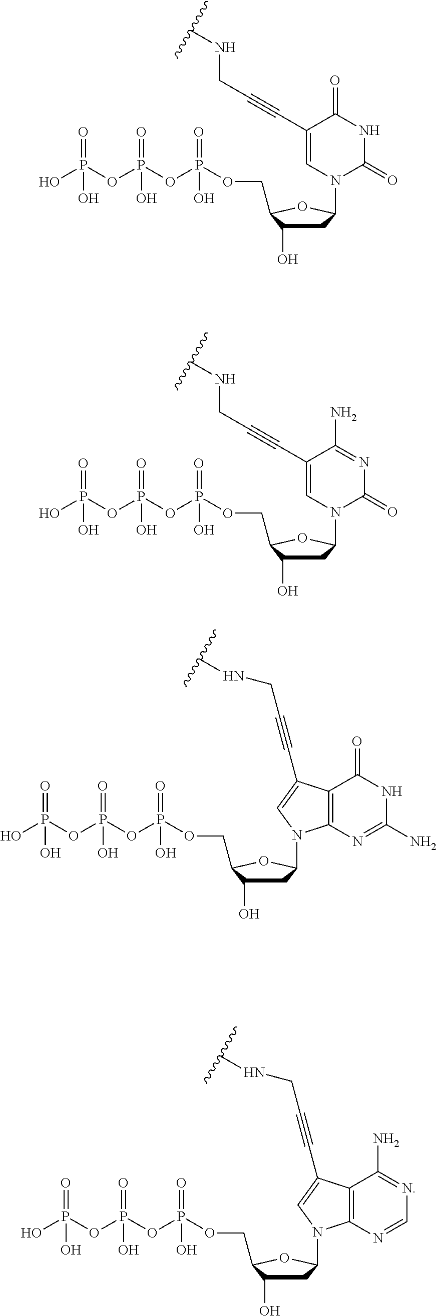

155. A molecule comprising the following structure: 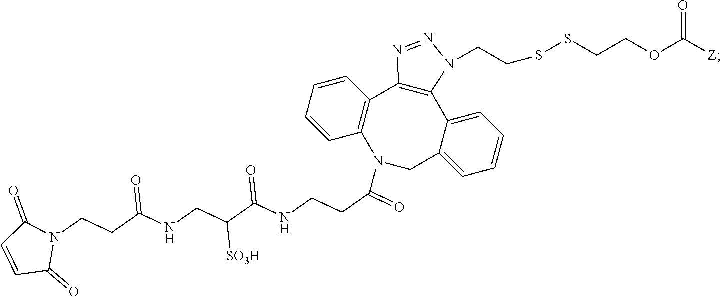
wherein Z is any one of the following:

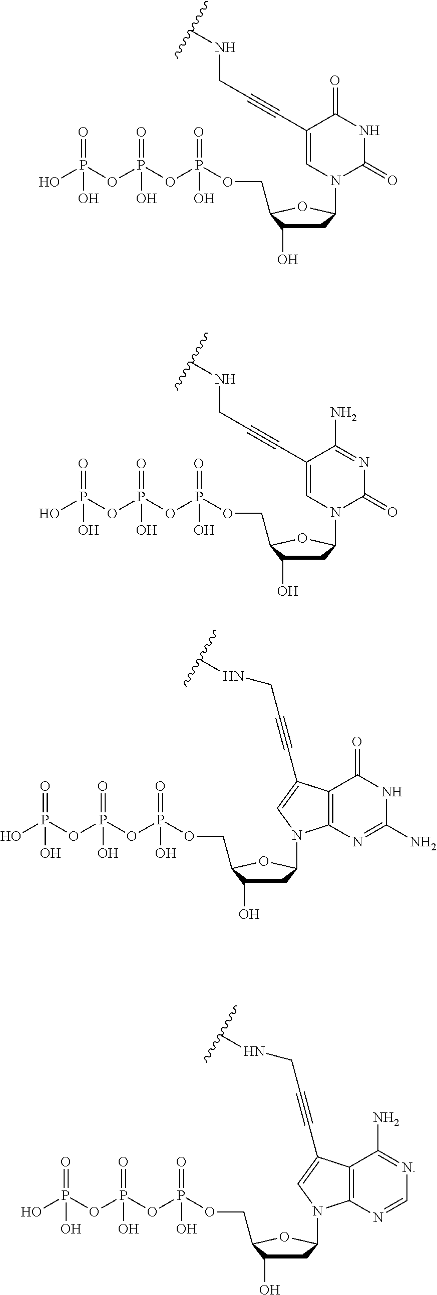

156. A molecule comprising the following structure: 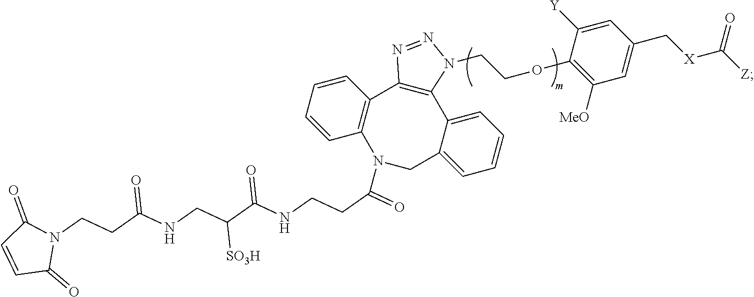
wherein X is O or NH;
wherein Y is H or OMe; and
wherein Z is any one of:

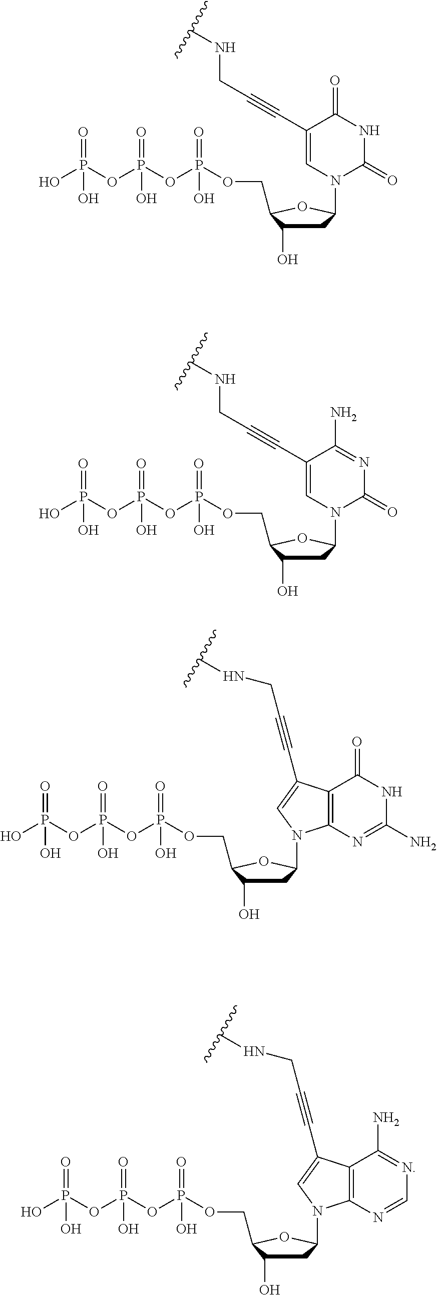

157. A molecule comprising the following structure: 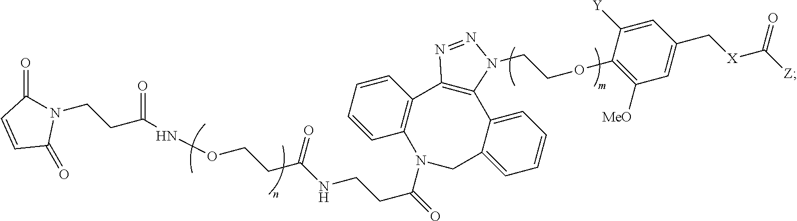
wherein X is O or NH;
wherein Y is H or OMe; and
wherein Z is any one of:

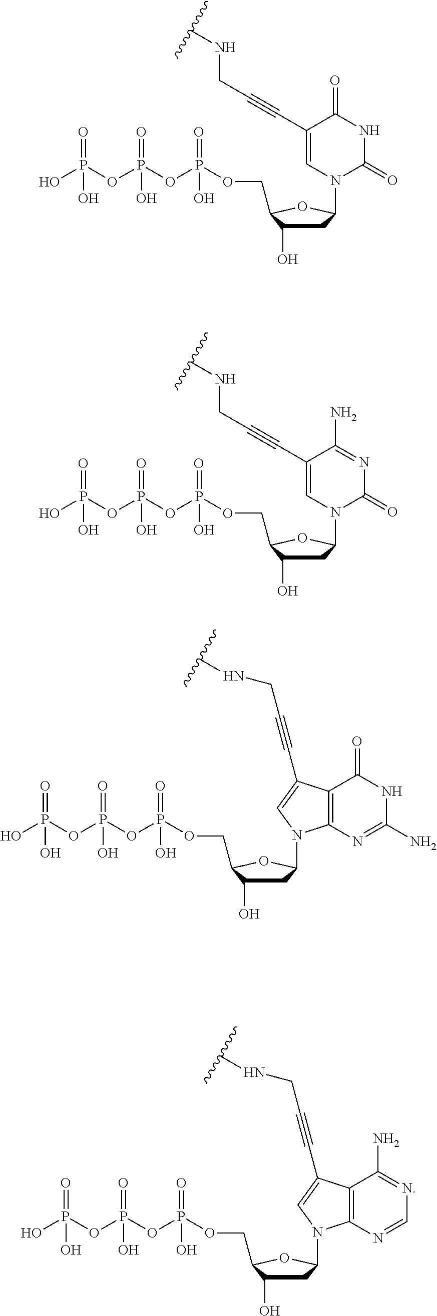

158. A molecule comprising the following structure: 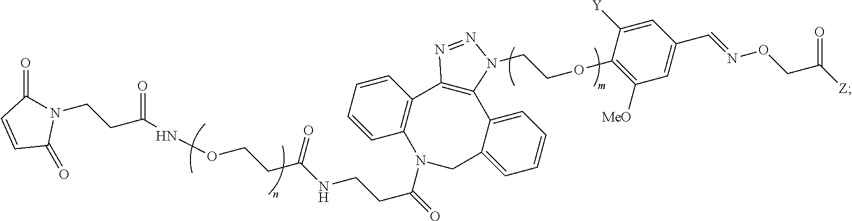
wherein Y is H or OMe; and
wherein Z is any one of:

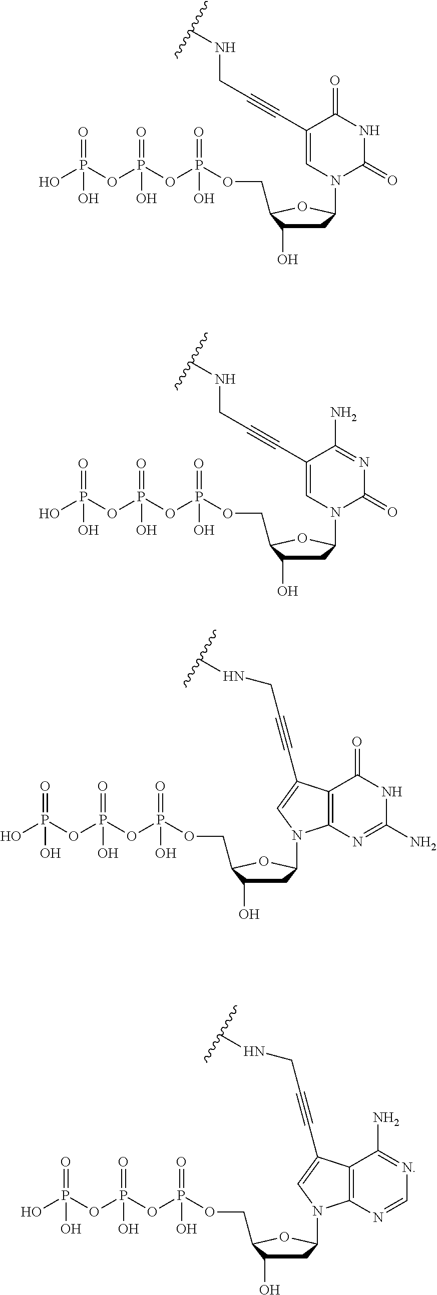

159. A molecule covalently linked to a tether, comprising the following structure: 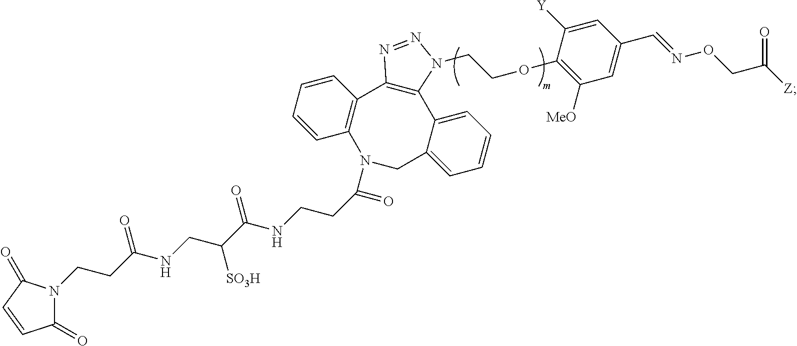
wherein Y is H or OMe; and
wherein Z is any one of:

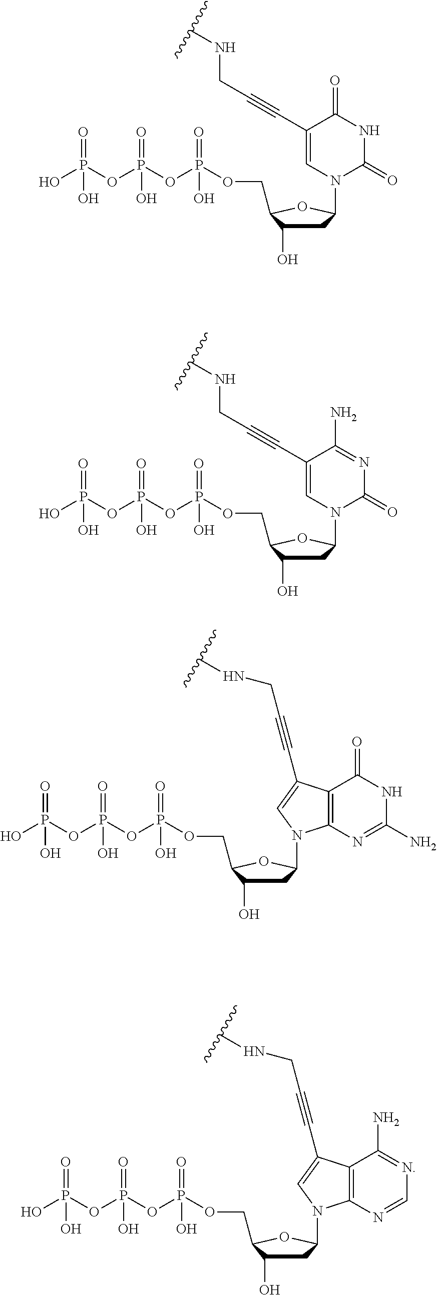

160. A molecule comprising any one of the following structures:

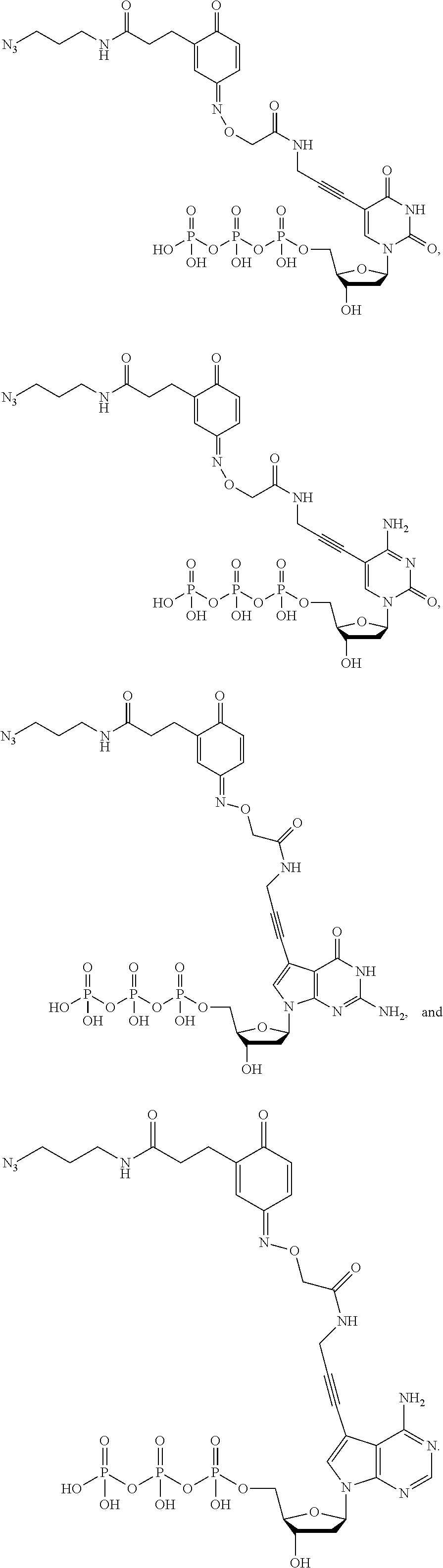

161. A molecule comprising any one of the following structures: 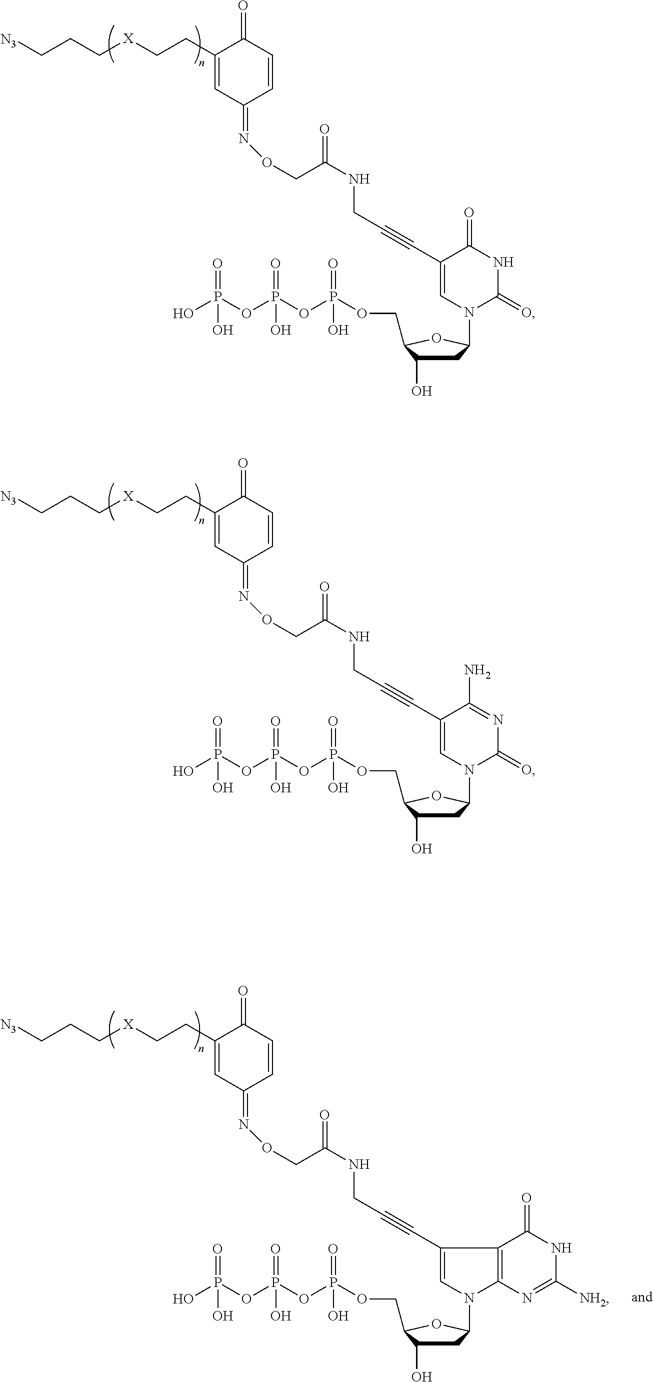 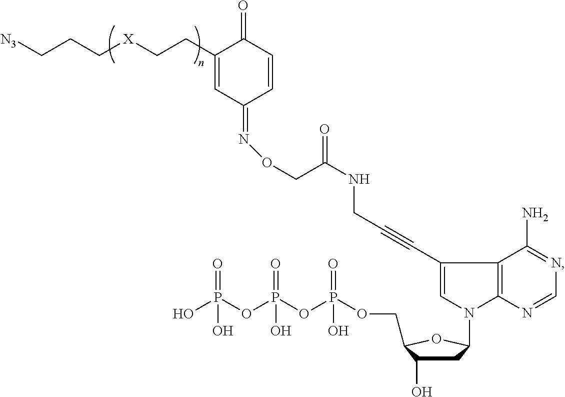
wherein X is C(O), C(O)NH, CH2, or O; and wherein n=1 for X═C(O), n=1 for C(O)NH, n=1 for CH2, and n=1-4 for X═O, wherein n is an integer.

162. A molecule comprising any one of the following structures:

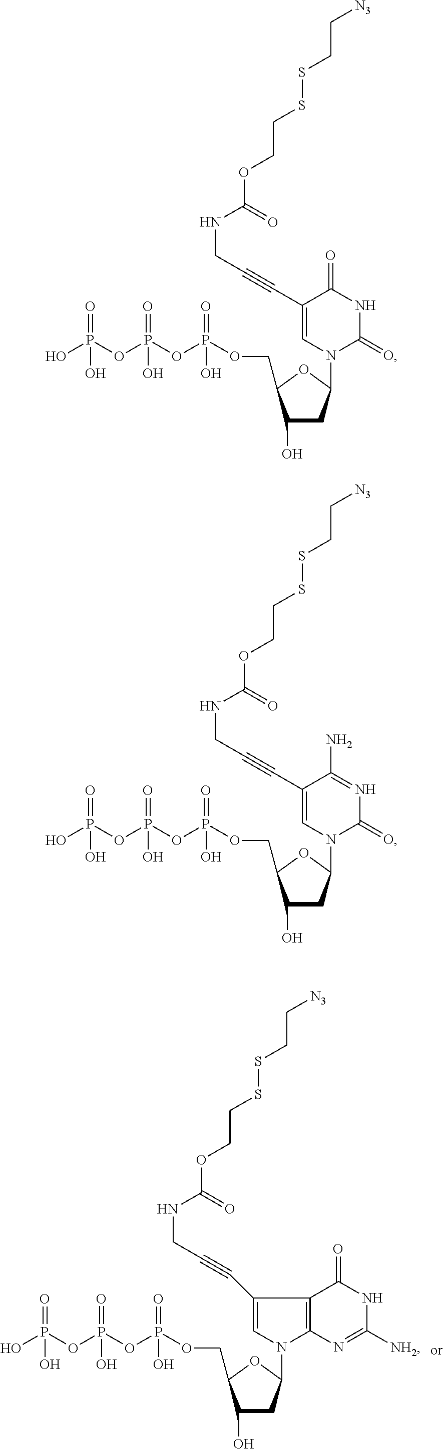 [SMILES: [N-]=[N+]=NCCSSCCOC(=O)NCC#Cc1cn([C@H]2C[C@@H](O)[C@@H](COP(=O)(O)OP(=O)(O)OP(=O)(O)O)O2)c(=O)[nH]c1=O ; [N-]=[N+]=NCCSSCCOC(=O)NCC#Cc1cn([C@H]2C[C@@H](O)[C@@H](COP(=O)(O)OP(=O)(O)OP(=O)(O)O)O2)c(=O)nc1N ; [N-]=[N+]=NCCSSCCOC(=O)NCC#Cc1cn([C@H]2C[C@@H](O)[C@@H](COP(=O)(O)OP(=O)(O)OP(=O)(O)O)O2)c2nc(N)[nH]c(=O)c12]

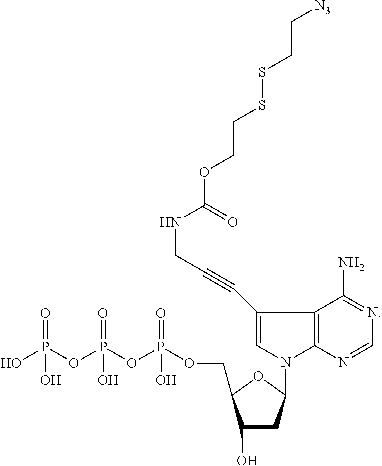 [SMILES: [N-]=[N+]=NCCSSCCOC(=O)NCC#Cc1cn([C@H]2C[C@@H](O)[C@@H](COP(=O)(O)OP(=O)(O)OP(=O)(O)O)O2)c2ncnc(N)c12]

163. A molecule comprising any one of the following structures: 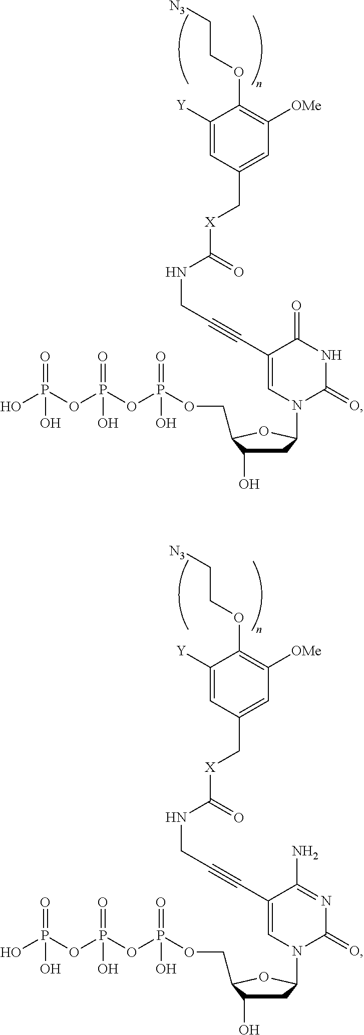 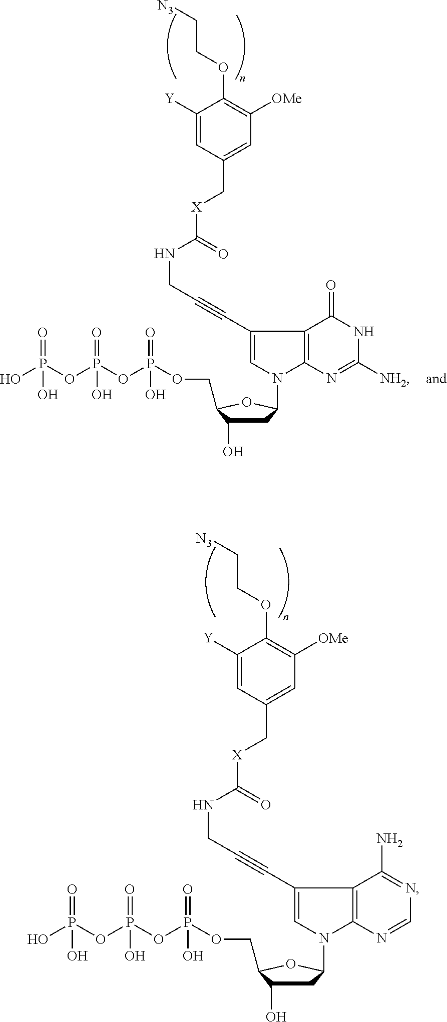
wherein X is O or NH, and wherein Y is H or OMe.

164. A molecule comprising any one of the following structures:

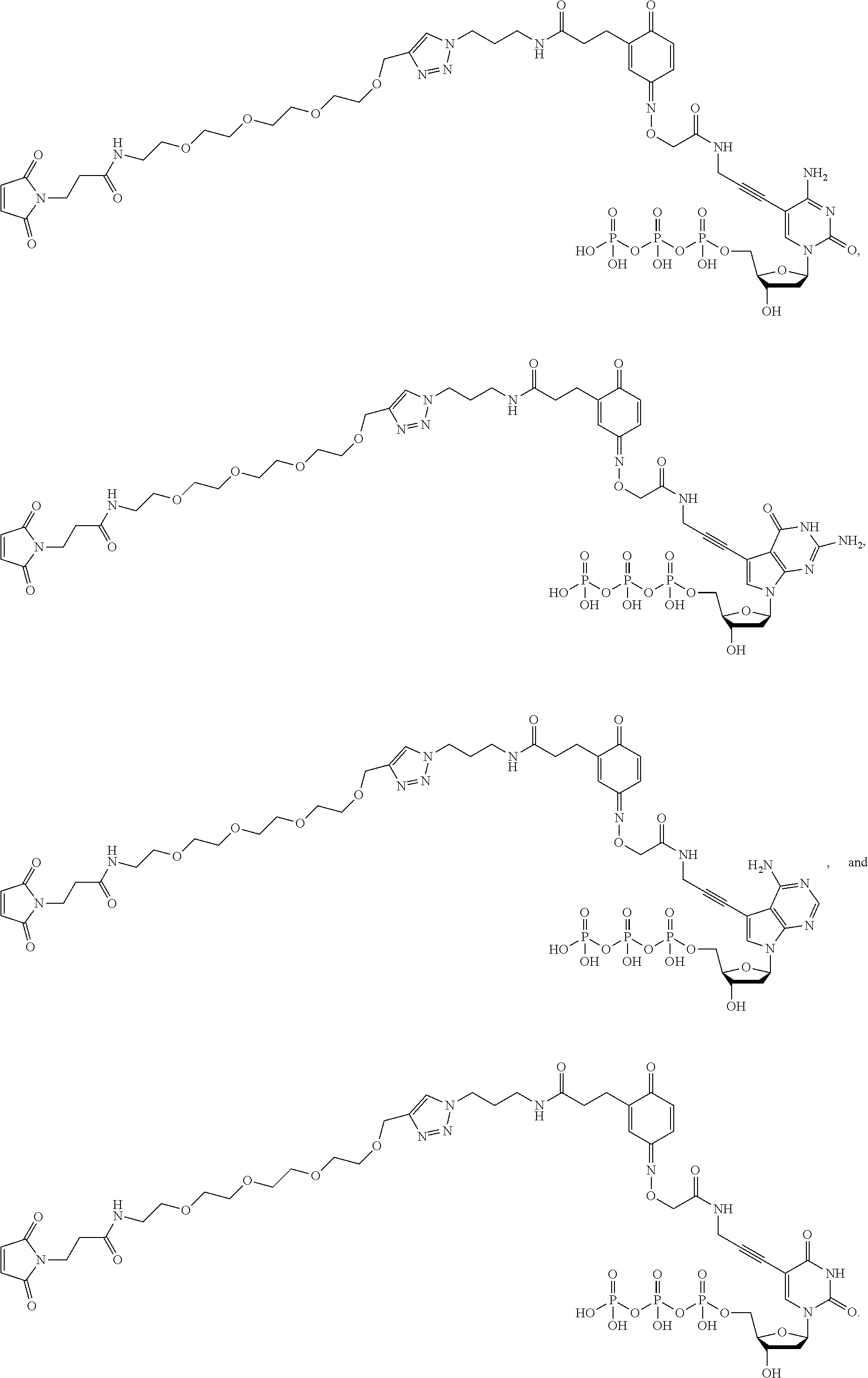

165. A molecule comprising any one of the following structures:

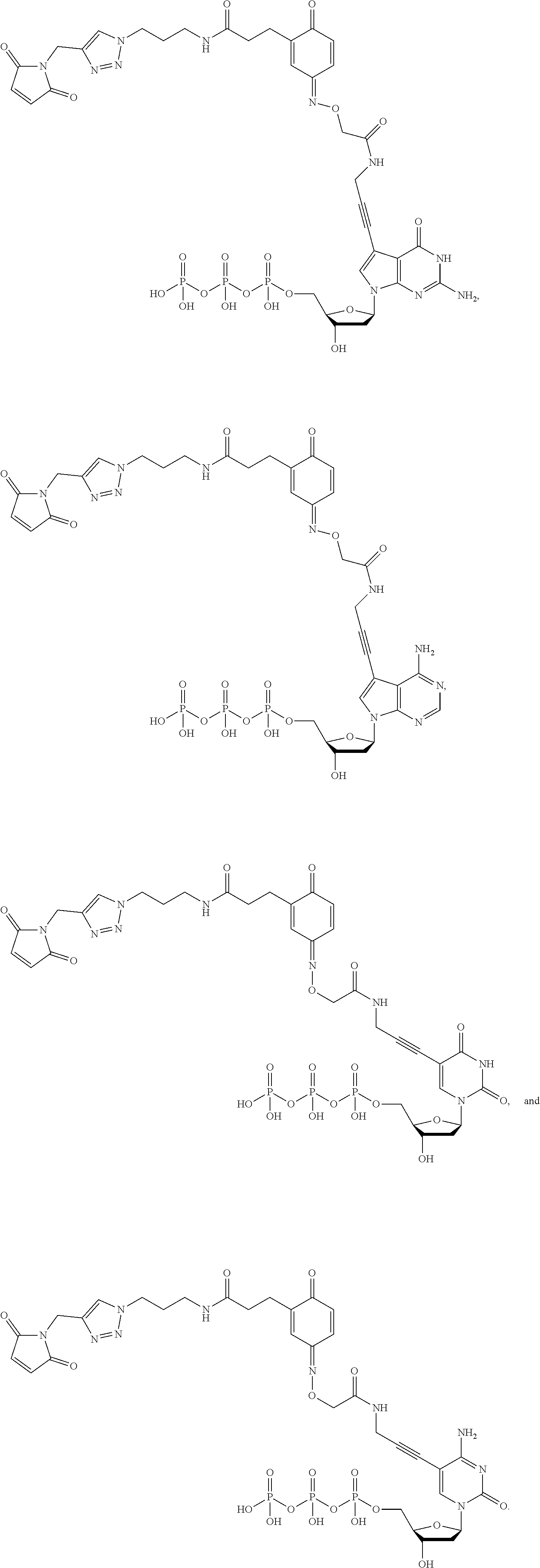

166. A molecule comprising any one of the following structures: 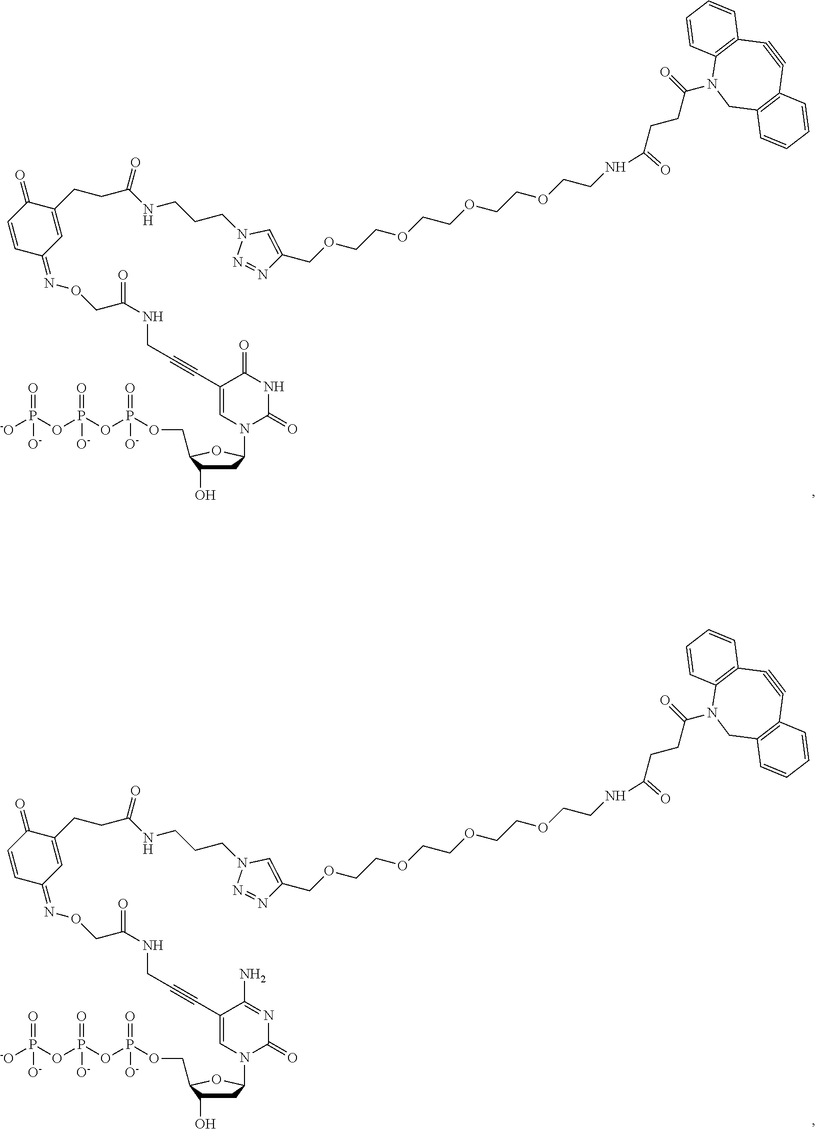 [chemical structure]
